(19) United States
(12) Patent Application Publication
(10) Pub. No.: US 2024/0115636 A1
(43) Pub. Date: Apr. 11, 2024

(54) ENCAPSULATED RNA POLYNUCLEOTIDES AND METHODS OF USE

(71) Applicant: ElevateBio Technologies, Inc., Waltham, MA (US)

(72) Inventors: Lorena LERNER, Andover, MA (US); Edward M. KENNEDY, Andover, MA (US); Christophe QUÉVA, Andover, MA (US); Jessica DETERLING, Andover, MA (US); Jeffrey David BRYANT, Cambridge, MA (US); Qi-Ying HU, Needham, MA (US); Tooba A. CHEEMA, Andover, MA (US); Sean ESSEX, Andover, MA (US)

(21) Appl. No.: 18/260,375

(22) PCT Filed: Jan. 6, 2022
(86) PCT No.: PCT/US2022/011450

§ 371 Date: Jul. 5, 2023

Related U.S. Application Data
(60) Provisional application No. 63/181,663, filed on Apr. 29, 2021.
(60) Provisional application No. 63/181,899, filed on Apr. 29, 2021.
(60) Provisional application No. 63/147,959, filed on Feb. 10, 2021.
(60) Provisional application No. 63/134,376, filed on Jan. 6, 2021.
(60) Provisional application No. 63/181,917, filed on Apr. 29, 2021.

(51) Int. Cl. A61K 35/768; A61K 9/127; A61P 35/00

(52) U.S. Cl. CPC A61K 35/768; A61K 9/1271; A61P 35/00; C12N 2770/32321; C12N 2770/32332; C12N 2770/32351

(57) ABSTRACT

The present disclosure relates to recombinant RNA molecules encoding an oncolytic virus genome. The present disclosure further relates to the encapsulation of the recombinant RNA molecules and the use of the recombinant RNA molecules and/or particles for the treatment and prevention of cancer.

### CROSS-REFERENCE TO RELATED APPLICATIONS

This application is a U.S. National Phase under 35 U.S.C. § 371 of International Application No. PCT/US2022/011450, filed on Jan. 6, 2022, which claims the benefit of U.S. Provisional Application No. 63/134,376, filed on Jan. 6, 2021, U.S. Provisional Application No. 63/147,959, filed on Feb. 10, 2021, U.S. Provisional Application No. 63/181,899, filed on Apr. 29, 2021, U.S. Provisional Application No. 63/181,917, filed on Apr. 29, 2021, and U.S. Provisional Application No. 63/181,663, filed on Apr. 29, 2021, the contents of each of which are herein incorporated by reference in their entireties.

### INCORPORATION OF THE TEXT FILE SUBMITTED ELECTRONICALLY

The contents of the text file submitted electronically herewith are incorporated herein by reference in their entirety. A computer readable format copy of the Sequence Listing (filename: ONCR_023_03US_SeqList_ST25.txt, date created: Jun. 29, 2023, file size: 536,833 bytes).

### FIELD

The present disclosure generally relates to the fields of immunology, inflammation, and cancer therapeutics. More specifically, the present disclosure relates to oncolytic virus strains, design of recombinant DNA molecules for viral genome expression, and particle-encapsulated viral genomes. The disclosure further relates to the treatment and prevention of proliferative disorders such as cancer.

### BACKGROUND

Oncolytic viruses are replication-competent viruses with lytic life-cycle able to infect and lyse tumor cells. Direct tumor cell lysis results not only in cell death, but also the generation of an innate and adaptive immune response against tumor antigens taken up and presented by local antigen presenting cells. Therefore, oncolytic viruses combat tumor cell growth through both direct cell lysis and by promoting antigen-specific adaptive responses capable of maintaining anti-tumor responses after viral clearance.

However, clinical use of replication-competent viruses poses several challenges. In general, viral exposure activates innate immune pathways, resulting in a broad, non-specific inflammatory response. If the patient has not been previously exposed to the virus, this initial innate immune response can lead to the development of an adaptive anti-viral response and the production of neutralizing antibodies. If a patient has been previously exposed to the virus, existing neutralizing anti-viral antibodies can prevent the desired lytic effects. In both instances, the presence of neutralizing antibodies not only prevents viral lysis of target cells, but also renders re-administration of the viral therapeutic ineffective. These factors limit the use of viral therapeutics in the treatment of metastatic cancers, as the efficacy of repeated systemic administration required for treatment of such cancers is hampered by naturally occurring anti-viral responses. Even in the absence of such obstacles, subsequent viral replication in non-diseased cells can result in substantial off-disease collateral damage to surrounding cells and tissues. In addition, different strains of oncolytic virus typically display significantly different tropism and potency in killing cancer cells.

There remains a long-felt and unmet need in the art for compositions and methods related to therapeutic use of replication-competent virus, engineering of virus and corresponding expression template, and selection of appropriate virus strains for different therapeutic indications. The present disclosure provides such compositions and methods, and more.

### SUMMARY

In one aspect, the present disclosure provides a lipid nanoparticle (LNP) comprising a synthetic RNA viral genome encoding an oncolytic Coxsackievirus virus, wherein the Coxsackievirus is a CVA21 strain selected from the EF strain and the KY strain. In some embodiments, the Coxsackievirus is the CVA21-KY strain, and wherein the CVA21-KY strain comprises a polynucleotide sequence having at least 80%, at least 85%, at least 90%, at least 95%, or 100% sequence identity to SEQ ID NO: 5. In some embodiments, the Coxsackievirus is the CVA21-EF strain, and wherein the CVA21-EF strain comprises a polynucleotide sequence having at least 80%, at least 85%, at least 90%, at least 95%, or 100% sequence identity to SEQ ID NO: 9. In some embodiments, the Coxsackievirus comprises a 5′ UTR (IRES) sequence having at least 80%, at least 85%, at least 90%, at least 95%, or 100% sequence identity to SEQ ID NO: 6 or 10. In some embodiments, the Coxsackievirus comprises a P1 sequence having at least 80%, at least 85%, at least 90%, at least 95%, or 100% sequence identity to SEQ ID NO: 7 or 11. In some embodiments, the Coxsackievirus comprises a 3D sequence having at least 80%, at least 85%, at least 90%, at least 95%, or 100% sequence identity to SEQ ID NO: 8 or 12. In some embodiments, the synthetic RNA viral genome does not comprise a polynucleotide sequence having more than 95%, more than 90%, more than 85%, or more than 80% sequence identity to SEQ ID NO: 1.

In one aspect, the present disclosure provides a lipid nanoparticle (LNP) comprising a synthetic RNA viral genome encoding an oncolytic Coxsackievirus virus, wherein the Coxsackievirus is a CVA21 Kuykendall strain.

In one aspect, the present disclosure provides a lipid nanoparticle (LNP) comprising a synthetic RNA viral genome encoding an oncolytic Seneca Valley Virus (SVV), wherein the synthetic RNA viral genome comprises a polynucleotide sequence having at least 80%, at least 85%, at least 90%, at least 95%, or 100% sequence identity to SEQ ID NO: 68. In some embodiments, the synthetic RNA viral genome comprises a 5′ UTR (IRES) sequence having at least 80%, at least 85%, at least 90%, at least 95%, or 100% sequence identity to nucleic acids 1-670 of SEQ ID NO: 68. In some embodiments, the synthetic RNA viral genome encodes a SVV VP2 protein comprising a S177A mutation.

In some embodiments, delivery of the LNP to a cell results in production of viral particles by the cell, and wherein the viral particles are infectious and lytic. In some embodiments, the synthetic RNA viral genome further comprises a heterologous polynucleotide encoding an exogenous payload protein. In some embodiments, the LNP further comprises a second recombinant RNA molecule encoding an exogenous payload protein.

In some embodiments, the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2). In some embodiments, the nitroreductase is NfsB or NfsA. In some embodiments, the reovirus FAST protein is ARV p14, BRV p15, or a p14-p15 hybrid. In some embodiments, the exogenous payload protein is a fluorescent protein, an enzymatic protein, a cytokine, a chemokine, an antigen-binding molecule capable of binding to a cell surface receptor, or a ligand for a cell-surface receptor. In some embodiments, the cytokine is selected from GM-CSF, IFNγ, IL-2, IL-7, IL-12, IL-18, IL-21, and IL-36γ. In some embodiments, the ligand for a cell-surface receptor is Flt3 ligand or TNFSF14. In some embodiments, the chemokine is selected from CXCL10, CCL4, CCL21, and CCL5. In some embodiments, the antigen-binding molecule is capable of binding to and inhibiting an immune checkpoint receptor. In some embodiments, the immune checkpoint receptor is PD-1. In some embodiments, the antigen-binding molecule is capable of binding to a tumor antigen. In some embodiments, the antigen binding molecule is a bispecific T cell engager molecule (BiTE) or a bispecific light T cell engager molecule (LiTE). In some embodiments, the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1. In some embodiments, the tumor antigen is DLL3 or EpCAM.

In some embodiments, the synthetic RNA viral genome and/or the recombinant RNA molecule comprises a microRNA (miRNA) target sequence (miR-TS) cassette, wherein the miR-TS cassette comprises one or more miRNA target sequences. In some embodiments, the one or more miRNAs are selected from miR-124, miR-1, miR-143, miR-128, miR-219, miR-219a, miR-122, miR-204, miR-217, miR-137, miR-142, and miR-126. In some embodiments, the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

In some embodiments, the LNP comprises a cationic lipid, a helper lipid, a structural lipid, and a PEG-lipid. In some embodiments, the cationic lipid is a compound of Formula (I):

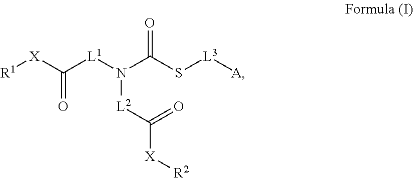

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

In some embodiments, the number of carbon atoms between the S of the thiolate and the closest N comprised in A is 2-4. In some embodiments, the cationic lipid is a compound of Formula (I-a):

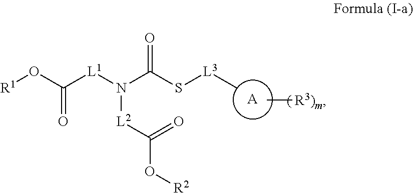

or a pharmaceutically acceptable salt or solvate thereof, wherein:
m is 0, 1, 2, 3, 4, 5, or 6.

In some embodiments, A is an optionally substituted 5-6-membered heterocyclyl ring. In some embodiments, the cationic lipid is

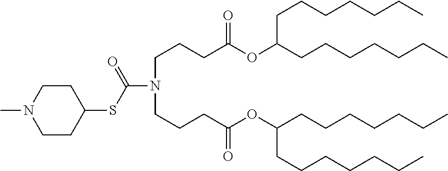 [SMILES: CCCCCCCC(CCCCCCC)OC(=O)CCCN(CCCC(=O)OC(CCCCCCC)CCCCCCC)C(=O)SC1CCN(C)CC1]

or a pharmaceutically acceptable salt or solvate thereof.

In some embodiments, the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

In some embodiments, the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE).

In some embodiments, the cationic lipid is 1,2-dioleoyl-3-trimethylammonium-propane (DOTAP), and wherein the helper lipid is 1,2-Dilauroyl-sn-glycero-3-phosphoethanolamine (DLPE) or 1,2-Dioleoyl-sn-glycero-3-phosphoethanolamine (DOPE).

In some embodiments, the structural lipid is cholesterol.

In some embodiments, the PEG-lipid is a compound of Formula (A″):

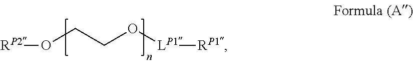

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃,
and wherein the LNP has a molar ratio of about 0.001% to about 5% PEG-lipid.

In some embodiments, L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, wherein L^{P1}″ is a bond. In some embodiments, R^{P2}″ is hydrogen. In some embodiments, the PEG-lipid is a compound of Formula (B):

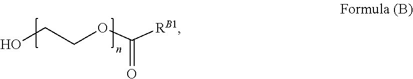

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

In some embodiments, the PEG-lipid is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

In some embodiments, the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

In some embodiments, the cationic lipid comprises COATSOME® SS—OC, wherein the helper lipid comprises DSPC, the structural lipid comprises cholesterol (Chol) and wherein the PEG-lipid comprises DPG-PEG2000.

In some embodiments, the cationic lipid comprises COATSOME® SS—OC, wherein the helper lipid comprises DSPC, the structural lipid comprises cholesterol (Chol) and wherein the PEG-lipid is a compound of Formula (A″):

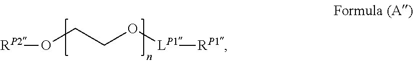

or a pharmaceutically acceptable salt thereof, wherein:
n is an integer between 10 to 200, inclusive of all endpoints;
L^{P1}″ is a bond;
R^{P1}″ is C₅-C₂₅ alkyl or C₅-C₂₅ alkenyl; and
R^{P2}″ is hydrogen, and
wherein the LNP has a molar ratio of about 0.001% to about 5% PEG-lipid.

In some embodiments, the PEG-lipid is selected from the group consisting of BRIJ™ S100, BRIJ™ S20, BRIJ™ 020 and BRIJ™ C₂₀. In some embodiments the PEG-lipid is BRIJ™ S100.

In some embodiments, the ratio of SS—OC:DSPC:Chol:PEG-lipid (as a percentage of total lipid content) is A:B:C:D, wherein A+B+C+D=100%, and wherein
e. A=40%-60%, B=10%-25%, C=20%-30%, and D=0.01%-3%;
f. A=45%-50%, B=20%-25%, C=25%-30%, and D=0.01%-1%; or
g. A=about 49%, B=about 22%, C=about 28%, and D=about 0.5%

In some embodiments, the ratio of SS—OC:DSPC:Chol:PEG-lipid (as a percentage of total lipid content) is A:B:C:D, wherein A+B+C+D=100%, and wherein
h. A=40%-60%, B=10%-30%, C=20%-45%, and D=0%-3%;
i. A=40%-60%, B=10%-30%, C=25%-45%, and D=0.01%-3%;
j. A=45%-55%, B=10%-20%, C=30%-40%, and D=1%-2%;
k. A=45%-50%, B=10%-15%, C=35%-40%, and D=1%-2%; or
l. A=about 49%, B=about 11%, C=about 38%, and D=about 1.5%.

In some embodiments, the ratio of SS—OC:DSPC:Chol:PEG-lipid (as a percentage of total lipid content) is about A:B:C:D, wherein A+B+C+D=100%, and wherein
m. A=45%-65%, B=5%-20%, C=20%-45%, and D=0%-3%;
n. A=50%-60%, B=5%-15%, C=30%-45%, and D=0.01%-3%;
o. A=55%-60%, B=5%-15%, C=30%-40%, and D=1%-2%;
p. A=55%-60%, B=5%-10%, C=30%-35%, and D=1%-2%; or
q. A=about 58%, B=about 7%, C=about 33%, and D=about 1.5%.

In one aspect, the disclosure provides a lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (A″):

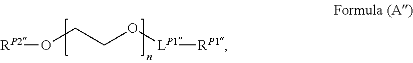

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

In one aspect, the disclosure provides a lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (A″):

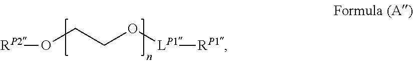

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

In some embodiments, R¹ is C₁₆-C₁₈ alkyl or C₁₆-C₁₈ alkenyl. In some embodiments, L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—. In some embodiments, L^{P1}″ is a bond. In some embodiments, R^{P2}″ is hydrogen. In some embodiments, the PEG-lipid is a compound of Formula (A″-f1), Formula (A″-f2), or Formula (A″-f3):

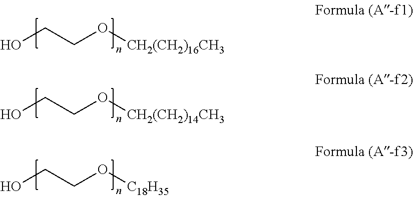

or a pharmaceutically acceptable salt thereof.

In one aspect, the disclosure provides a lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (B):

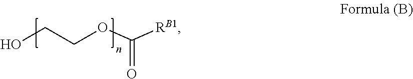

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
   wherein the LNP has a molar ratio of about 0.001% to about 5% PEG-lipid.

In one aspect, the disclosure provides a lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (B):

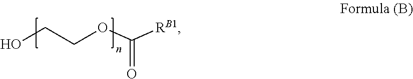

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
   wherein the LNP has a molar ratio of about 0.001% to about 5% PEG-lipid. In some embodiments, R¹ is C₁₅-C₁₇ alkyl or C₁₅-C₁₇ alkenyl.

In some embodiments, the PEG-lipid is a compound of Formula (B-a) or Formula

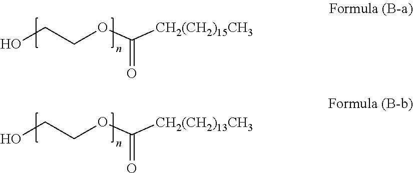

or a pharmaceutically acceptable salt thereof.

In some embodiments, n is on average about 20, about 40, about 50, or about 100. In some embodiments, n is on average about 100. In some embodiments, the PEG-lipid comprise a PEG moiety having an average molecular weight of about 200 daltons to about 10,000 daltons, about 500 daltons to about 7,000 daltons, or about 800 daltons to about 6,000 daltons.

In some embodiments, the PEG-lipid is selected from the group consisting of HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃.

In some embodiments, the LNP induces a reduced immune response in vivo as compared to a control LNP lacking the PEG-lipid of Formula (A″) and/or a ionizable lipid of Formula (I), optionally wherein a PEG-lipid in the control LNP is PEG2K-DPG or PEG2K-DMG. In some embodiments, the immune response is accelerated blood clearance (ABC) of the LNP and/or an anti-PEG IgM response.

In some embodiments, the cationic lipid is a compound of Formula (I):

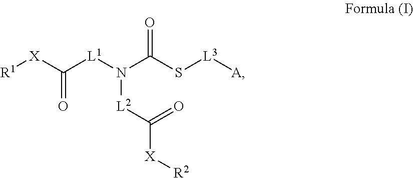

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

In some embodiments, the number of carbon atoms between the S of the thiolate and the closest N comprised in A is 2-4.

In some embodiments, the cationic lipid is a compound of Formula (I-a):

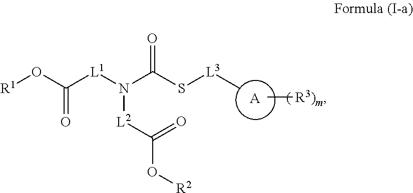

or a pharmaceutically acceptable salt or solvate thereof, wherein:
m is 0, 1, 2, 3, 4, 5, or 6.

In some embodiments, wherein A is an optionally substituted 5-6-membered heterocyclyl ring.

In some embodiments, the cationic lipid is

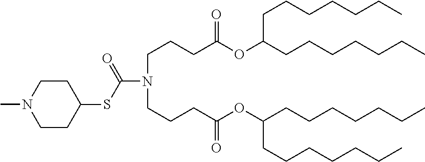 [SMILES: CCCCCCCC(CCCCCCC)OC(=O)CCCN(CCCC(=O)OC(CCCCCCC)CCCCCCC)C(=O)SC1CCN(C)CC1]

or a pharmaceutically acceptable salt or solvate thereof.

In some embodiments, the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME@SS—OP, Di((Z)-non-2-en-1-yl)9-((4-dimethylamino)butanoyl)oxy)heptadecanedioate (L-319), N-(2,3-dioleoyloxy)propyl)-N,N,N-trimethylammonium chloride (DOTAP), or a mixture thereof.

In some embodiments, the cationic lipid is a compound of Formula (TT-1a):

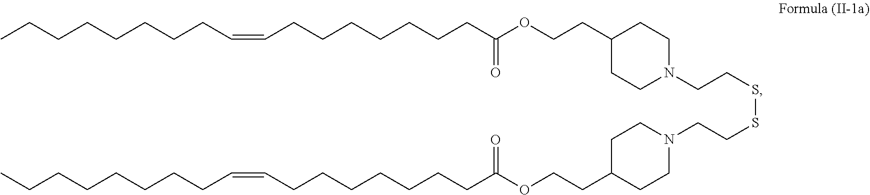 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

or a compound of Formula (II-2a):

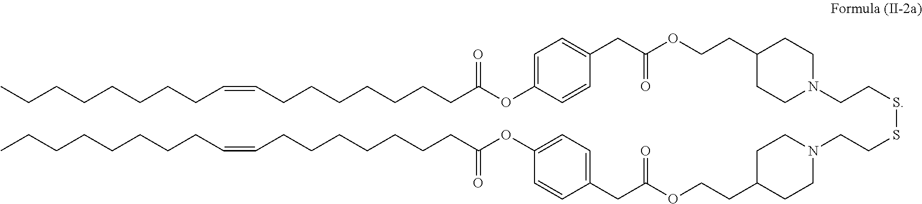 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In some embodiments, the cationic lipid is a compound of Formula (II-1a), the structural lipid is cholesterol, the helper lipid is DSPC, and the PEG-lipid is BRIJ™ S100.

In some embodiments, the cationic lipid is a compound of Formula (II-1a), the structural lipid is cholesterol, the helper lipid is DSPC, and the PEG-lipid is MYRJ™ S100, MYRJ™ S50, or MYRJ™ S40.

In some embodiments, the LNP comprises a molar ratio of about 0.1% to about 2% PEG-lipid, such as about 0.2% to about 0.8 mol %, about 0.4% to about 0.6 mol %, about 0.7% to about 1.3%, or about 1.2% to about 1.8% PEG-lipid. In some embodiments, the LNP comprises a molar ratio of about 0.2% to about 0.8%, or about 0.5% PEG-lipid. In some embodiments, n the LNP comprises a molar ratio of about 1.2% to about 1.8%, or about 1.5% PEG-lipid. In some embodiments, the LNP has a molar ratio of about 44% to about 54% cationic lipid, about 19% to about 25% helper lipid, about 24% to about 33% structural lipid, and about 0.2% to about 0.8% PEG-lipid.

In some embodiments, the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC PEG-lipid is 49:28.5:22:0.5.

In some embodiments, the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC PEG-lipid is 49:27.5:22:1.5.

In some embodiments, the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC PEG-lipid is 49:38.5:11:1.5.

In some embodiments, the LNP has a lipid-nitrogen-to-phosphate (N:P) ratio of about 1 to about 25. In some embodiments, the LNP has a N:P ratio of about 14. In some embodiments, hyaluronan is conjugated to the surface of the LNP.

In one aspect, the disclosure provides a pharmaceutical composition comprising a plurality of lipid nanoparticles of the disclosure. In some embodiments, the plurality of LNPs have an average diameter of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average diameter of about 50 nm to about 120 nm. In some embodiments, the plurality of LNPs have an average diameter of about 50 nm, 60 nm, 70 nm, 80 nm, 90 nm, 100 nm, 110 nm, or about 120 nm. In some embodiments, the plurality of LNPs have an average diameter of about 100 nm.

In some embodiments, the plurality of LNPs have an average zeta-potential of between about 40 mV to about −40 mV, about 20 mV to about −20 mV, about 10 mV to about −10 mV, about 5 mV to about −5 mV, or about 20 mV to about −40 mV. In some embodiments, the plurality of LNPs have an average zeta-potential of less than about 5 mV, less than about 0 mV, less than about −5 mV, less than about −10 mV, less than about −20 mV, less than about −30 mV, less than about −35 mV, or less than about −40 mV. In some embodiments, the plurality of LNPs have an average zeta-potential of between about −50 mV to about −20 mV, about −40 mV to about −20 mV, about −30 mV to about −10 mV, about −20 mV to about 0 mV, about −15 mV to about 5 mV, or about −10 mV to about 10 mV. In some embodiments, the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In some embodiments, administering the pharmaceutical composition to a subject delivers the recombinant RNA polynucleotide to a target cell of the subject, and wherein the recombinant RNA polynucleotide produces an infectious oncolytic virus capable of lysing the target cell of the subject. In some embodiments, the target cell is a cancerous cell. In some embodiments, the composition is formulated for intravenous and/or intratumoral delivery.

In some embodiments, the composition has a duration of therapeutic effect in vivo greater than that of a composition lacking the PEG-lipid of Formula (A″) and/or a ionizable lipid of Formula (I). In some embodiments, the composition has a duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer. the composition has a half-life and/or an AUC in vivo greater than or equal to that of a pre-determined threshold value.

In some embodiments, the encapsulation efficiency of the synthetic RNA viral genome by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, the composition has a total lipid concentration of about 10 mM, about 20 mM, about 30 mM, about 40 mM, or about 50 mM. In some embodiments, the composition is formulated at a pH of about 2.5, about 3, about 3.5, about 4, about 4.5, about 5, about 5.5, or about 6.

In some embodiments, the composition is formulated for multiple administrations. In some embodiments, a subsequent administration is administered at least 3 days, at least 5 days, at least 7 days, at least 9 days, at least 11 days, at least 14 days, or at least 21 days after a first administration. In some embodiments, the composition further comprises a pharmaceutically acceptable carrier.

In one aspect, the disclosure provides a recombinant RNA molecule comprising a synthetic RNA viral genome encoding an oncolytic Coxsackievirus virus, wherein the Coxsackievirus is a CVA21 strain selected from the EF strain and the KY strain. In some embodiments, the Coxsackievirus is the CVA21-KY strain, and wherein the CVA21-KY strain comprises a polynucleotide sequence having at least 80%, at least 85%, at least 90%, at least 95%, or 100% sequence identity according to SEQ ID NO: 5. In some embodiments, the Coxsackievirus is the CVA21-EF strain, and wherein the CVA21-EF strain comprises a polynucleotide sequence having at least 80%, at least 85%, at least 90%, at least 95%, or 100% sequence identity according to SEQ ID NO: 9. In some embodiments, the Coxsackievirus comprises a 5′ UTR (IRES) sequence having at least 80%, at least 85%, at least 90%, at least 95%, or 100% sequence identity according to SEQ ID NO: 6 or 10. In some embodiments, the Coxsackievirus comprises a P1 sequence having at least 80%, at least 85%, at least 90%, at least 95%, or 100% sequence identity according to SEQ ID NO: 7 or 11. In some embodiments, the Coxsackievirus comprises a 3D sequence having at least 80%, at least 85%, at least 90%, at least 95%, or 100% sequence identity according to SEQ ID NO: 8 or 12. In some embodiments, the synthetic RNA viral genome does not comprise a polynucleotide sequence having more than 95%, more than 90%, more than 85%, or more than 80% sequence identity according to SEQ ID NO: 1. In some embodiments, the recombinant RNA molecule does not comprise an RNA viral genome having 100% sequence identity to that of a wildtype Coxsackievirus virus.

In one aspect, the disclosure provides a recombinant RNA molecule comprising a synthetic RNA viral genome encoding an oncolytic Coxsackievirus virus, wherein the Coxsackievirus is a CVA21 Kuykendall strain.

In one aspect, the disclosure provides a recombinant RNA molecule comprising a synthetic RNA viral genome encoding a Seneca Valley virus (SVV), wherein the SVV comprises is a chimeric SVV, and wherein the synthetic RNA viral genome comprises a polynucleotide sequence having at least 80%, at least 85%, at least 90%, at least 95%, or 100% sequence identity to SEQ ID NO: 68.

In some embodiments, the recombinant RNA molecule further comprises a microRNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell. In some embodiments, the one or more miRNAs are selected from miR-124, miR-1, miR-143, miR-128, miR-219, miR-219a, miR-122, miR-204, miR-217, miR-137, miR-142, and miR-126. In some embodiments, the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

In some embodiments, the recombinant RNA molecule is capable of producing a replication-competent oncolytic virus when introduced into a cell by a non-viral delivery vehicle. In some embodiments, the cell is a mammalian cell. In some embodiments, the cell is a mammalian cell present in a mammalian subject. In some embodiments, the one or more miR-TS cassettes is incorporated into the 5′ untranslated region (UTR) or 3′ UTR of one or more viral genes. In some embodiments, the one or more miR-TS cassettes is incorporated into the open reading frame (ORF), the 5′ untranslated region (UTR), or the 3′ UTR of one or more viral genes. In some embodiments, the recombinant RNA molecule is inserted into a nucleic acid vector. In some embodiments, the nucleic acid vector is a replicon.

In some embodiments, the synthetic RNA viral genome further comprises a heterologous polynucleotide encoding an exogenous payload protein. In some embodiments, the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2). In some embodiments, the nitroreductase is NfsB or NfsA. In some embodiments, the reovirus FAST protein is ARV p14, BRV p15, or a p14-p15 hybrid. In some embodiments, the exogenous payload protein is a fluorescent protein, an enzymatic protein, a cytokine, a chemokine, an antigen-binding molecule capable of binding to a cell surface receptor, or a ligand capable of binding to a cell surface receptor. In some embodiments, the cytokine is selected from GM-CSF, IFNγ, IL-2, IL-7, IL-12, IL-18, IL-21, and IL-36γ. In some embodiments, the ligand for a cell-surface receptor is Flt3 ligand or TNFSF14. In some embodiments, the chemokine is selected from CXCL10, CCL4, CCL21, and CCL5. In some embodiments, the antigen-binding molecule is capable of binding to and inhibiting an immune checkpoint receptor. In some embodiments, the immune checkpoint receptor is PD-1. In some embodiments, the antigen-binding molecule is capable of binding to a tumor antigen. In some embodiments, the antigen binding molecule is a bispecific T cell engager molecule (BiTE) or a bispecific light T cell engager molecule (LiTE). In some embodiments, the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1. In some embodiments, the tumor antigen is DLL3 or EpCAM.

In one aspect, the disclosure provides a recombinant DNA template comprising from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence.

In one aspect, the disclosure provides a recombinant DNA molecule comprising from 5′ to 3′, a promoter sequence, a 5′ junctional cleavage sequence, a polynucleotide sequence encoding an RNA molecule of the disclosure comprising a synthetic RNA viral genome, a poly-A tail, and a 3′ junctional cleavage sequence.

In some embodiments, The recombinant DNA molecule comprises a leader sequence between the promoter sequence and the 5′ junctional cleavage sequence.

In one aspect, the disclosure provides a recombinant DNA molecule comprising from 5′ to 3′, a promoter sequence, a leader sequence, a 5′ junctional cleavage sequence, a polynucleotide sequence encoding a recombinant RNA molecule comprising a synthetic RNA viral genome, a poly-A tail, and a 3′ junctional cleavage sequence.

In some embodiments, the leader sequence is less than 100 bp in length. In some embodiments, the promoter sequence is a T7 promoter sequence. In some embodiments, the poly-A tail is about 50-90 bp in length or about 65-75 bp in length. In some embodiments, the poly-A tail is about 70 bp in length. In some embodiments, the poly-A tail is about 10-50 bp, or 25-35 bp in length.

In some embodiments, the 5′ junctional cleavage sequence comprises or consists of a ribozyme sequence and the 3′ junctional cleavage sequence comprises or consists of a ribozyme sequence. In some embodiments, the 5′ ribozyme sequence is a hammerhead ribozyme sequence and wherein the 3′ ribozyme sequence is a hepatitis delta virus ribozyme sequence. In some embodiments, the 5′ junctional cleavage sequence comprises or consists of an RNAseH primer binding sequence and the 3′ junctional cleavage sequence comprises or consists of a restriction enzyme recognition sequence. In some embodiments, the 5′ junctional cleavage sequence comprises or consists of a ribozyme sequence and the 3′ junctional cleavage sequence comprises or consists of a restriction enzyme recognition sequence. In some embodiments, the 5′ ribozyme sequence comprises or consists of a hammerhead ribozyme sequence, a Pistol ribozyme sequence, or a modified Pistol ribozyme sequence.

In some embodiments, the 3′ junctional cleavage sequence comprises or consists of a Type IIS restriction enzyme recognition sequence.

In some embodiments, the RNA molecule encodes the RNA viral genome of a Coxsackievirus (CVA). In some embodiments, the Coxsackievirus is a CVA21 strain. In some embodiments, the leader sequence comprises or consists of a polynucleotide sequence having at least 70%, at least 80%, at least 90%, at least 95%, or 100% sequence identity according to SEQ ID NO: 14 or 15. In some embodiments, the 5′ junctional cleavage sequence comprises or consists of a Pistol ribozyme sequence having at least 80%, at least 90%, or 100% sequence identity to SEQ ID NO: 18, and wherein the P2 motif of the 5′ ribozyme sequence has the polynucleotide sequence of “TTTT”. In some embodiments, the 5′ junctional cleavage sequence comprises or consists of a Pistol ribozyme sequence having at least 80%, at least 90%, or 100% sequence identity to SEQ ID NO: 17, and wherein the P2 motif of the 5′ ribozyme sequence has the polynucleotide sequence of “TTTA”. In some embodiments, the 3′ junctional cleavage sequence comprises or consists of a BsmBI recognition sequence. In some embodiments, the 3′ junctional cleavage sequence comprises or consists of a BsaI recognition sequence. In some embodiments, the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsmBI recognition sequence. In some embodiments, the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsaI recognition sequence.

In some embodiments, the RNA molecule encodes the RNA viral genome of a Seneca Valley virus (SVV). In some embodiments, the leader sequence comprises or consists of a polynucleotide sequence having at least 70%, at least 80%, at least 90%, at least 95%, or 100% sequence identity according to any one of SEQ ID NO: 53-63. In some embodiments, the leader sequence comprises or consists of a polynucleotide sequence having at least 70%, at least 80%, at least 90%, at least 95%, or 100% sequence identity according to SEQ ID NO: 58. In some embodiments, the 5′ ribozyme sequence is a Pistol ribozyme sequence having at least 80%, at least 85%, at least 90%, at least 95%, or 100% sequence identity to SEQ ID NO: 64 or 65, and wherein the P2 motif of the 5′ ribozyme sequence has the polynucleotide sequence of “TCAA” or “TTAA”. In some embodiments, the RNA viral genome comprises a 5′ UTR (IRES) sequence having at least 80%, at least 85%, at least 90%, at least 95%, or 100% sequence identity to nucleic acids 1-670 of SEQ ID NO: 68. In some embodiments, the 3′ junctional cleavage sequence comprises or consists of a SapI recognition sequence. In some embodiments, the promoter sequence is a T7 promoter sequence, wherein the leader sequence consists of a polynucleotide sequence according to SEQ ID NO: 53, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence. In some embodiments, the promoter sequence is a T7 promoter sequence, wherein the leader sequence consists of a polynucleotide sequence according to SEQ ID NO: 58, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence.

In some embodiments, the recombinant DNA molecule does not comprise additional nucleic acid within the region spanning the promoter sequence and the 3′ junctional cleavage sequence.

In one aspect, the disclosure provides a method of producing a recombinant RNA molecule, comprising in vitro transcription of the DNA molecule of the disclosure and purification of the resulting recombinant RNA molecule. In some embodiments, the recombinant RNA molecule comprises 5′ and 3′ ends that are native to the oncolytic virus encoded by the synthetic RNA viral genome.

In one aspect, the disclosure provides a composition comprising an effective amount of the recombinant RNA molecule of the disclosure and a carrier suitable for administration to a mammalian subject.

In one aspect, the disclosure provides a particle comprising the recombinant RNA molecule of the disclosure. In some embodiments, the particle is biodegradable. In some embodiments, the particle is selected from the group consisting of a nanoparticle, an exosome, a liposome, and a lipoplex. In some embodiments, the exosome is a modified exosome derived from an intact exosome or an empty exosome.

In one aspect, the disclosure provides a pharmaceutical composition comprising a plurality of particles of the disclosure. In some embodiments, the plurality of particles have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of particles have an average size of about 50 nm to about 120 nm. In some embodiments, the plurality of particles have an average size of about 50 nm, 60 nm, 70 nm, 80 nm, 90 nm, 100 nm, 110 nm, or about 120 nm. In some embodiments, the plurality of particles have an average size of about 100 nm.

In some embodiments, the plurality of particles have an average zeta-potential of between about 40 mV to about −40 mV, about 20 mV to about −20 mV, about 10 mV to about −10 mV, about 5 mV to about −5 mV, or about 20 mV to about −40 mV. In some embodiments, the plurality of particles have an average zeta-potential of less than about 5 mV, less than about 0 mV, less than about −5 mV, less than about −10 mV, less than about −20 mV, less than about −30 mV, less than about −35 mV, or less than about −40 mV. In some embodiments, the plurality of particles have an average zeta-potential of between about −50 mV to about −20 mV, about −40 mV to about −20 mV, about −30 mV to about −10 mV, about −20 mV to about 0 mV, about −15 mV to about 5 mV, or about −10 mV to about 10 mV. In some embodiments, the plurality of particles have an average zeta-potential of about −30 mV, about −31 mV, about −32 mV, about −33 mV, about −34 mV, about −35 mV, about −36 mV, about −37 mV, about −38 mV, about −39 mV, or about −40 mV.

In some embodiments, delivery of the composition to a subject delivers the encapsulated recombinant RNA molecule to a target cell, and wherein the encapsulated recombinant RNA molecule produces an infectious virus capable of lysing the target cell.

In one aspect, the disclosure provides an inorganic particle comprising the recombinant RNA molecule of the disclosure. In some embodiments, the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP). In some embodiments, the average diameter of the particles is less than about 500 nm, is between about 50 nm and 500 nm, is between about 250 nm and about 500 nm, or is about 350 nm.

In some embodiments, the LNP of the disclosure, the particle of the disclosure, or the inorganic particle of the disclosure further comprises a second recombinant RNA molecule encoding a payload molecule. In some embodiments, the second recombinant RNA molecule is a replicon.

In one aspect, the disclosure provides a pharmaceutical composition comprising, the LNP of the disclosure, the particle of the disclosure, or the inorganic particle of the disclosure, wherein the composition is formulated for intravenous and/or intratumoral delivery. In some embodiments, the target cell is a cancerous cell.

In one aspect, the disclosure provides a method of killing a cancerous cell comprising exposing the cancerous cell to the particle of the disclosure, the recombinant RNA molecule of the disclosure, or compositions thereof, under conditions sufficient for the intracellular delivery of the particle to said cancerous cell, wherein the replication-competent virus produced by the encapsulated polynucleotide results in killing of the cancerous cell. In some embodiments, the replication-competent virus is not produced in non-cancerous cells. In some embodiments, the method is performed in vivo, in vitro, or ex vivo.

In one aspect, the disclosure provides a method of treating a cancer in a subject comprising administering to a subject suffering from the cancer an effective amount of the particle of the disclosure, the recombinant RNA molecule of the disclosure, or compositions thereof. In some embodiments, the particle or composition thereof is administered intravenously, intranasally, intratumorally, intraperitoneally, or as an inhalant. In some embodiments, the particle or composition thereof is administered intratumorally and/or intravenously. In some embodiments, the particle or composition thereof is administered to the subject repeatedly. In some embodiments, the subject is a mouse, a rat, a rabbit, a cat, a dog, a horse, a non-human primate, or a human.

In some embodiments, the cancer is lung cancer, breast cancer, colon cancer, or pancreatic cancer, and wherein the synthetic RNA viral genome comprises a polynucleotide sequence derived from the KY strain. In some embodiments, the cancer is bladder cancer, renal cell carcinoma, ovarian cancer, gastric cancer or liver cancer, and wherein the synthetic RNA viral genome comprises a polynucleotide sequence derived from the EF strain.

In some embodiments, the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL). In some embodiments, the cancer is selected from the groups consisting of lung cancer, breast cancer, colon cancer, pancreatic cancer, bladder cancer, renal cell carcinoma, ovarian cancer, gastric cancer and liver cancer. In some embodiments, the cancer is renal cell carcinoma, lung cancer, or liver cancer. In some embodiments, the lung cancer is small cell lung cancer or non-small cell lung cancer (e.g., squamous cell lung cancer or lung adenocarcinoma). In some embodiments, the liver cancer is hepatocellular carcinoma (HCC) (e.g., Hepatitis B virus associated HCC). In some embodiments, the prostate cancer is treatment-emergent neuroendocrine prostate cancer. In some embodiments, the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma. In some embodiments, the cancer is small cell lung cancer (SCLC) or neuroblastoma.

In one aspect, the disclosure provides a method of treating a cancer in a subject in need thereof comprising administering an effective amount of a CVA21-EF virus to the subject.

In one aspect, the disclosure provides a method of treating a cancer in a subject in need thereof comprising administering an effective amount of a CVA21-KY virus to the subject

In one aspect, the disclosure provides a method of treating a cancer in a subject in need thereof comprising administering an effective amount of a CVA21-Kuykendall virus to the subject.

In some embodiments, the virus is administered intratumorally and/or intravenously.

In some embodiments, the method further comprises administering an immune checkpoint inhibitor to the subject. In some embodiments, the immune checkpoint inhibitor is an inhibitor of PD-1. In some embodiments, the method further comprises administering an engineered immune cell comprising an engineered antigen receptor.

In one aspect, the disclosure provides a method of treating a cancer in a subject in need thereof, comprising administering a therapeutically effective amount of an oncolytic Coxsackievirus, wherein the Coxsackievirus is a CVA21 strain, or a polynucleotide encoding the CVA21 to the subject, wherein the cancer is classified as sensitive to CVA21 infection based on the expression of ICAM-1 and/or the percentage of ICAM-1 positive cancer cells.

In one aspect, the disclosure provides a method of treating a cancer in a subject in need thereof, comprising:
(a) determining the expression level of ICAM1 and/or the percentage of ICAM-1 positive cancer cells in the cancer;
(b) classifying the cancer as sensitive to Coxsackievirus 21 (CVA21) infection based on the expression of ICAM-1 and/or the percentage of ICAM-1 positive cancer cells determined in (a); and
(c) administering a therapeutically effective amount of CVA21 or a polynucleotide encoding the CVA21 to the subject if the cancer is classified as sensitive to CVA21 infection in step (b).

In one aspect, the disclosure provides a method of selecting a subject suffering from a cancer for treatment with a Coxsackievirus 21 (CVA21) or a polynucleotide encoding the CVA21, comprising:
(a) determining the expression level of ICAM-1 and/or the percentage of ICAM-1 positive cancer cells in the cancer;
(b) classifying the cancer as sensitive to CVA21 infection based on the expression level of ICAM-1 and/or the percentage of ICAM1 positive cancer cells as determined in (a);
(c) selecting the subject for treatment with the CVA21 or the polynucleotide encoding the CVA21 if the cancer is classified as sensitive to CVA21 infection in (b); and
(d) administering the CVA21 or the polynucleotide encoding the CVA21 to the selected subjects

In some embodiments, the CVA21 strain is CVA21-KY.

### BRIEF DESCRIPTION OF THE DRAWINGS

FIG. 1 shows tumor volume in SK-MEL-28 tumor-bearing mice following intratumoral administration of PBS or CVA21-RNA molecules formulated with Lipofectamine or intravenous administration of LNPs comprising CVA21-Kuykendall strain RNA molecules (formulation ID: 70032-6C).

FIG. 2A shows an overview of an in vitro transcriptional approach to generate an authentic 3′ terminus for picornaviruses using 3′ Type IIS restriction enzyme recognition sites.

FIG. 2B shows electrophoresis of DNA digestion by BsmBI or BsaI restriction enzyme.

FIG. 3 shows an RNaseH approach for generating an authentic 5′ terminus for picornaviruses using 5′ DNA primers and an RNaseH enzyme.

FIG. 4 shows a ribozyme approach for generating authentic 5′ termini for picornaviruses.

FIG. 5A-FIG. 5B show hammerhead ribozymes for generation of discrete 5′ termini. FIG. 5A shows a structural model of a minimal hammerhead ribozyme (HHR) that anneals and cleaves at the 5′ terminus at the arrow (SEQ ID NO: 75). FIG. 5B shows a structural model of a ribozyme with a stabilized stem I (STBL) for cleavage of 5′ terminus at the arrow (SEQ ID NO: 76).

FIG. 6A-FIG. 6C show pistol ribozymes for generation of discrete 5′ termini. FIG. 6A shows a schematic of wild type Pistol ribozyme characteristics (SEQ ID NO: 77). FIG. 6B shows Pistol ribozyme from P. Polymyxa with a tetraloop added to fuse the P3 strands modeled by mFOLD. The dashed box is the area mutagenized to retain the fold of the ribozyme in the context of the viral sequence. The “GUC” sequence shown in the dashed box was mutated to “UCA” to generate Pistol 1 and the “GUC” sequence was mutated to “TTA” to generate Pistol 2. (SEQ ID NO: 78) FIG. 6C shows the sequence alignment of multiple Pistol ribozyme variants and the location of the P2 motif.

FIG. 7A-FIG. 7B shows production of infectious CVA21 virus from RNA polynucleotides. FIG. 7A shows effects of 5′UTR sequences on the production of infectious CVA21 from RNA polynucleotides. FIG. 7B shows production of infectious CVA21 from RNA polynucleotides comprising the 5′ UTR of SEQ ID NO: 2.

FIG. 8A-FIG. 8B illustrate the in vivo effects of CVA-LNP. FIG. 8A shows tumor measurements. FIG. 8B shows body weight changes over time. FIG. 8C shows the result of H1299 cell lysis due to CVA21 infection (left) and Western-based expression analysis of ICAM1 and DAF in the indicated cell lines (right). FIG. 8D are charts showing the average ICAM-1 mRNA and protein expression in 130 human cell lines based on their sensitivity/resistance to CVA21-KY infection. FIG. 8E is a diagram showing the correlation between ICAM-1 expression and sensitivity to CVA21-KY infection.

FIG. 9 shows a schematic representation of LNP/picornavirus RNA composition and mode of action. LNP/picornavirus RNA is systemically administered, and picornavirus RNA genomes are delivered to permissive tumor cells where they replicate and produce picornavirus virions. Picornavirus infection then spreads to neighboring tumor cells eliciting oncolysis and antiviral immune responses.

FIG. 10 shows the in vitro transcription process for CVA21-RNA and Neg-RNA. Autocatalytic cleavage of CVA21-RNA by 5′ and 3′ ribozyme (Rib) generated CVA21-RNA with discrete 5′ and 3′ ends required for replication. In contrast, the Neg-RNA construct lacks ribozyme sequence and was not capable of replication and virion production.

FIG. 11A shows a general schematic of using junctional cleavage sequences to remove non-viral RNA polynucleotides from the genome transcripts in order to maintain the native 5′ and 3′ discrete ends of the virus. FIG. 11B shows a schematic of using junctional cleavage sequences to remove non-viral RNA polynucleotides from the genome transcripts in order to maintain the native 5′ and 3′ discrete ends of the virus wherein the 3′ junctional cleavage sequence comprises a restriction enzyme recognition site.

FIG. 12A-FIG. 12B shows a summary diagram of the AUC obtained from a cytotoxicity screen for the three CVA21 strains (FIG. 12A) and their relative IC50 values for each individual cell line (FIG. 12B).

FIG. 13 shows the IC50 values of the three CVA21 strains for each individual cell line in various cancer types.

FIG. 14 shows results plotted as a ratio of the AUC values for EF and KY strains using lung and NSCLC cancer cell lines.

FIG. 15A-FIG. 15C shows results plotted as a ratio of the AUC values for EF and KY strains using breast (FIG. 15A), colon/GI (FIG. 15B), and pancreatic cancer (FIG. 15C) cell lines.

FIG. 16A shows ICAM-1 expression on human lung cancer microarray via immunohistochemistry. This includes the percentage of tumor cells that are ICAM-1 positive and H-score (formula includes percentage of cells that express +1, +2, +3 ICAM-1 intensity levels) FIG. 16B shows the oncolytic efficacy of EF and KY strains on various lung adenocarcinoma cancer cell lines. FIG. 16C shows the oncolytic efficacy of EF and KY strains on various large cell lung cancer cell lines.

FIG. 17A-FIG. 17E shows results plotted as a ratio of the AUC values for EF and KY strains using bladder (FIG. 17A), renal (FIG. 17B), liver (FIG. 17C), ovarian (FIG. 17D), and GBM (FIG. 17E) cancer cell lines.

FIG. 18A shows results plotted as a ratio of the AUC values for EF and KY strains using breast cancer cell lines. FIG. 18B shows results plotted as a ratio of the AUC values for EF and KY strains using renal cell carcinoma cell lines. FIG. 18C shows results plotted as a ratio of the AUC values for EF and KY strains using hepatocellular carcinoma cell lines.

FIG. 19 is a table summarizing the cell line sensitivity to KY and EF strains based on the TCID50 value.

FIG. 20A and FIG. 20B show diagrams plotting the copy numbers of KY and EF strains in human dissociated tumor cells from three donors at 24 hour and 72 hour time points post-infection.

FIG. 21 shows the change of tumor sizes over time in animals treated with the indicated LNPs based on an NCI-H1299 xenograft model and the calculated TGI percentage.

FIG. 22 shows the change of tumor sizes over time in animals treated with the indicated LNPs based on an NCI-H2122 xenograft model and the calculated TGI percentage.

FIG. 23 shows the change of tumor sizes over time in animals treated with the indicated LNPs based on a PC3 xenograft model and the calculated TGI percentage.

FIG. 24 shows the change of body weight over time in animals that received the indicated treatment (upper panel) and the liver chemistry changes (lower panels).

FIG. 25A-FIG. 25C shows the copy numbers of the CVA21 strand RNA based on RT-qPCR in various tissues of animals treated with LNPs comprising the indicated viral genome. FIG. 25A shows copy numbers of the CVA21 (−) strand RNA at 48 hours post treatment. FIG. 25B shows copy numbers of the CVA21 (−) strand RNA at 7 days post treatment. FIG. 25C shows a diagram plotting the copy numbers of the CVA21 (+) strand RNA based on RT-qPCR in various tissues of animals treated with LNPs comprising the indicated viral genome 48 hours or 7 days post infection.

FIG. 26 shows results of viral plaque assays of samples extracted from the indicated tissues.

FIG. 27A-FIG. 27C shows the cell survival of indicated cell lines pretreated with increasing amount of IFN before infection with the indicated virus strains. For the calculation of relative percentage of survived cells, the 100% value is set according to the mock infection group, whereas 0% value is set according to groups of CVA21 virus infection without IFN pretreatment. FIG. 27A shows results for H1299 cell lines. FIG. 27B shows results for H2122 cell lines. FIG. 27C shows results for HFF cell lines.

FIG. 28A shows the relative Ribavirin resistance frequency ratio of the indicated virus strains. FIG. 28B shows the amount of recombined viable viruses recovered from the indicated assay groups.

FIG. 29A shows the design of the template construct for leader sequence and 5′ ribozyme sequence analysis. FIG. 29B shows the gel electrophoresis result of the indicated in vitro transcription products based on different leader sequence designs.

FIG. 30 shows the gel electrophoresis result of the indicated in vitro transcription products based on different 5′ ribozyme designs.

FIG. 31A is a diagram plotting the change of tumor sizes over time in animals treated with the indicated LNPs comprising RNA viral genomes obtained from in vitro transcription of DNA templates with different designs (e.g., varying poly-A tail length and 5′ ribozyme sequences), using a NCI-H1299 xenograft model. FIG. 31B shows diagrams plotting the change of tumor sizes over time in animals treated with the indicated LNPs.

FIG. 32 shows domain organization schematics of the RNA viral genomes of three CVA21 strains (EF, KY, and Kuykendall) and the nucleic acid starting/ending positions of selective region.

FIG. 33 shows schematics of a non-limiting example of the CVA21 expression construct design and corresponding in vitro transcription process to generate synthetic RNA viral genomes with precise ends at 5′ and 3′.

FIG. 34 is a schematic showing the domain organization of SVV viral genome and construction of chimeric viruses.

FIG. 35 shows fluorescence microscopy images of NCI-H69AR cells infected with SVV-001 or SVV-IRES chimeric viruses carrying a GFP reporter gene.

FIG. 36 shows diagrams plotting the fluorescence intensities of NCI-H69AR and NCI-H69 cells infected with indicated SVV virions with or without IFNα pretreatment (left), and the representative fluorescence images 12-hour post-infection (right).

FIG. 37 shows fluorescence microscopy images of NCI-H69AR cells infected with SVV-001 or SVV-P1 chimeric viruses carrying a GFP reporter gene.

FIG. 38A and FIG. 38B show fluorescence microscopy images of NCI-H69AR cells infected with SVV-001 or SVV-P3 chimeric viruses carrying a GFP reporter gene.

FIG. 39A illustrates the template construct design for leader sequence and 5′ ribozyme sequence analysis for SVV template. FIG. 39B shows the gel electrophoresis result of the indicated in vitro transcription products based on different leader sequence designs for SVV template. FIG. 39C shows the result of viral plaque assay using H1299 cells. FIG. 39D shows RP-HPLC analysis of in vitro transcription products of viral genomes using different SVV leader sequences.

FIG. 40A-FIG. 40C show the in vivo dose titration efficacy study results athymic-nude mice bearing H446 tumors were treated with the indicated LNPs. FIG. 40A shows tumor sizes over time. FIG. 40B shows RT-qPCR measurements for SVV replication in tumor tissues of mice treated with 96062-1 LNP at 0.1 mg/kg. FIG. 40C shows a FISH assay for SVV replication.

FIG. 41 shows tumor sizes over time of mice bearing H446 tumors treated with LNPs

FIG. 42 shows the change of tumor sizes over time in animals treated with the indicated LNPs based on a H446 SCLC tumor model.

FIG. 43 shows the change of tumor sizes over time in animals treated with the indicated LNPs or SVV virions at the presence or absence of anti-SVV neutralizing antibody based on a H446 SCLC tumor model.

FIG. 44 shows the change of tumor sizes over time in animals treated with the indicated LNPs based on a H82 SCLC tumor model.

FIG. 45 shows the probability of survival over time in animals treated with the indicated LNPs based on a H82 SCLC orthotopic tumor model.

FIG. 46A is a chart showing quantitative analysis of tumor burden based on hDLL3 IHC. FIG. 46B show hDLL3 IHC images.

FIG. 47A and FIG. 47B show SVV treatment results of SCLC PDX tumor model. FIG. 47A shows the change of tumor sizes over time in animals treated with the indicated LNPs based on a SCLC PDX tumor model. FIG. 47B shows the results of RT-qPCR measurement.

FIG. 48 shows the change of tumor sizes over time in animals treated with the indicated LNPs comprising SVV RNA viral genome with different lengths of poly-A tail.

FIG. 49A-FIG. 49G shows immune cell infiltration and effects of combined SVV/LNP and anti-PD1 treatment in an N1E-115 syngeneic neuroblastoma model. FIG. 49A shows the number of NK, NKT, CD4, CD8, and Treg cells per mg of tumor. FIG. 49B shows the CD8/Treg ratio. FIG. 49C shows the number of CD8 T cells per mg of tumor that express CTLA-4 or PD-1. FIG. 49D shows the number of CD8 T cells per mg of tumor that are SLEC (CD127-KLRG1+) or MPEC (CD127+—KLRG1—). FIG. 49E shows ratio of M1/M2 macrophages. FIG. 49F shows the number of M1 and M2 macrophages. FIG. 49G shows the number of CD45− PD-L1+ cells per mg of tumor.

FIG. 50 shows the change of tumor sizes over time in animals treated with the indicated LNPs based on a N1E-115 syngeneic neuroblastoma model.

FIG. 51 shows the combined effects of SVV-LNP and anti-PD1 therapy in animals bearing N1E-115 syngeneic neuroblastoma model.

FIG. 52A is a chart depicting the results of a H446 mouse tumor model showing the growth of tumor upon repeat dose of the LNP compositions of the disclosure. FIG. 52B is a chart depicting the body weight change of the H446 mouse tumor model upon administration of the LNP composition.

FIG. 53A and FIG. 53B are charts depicting the results of PK study in mice of LNP compositions comprising PEG2k-DPG, PEG2k-DMG or BRIJ™ S100 as PEG-lipid.

FIG. 54A shows the UV A280 absorption profile of CVA21 viral genome with varying poly-A tail length using Oligo-dT chromatography. FIG. 54B shows the UV A280 absorption profile of SVV viral genome with varying poly-A tail length using Oligo-dT chromatography.

FIG. 55A-FIG. 55C illustrate particle characteristics of CAT4 and CAT5 LNP compositions. FIG. 55A depicts the particle sizes and FIG. 55B depicts the polydispersity index determined in a dynamic light scattering experiment of CAT4 and CAT5 LNP compositions made with various RNA acidifying buffers. FIG. 55C depicts the encapsulation efficiency of these LNP compositions measured by RiboGreen.

FIG. 56A-FIG. 56C depict particle characteristic of LNP compositions comprising the indicated CAT lipids. FIG. 56A depicts the particle sizes and FIG. 56B depicts the polydispersity index determined in a dynamic light scattering experiment of LNP compositions. FIG. 56C depicts the encapsulation efficiency of these LNP compositions measured by RiboGreen.

FIG. 57 shows a schematic representation of the formulation process for the LNP formulations.

FIG. 58A-FIG. 58F depict RNA levels measured by NanoLuc luciferase activation. FIG. 58A, FIG. 58B, and FIG. 58C depict RNA levels measured by NanoLuc luciferase activation 96 h post-dose of the respective LNP formulations. FIG. 58D, FIG. 58E, and FIG. 58F depict RNA levels measured by NanoLuc luciferase activation 72 h post-dose of the respective LNP formulations.

FIGS. 59A-FIG. 59E depict the tumor volume (left) and body weight change (right) over the days of treatment of the mice treated with the respective LNP formulations. FIG. 59A shows results for CAT1, 2, 3, 4, and 5 LNP formulations. FIG. 59B shows results for CAT6, 7, 8, 9, 10 LNP formulations. FIG. 59C shows results for CAT11, 12, 13, 14, 15, 16. FIG. 59D shows results for CAT17, 19, 20, 25, 31, and 7 LNP formulations. FIG. 59E shows results for CAT18, 21, 22, 23, 26, 28, 29, 32, 34 LNP formulations.

FIG. 60A-FIG. 60B shows in vivo RNA levels and effects on tumor volume and body weight for the indicated LNP formulations. FIG. 60A depicts the RNA levels measured by the luminescence produced by NanoLuc luciferase activation 72 h post-dose of the respective LNP formulations. FIG. 60B depicts the tumor volume (right) and body weight change (left) over the days of treatment of the mice treated with the respective LNP formulations.

FIGS. 61A-61D depict the concentration of the ionizable lipid comprised in the LNPs (SS—OC) in the plasma of the treated mice measured by LC-MS. FIG. 61A shows results for OC/Brij on a Q1W2 dosing schedule. FIG. 61B shows results for OC/Brij on a Q2W2 dosing schedule. FIG. 61C shows results for OC/DMG on a Q2W2 dosing schedule. FIG. 61D shows results for OC/DPG on a Q2W2 dosing schedule.

FIG. 62A-FIG. 62F depict the concentration of the ionizable lipid comprised in the LNPs (SS—OC or CAT7) in the plasma of the treated mice measured by LC-MS. FIG. 62A shows results for OC/Brij on a Q2W2 dosing schedule. FIG. 62B shows results for CAT7/DMG_6 on a Q2W2 dosing schedule. FIG. 62C shows results for CAT7/DMG_3 on a Q2W2 dosing schedule.

FIG. 62D shows results for CAT7/DMG_5 on a Q2W2 dosing schedule. FIG. 62E shows results for CAT7/DMG1_1 on a Q2W2 dosing schedule. FIG. 62F shows results for CAT7/CHM6_4 on a Q2W2 dosing schedule.

FIG. 63A-FIG. 63E depict the concentration of the ionizable lipid comprised in the LNPs (SS—OC, CAT7, or CAT11) in the plasma of the treated mice measured by LC-MS. FIG. 63A shows results for CAT7/Brij. FIG. 63B shows results for OC/DMG. FIG. 63C shows results for CAT7/CHM6_4. FIG. 63D shows results for CAT11/DMG. FIG. 63E shows results for CAT11/Birj.

FIG. 64A and FIG. 64B depict the IgM levels at the indicated timepoints of the mice treated with the respective LNP formulations measured by an ELISA assay.

FIG. 65A and FIG. 65B depict the IgG levels at the indicated timepoints of the mice treated with the respective LNP formulations measured by an ELISA assay.

FIG. 66A and FIG. 66B depict the plasma levels of the mRNA BiTE (FIG. 66A) or hEPO (FIG. 66B) measured by ECL assays

FIG. 67 depicts an A-optimal design of screening experiments for LNPs comprising CAT7.

FIG. 68 shows the prediction profilers modeled based on the design of experiment runs for LNPs comprising CAT7 and using the Self-Validated Ensemble Modeling method.

### DETAILED DESCRIPTION

There is a need in the art for selecting specific oncolytic virus strain with appropriate tropism and high potency in specific cancer types. Different strains of viruses, even those from the same species, vary greatly in terms of their efficacy in killing cancer cells and as toxicity. A chimeric virus may be generated by replacing part of the viral genome of one viral strain with the corresponding part from a different strain. In some embodiments, such chimeric virus may be more efficacious in killing cancer cells and/or have other advantageous properties. In addition, production of compositions comprising such viruses or corresponding viral genomes requires optimization of the design of vector template as well as the manufacturing process (e.g., expression, purification, encapsulation, and storage). In some embodiments, the present disclosure provides viral genomes and design of template vectors for viral expression in vitro. In some embodiments, the viral genomes are replication competent. The present disclosure also provides viral genomes that can be encapsulated in a non-immunogenic particle, such as a lipid nanoparticle, polymeric nanoparticle, or an exosome, which can be repeatedly administered to a subject. In some embodiments, the particle further encapsulates a polynucleotide encoding a payload molecule. Accordingly, the present disclosure enables the systemic delivery of a safe, efficacious recombinant polynucleotide vector, and provides methods for the treatment and prevention of a broad array of proliferative disorders (e.g., cancers).

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 10%, in either direction (greater than or less than) of the stated reference value unless otherwise stated or otherwise evident from the context (except where such number would exceed 100% of a possible value). In some embodiments, the term “approximately” or “about” refers to a range of values that fall within 10% in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99, or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. Unless noted otherwise, the term “sequence identity” in the claims refers to sequence identity as calculated by Clustal Omega® version 1.2.4 using default parameters.

The term “derived from” refers to a polypeptide or polynucleotide sequence that comprises all or a portion of a reference polypeptide or polynucleotide sequence. For example, an RNA polynucleotide encoding an SVV or CVA genome described herein may comprise a polynucleotide sequence derived from all or a portion of a reference SVV or CVA genome (e.g., a naturally occurring or modified SVV or CVA genome). A polypeptide or polynucleotide sequence “derived from” a reference polypeptide or polynucleotide sequence also includes polypeptide and/or polynucleotide sequences that comprise one more amino acid or nucleic acid mutations (e.g., substitutions, deletions, and/or insertions) relative to the reference polypeptide or polynucleotide sequence.

“Complementary” refers to the capacity for pairing, through base stacking and specific hydrogen bonding, between two sequences comprising naturally or non-naturally occurring (e.g., modified as described above) bases (nucleotides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

An “expression cassette” or “expression construct” refers to a polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject or contacting a composition with a cell and/or tissue.

“Treating” as used herein refers to delivering an agent or composition to a subject to affect a physiologic outcome. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state; and (c) curing the disease.

The term “effective amount” refers to the amount of an agent or composition required to result in a particular physiological effect (e.g., an amount required to increase, activate, and/or enhance a particular physiological effect). The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

“Effector function” refers to functions of an immune cell related to the generation, maintenance, and/or enhancement of an immune response against a target cell or target antigen.

The terms “microRNA,” “miRNA,” and “miR” are used interchangeably herein and refer to small non-coding endogenous RNAs of about 21-25 nucleotides in length that regulate gene expression by directing their target messenger RNAs (mRNA) for degradation or translational repression.

The term “composition” as used herein refers to a formulation of a recombinant RNA molecule or a particle-encapsulated recombinant RNA molecule described herein that is capable of being administered or delivered to a subject or cell.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals.

The term “replication-competent viral genome” refers to a viral genome encoding all of the viral genes necessary for viral replication and production of an infectious viral particle.

The term “oncolytic virus” refers to a virus that has been modified to, or naturally, preferentially infect cancer cells.

The term “vector” is used herein to refer to a nucleic acid molecule capable of transferring, encoding, or transporting another nucleic acid molecule.

The terms “corresponding to” or “correspond to”, as used herein in relation to the amino acid or nucleic acid position(s), refer to the position(s) in a first polypeptide/polynucleotide sequence that aligns with a given amino acid/nucleic acid in a reference polypeptide/polynucleotide sequence when the first and the reference polypeptide/polynucleotide sequences are aligned. Alignment is performed by one of skill in the art using software designed for this purpose, for example, Clustal Omega version 1.2.4 with the default parameters for that version.

The term “encapsulation efficiency” or “EE %” refers to the percentage of a target molecule (e.g., synthetic RNA viral genome) that is successfully entrapped into LNP. Encapsulation efficiency may be calculated using the formula:

(EE%)=(Wt/Wi)×100%

where Wt is the total amount of drug in the LNP suspension and Wi is the total quantity of drug added initially during preparation. As an illustrative example, if 97 mg of the target molecule are entrapped into LNPs out of a total 100 mg of the target molecule initially provided to the composition, the encapsulation efficiency may be given as 97%.

The term “lipid-nitrogen-to-phosphate ratio” or “(N:P)” refers to the ratio of positively-chargeable lipid amine groups to nucleic acid phosphate groups in a lipid nanoparticle.

The term “half-life” refers to a pharmacokinetic property of a molecule (e.g., a molecule encapsulated in a lipid nanoparticle). Half-life can be expressed as the time required to eliminate through biological processes (e.g., metabolism, excretion, accelerated blood clearance, etc.) fifty percent (50%) of a known quantity of a molecule in vivo, following its administration, from the subject's body (e.g., human patient or other mammal) or a specific compartment thereof, for example, as measured in serum, i.e., circulating half-life, or in other tissues. In general, an increase in half-life results in an increase in mean residence time (MRT) in circulation for the molecule administered.

The term “accelerated blood clearance” or “ABC” refers to a phenomenon in which certain pharmaceutical agents (e.g., PEG-containing LNPs) are rapidly cleared from the blood upon second and subsequent administrations. ABC has been observed for many lipid-delivery vehicles, including liposomes and LNPs.

As used herein, the term “ratio” when used in reference to lipid composition (e.g., as a percentage of total lipid content) refers to molar ratio, unless clearly indicated otherwise. The molar ratio as a percentage of total lipid content can also be represented by “mol %”. For example, “49:22:28.5:0.5 mol %” means a molar ratio of 49:22:28.5:0.5.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic,” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “aryl” used alone or as part of a larger moiety as in “aralkyl,” “aralkoxy,” or “aryloxyalkyl,” refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “haloaliphatic” refers to an aliphatic group that is substituted with one or more halogen atoms.

The term “haloalkyl” refers to a straight or branched alkyl group that is substituted with one or more halogen atoms.

The term “halogen” means F, Cl, Br, or I.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; CH═CHPh, which may be substituted with R^{∘}; —(CH₂)₀₋₄O(CH₂)₀₋₁-pyridyl which may be substituted with R^{∘}; NO₂; CN; N₃; —(CH₂)₀₋₄N(R^{∘})₂; —(CH₂)₀₋₄N(R^{∘})C(O)R^{∘}; N(R^{∘})C(S)R^{∘}; —(CH₂)₀₋₄N(R^{∘})C(O)NR^{∘}2; N(R^{∘})C(S)NR^{∘}2; —(CH₂)₀₋₄N(R^{∘})C(O)OR^{∘}; N(R^{∘})N(R^{∘})C(O)R^{∘}; —N(R^{∘})N(R^{∘})C(O)NR^{∘}2; N(R^{∘})N(R^{∘})C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}3; —(CH₂)₀₋₄OC(O)R^{∘}; OC(O)(CH₂)₀₋₄SR^{∘}, SC(S)SR^{∘}; —(CH₂)₀₋₄SC(O)R^{∘}; —(CH₂)₀₋₄C(O)NR^{∘}2; C(S)NR^{∘}2; —C(S)SR^{∘}; —SC(S)SR^{∘}, —(CH₂)₀₋₄OC(O)NR^{∘}2; C(O)N(OR^{∘})R^{∘}; C(O)C(O)R^{∘}; C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; N(R^{∘})S(O)₂NR^{∘}₂; N(R^{∘})S(O)₂R^{∘}; N(OR^{∘})R^{∘}; —C(NH)NR^{∘}₂; P(O)₂R^{∘}; P(O)R^{∘}₂; OP(O)R^{∘}₂; OP(O)(OR^{∘})₂; SiR^{∘}3; —(C₁₋₄ straight or branched alkylene)O—N(R^{∘})₂; or (C₁₋₄ straight or branched alkylene)C(O)O—N(R^{∘})₂, wherein each R^{∘} may be substituted as defined below and is independently hydrogen, C₁₋₆ aliphatic, —CH₂Ph, O(CH₂)₀₋₁Ph, CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘}(or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}2, NO₂, SiR^{•}₃, OSiR^{•}₃, —C(O)SR^{•}, (C₁₋₄ straight or branched alkylene)C(O)OR^{•}, or SSR^{•} wherein each R^{•} is unsubstituted or where preceded by “halo” is substituted only with one or more halogens, and is independently selected from C₁₋₄ aliphatic, CH₂Ph, O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)*, ═NNHC(O)OR*, ═NNHS(O)₂R*, ═NR*, ═NOR*, O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, R^{•}, -(haloR^{•}), —OH, —OR^{•}, —O(haloR^{•}), —CN, —C(O)OH, —C(O)OR^{•}, —NH₂, —NHR^{•}, —NR^{•}2, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The term “tertiary amine” is used to describe an amine (nitrogen atom) which is attached to three carbon-containing groups, each of the groups being covalently bonded to the amine group through a carbon atom within the group. A tertiary amine may be protonated or form a complex with a Lewis acid.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Unless otherwise stated, structures depicted herein are also meant to include all enantiomeric, diastereomeric, and geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### Synthetic RNA Viral Genomes

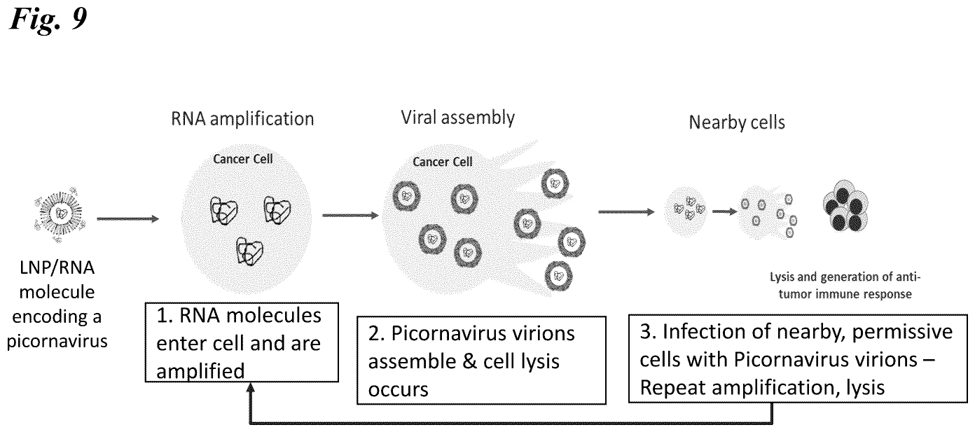

In some embodiments, the present disclosure provides a recombinant RNA molecule encoding an oncolytic virus (e.g., an RNA genome). Such recombinant RNA molecules are referred to herein as “synthetic viral genomes” or “synthetic RNA viral genomes”. In such embodiments, the synthetic RNA viral genome is capable of producing an infectious, lytic virus when introduced into a cell by a non-viral delivery vehicle and does not require additional exogenous genes or proteins to be present in the cell in order to replicate and produce an infectious virus. Rather, the endogenous translational mechanisms in the host cell mediate expression of the viral proteins from the synthetic RNA viral genome. The expressed viral proteins then mediate viral replication and assembly into an infectious viral particle (which may comprise a capsid protein, an envelope protein, and/or a membrane protein) comprising the RNA viral genome. As such, the RNA polynucleotides described herein (i.e., the synthetic RNA viral genomes), when introduced into a host cell, produce a virus that is capable of infecting another host cell. In some embodiments, the oncolytic virus is a picornavirus (see schematic in FIG. 9). In some embodiments, the picornavirus is a CVA21. In some embodiments, the picornavirus is an SVV.

In some embodiments, the synthetic viral genome is provided as a recombinant ribonucleic acid (RNA) (i.e., a synthetic RNA viral genome). In some embodiments, the synthetic RNA viral genomes comprise one or more nucleic acid analogues. Examples of nucleic acid analogues include 2′-O-methyl-substituted RNA, 2′-O-methoxy-ethyl bases, 2′ Fluoro bases, locked nucleic acids (LNAs), unlocked nucleic acids (UNA), bridged nucleic acids (BNA), morpholinos, and peptide nucleic acids (PNA). In some embodiments, the synthetic RNA viral genome is a replicon, a RNA viral genome encoding a transgene, an mRNA molecule, or a circular RNA molecule (circRNA). In some embodiments, the synthetic RNA viral genome comprises a single stranded RNA (ssRNA) viral genome. In some embodiments, the single-stranded genome may be a positive sense or negative sense genome.

In some embodiments, the recombinant RNA molecule is a circular RNA molecule (circRNA). CircRNA molecules lack the free ends necessary for exonuclease mediated degradation, thus extending the half-life of the RNA molecule and enabling more stable protein production over time (See e.g., Wesselhoeft et al., Engineering circular RNA for potent and stable translation in eukaryotic cells. Nature Communications. (2018) 9:2629). In order to produce a functional RNA virus from a circRNA molecule, it is necessary to “break open” the circular construct once inside a cell so that the linear RNA genome with the appropriate 3′ and 5′ native ends can be produced. Therefore, in some embodiments, the recombinant RNA molecule encoding the oncolytic virus is provided as a circRNA molecule and further comprises one or more additional RNA sequences that facilitate the linearization of the circRNA molecule inside a cell. Examples of such additional RNA sequences include siRNA target sites, miRNA target sites, and guide RNA target sites. The corresponding siRNA, miRNA, or gRNA can be co-formulated with the circRNA molecule. Alternatively, the miRNA target site can be selected based on the expression of the cognate miRNA in a target cell, such that cleavage of the circRNA molecule and initial expression of the encoded oncolytic virus is limited to target cells expressing a particular miRNA.

The synthetic RNA viral genomes described herein encode an oncolytic virus. Examples of oncolytic viruses are known in the art including, but not limited to a picornavirus (e.g., a coxsackievirus), a polio virus, a measles virus, a vesicular stomatitis virus, an orthomyxovirus, and a maraba virus. In some embodiments, the oncolytic virus encoded by the synthetic RNA viral genome is a virus in the family Picornaviridae family such as a coxsackievirus, a polio virus (including a chimeric polio virus such as PVS—RIPO and other chimeric Picornaviruses), or a Seneca valley virus, or any virus of chimeric origin from any multitude of picornaviruses, a virus in the Arenaviridae family such a lassa virus, a virus in the Retroviridae family such as a murine leukemia virus, a virus in the family Orthomyxoviridae such as influenza A virus, a virus in the family Paramyxoviridae such as Newcastle disease virus or measles virus, a virus in the Reoviridae family such as mammalian orthoreovirus, a virus in the Togaviridae family such as sindbis virus, or a virus in the Rhabdoviridae family such as vesicular stomatitis virus (VSV) or a maraba virus.

### Positive-Sense, Single-Stranded RNA Viruses

In some embodiments, the synthetic RNA viral genomes described herein encode a single-stranded RNA (ssRNA) viral genome. In some embodiments, the ssRNA virus is a positive-sense, ssRNA (+sense ssRNA) virus. Exemplary+sense ssRNA viruses include members of the Picoraviridae family (e.g. coxsackievirus, poliovirus, and Seneca Valley virus (SVV), including SVV-A), the Coronaviridae family (e.g., Alphacoronaviruses such as HCoV-229E and HCoV-NL63, Betacoronoaviruses such as HCoV-HKU1, HCoV-OC3, and MERS-CoV), the Retroviridae family (e.g., Murine leukemia virus), and the Togaviridae family (e.g., Alphaviruses such as the Semliki Forest virus, Sindbis virus, Ross River virus, or Chikungunya virus). Additional exemplary genera and species of positive-sense, ssRNA viruses are shown below in Table 1.

TABLE 1
Positive-sense ssRNA Viruses

| | | Natural | |
|---|---|---|---|
| Family/Subfamily | Genus | Host | Species |
| Picornaviridae | Cardiovirus | Human | |
| | Cosavirus | Human | |
| | Enterovirus | Human | Coxsackievirus |
| | | Human | Poliovirus |
| | Hepatovirus | Human | |
| | Kobuvirus | Human | |
| | Parechovirus | Human | |
| | Rosavirus | Human | |
| | Salivirus | Human | |
| | Pasivirus | Pigs | |
| | Senecavirus | Pigs | Seneca Valley |
| | | | Virus A |
| Caliciviridae | Sapovirus | Human | |
| | Norovirus | Human | |
| | Nebovirus | Bovine | |
| | Vesivirus | Felines/ | |
| | | Swine | |
| Hepeviridae | Orthohepevirus | | |
| Astroviridae | Mamastrovirus | Human | |
| | Avastrovirus | Birds | |
| Flaviviridae | Hepacivirus | Human | |
| | Flavivirus | Arthropod | |
| | Pegivirus | | |
| | Pestivirus | Mammals | |
| Coronaviridae/ | Alphacoronavirus | | HCoV-229E |
| Coronavirinae | | | HCoV-NL63 |
| | Betacoronavirus | | HCoV-HKU1 |
| | | | HCoV-OC3 |
| | | | MERS-CoV |
| | Deltacoronavirus | | |
| | Gammacoronavirus | | |
| Coronaviridae/ | Bafinivirus | | |
| Torovirinae | Torovirus | | |
| Retroviridae | Gammaretrovirus | | Murine leukemia |
| | | | virus |
| Togaviridae | Alphavirus | | Sindbis virus |
| | | | Semliki Forest |
| | | | virus |
| | | | Ross River virus |
| | | | Chikungunya |
| | | | virus |
| | | | Venezuelan |
| | | | equine |
| | | | encephalitis virus |

In some embodiments, the recombinant RNA molecules described herein encode a Picornavirus selected from a coxsackievirus, poliovirus, and Seneca Valley virus (SVV). In some embodiments, the recombinant RNA molecules described herein encode a coxsackievirus. In some aspects of this embodiment, the recombinant RNA molecules a coxsackievirus and comprise the 5′ UTR sequence of SEQ ID NO: 2 (See e.g., Brown et al., Complete Genomic Sequencing Shows that Polioviruses and Members of Human Enterovirus Species C Are Closely Related in the Noncapsid Coding Region. Journal of Virology, (2003)77:16, p. 8973-8984. Genflank Accession No. AF546702). In such embodiments, the 5′ UTR sequence of SEQ TD NO: 2 unexpectedly increases the production of a functional coxsackievirus compared to other previously described 5′ UTR sequences (See e.g., Newcombe et al., Cellular receptor interactions of C-cluster human group A coxsackieviruses Journal of General Virology (2003), 84, 3041-3050. Genflank Accession No. AF465515). In some aspects of this embodiment, the recombinant RNA molecules encode a coxsackievirus and comprise the sequence of SEQ ID NO: 1.

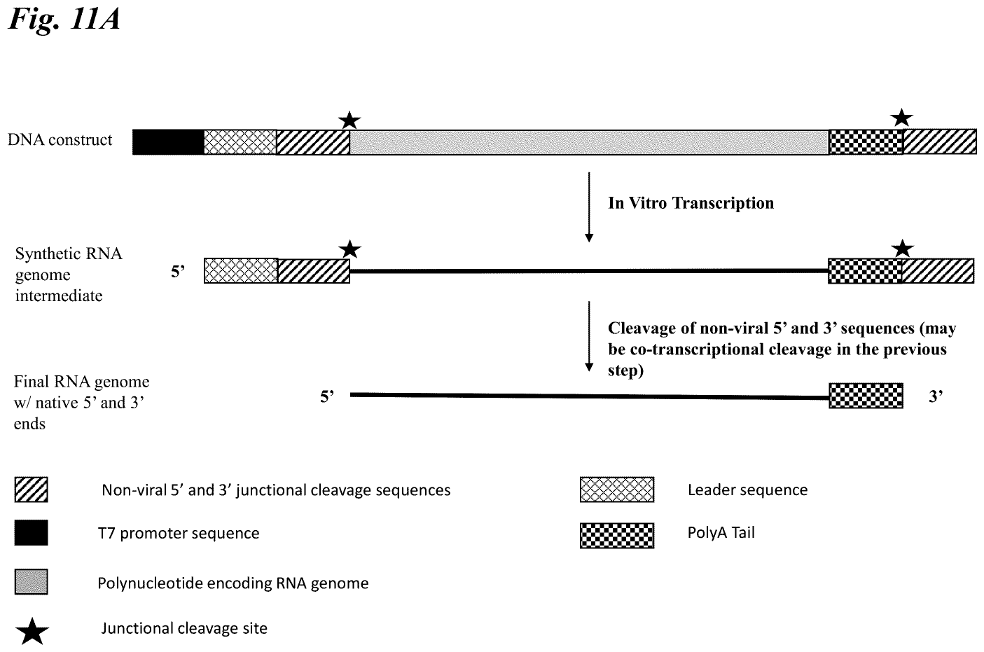

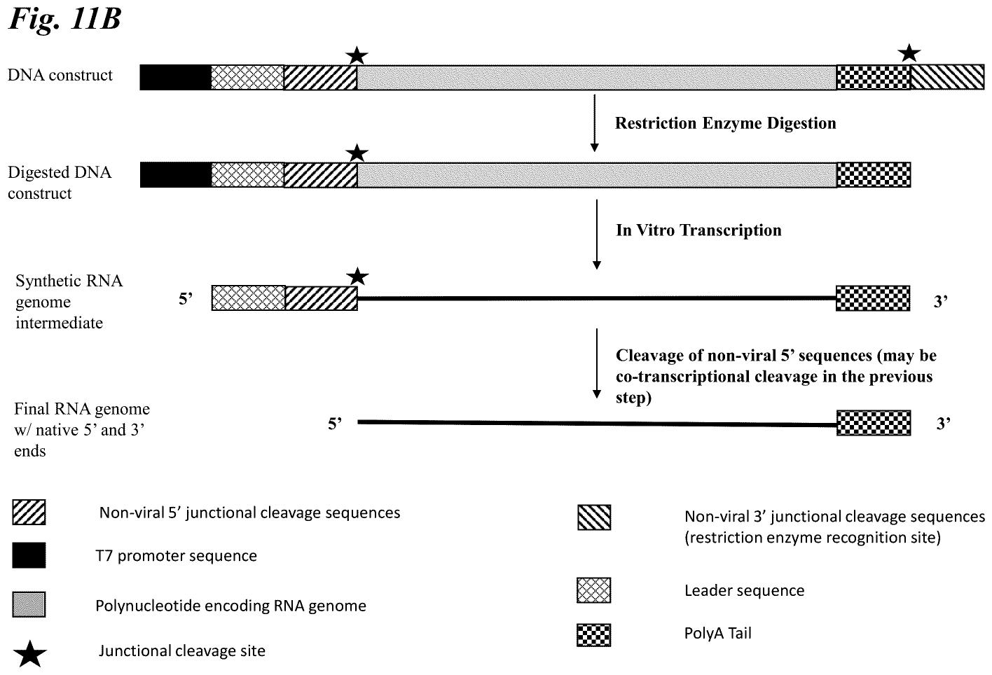

In some embodiments, the synthetic RNA viral genomes described herein encode a coxsackievirus. In some embodiments, the coxsackievirus is selected from CVB3, CVA21, and CVA9. The nucleic acid sequences of exemplary coxsackieviruses are provided GenBank Reference No. M33854.1 (CVB3), GenBank Reference No. KT161266.1 (CVA21), and GenBank Reference No. D00627.1 (CVA9). In some embodiments, the synthetic RNA viral genomes described herein encode a modified CVA21 virus comprising SEQ ID NO: 1, which is a Kuykendall (Kuyk) strain. In some embodiments, the sequence of the viral genome of the Kuykendall strain is according to GenBank Accession Number AF465515.1 or AF546702.1. In some embodiments, the synthetic RNA viral genomes described herein encode a chimeric coxsackievirus. In some embodiments, the synthetic RNA viral genomes described herein encode a CVA21 strain selected from the CVA21-EF strain and the CVA21-KY strain. In some embodiments, the synthetic RNA viral genomes described herein encode a CVA21-EF strain. An exemplary sequence of the viral genome of the EF strain is according to GenBank Accession Number EF015029.1. In some embodiments, the synthetic RNA viral genomes described herein encode a CVA21-KY strain. An exemplary sequence of the viral genome of the KY strain is according to GenBank Accession Number KY284011.1. As shown in FIGS. 11-26, the EF and KY strains provide therapeutic benefits over the Kuykendall lab strain and previously described synthetic picornavirus compositions.

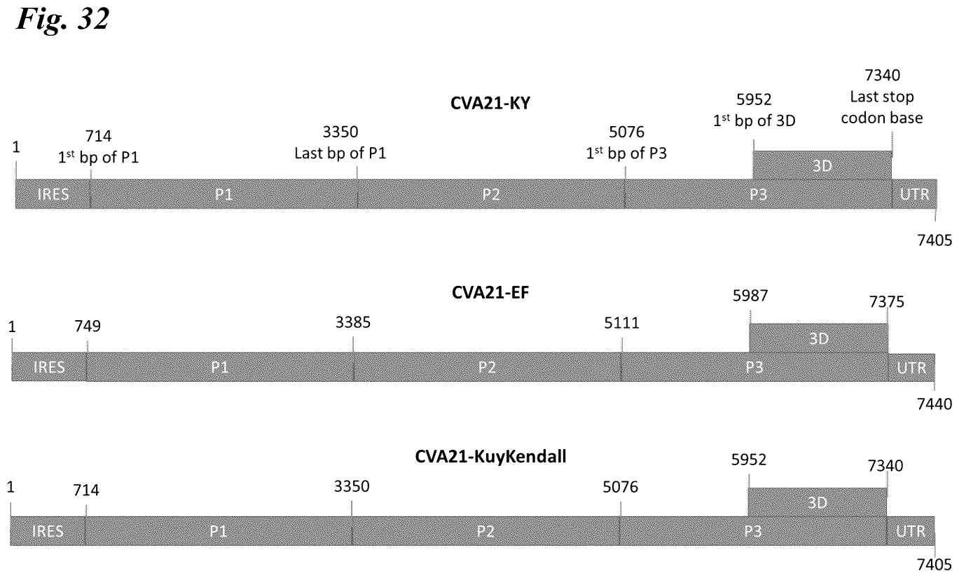

The domain organization of the three CVA21 strains (EF, KY, and Kuykendall) are provided in FIG. 32 and the sequence identities between various regions of these three strains are provided below in Table 2.

TABLE 2
Sequence Identity between the Corresponding Regions
of Different CVA21 Strains

| | Strain 1 | Strain 2 | Sequence |
|---|---|---|---|
| Region | (Sequence) | (Sequence) | Identity |
| Whole Virus | CVA21-KY | CVA21-Kuykendall | 88.8% |
| | (SEQ ID NO: 5) | (SEQ ID NO: 1) | |
| | CVA21-EF | CVA21-Kuykendall | 79.6% |
| | (SEQ ID NO: 9) | (SEQ ID NO: 1) | |
| | CVA21-EF | CVA21-KY | 79.4% |
| | (SEQ ID NO: 9) | (SEQ ID NO: 5) | |
| 5′ UTR | CVA21-KY | CVA21-Kuykendall | 91.2% |
| (IRES) | (SEQ ID NO: 6) | (SEQ ID NO: 2) | |
| | CVA21-EF | CVA21-Kuykendall | 85.0% |
| | (SEQ ID NO: 10) | (SEQ ID NO: 2) | |
| | CVA21-EF | CVA21-KY | 86.1% |
| | (SEQ ID NO: 10) | (SEQ ID NO: 6) | |
| P1 | CVA21-KY | CVA21-Kuykendall | 87.5% |
| | (SEQ ID NO: 7) | (SEQ ID NO: 3) | |
| | CVA21-EF | CVA21-Kuykendall | 78.6% |
| | (SEQ ID NO: 11) | (SEQ ID NO: 3) | |
| | CVA21-EF | CVA21-KY | 78.3% |
| | (SEQ ID NO: 11) | (SEQ ID NO: 7) | |
| 3D | CVA21-KY | CVA21-Kuykendall | 89.9% |
| | (SEQ ID NO: 8) | (SEQ ID NO: 4) | |
| | CVA21-EF | CVA21-Kuykendall | 82.7% |
| | (SEQ ID NO: 12) | (SEQ ID NO: 4) | |
| | CVA21-EF | CVA21-KY | 82.5% |
| | (SEQ ID NO: 12) | (SEQ ID NO: 8) | |

One or more specific regions in the viral genome of the CVA21 EF or KY strain may contribute to the beneficial therapeutic effect observed for the EF and KY strains over the Kuykendall lab strain. In some embodiments, the one or more specific regions are selected from the group consisting of the 5′ UTR (IRES) region, the P1 region, and the 3D region. The nucleic acid positions of each of these specific regions for the virus strains described herein are as follows:
(a) The 5′ UTR (IRES) region of CVA21-Kuykendall encompasses nucleic acids 1-713 of SEQ ID NO: 1. The 5′ UTR (IRES) region of CVA21-KY encompasses nucleic acids 1-713 of SEQ ID NO: 5. The 5′ UTR (IRES) region of CVA21-EF encompasses nucleic acids 1-748 of SEQ ID NO: 9.
(b) The P1 region of CVA21-Kuykendall encompasses nucleic acids 714-3350 of SEQ ID NO: 1. The P1 region of CVA21-KY encompasses nucleic acids 714-3350 of SEQ ID NO: 5. The P1 region of CVA21-EF encompasses nucleic acids 749-3385 of SEQ ID NO: 9.
(c) The 3D region of CVA21-Kuykendall encompasses nucleic acids 5952-7340 of SEQ ID NO: 1. The 3D region of CVA21-KY encompasses nucleic acids 5952-7340 of SEQ ID NO: 5. The 3D region of CVA21-EF encompasses nucleic acids 5987-7375 of SEQ ID NO: 9.

In some embodiments, the synthetic RNA viral genome described herein encodes a CVA21-KY strain. In some embodiments, the synthetic RNA viral genome encoding the CVA21-KY strain comprises a polynucleotide sequence according to SEQ ID NO: 5. In some embodiments, the synthetic RNA viral genome encoding the CVA21-KY strain comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 5. In some embodiments, the synthetic RNA viral genome encoding the CVA21-KY strain comprises a polynucleotide sequences that is less than 95%, less than 90%, less than 85%, or less than 80% identical (including all ranges and subranges therebetween) to SEQ ID NO: 1.

In some embodiments, the synthetic RNA viral genome described herein encodes a CVA21-KY strain and comprises a 5′ UTR (IRES) sequence according to SEQ ID NO: 6 (corresponding to nucleic acids 1-713 of SEQ ID NO: 5). In some embodiments, the synthetic RNA viral genome described herein encodes a CVA21-KY strain and comprises a 5′ UTR (IRES) sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 6. In some embodiments, the synthetic RNA viral genome encoding the CVA21-KY strain comprises a 5′ UTR (IRES) sequence that is less than 95%, less than 90%, less than 85%, or less than 80% (including all ranges and subranges therebetween) identical to SEQ ID NO: 2.

In some embodiments, the synthetic RNA viral genome described herein encodes a CVA21-KY strain and comprises a P1 sequence according to SEQ ID NO: 7 (corresponding to nucleic acids 714-3350 of SEQ ID NO: 5). In some embodiments, the synthetic RNA viral genome described herein encodes a CVA21-KY strain and comprises a P1 sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 7. In some embodiments, the synthetic RNA viral genome encoding the CVA21-KY strain comprises a P1 sequence that is less than 95%, less than 90%, less than 85%, or less than 80% (including all ranges and subranges therebetween) identical to SEQ ID NO: 3.

In some embodiments, the synthetic RNA viral genome described herein encodes a CVA21-KY strain and comprises a 3D sequence according to SEQ ID NO: 8 (corresponding to nucleic acids 5952-7340 of SEQ ID NO: 5). In some embodiments, the synthetic RNA viral genome described herein encodes a CVA21-KY strain and comprises a 3D sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 8. In some embodiments, the synthetic RNA viral genome encoding the CVA21-KY strain comprises a 3D sequence that is less than 95%, less than 90%, less than 85%, or less than 80% (including all ranges and subranges therebetween) identical to SEQ ID NO: 4.

In some embodiments, the synthetic RNA viral genome described herein encodes a CVA21-EF strain. In some embodiments, the synthetic RNA viral genome encoding the CVA21-EF strain comprises a polynucleotide sequence according to SEQ ID NO: 9. In some embodiments, the synthetic RNA viral genome encoding the CVA21-EF strain comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 9. In some embodiments, the synthetic RNA viral genome encoding the CVA21-EF strain comprises a polynucleotide sequences that is less than 95%, less than 90%, less than 85%, or less than 80% (including all ranges and subranges therebetween) identical to SEQ ID NO: 1.

In some embodiments, the synthetic RNA viral genome described herein encodes a CVA21-EF strain and comprises a 5′ UTR (IRES) sequence according to SEQ ID NO: 10 (corresponding to nucleic acids 1-748 of SEQ ID NO: 9). In some embodiments, the synthetic RNA viral genome described herein encodes a CVA21-EF strain and comprises a 5′ UTR (IRES) sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 10. In some embodiments, the synthetic RNA viral genome encoding the CVA21-EF strain comprises a 5′ UTR (IRES) sequence that is less than 95%, less than 90%, less than 85%, or less than 80% (including all ranges and subranges therebetween) identical to SEQ ID NO: 2.

In some embodiments, the synthetic RNA viral genome described herein encodes a CVA21-EF strain and comprises a P1 sequence according to SEQ ID NO: 11 (corresponding to nucleic acids 749-3385 of SEQ ID NO: 9). In some embodiments, the synthetic RNA viral genome described herein encodes a CVA21-EF strain and comprises a P1 sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 11. In some embodiments, the synthetic RNA viral genome encoding the CVA21-EF strain comprises a P1 sequence that is less than 95%, less than 90%, less than 85%, or less than 80% (including all ranges and subranges therebetween) identical to SEQ ID NO: 3.

In some embodiments, the synthetic RNA viral genome described herein encodes a CVA21-EF strain and comprises a 3D sequence according to SEQ ID NO: 12 (corresponding to nucleic acids 5987-7375 of SEQ ID NO: 9). In some embodiments, the synthetic RNA viral genome described herein encodes a CVA21-EF strain and comprises a 3D sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 12. In some embodiments, the synthetic RNA viral genome encoding the CVA21-EF strain comprises a 3D sequence that is less than 95%, less than 90%, less than 85%, or less than 80% (including all ranges and subranges therebetween) identical to SEQ ID NO: 4.

In some embodiments, the CVA21 RNA viral genome described herein does not comprise the nucleotide sequence CGUCUC (SEQ ID NO: 83) or GAGACG (SEQ ID NO: 84). The corresponding, complementary DNA sequences, CGTCTC (SEQ ID NO: 85) and GAGACG (SEQ ID NO: 86), are BsmBI restriction enzyme recognition sites.

In some embodiments, the CVA21 RNA viral genome described herein does not comprise the nucleotide sequence GGUCUC (SEQ ID NO: 87) or GAGACC (SEQ ID NO: 88). The corresponding, complementary DNA sequences, GGTCTC (SEQ ID NO: 89) and GAGACC (SEQ ID NO: 90), are BsaI restriction enzyme recognition sites.

In some embodiments, the synthetic RNA viral genomes described herein encode a Seneca Valley virus (SVV). In some embodiments, the SVV is selected from a wild-type SVV (such as SVV-001, SEQ ID NO: 25) or a mutant SVV or a chimeric SVV (such as SVV-001-S177A encoded by SEQ ID NO: 26; or SVV-IRES2-S177A encoded by SEQ ID NO: 68 or SEQ ID NO: 73).

In some embodiments, the SVV is a SVV-S177 mutant. In some embodiments, the SVV is an SVV-S177A mutant. As used herein in relation to the SVV viral genome, the term “S177 mutant” refers to a SVV viral genome encoding a VP2 protein comprising a mutation at amino acid S177 of the wildtype protein (amino acid numbering according to the VP2 protein encoded by SEQ ID NO: 25). Accordingly, the term “S177A mutant” refers to a SVV mutant having an amino acid substitution of S177A of the VP2 protein. In SEQ ID NO: 25, the VP2 S177 residue is encoded by the codon “UCU” at nucleic acid position 1645-1647. Accordingly, the SVV-S177 mutant comprises a nucleic acid mutation within the region corresponding to nucleic acid position 1645-1647 of SEQ ID NO: 25. In some embodiments, the SVV-S177A mutant comprises the codon sequence “GCU”, “GCC”, “GCA” or “GCG” at the region corresponding to nucleic acid position 1645-1647 of SEQ ID NO: 25. In some embodiments, the SVV-S177A mutant comprises the codon sequence “GCG” at the region corresponding to nucleic acid position 1645-1647 of SEQ ID NO: 25.

In some embodiments, the SVV RNA viral genome described herein does not comprise the nucleotide sequence GCUCUUC (SEQ ID NO: 79) or GAAGAGC (SEQ ID NO: 80). The corresponding, complementary DNA sequences, GCTCTTC (SEQ ID NO: 81) and GAAGAGC (SEQ ID NO: 82), are SapI restriction enzyme recognition sites. In some embodiments, a wildtype SVV RNA viral genome comprises SEQ ID NO: 79 at the position corresponding to nucleic acids 1504-1510 and/or nucleic acids 5293-5299 of SEQ ID NO: 25. In some embodiments, the SVV RNA viral genome of the disclosure comprises at least 1 nucleotide substitution as compared to SEQ ID NO: 79 within the region corresponding to nucleic acids 1504-1510 and/or nucleic acids 5293-5299 of SEQ ID NO: 25. In some embodiments, the at least 1 nucleotide substitution is a silent mutation that does not change the amino acids encoded by the corresponding region of the DNA. In some embodiments, the SVV RNA viral genome of the disclosure comprises a cytidine (“C”) at the position corresponding to nucleic acid 1509 and/or 5298 of SEQ ID NO: 25.

In some embodiments, the SVV RNA viral genome described herein does not comprise the nucleotide sequence GGUCUC (SEQ ID NO: 87) or GAGACC (SEQ ID NO: 88). The corresponding, complementary DNA sequences, GGTCTC (SEQ ID NO: 89) and GAGACC (SEQ ID NO: 90), are BsaI restriction enzyme recognition sites.

In some embodiments, the synthetic RNA viral genomes described herein encode a chimeric picornavirus (e.g., encode a virus comprising one portion, such as a capsid protein or an IRES, derived from a first picornavirus and another portion, such as a non-structural gene such as a protease or polymerase derived from a second picornavirus). In some embodiments, the synthetic RNA viral genomes described herein encode a chimeric SVV.

In some embodiments, the synthetic RNA viral genome described herein encodes a SVV comprising one or more specific regions derived from an SVV strain selected from the group consisting of SVV-001 (SEQ ID NO: 25 or SEQ ID NO: 72 (Genbank ID No.: DQ641257.1)), SVA/BRA/MG2/2015 (SEQ ID NO: 69; GenBank ID No.: KR063108.1), SVA/Canada/MB/NCFAD-104/2015 (SEQ ID NO: 70; GenBank ID No.: KY486156.1), and SVV-MN15-308 (SEQ ID NO: 71; GenBank ID No.: KU359214.1). In some embodiments, the one or more specific regions are selected from the group consisting of the 5′ UTR (IRES) region, the P1 region, and the P3 region. The nucleic acid positions of each of these specific regions for the virus strains described herein are as follows:
(a) The 5′ UTR (IRES) region of SVV-001 encompasses nucleic acids 1-668 of SEQ ID NO: 25. The 5′ UTR (IRES) region of SVA/BRA/MG2/2015 encompasses nucleic acids 1-656 of SEQ ID NO: 69. The 5′ UTR (IRES) region of SVA/Canada/MB/NCFAD-104/2015 encompasses nucleic acids 1-612 of SEQ ID NO: 70. The 5′ UTR (IRES) region of SVV-MN15-308 encompasses nucleic acids 1-610 of SEQ ID NO: 71.
(b) The P1 region of SVV-001 encompasses nucleic acids 669-3477 of SEQ ID NO: 25. The P1 region of SVA/BRA/MG2/2015 encompasses nucleic acids 657-3465 of SEQ ID NO: 69. The P1 region of SVA/Canada/MB/NCFAD-104/2015 encompasses nucleic acids 613-3421 of SEQ ID NO: 70. The P1 region of SVV-MN15-308 encompasses nucleic acids 611-3419 of SEQ ID NO: 71.
(c) The P3 region of SVV-001 encompasses nucleic acids 4855-7212 of SEQ ID NO: 25. The P3 region of SVA/BRA/MG2/2015 encompasses nucleic acids 4843-7200 of SEQ ID NO: 69. The P3 region of SVA/Canada/MB/NCFAD-104/2015 encompasses nucleic acids 4799-7156 of SEQ ID NO: 70. The P3 region of SVV-MN15-308 encompasses nucleic acids 4797-7154 of SEQ ID NO: 71.

In some embodiments, the synthetic RNA viral genome described herein encodes a SVV comprising a 5′ UTR (IRES) region derived from SVA/BRA/MG2/2015 (nucleic acids 1-656 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-656 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a 5′ UTR (IRES) region derived from SVA/Canada/MB/NCFAD-104/2015 (nucleic acids 1-612 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-612 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a 5′ UTR (IRES) region derived from SVV-MN15-308 (nucleic acids 1-610 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 1-610 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVA/BRA/MG2/2015 (nucleic acids 657-3465 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 657-3465 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVA/Canada/MB/NCFAD-104/2015 (nucleic acids 613-3421 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 613-3421 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P1 region derived from SVV-MN15-308 (nucleic acids 611-3419 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 611-3419 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVA/BRA/MG2/2015 (nucleic acids 4843-7200 of SEQ ID NO: 69). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4843-7200 of SEQ ID NO: 69. In some embodiments, other than the one or more regions derived from SVA/BRA/MG2/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVA/Canada/MB/NCFAD-104/2015 (nucleic acids 4799-7156 of SEQ ID NO: 70). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4799-7156 of SEQ ID NO: 70. In some embodiments, other than the one or more regions derived from SVA/Canada/MB/NCFAD-104/2015, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a SVV comprising a P3 region derived from SVV-MN15-308 (nucleic acids 4797-7154 of SEQ ID NO: 71). In some embodiments, the synthetic RNA viral genome encoding the SVV comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to nucleic acids 4797-7154 of SEQ ID NO: 71. In some embodiments, other than the one or more regions derived from SVV-MN15-308, the rest of the SVV viral genome is derived from SVV-001 and comprises a polynucleotide sequence having at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to the corresponding region of SEQ ID NO: 25. In some embodiments, the SVV is a SVV-S177 mutant (e.g., a S177A mutant).

In some embodiments, the synthetic RNA viral genome described herein encodes a chimeric SVV comprising a 5′ UTR (IRES) region derived from SVA/Canada/MB/NCFAD-104/2015 (SEQ ID NO: 70) and the rest of the viral genome derived from SVV-001 (SEQ ID NO: 25). In some embodiments, the SVV is an SVV-S177 mutant (e.g., a S177A mutant). In some embodiments, the synthetic RNA viral genome has at least 80%, at least 85%, at least 90%, at least 91%, at least 92%, at least 93%, at least 94%, at least 95%, at least 96%, at least 97%, at least 98%, at least 99%, at least 99.5%, at least 99.8%, at least 99.9%, or 100% (including all ranges and subranges therebetween) sequence identity to SEQ ID NO: 68.

In some embodiments, the synthetic RNA viral genome has been engineered and comprises less than 100% sequence identity to that of a wildtype virus (e.g., a wildtype CVA21 or a wildtype SVV). In some embodiments, the synthetic RNA viral genome comprises less than 99.9%, less than 99.8%, less than 99.7%, less than 99.6%, less than 99.5%, less than 99%, less than 98%, less than 97%, less than 96%, less than 95%, less than 94%, less than 93%, less than 92%, less than 91%, or less than 90%, sequence identity to that of a corresponding wildtype virus.

In some embodiments, the synthetic RNA viral genome comprises a microRNA (miRNA) target sequence (miR-TS) cassette, wherein the miR-TS cassette comprises one or more miRNA target sequences, and wherein expression of one or more of the corresponding miRNAs in a cell inhibits replication of the encoded oncolytic virus in the cell. In some embodiments, the one or more miRNAs are selected from miR-124, miR-1, miR-143, miR-128, miR-219, miR-219a, miR-122, miR-204, miR-217, miR-137, miR-142, and miR-126. In some embodiments, the miR-TS cassette comprises one or more copies of a miR-124 target sequence, one or more copies of a miR-1 target sequence, and one or more copies of a miR-143 target sequence. In some embodiments, the miR-TS cassette comprises one or more copies of a miR-128 target sequence, one or more copies of a miR-219a target sequence, and one or more copies of a miR-122 target sequence. In some embodiments, the miR-TS cassette comprises one or more copies of a miR-128 target sequence, one or more copies of a miR-204 target sequence, and one or more copies of a miR-219 target sequence. In some embodiments, the miR-TS cassette comprises one or more copies of a miR-217 target sequence, one or more copies of a miR-137 target sequence, and one or more copies of a miR-126 target sequence.

In some embodiments, the synthetic RNA viral genome comprises one or more miR-TS cassettes is incorporated into the 5′ untranslated region (UTR) or 3′ UTR of one or more essential viral genes. In some embodiments, the synthetic RNA viral genome comprises one or more miR-TS cassettes is incorporated into the 5′ untranslated region (UTR) or 3′ UTR of one or more non-essential genes. In some embodiments, the synthetic RNA viral genome comprises one or more miR-TS cassettes is incorporated 5′ or 3′ of one or more essential viral genes.

In some embodiments, the synthetic RNA viral genome comprises a heterologous polynucleotide encoding a payload molecule. In such embodiments, the synthetic RNA viral genome drives production of an infectious oncolytic virus as well as expression of the payload molecule. In some embodiments, the expression of the payload molecule can increase the therapeutic efficacy of the oncolytic virus. In some embodiments, the payload molecule is selected from IL-12, GM-CSF, CXCL10, IL-36γ, CCL21, IL-18, IL-2, CCL4, CCL5, an anti-CD3-anti-FAP BiTE, an antigen binding molecule that binds DLL3, or an antigen binding molecule that binds EpCAM. In some embodiments, the payload molecule comprises or consists of MLKL 4HB domain. In some embodiments, the payload molecule comprises or consists of Gasdermin D N-terminal fragment. In some embodiments, the payload molecule comprises or consists of Gasdermin E N-terminal fragment. In some embodiments, the payload molecule comprises or consists of HMGB1 Box B domain. In some embodiments, the payload molecule comprises or consists of SMAC/Diablo. In some embodiments, the payload molecule comprises or consists of Melittin. In some embodiments, the payload molecule comprises or consists of L-amino-acid oxidase (LAAO). In some embodiments, the payload molecule comprises or consists of disintegrin. In some embodiments, the payload molecule comprises or consists of TRAIL (TNFSF10). In some embodiments, the payload molecule comprises or consists of a nitroreductase (e.g., E. coli NfsB or NfsA). In some embodiments, the payload molecule comprises or consists of a reovirus FAST protein (e.g., ARV p14, BRV p15, or p14-p15 hybrid). In some embodiments, the payload molecule comprises or consists of a leptin/FOSL2. In some embodiments, the payload molecule comprises or consists of an α-1,3-galactosyltransferase. In some embodiments, the payload molecule comprises or consists of an adenosine deaminase 2 (ADA2). In some embodiments, the paylod molecule comprises or consists of a cytokine selected from IL-IL-36γ, IL-7, IL-12, IL-18, IL-21, IL2 or IFNγ. Further description of the types of payload molecules suitable for use in these embodiments is provided below.

### Methods of Producing Recombinant RNA Viral Genomes

In some embodiments, the disclosure provides recombinant DNA molecules encoding the synthetic RNA viral genomes described herein. Such recombinant DNA molecules are referred to herein as “DNA templates” or “recombinant DNA templates”. In some embodiments, the recombinant DNA molecules are used as templates for in vitro transcription of the encoded synthetic RNA viral genomes. In some embodiments, the recombinant DNA molecules (e.g., DNA templates) comprises, from 5′ to 3′, one or more of the following elements: (i) a promoter; (ii) a 5′ leader sequence; (iii) a 5′ junctional cleavage sequence; (iv) a DNA polynucleotide sequence encoding the synthetic RNA genome; (v) a polyA tail; and/or (vi) a 3′ junctional cleavage sequence. In some embodiments, the recombinant DNA molecules (e.g., DNA templates) encoding the recombinant RNA molecule comprises each of the following elements: (i) a promoter; (ii) a 5′ leader sequence; (iii) a 5′ junctional cleavage sequence; (iv) a DNA polynucleotide sequence encoding the synthetic RNA genome; (v) a polyA tail; and (vi) a 3′ junctional cleavage sequence. Each of these elements are described in detail below. The description provided for each individual element is such that the specific embodiments of each element can be combined into the final recombinant DNA molecules (e.g., DNA templates). For example, the disclosure of a specific leader sequence can be combined with the disclosure of a specific 5′ junctional cleavage sequence, etc.

In some embodiments, the recombinant DNA molecules (e.g., DNA templates) do not comprise additional nucleic acids between two adjacent elements but may comprise additional nucleic acids upstream to the promoter sequence or downstream to the 3′ junctional cleavage sequence. In some embodiments, the promoter sequence is a T7 promoter sequence. In some embodiments, the T7 promoter sequence comprises or consists of SEQ ID NO: 91.

In some embodiments, the synthetic RNA viral genomes described herein are produced in vitro using one or more recombinant DNA templates comprising a polynucleotide encoding the synthetic RNA viral genomes. In other words, the recombinant DNA templates are vectors comprising the polynucleotide encoding the synthetic RNA viral genomes. The term “vector” is used herein to refer to a nucleic acid molecule capable of transferring, encoding, or transporting another nucleic acid molecule. The transferred nucleic acid is generally inserted into the vector nucleic acid molecule. A vector may include sequences that direct autonomous replication in a cell and/or may include sequences sufficient to allow integration into host cell DNA. In some embodiments, the recombinant RNA molecule encoding an oncolytic virus described herein is produced using one or more DNA vectors.

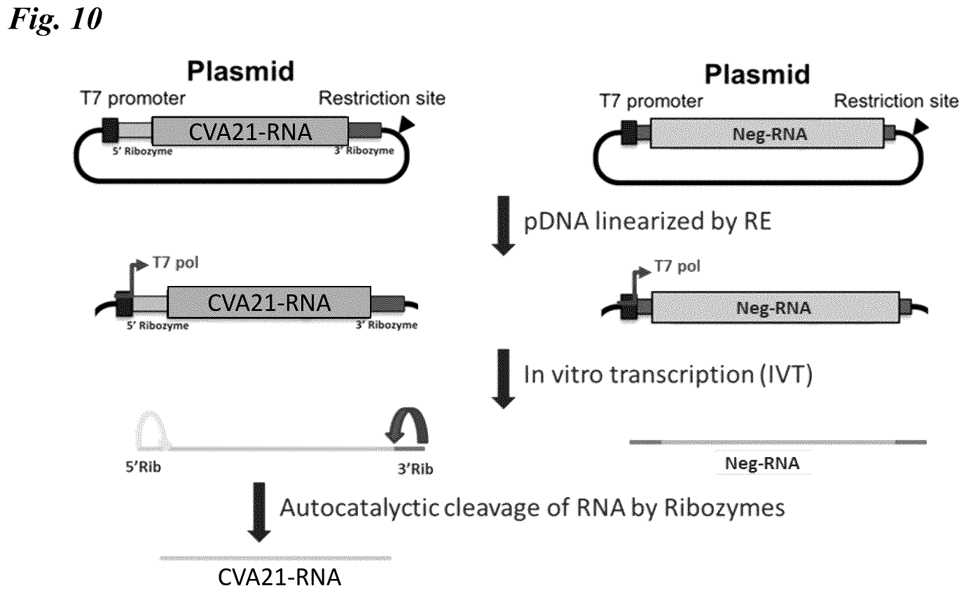

In some embodiments, the synthetic RNA viral genomes described herein are produced by introducing a recombinant DNA molecule (e.g., DNA template) comprising a polynucleotide encoding the recombinant RNA molecule (e.g., by means of transfection, transduction, electroporation, and the like) into a suitable host cell in vitro. Suitable host cells include insect and mammalian cell lines. The host cells are cultured for an appropriate amount of time to allow expression of the polynucleotides and production of the synthetic RNA viral genomes. The synthetic RNA viral genomes are then isolated from the host cell and formulated for therapeutic use (e.g., encapsulated in a particle). A schematic of the in vitro synthesis of the CVA21 RNA viral genomes with 3′ and 5′ ribozymes is shown in FIG. 10. The same schematic applies to the synthesis of RNA viral genomes (e.g., CVA21 or SVV viral genomes) using other combinations of junctional cleavage sequences (See e.g. FIG. 11A). When the 3′ junctional cleavage sequence comprises or consists of a restriction enzyme recognition site, the recombinant DNA molecule (e.g., DNA template) may be digested with the corresponding restriction enzyme before the in vitro transcription process, as shown in FIG. 11B.

### T7 Promoter

In some embodiments, the recombinant DNA molecule (e.g., DNA template) comprises a T7 promoter. In some embodiments, the T7 promoter comprises or consists of a polynucleotide sequence of SEQ ID NO: 91. In some embodiments, the T7 promoter comprises or consists of a polynucleotide sequence of SEQ ID NO: 91 with at most 1, 2, 3, or 4 mutations.

In some embodiments, the T7 promoter is placed immediately before the leader sequence, with no additional nucleotides in between. In some embodiments, the T7 promoter is placed immediately before the 5′ junctional cleavage sequence, with no additional nucleotides in between. In some embodiments, the viral genome encodes CVA21 or SVV.

### Junctional Cleavage Sequences

In some embodiments, the recombinant RNA molecules comprising the synthetic RNA viral genomes described herein require discrete 5′ and 3′ ends that are native to the virus. The RNA transcripts produced by T7 RNA polymerase in vitro or by mammalian RNA Pol II contain mammalian 5′ and 3′ UTRs do not contain the discrete, native ends required for production of an infectious RNA virus. For example, the T7 RNA polymerase requires a guanosine residue on the 5′ end of the template polynucleotide in order to initiate transcription. However, SVV begins with a uridine residue on its 5′ end. Thus, the T7 leader sequence, which is required for in vitro transcription of the SVV transcript must be removed to generate the native 5′ SVV terminus required for production of a functional infectious SVV. Therefore, in some embodiments, recombinant DNA molecules (e.g., DNA templates) suitable for use in the production of the synthetic RNA viral genomes described herein require additional non-viral 5′ and 3′ sequences that enable generation of the discrete 5′ and 3′ ends native to the virus. Such sequences are referred to herein as junctional cleavage sequences (JCS). In some embodiments, the junctional cleavage sequences act to cleave the T7 RNA polymerase or Pol II-encoded RNA transcript at the junction of the viral RNA and the mammalian mRNA sequence such that the non-viral RNA polynucleotides are removed from the transcript in order to maintain the endogenous 5′ and 3′ discrete ends of the virus (See schematic shown in FIG. 11A). In some embodiments, the junctional cleavage sequences act to generate the appropriate ends during the linearization of the DNA plasmid encoding the synthetic viral genome (e.g., the use of 3′ restriction enzyme recognition sequences to produce the appropriate 3′ end upon linearization of the plasmid template and prior to in vitro transcription of the synthetic RNA genome).

In some embodiments, the recombinant DNA molecules (e.g., DNA templates) suitable for use in the production of the synthetic RNA viral genomes described herein comprise at least one 5′ junctional cleavage sequence and at least one 3′ junctional cleavage sequence. In some embodiments, the recombinant DNA molecules (e.g., DNA templates) suitable for use in the production of the synthetic RNA viral genomes described herein comprise one or more 5′ junctional cleavage sequences and at least one 3′ junctional cleavage sequence. In some embodiments, the recombinant DNA molecules (e.g., DNA templates) suitable for use in the production of the synthetic RNA viral genomes described herein comprise at least one 5′ junctional cleavage sequence and one or more 3′ junctional cleavage sequences. In some embodiments, the recombinant DNA molecules (e.g., DNA templates) suitable for use in the production of the synthetic RNA viral genomes described herein comprise one or more 5′ junctional cleavage sequences and one or more 3′ junctional cleavage sequences. In some embodiments, the recombinant DNA molecules (e.g., DNA templates) suitable for use in the production of the synthetic RNA viral genomes described herein comprise two 5′ junctional cleavage sequences and at least one 3′ junctional cleavage sequence. In some embodiments, the recombinant DNA molecules (e.g., DNA templates) suitable for use in the production of the synthetic RNA viral genomes described herein comprise at least one 5′ junctional cleavage sequence and two 3′ junctional cleavage sequences.

The nature of the junctional cleavage sequences and the removal of the non-viral RNA from the viral genome transcript can be accomplished by a variety of methods. For example, in some embodiments, the junctional cleavage sequences are targets for RNA interference (RNAi) molecules. “RNA interference molecule” as used herein refers to an RNA polynucleotide that mediates degradation of a target mRNA sequence through endogenous gene silencing pathways (e.g., Dicer and RNA-induced silencing complex (RISC)). Exemplary RNA interference agents include micro RNAs (miRNAs), artificial miRNA (amiRNAs), short hairpin RNAs (shRNAs), and small interfering RNAs (siRNAs). Further, any system for cleaving an RNA transcript at a specific site currently known the art or to be defined in the future can be used to generate the discrete ends native to the virus.

In some embodiments, the RNAi molecule is a miRNA. A miRNA refers to a naturally-occurring, small non-coding RNA molecule of about 18-25 nucleotides in length that is at least partially complementary to a target mRNA sequence. In animals, genes for miRNAs are transcribed to a primary miRNA (pri-miRNA), which is double stranded and forms a stem-loop structure. Pri-miRNAs are then cleaved in the nucleus by a microprocessor complex comprising the class 2 RNase III, Drosha, and the microprocessor subunit, DCGR8, to form a 70-100 nucleotide precursor miRNA (pre-miRNA). The pre-miRNA forms a hairpin structure and is transported to the cytoplasm where it is processed by the RNase III enzyme, Dicer, into a miRNA duplex of ˜18-25 nucleotides. Although either strand of the duplex may potentially act as a functional miRNA, typically one strand of the miRNA is degraded and only one strand is loaded onto the Argonaute (AGO) nuclease to produce the effector RNA-induced silencing complex (RISC) in which the miRNA and its mRNA target interact (Wahid et al., 1803:11, 2010, 1231-1243). In some embodiments, the 5′ and/or 3′ junctional cleavage sequences are miRNA target sequences.

In some embodiments, the RNAi molecule is an artificial miRNA (amiRNA) derived from a synthetic miRNA-embedded in a Pol II transcript. (See e.g., Liu et al., Nucleic Acids Res (2008) 36:9; 2811-2834; Zeng et al., Molecular Cell (2002), 9; 1327-1333; Fellman et al., Cell Reports (2013) 5; 1704-1713). In some embodiments, the 5′ and/or 3′ junctional cleavage sequences are amiRNA target sequences.

In some embodiments, the RNAi molecule is an siRNA molecule. siRNAs refer to double stranded RNA molecules typically about 21-23 nucleotides in length. The duplex siRNA molecule is processed in the cytoplasm by the associates with a multi protein complex called the RNA-induced silencing complex (RISC), during which the “passenger” sense strand is enzymatically cleaved from the duplex. The antisense “guide” strand contained in the activated RISC then guides the RISC to the corresponding mRNA by virtue of sequence complementarity and the AGO nuclease cuts the target mRNA, resulting in specific gene silencing. In some embodiments, the siRNA molecule is derived from an shRNA molecule. shRNAs are single stranded artificial RNA molecules ˜50-70 nucleotides in length that form stem-loop structures. Expression of shRNAs in cells is accomplished by introducing a DNA polynucleotide encoding the shRNA by plasmid or viral vector. The shRNA is then transcribed into a product that mimics the stem-loop structure of a pre-miRNA, and after nuclear export the hairpin is processed by Dicer to form a duplex siRNA molecule which is then further processed by the RISC to mediate target-gene silencing. In some embodiments, the 5′ and/or 3′ junctional cleavage sequences are siRNA target sequences.

In some embodiments, the junctional cleavage sequences are guide RNA (gRNA) target sequences. In such embodiments, gRNAs can be designed and introduced with a Cas endonuclease with RNase activity (e.g., Cas13) to mediate cleavage of the viral genome transcript at the precise junctional site. In some embodiments, the 5′ and/or 3′ junctional cleavage sequences are gRNA target sequences.

In some embodiments, the junctional cleavage sequences are pri-miRNA-encoding sequences. Upon transcription of the polynucleotide encoding the viral genome (e.g., the recombinant RNA molecule), these sequences form the pri-miRNA stem-loop structure which is then cleaved in the nucleus by Drosha to cleave the transcript at the precise junctional site. In some embodiments, the 5′ and/or 3′ junctional cleavage sequences are pri-mRNA target sequences.

In some embodiments, the junctional cleavage sequences are primer binding sequences that facilitate cleavage by the endoribonuclease, RNAseH. In such embodiments, a primer that anneals to the 5′ and/or 3′ junctional cleavage sequence is added to the in vitro reaction along with an RNAseH enzyme. RNAseH specifically hydrolyzes the phosphodiester bonds of RNA which is hybridized to DNA, therefore enabling cleavage of the synthetic RNA genome intermediates at the precise junctional cleavage sequence to produce the required 5′ and 3′ native ends.

In some embodiments, the junctional cleavage sequences comprise or consist of restriction enzyme recognition sites and result in the generation of discrete ends of viral transcripts during linearization of the plasmid template runoff RNA synthesis with T7 RNA Polymerase. In some embodiments, the junctional cleavage sequences are Type IIS restriction enzyme recognition sites. Type IIS restriction enzymes comprise a specific group of enzymes which recognize asymmetric DNA sequences and cleave at a defined distance outside of their recognition sequence, usually within 1 to 20 nucleotides. Exemplary Type IIS restriction enzymes include AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBi, BsmFI, BsmI, BspCNI, BspMI, BspQI, BsrDI, BsrI, BtgZI, BtsCI, BstI, CaspCI, Earl, EciI, Esp3I, FauI, FokI, HgaI, HphI, HpyAV, MbolI, MlyI, MmeI, MnlL, NmeAIII, PleI, SapI, and SfaNI. The recognition sequences for these Type IIS restriction enzymes are known in the art. See the New England Biolabs website located at neb.com/tools-and-resources/selection-charts/type-iis-restriction-enzymes. In some embodiments, the junctional cleavage sequence comprises a SapI restriction enzyme recognition site. In some embodiments, the junctional cleavage sequence comprises a BsmBI restriction enzyme recognition site. In some embodiments, the junctional cleavage sequence comprises a BsaI restriction enzyme recognition site. A skilled person would understand that, because the cleavage site of the Type IIS restriction enzymes is typically outside the enzyme recognition site (e.g., offset by 1-5 nucleotides), the corresponding junctional cleavage sequence may also comprise the additional nucleotide(s) required by the corresponding restriction enzyme to create the discrete end of the viral transcript (e.g., the poly-A tail at 3′ end).

In some embodiments, the junctional cleavage sequences are sequences encoding ligand-inducible self-cleaving ribozymes, referred to as “aptazymes”. Aptazymes are ribozyme sequences that contain an integrated aptamer domain specific for a ligand. Ligand binding to the apatmer domain triggers activation of the enzymatic activity of the ribozyme, thereby resulting in cleavage of the RNA transcript. Exemplary aptazymes include theophylline-dependent aptazymes (e.g., hammerhead ribozyme linked to a theophylline-dependent apatmer, described in Auslander et al., Mol BioSyst. (2010) 6, 807-814), tetracycline-dependent aptazymes (e.g., hammerhead ribozyme linked to a Tet-dependent aptamer, described by Zhong et al., eLife 2016; 5:e18858 DOI: 10.7554/eLife.18858; Win and Smolke, PNAS (2007) 104; 14283-14288; Whittmann and Suess, Mol Biosyt (2011) 7; 2419-2427; Xiao et al., Chem & Biol (2008) 15; 125-1137; and Beilstein et al., ACS Syn Biol (2015) 4; 526-534), guanine-dependent aptazymes (e.g., hammerhead ribozyme linked to a guanine-dependent aptamer, described by Nomura et al., Chem Commun., (2012) 48(57); 7215-7217). In some embodiments, the 5′ and/or 3′ junctional cleavage sequences are aptazyme-encoding sequences.

In some embodiments, the junctional cleavage sequences are target sequences for an RNAi molecule (e.g., an siRNA molecule, an shRNA molecule, an miRNA molecule, or an amiRNA molecule), a gRNA molecule, or an RNAseH primer. In such embodiments, the junctional cleavage sequence is at least partially complementary to the sequence of the RNAi molecule, gRNA molecule, or primer molecule. Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In some embodiments, the 5′ junctional cleavage sequence and 3′ junctional cleavage sequence are from the same group (e.g., are both RNAi target sequences, both ribozyme-encoding sequences, etc.). For example, in some embodiments, the junctional cleavage sequences are RNAi target sequences (e.g., siRNA, shRNA, amiRNA, or miRNA target sequences) and are incorporated into the 5′ and 3′ ends of the polynucleotide encoding the viral genome (e.g., the recombinant RNA molecule). In such embodiments, the 5′ and 3′ RNAi target sequence may be the same (i.e., targets for the same siRNA, amiRNA, or miRNA) or different (i.e., the 5′ sequence is a target for one siRNA, shmiRNA, or miRNA and the 3′ sequence is a target for another siRNA, amiRNA, or miRNA). In some embodiments, the junctional cleavage sequences are guide RNA target sequences and are incorporated into the 5′ and 3′ ends of the polynucleotide encoding the viral genome (e.g., the recombinant RNA molecule). In such embodiments, the 5′ and 3′ gRNA target sequences may be the same (i.e., targets for the same gRNA) or different (i.e., the 5′ sequence is a target for one gRNA and the 3′ sequence is a target for another gRNA). In some embodiments, the junctional cleavage sequences are pri-mRNA-encoding sequences and are incorporated into the 5′ and 3′ ends of the polynucleotide encoding the viral genome (e.g., the recombinant RNA molecule). In some embodiments, the junctional cleavage sequences are ribozyme-encoding sequences and are incorporated immediately 5′ and 3′ of the polynucleotide sequence encoding the viral genome (e.g., the recombinant RNA molecule).

In some embodiments, the 5′ junctional cleavage sequence and 3′ junctional cleavage sequence are from the same group but are different variants or types. For example, in some embodiments, the 5′ and 3′ junctional cleavage sequences may be target sequences for an RNAi molecule, wherein the 5′ junctional cleavage sequence is an siRNA target sequence and the 3′ junctional cleavage sequence is a miRNA target sequence (or vis versa). In some embodiments, the 5′ and 3′ junctional cleavage sequences may be ribozyme-encoding sequences, wherein the 5′ junctional cleavage sequence is a hammerhead ribozyme-encoding sequence and the 3′ junctional cleavage sequence is a hepatitis delta virus ribozyme-encoding sequence.

In some embodiments, the 5′ junctional cleavage sequence and 3′ junctional cleavage sequence are different types. For example, in some embodiments, the 5′ junctional cleavage sequence is an RNAi target sequence (e.g., an siRNA, an amiRNA, or a miRNA target sequence) and the 3′ junctional cleavage sequence is a ribozyme sequence, an aptazyme sequence, a pri-miRNA sequence, or a gRNA target sequence. In some embodiments, the 5′ junctional cleavage sequence is a ribozyme sequence and the 3′ junctional cleavage sequence is an RNAi target sequence (e.g., an siRNA, an amiRNA, or a miRNA target sequence), an aptazyme sequence, a pri-miRNA-encoding sequence, or a gRNA target sequence. In some embodiments, the 5′ junctional cleavage sequence is an aptazyme sequence and the 3′ junctional cleavage sequence is an RNAi target sequence (e.g., an siRNA, an amiRNA, or a miRNA target sequence), a ribozyme sequence, a pri-miRNA sequence, or a gRNA target sequence. In some embodiments, the 5′ junctional cleavage sequence is a pri-miRNA sequence and the 3′ junctional cleavage sequence is an RNAi target sequence (e.g., an siRNA, an amiRNA, or a miRNA target sequence), a ribozyme sequence, an aptazyme sequence, or a gRNA target sequence. In some embodiments, the 5′ junctional cleavage sequence is a gRNA target sequence and the 3′ junctional cleavage sequence is an RNAi target sequence (e.g., an siRNA, an amiRNA, or a miRNA target sequence), a ribozyme sequence, a pri-miRNA sequence, or an aptazyme sequence.

Exemplary arrangements of the junctional cleavage sequences relative to the polynucleotide encoding the synthetic viral genome are shown below in Tables 3 and 4.

TABLE 3
Symmetrical Junctional Cleavage Sequence (JSC) Arrangements

| | | | |
|---|---|---|---|
| 5′ | JCS | JCS | 3′ |

| | | |
|---|---|---|
| siRNA TS | synthetic genome | siRNA TS |
| miR TS | synthetic genome | miR TS |
| AmiR TS | synthetic genome | AmiR TS |
| gRNA TS | synthetic genome | gRNA TS |
| pri-miR | synthetic genome | pri-miR |
| ribozyme | synthetic genome | ribozyme |
| aptazyme | synthetic genome | aptazyme |
| RNAseH primer TS | synthetic genome | RNAseH primer TS |

TABLE 4
Asymmetrical JCS Arrangements

| | | | |
|---|---|---|---|
| 5′ | JCS | JCS | 3′ |

| | | |
|---|---|---|
| siRNA TS | synthetic genome | miR TS |
| siRNA TS | synthetic genome | AmiR TS |
| siRNA TS | synthetic genome | gRNA TS |
| siRNA TS | synthetic genome | pri-miR |
| siRNA TS | synthetic genome | ribozyme |
| siRNA TS | synthetic genome | aptazyme |
| siRNA TS | synthetic genome | RNAseH primer TS |
| siRNA TS | synthetic genome | Restr Enz RS |
| miR TS | synthetic genome | siRNA TS |
| miR TS | synthetic genome | AmiR TS |
| miR TS | synthetic genome | gRNA TS |
| miR TS | synthetic genome | pri-miR |
| miR TS | synthetic genome | ribozyme |
| miR TS | synthetic genome | aptazyme |
| miR TS | synthetic genome | RNAseH primer TS |
| miR TS | synthetic genome | Restr Enz RS |
| AmiR TS | synthetic genome | siRNA TS |
| AmiR TS | synthetic genome | miR TS |
| AmiR TS | synthetic genome | gRNA TS |
| AmiR TS | synthetic genome | pri-miR |
| AmiR TS | synthetic genome | ribozyme |
| AmiR TS | synthetic genome | aptazyme |
| AmiR TS | synthetic genome | RNAseH primer TS |
| AmiR TS | synthetic genome | Restr Enz RS |
| gRNA TS | synthetic genome | siRNA TS |
| gRNA TS | synthetic genome | miR TS |
| gRNA TS | synthetic genome | AmiR TS |
| gRNA TS | synthetic genome | pri-miR |
| gRNA TS | synthetic genome | ribozyme |
| gRNA TS | synthetic genome | aptazyme |
| gRNA TS | synthetic genome | RNAseH primer TS |
| gRNA TS | synthetic genome | Restr Enz RS |
| pri-miR | synthetic genome | siRNA TS |
| pri-miR | synthetic genome | miR TS |
| pri-miR | synthetic genome | AmiR TS |
| pri-miR | synthetic genome | gRNA TS |
| pri-miR | synthetic genome | ribozyme |
| pri-miR | synthetic genome | aptazyme |
| pri-miR | synthetic genome | RNAseH primer TS |
| pri-miR | synthetic genome | Restr Enz RS |
| ribozyme | synthetic genome | siRNA TS |
| ribozyme | synthetic genome | miR TS |
| ribozyme | synthetic genome | AmiR TS |
| ribozyme | synthetic genome | gRNA TS |
| ribozyme | synthetic genome | pri-miR |
| ribozyme | synthetic genome | aptazyme |
| ribozyme | synthetic genome | RNAseH primer TS |
| ribozyme | synthetic genome | Restr Enz RS |
| aptazyme | synthetic genome | siRNA TS |
| aptazyme | synthetic genome | miR TS |
| aptazyme | synthetic genome | AmiR TS |
| aptazyme | synthetic genome | gRNA TS |
| aptazyme | synthetic genome | pri-miR |
| aptazyme | synthetic genome | ribozyme |
| aptazyme | synthetic genome | RNAseH primer TS |
| aptazyme | synthetic genome | Restr Enz RS |
| RNAseH primer TS | synthetic genome | siRNA TS |
| RNAseH primer TS | synthetic genome | miR TS |
| RNAseH primer TS | synthetic genome | AmiR TS |
| RNAseH primer TS | synthetic genome | gRNA TS |
| RNAseH primer TS | synthetic genome | pri-miR |
| RNAseH primer TS | synthetic genome | ribozyme |
| RNAseH primer TS | synthetic genome | aptazyme |
| RNAseH primer TS | synthetic genome | Restr Enz RS |

*“Restr Enz RS” refers to restriction enzyme recognition site

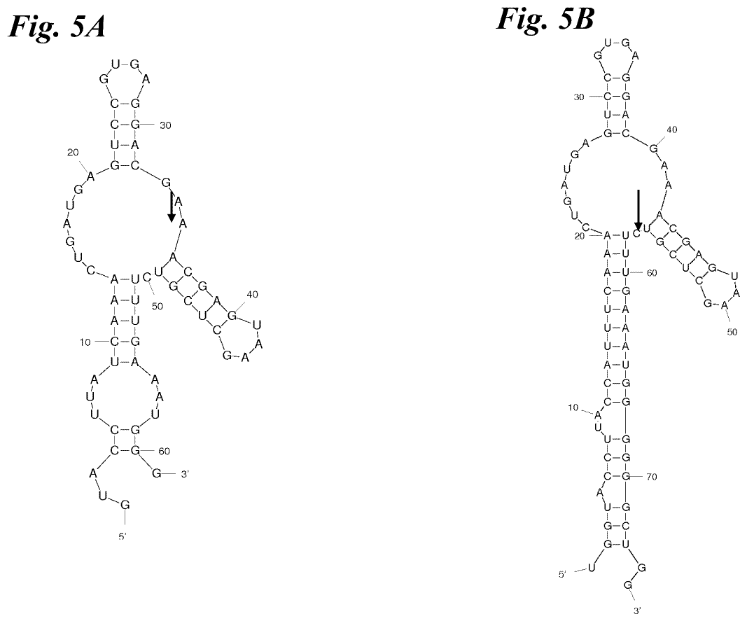

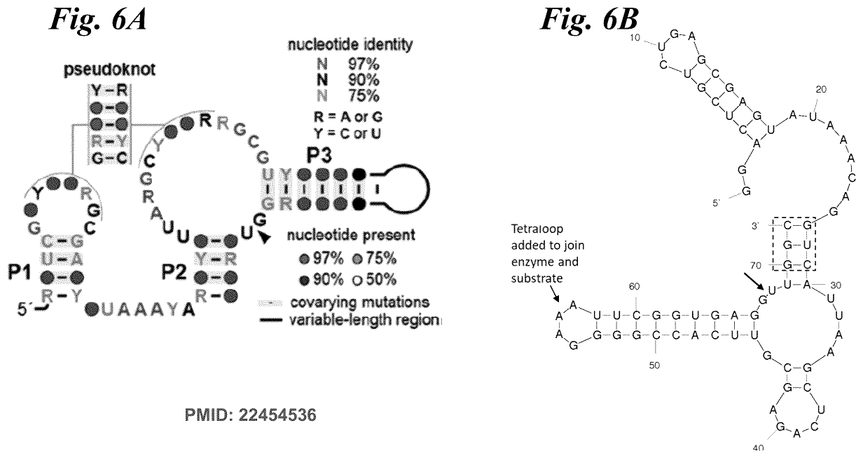

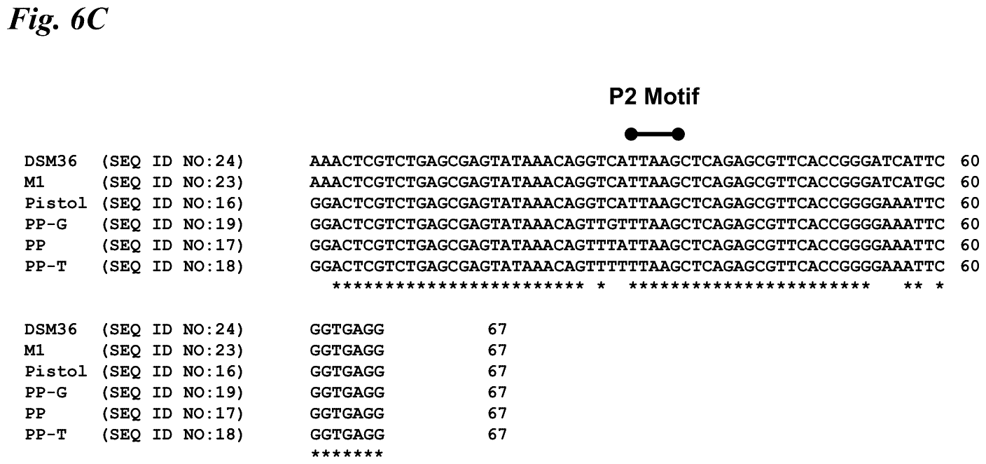

In some embodiments, the junctional cleavage sequences are ribozyme-encoding sequences and mediate self-cleavage of the synthetic RNA genome intermediates to produce the native discrete 5′ and/or 3′ ends of required for the final synthetic viral RNA genome and subsequent production of infectious RNA viruses. Exemplary ribozymes include the Hammerhead ribozyme (e.g., the Hammerhead ribozymes shown in FIG. 5A), the Varkud satellite (VS) ribozyme, the hairpin ribozyme, the GIR1 branching ribozyme, the glmS ribozyme, the twister ribozyme, the twister sister ribozyme (e.g., twister sister 1 or twister sister 2), the pistol ribozyme (e.g., the pistol ribozymes shown in FIGS. 6A-6C, the Env25 pistol ribozyme, or the Alistipes Putredinis Pistol Ribozyme), the hatchet ribozyme, and the Hepatitis delta virus ribozyme. In some embodiments, the 5′ and/or 3′ junctional cleavage sequences are ribozyme encoding sequences.

In some embodiments, the 5′ junctional cleavage sequence comprises or consists of a ribozyme sequence. In some embodiments, the 5′ ribozyme sequence are selected from a Hammerhead ribozyme sequence, a Pistol ribozyme sequence, or a Twister Sister ribozyme sequence.

In some embodiments, the 5′ junctional cleavage sequence comprises or consists of a 5′ Pistol ribozyme sequence. In some embodiments, the 5′ Pistol ribozyme sequence is derived from P. polymyxa. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to any one of SEQ ID NO: 16-19 and 23-24. In some embodiments, the 5′ Pistol ribozyme sequence comprises a P2 motif as indicated in FIGS. 6A and 6C, which is four nucleotides in length and locates at the region corresponding to nucleic acid positions 27-30 of SEQ ID NO: 16-19 and 23-24. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 17, wherein the polynucleotide sequence of its P2 motif is “TTTA”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 18, wherein the polynucleotide sequence of its P2 motif is “TTTT”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 19, wherein the polynucleotide sequence of its P2 motif is “TTGT”. In some embodiments, the 5′ Pistol ribozyme sequence is incorporated into the recombinant DNA molecule for in vitro transcription of a Coxsackievirus (e.g., CVA21) RNA viral genome.

In some embodiments, the 5′ junctional cleavage sequence comprises or consists of a 5′ Pistol ribozyme sequence. In some embodiments, the 5′ Pistol ribozyme sequence is derived from P. polymyxa. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 64 or 65. In some embodiments, the 5′ Pistol ribozyme sequence comprises a P2 motif, which is four nucleotides in length and locates at the region corresponding to nucleic acid positions 27-30 of SEQ ID NO: 64 or 65. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 64, wherein the polynucleotide sequence of its P2 motif is “TCAA”. In some embodiments, the 5′ Pistol ribozyme sequence derived from P. polymyxa comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 65, wherein the polynucleotide sequence of its P2 motif is “TTAA”. In some embodiments, the 5′ Pistol ribozyme sequence is incorporated into the recombinant DNA molecule for in vitro transcription of an SVV (e.g., SVV-IRES2) RNA viral genome.

In some embodiments, the 5′ junctional cleavage sequence comprises or consists of a Env25 Pistol Ribozyme. In some embodiments, the DNA sequence encoding the Env25 Pistol ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 96. In some embodiments, the Env25 Pistol ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 100.

In some embodiments, the 5′ junctional cleavage sequence comprises or consists of a Alistipes Putredinis Pistol Ribozyme. In some embodiments, the DNA sequence encoding the Alistipes Putredinis Pistol Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 97. In some embodiments, the Alistipes Putredinis Pistol Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 101.

In some embodiments, the 5′ junctional cleavage sequence comprises or consists of a Twister Sister 1 Ribozyme. In some embodiments, the DNA sequence encoding the Twister Sister 1 Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 98. In some embodiments, the Twister Sister1 Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 102.

In some embodiments, the 5′ junctional cleavage sequence comprises or consists of a Twister Sister 2 Ribozyme. In some embodiments, the DNA sequence encoding the Twister Sister 2 Ribozyme comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 99. In some embodiments, the Twister Sister2 Ribozyme RNA sequence comprises or consists of a sequence having at least 80%, at least 85%, at least 90%, at least 95%, at least 96%, at least 97%, at least 98%, at least 99%, or 100% (including all ranges and subranges therebetween) sequence identity to SEQ ID NO: 103.

### Leader Sequence

In some embodiments, the recombinant DNA molecule (e.g., DNA template) comprises a leader sequence in between the promoter sequence and the 5′ junctional cleavage sequence. In some embodiments, the presence of the leader sequence promotes or ensures the proper folding of the downstream 5′ junctional cleavage sequence (e.g., a 5′ ribozyme sequence).

In some embodiments, the leader sequence is about 5 bp, about 10 bp, about 15 bp, about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, or about 100 bp in length, including all ranges and subranges therebetween. In some embodiments, the leader sequence is at least 5 bp, at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, at least 95 bp, or at least 100 bp in length, including all ranges and subranges therebetween. In some embodiments, the leader sequence is less than 5 bp, less than 10 bp, less than 15 bp, less than 20 bp, less than 25 bp, less than 30 bp, less than 35 bp, less than 40 bp, less than 45 bp, less than 50 bp, less than 55 bp, less than 60 bp, less than 65 bp, less than 70 bp, less than 75 bp, less than 80 bp, less than 85 bp, less than 90 bp, less than 95 bp, or less than 100 bp in length, including all ranges and subranges therebetween. In some embodiments, the leader sequence is about 50-70 bp, about 40-60 bp, about 60-80 bp, about 40-80 bp, about 30-70 bp, about 50-90 bp, about 30-90 bp, about 20-60 bp, or about 60-100 bp in length, including all ranges and subranges therebetween. In some embodiments, the leader sequence is about 57 bp or about 55-60 bp in length.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 13-15. In some embodiments, the leader sequence comprises or consists of a polynucleotide sequence according to any one of SEQ ID NO: 13-15. In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 15. In some embodiments, the leader sequence comprises or consists of a polynucleotide sequence according to SEQ ID NO: 15. In some embodiment, the leader sequence is followed, or immediately followed, by a 5′ Pistol ribozyme sequence (e.g., a Pistol ribozyme from P. Polymyxa or a variant thereof). In some embodiments, the leader sequence is incorporated into a recombinant DNA molecule (e.g., DNA template) for in vitro transcription of a CVA21 RNA viral genome.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 53-63. In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to any one of SEQ ID NO: 53-60 and 62-63. In some embodiments, the leader sequence comprises or consists of a polynucleotide sequence according to any one of SEQ ID NO: 53-60 and 62-63.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 53. In some embodiments, the leader sequence comprises or consists of a polynucleotide sequence according to SEQ ID NO: 53.

In some embodiments, the leader sequence comprises or consists of a polynucleotide sequence having at least 60%, at least 65%, at least 70%, at least 75%, at least 80%, at least 85%, at least 90%, at least 95%, at least 96%, at least 97%, at least 98%, at least 99%, or 100% (including all ranges and subranges therebetween) sequence identity according to SEQ ID NO: 58. In some embodiments, the leader sequence comprises or consists of a polynucleotide sequence according to SEQ ID NO: 58.

In some embodiment, the leader sequence is followed, or immediately followed, by a 5′ Pistol ribozyme sequence (e.g., a Pistol ribozyme according to SEQ ID NO: 64 or 65 or a variant thereof). In some embodiments, the leader sequence is incorporated into a recombinant DNA molecule (e.g., DNA template) for in vitro transcription of a SVV RNA viral genome.

### Poly-A Tail

In some embodiments, the recombinant DNA molecule (e.g., DNA template) comprises a sequence encoding a polyA tail. In some embodiments, a poly-A tail is attached to the 3′ end of the synthetic RNA viral genome. In some embodiments, the poly-A tail is 2-500 bp in length (i.e., 2-500 pA). In some embodiments, the poly-A tail is 2-100, 2-150, 2-200, 2-250, 2-300, 2-400, or 2-500 bp in length, including all ranges and subranges therebetween. In some embodiments, the poly-A tail is about 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 bp in length, including all ranges and subranges therebetween. In some embodiments, the poly-A tail is about 10-30, 20-40, 30-50, 40-60, 50-70, 60-80, 70-90, 80-100, 90-110, 100-120, 110-130, 120-140, 130-150, 140-160, 150-170, 160-180, 170-190, or 180-200 bp in length, including all ranges and subranges therebetween. In some embodiments, the poly-A tail is about 65-75, 60-80, 55-85, 50-90, 45-95, or 40-100 bp in length, including all ranges and subranges therebetween. In some embodiments, the poly-A tail is about 70 bp in length. In some embodiments, a longer poly-A tail (e.g., about 70 bp in length) improves the loading capacity of the synthetic RNA viral genome on an Oligo-dT chromatography as compared to a corresponding synthetic RNA viral genome with a shorter poly-A tail (e.g., about 30 bp in length). In some embodiments, the loading capacity is improved by at least 10%, at least 20%, at least 30%, at least 40%, at least 50%, at least 60%, at least 70%, at least 80%, at least 90%, at least 1-fold, at least 2-fold, at least 3-fold, at least 4-fold, at least 5-fold, at least 7-fold, or at least 10-fold, as compared to the synthetic RNA viral genome with a poly-A tail of about 30 bp in length.

### Non-Limiting Examples of Recombinant DNA Molecule Designs

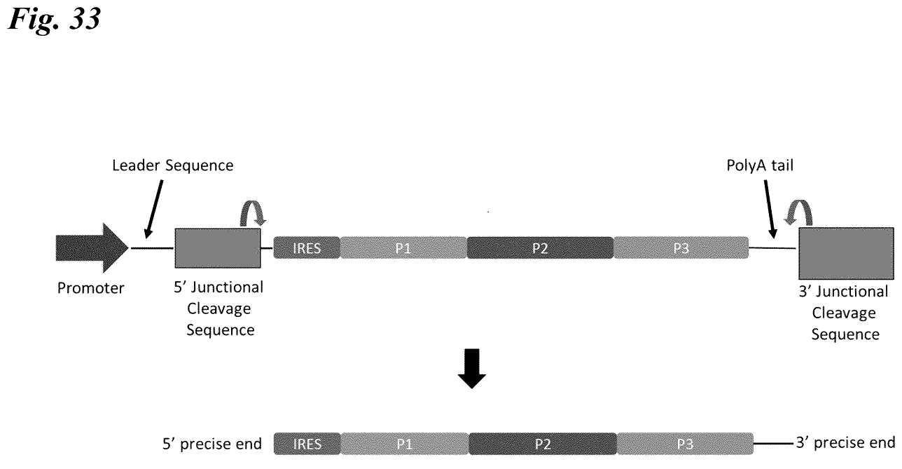

In some embodiments, the synthetic RNA viral genomes described herein are produced in vitro by in vitro RNA transcription (See, e.g., schematic in FIG. 10, FIG. 11A, FIG. 11B and FIG. 33). The synthetic RNA viral genomes are then purified and formulated for therapeutic use (e.g., encapsulated into a lipid nanoparticle).

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising or consisting of a ribozyme sequence. In some embodiments, the recombinant DNA molecule (e.g., DNA template) comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ hepatitis delta virus ribozyme sequence.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template) comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a ribozyme sequence; (iii) a polynucleotide encoding the synthetic RNA viral genome; and (iv) a 3′ junctional cleavage sequence comprising or consisting of a restriction enzyme recognition site. In some embodiments, the DNA template comprises, from 5′ to 3′: (i) a promoter sequence (e.g., a T7 polymerase promoter); (ii) a 5′ Hammerhead ribozyme sequence (e.g., a wild type HHR or a modified HHR such as that provided in FIG. 5A and FIG. 5B); (iii) a polynucleotide encoding the synthetic RNA viral genome; and (iv) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ Pistol ribozyme sequence (e.g., a Pistol 1 or a Pistol 2 ribozyme sequence shown in FIG. 6B); (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ Pistol ribozyme sequence (e.g., a Pistol 1 or a Pistol 2 ribozyme sequence shown in FIG. 6B); (iii) a polynucleotide encoding the synthetic RNA viral genome; and (iv) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template)comprises a polynucleotide comprising, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising a restriction enzyme recognition site. In some embodiments, the recombinant DNA molecule (e.g., DNA template) comprises a polynucleotide comprising, from 5′ to 3′: (i) a promoter sequence (e.g., a T7 polymerase promoter); (ii) a 5′ junctional cleavage sequence comprising or consisting of a 5′ RNAseH primer binding site; (iii) a polynucleotide encoding the synthetic RNA viral genome; and (iv) a 3′ junctional cleavage sequence comprising or consisting of a BsaI restriction enzyme recognition site.

In some embodiments, the synthetic RNA viral genome is a Coxsackievirus (CVA) genome. In some embodiments, the Coxsackievirus is a CVA21 strain. In some embodiments, the CVA21 strain is an EF strain. In some embodiments, the CVA21 strain is a KY strain.

In some embodiments, the recombinant DNA molecule (e.g., DNA template) comprises, from 5′ to 3′: (i) a promoter sequence (e.g., a T7 polymerase promoter); (ii) an optional leader sequence; (iii) a 5′ junctional cleavage sequence comprising or consisting of a 5′ Pistol ribozyme sequence (e.g., a Pistol ribozyme from P. Polymyxa or a variant thereof); (iv) a polynucleotide encoding the synthetic RNA viral genome; (v) a poly-A tail (e.g., about 20-80 bp in length, or about 30-70 bp in length), and (vi) a 3′ junctional cleavage sequence comprising or consisting of a restriction enzyme recognition site (e.g., for BsmBI or BsaI restriction enzyme).

In some embodiments, the recombinant DNA molecule (e.g., DNA template) comprises, from 5′ to 3′: (i) a promoter sequence (e.g., a T7 polymerase promoter); (ii) a leader sequence (e.g., SEQ ID NO: 14 or 15); (iii) a 5′ junctional cleavage sequence comprising or consisting of a 5′ Pistol ribozyme sequence (e.g., a Pistol ribozyme from P. Polymyxa or a variant thereof); (iv) a polynucleotide encoding a CVA21 synthetic RNA viral genome; (v) a poly-A tail (e.g., about 20-80 bp in length, or about 30-70 bp in length), and (vi) a 3′ junctional cleavage sequence comprising or consisting of a BsmBI restriction enzyme recognition site.

In some embodiments, the recombinant DNA molecule (e.g., DNA template) comprises, from 5′ to 3′: (i) a promoter sequence (e.g., a T7 polymerase promoter); (ii) a leader sequence (e.g., SEQ ID NO: 14 or 15); (iii) a 5′ junctional cleavage sequence comprising or consisting of a 5′ Pistol ribozyme sequence (e.g., a Pistol ribozyme from P. Polymyxa or a variant thereof); (iv) a polynucleotide encoding a CVA21 synthetic RNA viral genome; (v) a poly-A tail (e.g., about 20-80 bp in length, or about 30-70 bp in length), and (vi) a 3′ junctional cleavage sequence comprising or consisting of a BsaI restriction enzyme recognition site.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 15; (iii) a 5′ junctional cleavage sequence comprising or consisting of a 5′ Pistol ribozyme sequence; (iv) a polynucleotide encoding a CVA21 synthetic RNA viral genome; (v) a poly-A tail, and (vi) a 3′ junctional cleavage sequence comprising or consisting of a BsmBI restriction enzyme recognition site, wherein the combination of the 5′ Pistol ribozyme sequence and the poly-A tail is selected from one of Embodiments E1-E68 provided in Table 5A below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 15; (iii) a 5′ junctional cleavage sequence comprising or consisting of a 5′ Pistol ribozyme sequence; (iv) a polynucleotide encoding a CVA21 synthetic RNA viral genome; (v) a poly-A tail, and (vi) a 3′ junctional cleavage sequence comprising or consisting of a BsaI restriction enzyme recognition site, wherein the combination of the 5′ Pistol ribozyme sequence and the poly-A tail is selected from one of Embodiments E1-E68 provided in Table 5A below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 14; (iii) a 5′ junctional cleavage sequence comprising or consisting of a 5′ Pistol ribozyme sequence; (iv) a polynucleotide encoding a CVA21 synthetic RNA viral genome; (v) a poly-A tail, and (vi) a 3′ junctional cleavage sequence comprising or consisting of a BsmBI restriction enzyme recognition site, wherein the combination of the 5′ Pistol ribozyme sequence and the poly-A tail is selected from one of Embodiments E1-E68 provided in Table 5A below.

In some embodiments, the recombinant DNA molecule (e.g., DNA template) comprises, from 5′ to 3′: (i) a promoter sequence (e.g., a T7 polymerase promoter); (ii) a leader sequence according to SEQ ID NO: 14; (iii) a 5′ junctional cleavage sequence comprising or consisting of a 5′ Pistol ribozyme sequence; (iv) a polynucleotide encoding a CVA21 synthetic RNA viral genome; (v) a poly-A tail, and (vi) a 3′ junctional cleavage sequence comprising or consisting of a BsaI restriction enzyme recognition site, wherein the combination of the 5′ Pistol ribozyme sequence and the poly-A tail is selected from one of Embodiments E1-E68 provided in Table 5A below.

TABLE 5A
Non-limiting Embodiments of Leader Sequence, 5′ Ribozyme Sequence,
and Poly-A Tail in the DNA template for Expressing CVA21 Viral Genome

| | 5′ Pistol Ribozyme | 5′ Pistol Ribozyme |
|---|---|---|
| | with P2 motif of | with P2 motif of |
| | “TTTA”. | “TTTT”. |
| Leader sequence according to SEQ ID NO: 14; | Embodiment E1 | Embodiment E2 |
| poly-A tail about 100 pA in length | | |
| Leader sequence according to SEQ ID NO: 14; | Embodiment E3 | Embodiment E4 |
| poly-A tail about 90 pA in length | | |
| Leader sequence according to SEQ ID NO: 14; | Embodiment E5 | Embodiment E6 |
| poly-A tail about 80 pA in length | | |
| Leader sequence according to SEQ ID NO: 14; | Embodiment E7 | Embodiment E8 |
| poly-A tail about 70 pA in length | | |
| Leader sequence according to SEQ ID NO: 14; | Embodiment E9 | Embodiment E10 |
| poly-A tail about 60 pA in length | | |
| Leader sequence according to SEQ ID NO: 14; | Embodiment E11 | Embodiment E12 |
| poly-A tail about 50 pA in length | | |
| Leader sequence according to SEQ ID NO: 14; | Embodiment E13 | Embodiment E14 |
| poly-A tail about 40 pA in length | | |
| Leader sequence according to SEQ ID NO: 14; | Embodiment E15 | Embodiment E16 |
| poly-A tail about 30 pA in length | | |
| Leader sequence according to SEQ ID NO: 14; | Embodiment E17 | Embodiment E18 |
| poly-A tail about 20 pA in length | | |
| Leader sequence according to SEQ ID NO: 14; | Embodiment E19 | Embodiment E20 |
| poly-A tail about 80-100 pA in length | | |
| Leader sequence according to SEQ ID NO: 14; | Embodiment E21 | Embodiment E22 |
| poly-A tail about 70-90 pA in length | | |
| Leader sequence according to SEQ ID NO: 14; | Embodiment E23 | Embodiment E24 |
| poly-A tail about 60-80 pA in length | | |
| Leader sequence according to SEQ ID NO: 14; | Embodiment E25 | Embodiment E26 |
| poly-A tail about 50-70 pA in length | | |
| Leader sequence according to SEQ ID NO: 14; | Embodiment E27 | Embodiment E28 |
| poly-A tail about 40-60 pA in length | | |
| Leader sequence according to SEQ ID NO: 14; | Embodiment E29 | Embodiment E30 |
| poly-A tail about 30-50 pA in length | | |
| Leader sequence according to SEQ ID NO: 14; | Embodiment E31 | Embodiment E32 |
| poly-A tail about 20-40 pA in length | | |
| Leader sequence according to SEQ ID NO: 14; | Embodiment E33 | Embodiment E34 |
| poly-A tail about 10-30 pA in length | | |
| Leader sequence according to SEQ ID NO: 15; | Embodiment E35 | Embodiment E36 |
| poly-A tail about 100 pA in length | | |
| Leader sequence according to SEQ ID NO: 15; | Embodiment E37 | Embodiment E38 |
| poly-A tail about 90 pA in length | | |
| Leader sequence according to SEQ ID NO: 15; | Embodiment E39 | Embodiment E40 |
| poly-A tail about 80 pA in length | | |
| Leader sequence according to SEQ ID NO: 15; | Embodiment E41 | Embodiment E42 |
| poly-A tail about 70 pA in length | | |
| Leader sequence according to SEQ ID NO: 15; | Embodiment E43 | Embodiment E44 |
| poly-A tail about 60 pA in length | | |
| Leader sequence according to SEQ ID NO: 15; | Embodiment E45 | Embodiment E46 |
| poly-A tail about 50 pA in length | | |
| Leader sequence according to SEQ ID NO: 15; | Embodiment E47 | Embodiment E48 |
| poly-A tail about 40 pA in length | | |
| Leader sequence according to SEQ ID NO: 15; | Embodiment E49 | Embodiment E50 |
| poly-A tail about 30 pA in length | | |
| Leader sequence according to SEQ ID NO: 15; | Embodiment E51 | Embodiment E52 |
| poly-A tail about 20 pA in length | | |
| Leader sequence according to SEQ ID NO: 15; | Embodiment E53 | Embodiment E54 |
| poly-A tail about 80-100 pA in length | | |
| Leader sequence according to SEQ ID NO: 15; | Embodiment E55 | Embodiment E56 |
| poly-A tail about 70-90 pA in length | | |
| Leader sequence according to SEQ ID NO: 15; | Embodiment E57 | Embodiment E58 |
| poly-A tail about 60-80 pA in length | | |
| Leader sequence according to SEQ ID NO: 15; | Embodiment E59 | Embodiment E60 |
| poly-A tail about 50-70 pA in length | | |
| Leader sequence according to SEQ ID NO: 15; | Embodiment E61 | Embodiment E62 |
| poly-A tail about 40-60 pA in length | | |
| Leader sequence according to SEQ ID NO: 15; | Embodiment E63 | Embodiment E64 |
| poly-A tail about 30-50 pA in length | | |
| Leader sequence according to SEQ ID NO: 15; | Embodiment E65 | Embodiment E66 |
| poly-A tail about 20-40 pA in length | | |
| Leader sequence according to SEQ ID NO: 15; | Embodiment E67 | Embodiment E68 |
| poly-A tail about 10-30 pA in length | | |

In some embodiments, the recombinant DNA molecule (e.g., DNA template) comprises, from 5′ to 3′: (i) a T7 polymerase promoter sequence; (ii) a leader sequence according to SEQ ID NO: 15; (iii) a 5′ junctional cleavage sequence comprising or consisting of a 5′ Pistol ribozyme sequence according to SEQ ID NO: 18; (iv) a polynucleotide encoding a CVA21 synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsmBI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 15; (iii) a 5′ junctional cleavage sequence comprising or consisting of a 5′ Pistol ribozyme sequence according to SEQ ID NO: 18; (iv) a polynucleotide encoding a CVA21 synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsaI restriction enzyme recognition site.

In some embodiments, the recombinant DNA molecule (e.g., DNA template) comprises a DNA polynucleotide which encodes a CVA21-KY strain viral genome and comprises or consists of a sequence according to SEQ ID NO: 20. In some embodiments, the DNA polynucleotide encoding the CVA21-KY strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 20.

In some embodiments, the recombinant DNA molecule (e.g., DNA template) comprises a DNA polynucleotide which encodes a CVA21-KY strain viral genome and comprises or consists of a sequence according to SEQ ID NO: 93. In some embodiments, the DNA polynucleotide encoding the CVA21-KY strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 93.

In some embodiments, the recombinant DNA molecule (e.g., DNA template) comprises a DNA polynucleotide which encodes a CVA21-EF strain viral genome and comprises or consists of a sequence according to SEQ ID NO: 21. In some embodiments, the DNA polynucleotide encoding the CVA21-EF strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 21.

In some embodiments, the recombinant DNA molecule (e.g., DNA template) comprises a DNA polynucleotide which encodes a CVA21-EF strain viral genome and comprises or consists of a sequence according to SEQ ID NO: 95. In some embodiments, the DNA polynucleotide encoding the CVA21-EF strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 95.

In some embodiments, the recombinant DNA molecule (e.g., DNA template) comprises a DNA polynucleotide which encodes a CVA21-Kuykendall strain viral genome and comprises or consists of a sequence according to SEQ ID NO: 22. In some embodiments, the DNA polynucleotide encoding the CVA21-Kuykendall strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 22.

In some embodiments, the recombinant DNA molecule (e.g., DNA template) comprises a DNA polynucleotide which encodes a CVA21-Kuykendall strain viral genome and comprises or consists of a sequence according to SEQ ID NO: 94. In some embodiments, the DNA polynucleotide encoding the CVA21-Kuykendall strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 94.

In some embodiments, the synthetic RNA viral genome is a Seneca Valley virus (SVV) genome.

In some embodiments, the recombinant DNA molecule (e.g., DNA template) comprises, from 5′ to 3′: (i) a promoter sequence (e.g., a T7 polymerase promoter); (ii) an optional leader sequence; (iii) a 5′ junctional cleavage sequence comprising or consisting of a 5′ Pistol ribozyme sequence (e.g., a Pistol ribozyme from P. Polymyxa or a variant thereof); (iv) a polynucleotide encoding the synthetic RNA viral genome; (v) a poly-A tail (e.g., about 20-80 bp in length, or about 30-70 bp in length), and (vi) a 3′ junctional cleavage sequence comprising or consisting of a restriction enzyme recognition site (e.g., for SapI or BsaI restriction enzyme).

In some embodiments, the recombinant DNA molecule (e.g., DNA template) comprises, from 5′ to 3′: (i) a promoter sequence (e.g., a T7 polymerase promoter); (ii) a leader sequence (e.g., any one of SEQ ID NO: 53-60 and 62-63); (iii) a 5′ junctional cleavage sequence comprising or consisting of a 5′ Pistol ribozyme sequence (e.g., according to SEQ ID NO: 64 or 65, or a variant thereof); (iv) a polynucleotide encoding a SVV synthetic RNA viral genome; (v) a poly-A tail (e.g., about 20-80 bp in length, or about 30-70 bp in length), and (vi) a 3′ junctional cleavage sequence comprising or consisting of a SapI restriction enzyme recognition site.

In some embodiments, the recombinant DNA molecule (e.g., DNA template) comprises, from 5′ to 3′: (i) a promoter sequence (e.g., a T7 polymerase promoter); (ii) a leader sequence (e.g., any one of SEQ ID NO: 53-60 and 62-63); (iii) a 5′ junctional cleavage sequence comprising or consisting of a 5′ Pistol ribozyme sequence (e.g., according to SEQ ID NO: 64 or 65, or a variant thereof); (iv) a polynucleotide encoding a SVV synthetic RNA viral genome; (v) a poly-A tail (e.g., about 20-80 bp in length, or about 30-70 bp in length), and (vi) a 3′ junctional cleavage sequence comprising or consisting of a BsaI restriction enzyme recognition site.

In some embodiments, the recombinant DNA molecule (e.g., DNA template) comprises a 3′ SapI restriction enzyme recognition site after the poly-A tail and does not comprise an SapI restriction enzyme recognition site within the polynucleotide encoding a SVV synthetic RNA viral genome.

In some embodiments, the recombinant DNA molecule (e.g., DNA template) comprises a 3′ BsaI restriction enzyme recognition site after the poly-A tail and does not comprise an BsaI restriction enzyme recognition site within the polynucleotide encoding a SVV synthetic RNA viral genome.

In some embodiments, the recombinant DNA molecule (e.g., DNA template) comprises, from 5′ to 3′: (i) a promoter sequence (e.g., a T7 polymerase promoter); (ii) a leader sequence according to any one of SEQ ID NO: 53-60 and 62-63; (iii) a 5′ junctional cleavage sequence comprising or consisting of a 5′ Pistol ribozyme sequence; (iv) a polynucleotide encoding a SVV synthetic RNA viral genome; (v) a poly-A tail, and (vi) a 3′ junctional cleavage sequence comprising or consisting of a SapI restriction enzyme recognition site, wherein the combination of the leader sequence, the 5′ Pistol ribozyme sequence and the poly-A tail is selected from one of Embodiments S1-S340 provided in Table 5B below.

In some embodiments, the recombinant DNA molecule (e.g., DNA template) comprises, from 5′ to 3′: (i) a promoter sequence (e.g., a T7 polymerase promoter); (ii) a leader sequence according to any one of SEQ ID NO: 53-60 and 62-63; (iii) a 5′ junctional cleavage sequence comprising or consisting of a 5′ Pistol ribozyme sequence; (iv) a polynucleotide encoding a SVV synthetic RNA viral genome; (v) a poly-A tail, and (vi) a 3′ junctional cleavage sequence comprising or consisting of a BsaI restriction enzyme recognition site, wherein the combination of the leader sequence, the 5′ Pistol ribozyme sequence and the poly-A tail is selected from one of Embodiments S1-S340 provided in Table 5B below.

TABLE 5B
Non-limiting Embodiments of Leader Sequence, 5′ Ribozyme Sequence,
and Poly-A Tail in the DNA template for Expressing SVV Viral Genome

| | 5′ Pistol Ribozyme | 5′ Pistol Ribozyme |
|---|---|---|
| | with P2 motif of | with P2 motif of |
| | “TCAA”. | “TTAA”. |
| Leader sequence according to SEQ ID NO: 53; | Embodiment S1 | Embodiment S2 |
| poly-A tail about 100 pA in length | | |
| Leader sequence according to SEQ ID NO: 53; | Embodiment S3 | Embodiment S4 |
| poly-A tail about 90 pA in length | | |
| Leader sequence according to SEQ ID NO: 53; | Embodiment S5 | Embodiment S6 |
| poly-A tail about 80 pA in length | | |
| Leader sequence according to SEQ ID NO: 53; | Embodiment S7 | Embodiment S8 |
| poly-A tail about 70 pA in length | | |
| Leader sequence according to SEQ ID NO: 53; | Embodiment S9 | Embodiment S10 |
| poly-A tail about 60 pA in length | | |
| Leader sequence according to SEQ ID NO: 53; | Embodiment S11 | Embodiment S12 |
| poly-A tail about 50 pA in length | | |
| Leader sequence according to SEQ ID NO: 53; | Embodiment S13 | Embodiment S14 |
| poly-A tail about 40 pA in length | | |
| Leader sequence according to SEQ ID NO: 53; | Embodiment S15 | Embodiment S16 |
| poly-A tail about 30 pA in length | | |
| Leader sequence according to SEQ ID NO: 53; | Embodiment S17 | Embodiment S18 |
| poly-A tail about 20 pA in length | | |
| Leader sequence according to SEQ ID NO: 53; | Embodiment S19 | Embodiment S20 |
| poly-A tail about 80-100 pA in length | | |
| Leader sequence according to SEQ ID NO: 53; | Embodiment S21 | Embodiment S22 |
| poly-A tail about 70-90 pA in length | | |
| Leader sequence according to SEQ ID NO: 53; | Embodiment S23 | Embodiment S24 |
| poly-A tail about 60-80 pA in length | | |
| Leader sequence according to SEQ ID NO: 53; | Embodiment S25 | Embodiment S26 |
| poly-A tail about 50-70 pA in length | | |
| Leader sequence according to SEQ ID NO: 53; | Embodiment S27 | Embodiment S28 |
| poly-A tail about 40-60 pA in length | | |
| Leader sequence according to SEQ ID NO: 53; | Embodiment S29 | Embodiment S30 |
| poly-A tail about 30-50 pA in length | | |
| Leader sequence according to SEQ ID NO: 53; | Embodiment S31 | Embodiment S32 |
| poly-A tail about 20-40 pA in length | | |
| Leader sequence according to SEQ ID NO: 53; | Embodiment S33 | Embodiment S34 |
| poly-A tail about 10-30 pA in length | | |
| Leader sequence according to SEQ ID NO: 54; | Embodiment S35 | Embodiment S36 |
| poly-A tail about 100 pA in length | | |
| Leader sequence according to SEQ ID NO: 54; | Embodiment S37 | Embodiment S38 |
| poly-A tail about 90 pA in length | | |
| Leader sequence according to SEQ ID NO: 54; | Embodiment S39 | Embodiment S40 |
| poly-A tail about 80 pA in length | | |
| Leader sequence according to SEQ ID NO: 54; | Embodiment S41 | Embodiment S42 |
| poly-A tail about 70 pA in length | | |
| Leader sequence according to SEQ ID NO: 54; | Embodiment S43 | Embodiment S44 |
| poly-A tail about 60 pA in length | | |
| Leader sequence according to SEQ ID NO: 54; | Embodiment S45 | Embodiment S46 |
| poly-A tail about 50 pA in length | | |
| Leader sequence according to SEQ ID NO: 54; | Embodiment S47 | Embodiment S48 |
| poly-A tail about 40 pA in length | | |
| Leader sequence according to SEQ ID NO: 54; | Embodiment S49 | Embodiment S50 |
| poly-A tail about 30 pA in length | | |
| Leader sequence according to SEQ ID NO: 54; | Embodiment S51 | Embodiment S52 |
| poly-A tail about 20 pA in length | | |
| Leader sequence according to SEQ ID NO: 54; | Embodiment S53 | Embodiment S54 |
| poly-A tail about 80-100 pA in length | | |
| Leader sequence according to SEQ ID NO: 54; | Embodiment S55 | Embodiment S56 |
| poly-A tail about 70-90 pA in length | | |
| Leader sequence according to SEQ ID NO: 54; | Embodiment S57 | Embodiment S58 |
| poly-A tail about 60-80 pA in length | | |
| Leader sequence according to SEQ ID NO: 54; | Embodiment S59 | Embodiment S60 |
| poly-A tail about 50-70 pA in length | | |
| Leader sequence according to SEQ ID NO: 54; | Embodiment S61 | Embodiment S62 |
| poly-A tail about 40-60 pA in length | | |
| Leader sequence according to SEQ ID NO: 54; | Embodiment S63 | Embodiment S64 |
| poly-A tail about 30-50 pA in length | | |
| Leader sequence according to SEQ ID NO: 54; | Embodiment S65 | Embodiment S66 |
| poly-A tail about 20-40 pA in length | | |
| Leader sequence according to SEQ ID NO: 54; | Embodiment S67 | Embodiment S68 |
| poly-A tail about 10-30 pA in length | | |
| Leader sequence according to SEQ ID NO: 55; | Embodiment S69 | Embodiment S70 |
| poly-A tail about 100 pA in length | | |
| Leader sequence according to SEQ ID NO: 55; | Embodiment S71 | Embodiment S72 |
| poly-A tail about 90 pA in length | | |
| Leader sequence according to SEQ ID NO: 55; | Embodiment S73 | Embodiment S74 |
| poly-A tail about 80 pA in length | | |
| Leader sequence according to SEQ ID NO: 55; | Embodiment S75 | Embodiment S76 |
| poly-A tail about 70 pA in length | | |
| Leader sequence according to SEQ ID NO: 55; | Embodiment S77 | Embodiment S78 |
| poly-A tail about 60 pA in length | | |
| Leader sequence according to SEQ ID NO: 55; | Embodiment S79 | Embodiment S80 |
| poly-A tail about 50 pA in length | | |
| Leader sequence according to SEQ ID NO: 55; | Embodiment S81 | Embodiment S82 |
| poly-A tail about 40 pA in length | | |
| Leader sequence according to SEQ ID NO: 55; | Embodiment S83 | Embodiment S84 |
| poly-A tail about 30 pA in length | | |
| Leader sequence according to SEQ ID NO: 55; | Embodiment S85 | Embodiment S86 |
| poly-A tail about 20 pA in length | | |
| Leader sequence according to SEQ ID NO: 55; | Embodiment S87 | Embodiment S88 |
| poly-A tail about 80-100 pA in length | | |
| Leader sequence according to SEQ ID NO: 55; | Embodiment S89 | Embodiment S90 |
| poly-A tail about 70-90 pA in length | | |
| Leader sequence according to SEQ ID NO: 55; | Embodiment S91 | Embodiment S92 |
| poly-A tail about 60-80 pA in length | | |
| Leader sequence according to SEQ ID NO: 55; | Embodiment S93 | Embodiment S94 |
| poly-A tail about 50-70 pA in length | | |
| Leader sequence according to SEQ ID NO: 55; | Embodiment S95 | Embodiment S96 |
| poly-A tail about 40-60 pA in length | | |
| Leader sequence according to SEQ ID NO: 55; | Embodiment S97 | Embodiment S98 |
| poly-A tail about 30-50 pA in length | | |
| Leader sequence according to SEQ ID NO: 55; | Embodiment S99 | Embodiment S100 |
| poly-A tail about 20-40 pA in length | | |
| Leader sequence according to SEQ ID NO: 55; | Embodiment S101 | Embodiment S102 |
| poly-A tail about 10-30 pA in length | | |
| Leader sequence according to SEQ ID NO: 56; | Embodiment S103 | Embodiment S104 |
| poly-A tail about 100 pA in length | | |
| Leader sequence according to SEQ ID NO: 56; | Embodiment S105 | Embodiment S106 |
| poly-A tail about 90 pA in length | | |
| Leader sequence according to SEQ ID NO: 56; | Embodiment S107 | Embodiment S108 |
| poly-A tail about 80 pA in length | | |
| Leader sequence according to SEQ ID NO: 56; | Embodiment S109 | Embodiment S110 |
| poly-A tail about 70 pA in length | | |
| Leader sequence according to SEQ ID NO: 56; | Embodiment S111 | Embodiment S112 |
| poly-A tail about 60 pA in length | | |
| Leader sequence according to SEQ ID NO: 56; | Embodiment S113 | Embodiment S114 |
| poly-A tail about 50 pA in length | | |
| Leader sequence according to SEQ ID NO: 56; | Embodiment S115 | Embodiment S116 |
| poly-A tail about 40 pA in length | | |
| Leader sequence according to SEQ ID NO: 56; | Embodiment S117 | Embodiment S118 |
| poly-A tail about 30 pA in length | | |
| Leader sequence according to SEQ ID NO: 56; | Embodiment S119 | Embodiment S120 |
| poly-A tail about 20 pA in length | | |
| Leader sequence according to SEQ ID NO: 56; | Embodiment S121 | Embodiment S122 |
| poly-A tail about 80-100 pA in length | | |
| Leader sequence according to SEQ ID NO: 56; | Embodiment S123 | Embodiment S124 |
| poly-A tail about 70-90 pA in length | | |
| Leader sequence according to SEQ ID NO: 56; | Embodiment S125 | Embodiment S126 |
| poly-A tail about 60-80 pA in length | | |
| Leader sequence according to SEQ ID NO: 56; | Embodiment S127 | Embodiment S128 |
| poly-A tail about 50-70 pA in length | | |
| Leader sequence according to SEQ ID NO: 56; | Embodiment S129 | Embodiment S130 |
| poly-A tail about 40-60 pA in length | | |
| Leader sequence according to SEQ ID NO: 56; | Embodiment S131 | Embodiment S132 |
| poly-A tail about 30-50 pA in length | | |
| Leader sequence according to SEQ ID NO: 56; | Embodiment S133 | Embodiment S134 |
| poly-A tail about 20-40 pA in length | | |
| Leader sequence according to SEQ ID NO: 56; | Embodiment S135 | Embodiment S136 |
| poly-A tail about 10-30 pA in length | | |
| Leader sequence according to SEQ ID NO: 57; | Embodiment S137 | Embodiment S138 |
| poly-A tail about 100 pA in length | | |
| Leader sequence according to SEQ ID NO: 57; | Embodiment S139 | Embodiment S140 |
| poly-A tail about 90 pA in length | | |
| Leader sequence according to SEQ ID NO: 57; | Embodiment S141 | Embodiment S142 |
| poly-A tail about 80 pA in length | | |
| Leader sequence according to SEQ ID NO: 57; | Embodiment S143 | Embodiment S144 |
| poly-A tail about 70 pA in length | | |
| Leader sequence according to SEQ ID NO: 57; | Embodiment S145 | Embodiment S146 |
| poly-A tail about 60 pA in length | | |
| Leader sequence according to SEQ ID NO: 57; | Embodiment S147 | Embodiment S148 |
| poly-A tail about 50 pA in length | | |
| Leader sequence according to SEQ ID NO: 57; | Embodiment S149 | Embodiment S150 |
| poly-A tail about 40 pA in length | | |
| Leader sequence according to SEQ ID NO: 57; | Embodiment S151 | Embodiment S152 |
| poly-A tail about 30 pA in length | | |
| Leader sequence according to SEQ ID NO: 57; | Embodiment S153 | Embodiment S154 |
| poly-A tail about 20 pA in length | | |
| Leader sequence according to SEQ ID NO: 57; | Embodiment S155 | Embodiment S156 |
| poly-A tail about 80-100 pA in length | | |
| Leader sequence according to SEQ ID NO: 57; | Embodiment S157 | Embodiment S158 |
| poly-A tail about 70-90 pA in length | | |
| Leader sequence according to SEQ ID NO: 57; | Embodiment S159 | Embodiment S160 |
| poly-A tail about 60-80 pA in length | | |
| Leader sequence according to SEQ ID NO: 57; | Embodiment S161 | Embodiment S162 |
| poly-A tail about 50-70 pA in length | | |
| Leader sequence according to SEQ ID NO: 57; | Embodiment S163 | Embodiment S164 |
| poly-A tail about 40-60 pA in length | | |
| Leader sequence according to SEQ ID NO: 57; | Embodiment S165 | Embodiment S166 |
| poly-A tail about 30-50 pA in length | | |
| Leader sequence according to SEQ ID NO: 57; | Embodiment S167 | Embodiment S168 |
| poly-A tail about 20-40 pA in length | | |
| Leader sequence according to SEQ ID NO: 57; | Embodiment S169 | Embodiment S170 |
| poly-A tail about 10-30 pA in length | | |
| Leader sequence according to SEQ ID NO: 58; | Embodiment S171 | Embodiment S172 |
| poly-A tail about 100 pA in length | | |
| Leader sequence according to SEQ ID NO: 58; | Embodiment S173 | Embodiment S174 |
| poly-A tail about 90 pA in length | | |
| Leader sequence according to SEQ ID NO: 58; | Embodiment S175 | Embodiment S176 |
| poly-A tail about 80 pA in length | | |
| Leader sequence according to SEQ ID NO: 58; | Embodiment S177 | Embodiment S178 |
| poly-A tail about 70 pA in length | | |
| Leader sequence according to SEQ ID NO: 58; | Embodiment S179 | Embodiment S180 |
| poly-A tail about 60 pA in length | | |
| Leader sequence according to SEQ ID NO: 58; | Embodiment S181 | Embodiment S182 |
| poly-A tail about 50 pA in length | | |
| Leader sequence according to SEQ ID NO: 58; | Embodiment S183 | Embodiment S184 |
| poly-A tail about 40 pA in length | | |
| Leader sequence according to SEQ ID NO: 58; | Embodiment S185 | Embodiment S186 |
| poly-A tail about 30 pA in length | | |
| Leader sequence according to SEQ ID NO: 58; | Embodiment S187 | Embodiment S188 |
| poly-A tail about 20 pA in length | | |
| Leader sequence according to SEQ ID NO: 58; | Embodiment S189 | Embodiment S190 |
| poly-A tail about 80-100 pA in length | | |
| Leader sequence according to SEQ ID NO: 58; | Embodiment S191 | Embodiment S192 |
| poly-A tail about 70-90 pA in length | | |
| Leader sequence according to SEQ ID NO: 58; | Embodiment S193 | Embodiment S194 |
| poly-A tail about 60-80 pA in length | | |
| Leader sequence according to SEQ ID NO: 58; | Embodiment S195 | Embodiment S196 |
| poly-A tail about 50-70 pA in length | | |
| Leader sequence according to SEQ ID NO: 58; | Embodiment S197 | Embodiment S198 |
| poly-A tail about 40-60 pA in length | | |
| Leader sequence according to SEQ ID NO: 58; | Embodiment S199 | Embodiment S200 |
| poly-A tail about 30-50 pA in length | | |
| Leader sequence according to SEQ ID NO: 58; | Embodiment S201 | Embodiment S202 |
| poly-A tail about 20-40 pA in length | | |
| Leader sequence according to SEQ ID NO: 58; | Embodiment S203 | Embodiment S204 |
| poly-A tail about 10-30 pA in length | | |
| Leader sequence according to SEQ ID NO: 59; | Embodiment S205 | Embodiment S206 |
| poly-A tail about 100 pA in length | | |
| Leader sequence according to SEQ ID NO: 59; | Embodiment S207 | Embodiment S208 |
| poly-A tail about 90 pA in length | | |
| Leader sequence according to SEQ ID NO: 59; | Embodiment S209 | Embodiment S210 |
| poly-A tail about 80 pA in length | | |
| Leader sequence according to SEQ ID NO: 59; | Embodiment S211 | Embodiment S212 |
| poly-A tail about 70 pA in length | | |
| Leader sequence according to SEQ ID NO: 59; | Embodiment S213 | Embodiment S214 |
| poly-A tail about 60 pA in length | | |
| Leader sequence according to SEQ ID NO: 59; | Embodiment S215 | Embodiment S216 |
| poly-A tail about 50 pA in length | | |
| Leader sequence according to SEQ ID NO: 59; | Embodiment S217 | Embodiment S218 |
| poly-A tail about 40 pA in length | | |
| Leader sequence according to SEQ ID NO: 59; | Embodiment S219 | Embodiment S220 |
| poly-A tail about 30 pA in length | | |
| Leader sequence according to SEQ ID NO: 59; | Embodiment S221 | Embodiment S222 |
| poly-A tail about 20 pA in length | | |
| Leader sequence according to SEQ ID NO: 59; | Embodiment S223 | Embodiment S224 |
| poly-A tail about 80-100 pA in length | | |
| Leader sequence according to SEQ ID NO: 59; | Embodiment S225 | Embodiment S226 |
| poly-A tail about 70-90 pA in length | | |
| Leader sequence according to SEQ ID NO: 59; | Embodiment S227 | Embodiment S228 |
| poly-A tail about 60-80 pA in length | | |
| Leader sequence according to SEQ ID NO: 59; | Embodiment S229 | Embodiment S230 |
| poly-A tail about 50-70 pA in length | | |
| Leader sequence according to SEQ ID NO: 59; | Embodiment S231 | Embodiment S232 |
| poly-A tail about 40-60 pA in length | | |
| Leader sequence according to SEQ ID NO: 59; | Embodiment S233 | Embodiment S234 |
| poly-A tail about 30-50 pA in length | | |
| Leader sequence according to SEQ ID NO: 59; | Embodiment S235 | Embodiment S236 |
| poly-A tail about 20-40 pA in length | | |
| Leader sequence according to SEQ ID NO: 59; | Embodiment S237 | Embodiment S238 |
| poly-A tail about 10-30 pA in length | | |
| Leader sequence according to SEQ ID NO: 60; | Embodiment S239 | Embodiment S240 |
| poly-A tail about 100 pA in length | | |
| Leader sequence according to SEQ ID NO: 60; | Embodiment S241 | Embodiment S242 |
| poly-A tail about 90 pA in length | | |
| Leader sequence according to SEQ ID NO: 60; | Embodiment S243 | Embodiment S244 |
| poly-A tail about 80 pA in length | | |
| Leader sequence according to SEQ ID NO: 60; | Embodiment S245 | Embodiment S246 |
| poly-A tail about 70 pA in length | | |
| Leader sequence according to SEQ ID NO: 60; | Embodiment S247 | Embodiment S248 |
| poly-A tail about 60 pA in length | | |
| Leader sequence according to SEQ ID NO: 60; | Embodiment S249 | Embodiment S250 |
| poly-A tail about 50 pA in length | | |
| Leader sequence according to SEQ ID NO: 60; | Embodiment S251 | Embodiment S252 |
| poly-A tail about 40 pA in length | | |
| Leader sequence according to SEQ ID NO: 60; | Embodiment S253 | Embodiment S254 |
| poly-A tail about 30 pA in length | | |
| Leader sequence according to SEQ ID NO: 60; | Embodiment S255 | Embodiment S256 |
| poly-A tail about 20 pA in length | | |
| Leader sequence according to SEQ ID NO: 60; | Embodiment S257 | Embodiment S258 |
| poly-A tail about 80-100 pA in length | | |
| Leader sequence according to SEQ ID NO: 60; | Embodiment S259 | Embodiment S260 |
| poly-A tail about 70-90 pA in length | | |
| Leader sequence according to SEQ ID NO: 60; | Embodiment S261 | Embodiment S262 |
| poly-A tail about 60-80 pA in length | | |
| Leader sequence according to SEQ ID NO: 60; | Embodiment S263 | Embodiment S264 |
| poly-A tail about 50-70 pA in length | | |
| Leader sequence according to SEQ ID NO: 60; | Embodiment S265 | Embodiment S266 |
| poly-A tail about 40-60 pA in length | | |
| Leader sequence according to SEQ ID NO: 60; | Embodiment S267 | Embodiment S268 |
| poly-A tail about 30-50 pA in length | | |
| Leader sequence according to SEQ ID NO: 60; | Embodiment S269 | Embodiment S270 |
| poly-A tail about 20-40 pA in length | | |
| Leader sequence according to SEQ ID NO: 60; | Embodiment S271 | Embodiment S272 |
| poly-A tail about 10-30 pA in length | | |
| Leader sequence according to SEQ ID NO: 62; | Embodiment S273 | Embodiment S274 |
| poly-A tail about 100 pA in length | | |
| Leader sequence according to SEQ ID NO: 62; | Embodiment S275 | Embodiment S276 |
| poly-A tail about 90 pA in length | | |
| Leader sequence according to SEQ ID NO: 62; | Embodiment S277 | Embodiment S278 |
| poly-A tail about 80 pA in length | | |
| Leader sequence according to SEQ ID NO: 62; | Embodiment S279 | Embodiment S280 |
| poly-A tail about 70 pA in length | | |
| Leader sequence according to SEQ ID NO: 62; | Embodiment S281 | Embodiment S282 |
| poly-A tail about 60 pA in length | | |
| Leader sequence according to SEQ ID NO: 62; | Embodiment S283 | Embodiment S284 |
| poly-A tail about 50 pA in length | | |
| Leader sequence according to SEQ ID NO: 62; | Embodiment S285 | Embodiment S286 |
| poly-A tail about 40 pA in length | | |
| Leader sequence according to SEQ ID NO: 62; | Embodiment S287 | Embodiment S288 |
| poly-A tail about 30 pA in length | | |
| Leader sequence according to SEQ ID NO: 62; | Embodiment S289 | Embodiment S290 |
| poly-A tail about 20 pA in length | | |
| Leader sequence according to SEQ ID NO: 62; | Embodiment S291 | Embodiment S292 |
| poly-A tail about 80-100 pA in length | | |
| Leader sequence according to SEQ ID NO: 62; | Embodiment S293 | Embodiment S294 |
| poly-A tail about 70-90 pA in length | | |
| Leader sequence according to SEQ ID NO: 62; | Embodiment S295 | Embodiment S296 |
| poly-A tail about 60-80 pA in length | | |
| Leader sequence according to SEQ ID NO: 62; | Embodiment S297 | Embodiment S298 |
| poly-A tail about 50-70 pA in length | | |
| Leader sequence according to SEQ ID NO: 62; | Embodiment S299 | Embodiment S300 |
| poly-A tail about 40-60 pA in length | | |
| Leader sequence according to SEQ ID NO: 62; | Embodiment S301 | Embodiment S302 |
| poly-A tail about 30-50 pA in length | | |
| Leader sequence according to SEQ ID NO: 62; | Embodiment S303 | Embodiment S304 |
| poly-A tail about 20-40 pA in length | | |
| Leader sequence according to SEQ ID NO: 62; | Embodiment S305 | Embodiment S306 |
| poly-A tail about 10-30 pA in length | | |
| Leader sequence according to SEQ ID NO: 63; | Embodiment S307 | Embodiment S308 |
| poly-A tail about 100 pA in length | | |
| Leader sequence according to SEQ ID NO: 63; | Embodiment S309 | Embodiment S310 |
| poly-A tail about 90 pA in length | | |
| Leader sequence according to SEQ ID NO: 63; | Embodiment S311 | Embodiment S312 |
| poly-A tail about 80 pA in length | | |
| Leader sequence according to SEQ ID NO: 63; | Embodiment S313 | Embodiment S314 |
| poly-A tail about 70 pA in length | | |
| Leader sequence according to SEQ ID NO: 63; | Embodiment S315 | Embodiment S316 |
| poly-A tail about 60 pA in length | | |
| Leader sequence according to SEQ ID NO: 63; | Embodiment S317 | Embodiment S318 |
| poly-A tail about 50 pA in length | | |
| Leader sequence according to SEQ ID NO: 63; | Embodiment S319 | Embodiment S320 |
| poly-A tail about 40 pA in length | | |
| Leader sequence according to SEQ ID NO: 63; | Embodiment S321 | Embodiment S322 |
| poly-A tail about 30 pA in length | | |
| Leader sequence according to SEQ ID NO: 63; | Embodiment S323 | Embodiment S324 |
| poly-A tail about 20 pA in length | | |
| Leader sequence according to SEQ ID NO: 63; | Embodiment S325 | Embodiment S326 |
| poly-A tail about 80-100 pA in length | | |
| Leader sequence according to SEQ ID NO: 63; | Embodiment S327 | Embodiment S328 |
| poly-A tail about 70-90 pA in length | | |
| Leader sequence according to SEQ ID NO: 63; | Embodiment S329 | Embodiment S330 |
| poly-A tail about 60-80 pA in length | | |
| Leader sequence according to SEQ ID NO: 63; | Embodiment S331 | Embodiment S332 |
| poly-A tail about 50-70 pA in length | | |
| Leader sequence according to SEQ ID NO: 63; | Embodiment S333 | Embodiment S334 |
| poly-A tail about 40-60 pA in length | | |
| Leader sequence according to SEQ ID NO: 63; | Embodiment S335 | Embodiment S336 |
| poly-A tail about 30-50 pA in length | | |
| Leader sequence according to SEQ ID NO: 63; | Embodiment S337 | Embodiment S338 |
| poly-A tail about 20-40 pA in length | | |
| Leader sequence according to SEQ ID NO: 63; | Embodiment S339 | Embodiment S340 |
| poly-A tail about 10-30 pA in length | | |

In some embodiments, the recombinant DNA molecule (e.g., DNA template) comprises, from 5′ to 3′: (i) a T7 polymerase promoter sequence; (ii) a leader sequence according to SEQ ID NO: 53; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a SapI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 53; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsaI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 58; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a SapI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 58; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail (e.g., a poly-A tail about 70 bp, about 60-80 bp, or about 50-90 bp in length), and (vi) a 3′ junctional cleavage sequence comprising or consisting of a BsaI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 58; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail about 70 bp in length, and (vi) a 3′ junctional cleavage sequence comprising or consisting of a SapI restriction enzyme recognition site.

In some embodiments, the recombinant DNA molecule (e.g., DNA template) comprises, from 5′ to 3′: (i) a T7 polymerase promoter sequence; (ii) a leader sequence according to SEQ ID NO: 58; (iii) a 5′ junctional cleavage sequence comprising or consisting of a 5′ Pistol ribozyme sequence according to SEQ ID NO: 64; (iv) a polynucleotide encoding a SVV synthetic RNA viral genome; (v) a poly-A tail about 70 bp in length, and (vi) a 3′ junctional cleavage sequence comprising or consisting of a BsaI restriction enzyme recognition site.

In some embodiments, the recombinant DNA molecule (e.g., DNA template) comprises a DNA polynucleotide which encodes a SVV viral genome and comprises or consists of a sequence according to SEQ ID NO: 52. In some embodiments, the DNA polynucleotide encoding the SVV strain viral genome comprises or consists of a sequence having at least 80%, at least 85%, at least 90%, at least 95%, at least 96%, at least 97%, at least 98%, at least 99%, or 100% (including all ranges and subranges therebetween) sequence identity according to SEQ ID NO: 52.

In some embodiments, the recombinant DNA molecule (e.g., DNA template) comprises a DNA polynucleotide which encodes a SVV viral genome and comprises or consists of a sequence according to SEQ TD NO: 92. In some embodiments, the DNA polynucleotide encoding the SVV strain viral genome comprises or consists of a sequence having at least 80, at least 85%, at least 90%, at least 95%, at least 96%, at least 97%, at least 98%, at least 99%, or 100% (including all ranges and subranges therebetween) sequence identity according to SEQ TD NO: 92.

Exemplary embodiments of DNA templates encoding SVV or CVA genomes are provided below in Table 18.

TABLE 18
Exemplary DNA Template Structures

| | | | Encoded | PolyA | | Full |
|---|---|---|---|---|---|---|
| Promoter | Leader | 5′ JCS | Genome | Tail | 3′ JCS | Template |
| T7 | SEQ ID NO: 15 | SEQ ID NO: 18 | CVA21 KY | 70 AA | SEQ ID NO: 86 | SEQ ID NO: 20 |
| Promoter | (CVA21-L6) | | Strain | | (BsmBI) | |
| T7 | SEQ ID NO: 15 | SEQ ID NO: 18 | CVA21 EF | 70 AA | SEQ ID NO: 86 | SEQ ID NO: 21 |
| Promoter | (CVA21-L6) | | Strain | | (BsmBI) | |
| T7 | SEQ ID NO: 15 | SEQ ID NO: 18 | CVA21 | 70 AA | SEQ ID NO: 86 | SEQ ID NO: 22 |
| Promoter | (CVA21-L6) | | Kuykendall | | (BsmBI) | |
| | | | Strain | | | |
| T7 | SEQ ID NO: 15 | SEQ ID NO: 18 | CVA21 KY | 70 AA | SEQ ID NO: 90 | SEQ ID NO: 93 |
| Promoter | (CVA21-L6) | | Strain | | (BsaI) | |
| T7 | SEQ ID NO: 15 | SEQ ID NO: 18 | CVA21 EF | 70 AA | SEQ ID NO: 90 | SEQ ID NO: 95 |
| Promoter | (CVA21-L6) | | Strain | | (BsaI) | |
| T7 | SEQ ID NO: 15 | SEQ ID NO: 18 | CVA21 | 70 AA | SEQ ID NO: 90 | SEQ ID NO: 94 |
| Promoter | (CVA21-L6) | | Kuykendall | | (BsaI) | |
| | | | Strain | | | |
| T7 | SEQ ID NO: 53 | SEQ ID NO: 64 | SVV | 70 AA | SEQ ID NO: 82 | SEQ ID NO: 52 |
| Promoter | (SVV-L0) | | | | (SapI) | |
| T7 | SEQ ID NO: 58 | SEQ ID NO: 64 | SVV | 70 AA | SEQ ID NO: 82 | SEQ ID NO: 92 |
| Promoter | (SVV-L5) | | | | (SapI) | |

### Particles Comprising Synthetic RNA Genomes

In some embodiments, the synthetic RNA genomes described herein are encapsulated in “particles.” As used herein, a particle refers to a non-tissue derived composition of matter such as liposomes, lipoplexes, nanoparticles, nanocapsules, microparticles, microspheres, lipid particles, exosomes, vesicles, and the like. In certain embodiments, the particles are non-proteinaceous and non-immunogenic. In such embodiments, encapsulation of the synthetic RNA genomes described herein allows for delivery of a viral genome without the induction of a systemic, anti-viral immune response and mitigates the effects of neutralizing anti-viral antibodies. Further, encapsulation of the synthetic RNA genomes described herein shields the genomes from degradation and facilitates the introduction into target host cells. In some embodiments, the present disclosure provides a nanoparticle comprising a synthetic RNA genome described herein. In some embodiments, the nanoparticle is a lipid nanoparticle. In some embodiments, the nanoparticle further comprises a second RNA molecule encoding a payload molecule.

In some embodiments, the particle is biodegradable in a subject. In such embodiments, multiple doses of the particles can be administered to a subject without an accumulation of particles in the subject. Examples of suitable particles include polystyrene particles, poly(lactic-co-glycolic acid) PLGA particles, polypeptide-based cationic polymer particles, cyclodextrin particles, chitosan, N,N,N-trimethyl chitosan particles, lipid based particles, poly(O-amino ester) particles, low-molecular-weight polyethylenimine particles, polyphosphoester particles, disulfide cross-linked polymer particles, polyamidoamine particles, polyethylenimine (PEI) particles, and PLURIONICS stabilized polypropylene sulfide particles.

In some embodiments, the polynucleotides described herein are encapsulated in inorganic particles. In some embodiments, the inorganic particles are gold nanoparticles (GNP), gold nanorods (GNR), magnetic nanoparticles (MNP), magnetic nanotubes (MNT), carbon nanohorns (CNH), carbon fullerenes, carbon nanotubes (CNT), calcium phosphate nanoparticles (CPNP), mesoporous silica nanoparticles (MSN), silica nanotubes (SNT), or a starlike hollow silica nanoparticles (SHNP).

Preferably, the particles described herein are nanoscopic in size, in order to enhance solubility, avoid clearance by phagocytic cells and possible complications caused by aggregation in vivo and to facilitate pinocytosis. In some embodiments, the particle has an average diameter of about less than about 1000 nm. In some embodiments, the particle has an average diameter of less than about 500 nm. In some embodiments, the particle has an average diameter of between about 30 and about 100 nm, between about 50 and about 100 nm, or between about 75 and about 100 nm. In some embodiments, the particle has an average diameter of between about 30 and about 75 nm or between about 30 and about 50 nm. In some embodiments, the particle has an average diameter between about 100 and about 500 nm. In some embodiments, the particle has an average diameter between about 200 and 400 nm. In some embodiments, the particle has an average size of about 350 nm.

### Exosomes

In some embodiments, the synthetic RNA genomes described herein are encapsulated in exosomes. Exosomes are small membrane vesicles of endocytic origin that are released into the extracellular environment following fusion of multivesicular bodies with the plasma membrane of the parental cell (e.g., the cell from which the exosome is released, also referred to herein as a donor cell). The surface of an exosome comprises a lipid bilayer derived from the parental cell's cell membrane and can further comprise membrane proteins expressed on the parental cell surface. In some embodiments, exosomes may also contain cytosol from the parental cell. Exosomes are produced by many different cell types including epithelial cells, B and T lymphocytes, mast cells (MC), and dendritic cells (DC) and have been identified in blood plasma, urine, bronchoalveolar lavage fluid, intestinal epithelial cells, and tumor tissues. Because the composition of an exosome is dependent on the parental cell type from which they are derived, there are no “exosome-specific” proteins. However, many exosomes comprise proteins associated with the intracellular vesicles from which the exosome originated in the parental cells (e.g., proteins associated with and/or expressed by endosomes and lysosomes). For example, exosomes can be enriched in antigen presentation molecules such as major histocompatibility complex I and II (MHC-I and MHC-II), tetraspanins (e.g., CD63), several heat shock proteins, cytoskeletal components such as actins and tubulins, proteins involved in intracellular membrane fusion, cell-cell interactions (e.g. CD54), signal transduction proteins, and cytosolic enzymes.

Exosomes may mediate transfer of cellular proteins from one cell (e.g., a parental cells) to a target or recipient cell by fusion of the exosomal membrane with the plasma membrane of the target cell. As such, modifying the material that is encapsulated by the exosome provides a mechanism by which exogenous agents, such as the polynucleotides described herein, may be introduced to a target cell. Exosomes that have been modified to contain one or more exogenous agents (e.g., a polynucleotide described herein) are referred to herein as “modified exosomes”. In some embodiments, modified exosomes are produced by introduction of the exogenous agent (e.g., a polynucleotide described herein) are introduced into a parental cell. In such embodiments, an exogenous nucleic acid is introduced into the parental, exosome-producing cells such that the exogenous nucleic acid itself, or a transcript of the exogenous nucleic acid is incorporated into the modified exosomes produced from the parental cell. The exogenous nucleic acids can be introduced to the parental cell by means known in the art, for example transduction, transfection, transformation, electroporation and/or microinjection of the exogenous nucleic acids.

In some embodiments, modified exosomes are produced by directly introducing a synthetic RNA genome described herein into an exosome. In some embodiments, a synthetic RNA genome described herein is introduced into an intact exosome. “Intact exosomes” refer to exosomes comprising proteins and/or genetic material derived from the parental cell from which they are produced. Methods for obtaining intact exosomes are known in the art (See e.g., Alvarez-Erviti L. et al., Nat Biotechnol. 2011 April; 29(4):34-5; Ohno S, et al., Mol Ther 2013 January; 21(1):185-91; and EP Patent Publication No. 2010663).

In particular embodiments, synthetic RNA genomes are introduced into empty exosomes. “Empty exosomes” refer to exosomes that lack proteins and/or genetic material (e.g., DNA or RNA) derived from the parental cell. Methods to produce empty exosomes (e.g., lacking parental cell-derived genetic material) are known in the art including UV-exposure, mutation/deletion of endogenous proteins that mediate loading of nucleic acids into exosomes, as well as electroporation and chemical treatments to open pores in the exosomal membranes such that endogenous genetic material passes out of the exosome through the open pores. In some embodiments, empty exosomes are produced by opening the exosomes by treatment with an aqueous solution having a pH from about 9 to about 14 to obtain exosomal membranes, removing intravesicular components (e.g., intravesicular proteins and/or nucleic acids), and reassembling the exosomal membranes to form empty exosomes. In some embodiments, intravesicular components (e.g., intravesicular proteins and/or nucleic acids) are removed by ultracentrifugation or density gradient ultracentrifugation. In some embodiments, the membranes are reassembled by sonication, mechanical vibration, extrusion through porous membranes, electric current, or combinations of one or more of these techniques. In particular embodiments, the membranes are reassembled by sonication.

In some embodiments, loading of intact or empty exosomes with a synthetic RNA genome described herein to produce a modified exosome can be achieved using conventional molecular biology techniques such as in vitro transformation, transfection, and/or microinjection. In some embodiments, the exogenous agents (e.g., the polynucleotides described herein) are introduced directly into intact or empty exosomes by electroporation. In some embodiments, the exogenous agents (e.g., the polynucleotides described herein) are introduced directly into intact or empty exosomes by lipofection (e.g., transfection). Lipofection kits suitable for use in the production of exosome according to the present disclosure are known in the art and are commercially available (e.g., FuGENE® HD Transfection Reagent from Roche, and LIPOFECTAMINE™ 2000 from Invitrogen). In some embodiments, the exogenous agents (e.g., the polynucleotides described herein) are introduced directly into intact or empty exosomes by transformation using heat shock. In such embodiments, exosomes isolated from parental cells are chilled in the presence of divalent cations such as Ca²⁺ (in CaCl₂)) in order to permeabilize the exosomal membrane. The exosomes can then be incubated with the exogenous nucleic acids and briefly heat shocked (e.g., incubated at 42° C. for 30-120 seconds). In particular embodiments, loading of empty exosomes with exogenous agents (e.g., the polynucleotides described herein) can be achieved by mixing or co-incubation of the agents with the exosomal membranes after the removal of intravesicular components. The modified exosomes reassembled from the exosomal membranes will, therefore, incorporate the exogenous agents into the intravesicular space. Additional methods for producing exosome encapsulated nucleic acids are known in the art (See e.g., U.S. Pat. Nos. 9,889,210; 9,629,929; and 9,085,778; International PCT Publication Nos. WO 2017/161010 and WO 2018/039119).

Exosomes can be obtained from numerous different parental cells, including cell lines, bone-marrow derived cells, and cells derived from primary patient samples. Exosomes released from parental cells can be isolated from supernatants of parental cell cultures by means known in the art. For example, physical properties of exosomes can be employed to separate them from a medium or other source material, including separation on the basis of electrical charge (e.g., electrophoretic separation), size (e.g., filtration, molecular sieving, etc.), density (e.g., regular or gradient centrifugation) and Svedberg constant (e.g., sedimentation with or without external force, etc). Alternatively, or additionally, isolation can be based on one or more biological properties, and include methods that can employ surface markers (e.g., for precipitation, reversible binding to solid phase, FACS separation, specific ligand binding, non-specific ligand binding, etc.). Analysis of exosomal surface proteins can be determined by flow cytometry using fluorescently labeled antibodies for exosome-associated proteins such as CD63. Additional markers for characterizing exosomes are described in International PCT Publication No. WO 2017/161010. In yet further contemplated methods, the exosomes can also be fused using chemical and/or physical methods, including PEG-induced fusion and/or ultrasonic fusion.

In some embodiments, size exclusion chromatography can be utilized to isolate the exosomes. In some embodiments, the exosomes can be further isolated after chromatographic separation by centrifugation techniques (of one or more chromatography fractions), as is generally known in the art. In some embodiments, the isolation of exosomes can involve combinations of methods that include, but are not limited to, differential centrifugation as previously described (See Raposo, G. et al., J. Exp. Med. 183, 1161-1172 (1996)), ultracentrifugation, size-based membrane filtration, concentration, and/or rate zonal centrifugation.

In some embodiments, the exosomal membrane comprises one or more of phospholipids, glycolipids, fatty acids, sphingolipids, phosphoglycerides, sterols, cholesterols, and phosphatidylserine. In addition, the membrane can comprise one or more polypeptides and one or more polysaccharides, such as glycans. Exemplary exosomal membrane compositions and methods for modifying the relative amount of one or more membrane component are described in International PCT Publication No. WO 2018/039119.

In some embodiments, the particles are exosomes and have a diameter between about 30 and about 100 nm, between about 30 and about 200 nm, or between about 30 and about 500 nm. In some embodiments, the particles are exosomes and have a diameter between about 10 nm and about 100 nm, between about 20 nm and about 100 nm, between about 30 nm and about 100 nm, between about 40 nm and about 100 nm, between about 50 nm and about 100 nm, between about 60 nm and about 100 nm, between about 70 nm and about 100 nm, between about 80 nm and about 100 nm, between about 90 nm and about 100 nm, between about 100 nm and about 200 nm, between about 100 nm and about 150 nm, between about 150 nm and about 200 nm, between about 100 nm and about 250 nm, between about 250 nm and about 500 nm, or between about 10 nm and about 1000 nm. In some embodiments, the particles are exosomes and have a diameter between about 20 nm and 300 nm, between about 40 nm and 200 nm, between about 20 nm and 250 nm, between about 30 nm and 150 nm, or between about 30 nm and 100 nm.

### Compounds

### Compounds of Formula (I)

In various embodiments, provided herein are compounds of Formula (I):

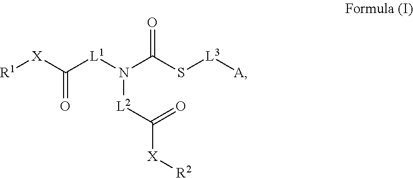

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain;
L³ is a bond, an optionally substituted C₁-C₆ alkylene chain, or a bivalent optionally substituted C₃-C₇ cycloalkylene.

In some embodiments, when A is —N(CH₃)(CH₃) and X is O, L³ is not a C₁-C₆ alkylene chain.

In some embodiments, the present disclosure includes a compound of Formula (I-a):

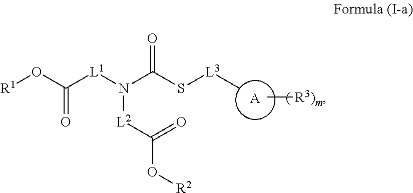

or a pharmaceutically acceptable salt or solvate thereof, wherein m is 0, 1, 2, 3, 4, 5, or 6.

In some embodiments, the present disclosure includes a compound of Formula (I-b):

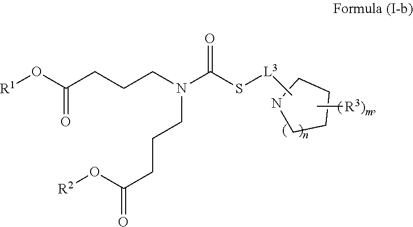

or a pharmaceutically acceptable salt or solvate thereof, wherein n is 0, 1, 2, or 3; and m is 0, 1, 2, 3, 4, 5, or 6.

In some embodiments, the present disclosure includes a compound of Formula (I-bi):

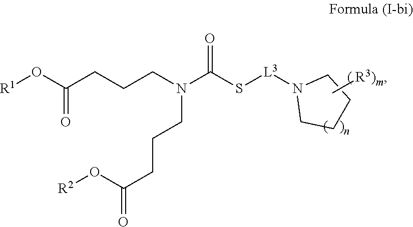

or a pharmaceutically acceptable salt or solvate thereof.

In some embodiments, the present disclosure includes a compound of Formula (I-bii):

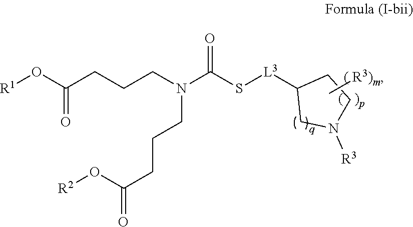

or a pharmaceutically acceptable salt or solvate thereof, wherein m is 0, 1, 2, or 3; and p and q are each 0, 1, 2, or 3, and wherein q+p is less than or equal to 3.

In some embodiments, the present disclosure includes a compound of Formula (I-biii):

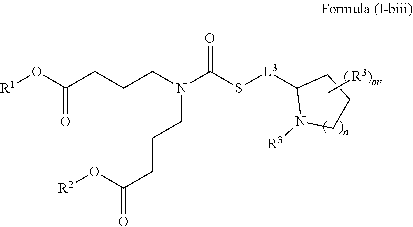

or a pharmaceutically acceptable salt or solvate thereof.

In some embodiments, the present disclosure includes a compound of Formula (I-c):

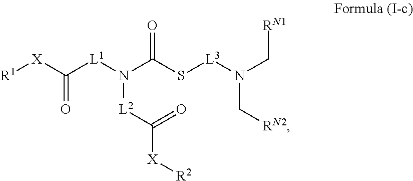

or a pharmaceutically acceptable salt or solvate thereof.

In some embodiments, A is —N(CH₂R^{N1})(CH₂R^{N2}) or an optionally substituted 4-7-membered heterocyclyl ring containing at least one N.

In some embodiments, A is —N(CH₂R^{N1})(CH₂R^{N2}). In some embodiments, R^{N1} and R^{N2} are each independently selected from hydrogen, hydroxy-C₁-C₃ alkylene, C₂-C₄ alkenyl, or C₃-C₄ cycloalkyl.).

In some embodiments, R^{N}₁ and R^{N2} are each independently selected from hydrogen, —CH₂CH═CH₂, —CH₂CH₂OH,

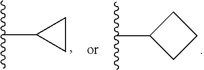

In some embodiments, R^{N}₁ and R^{N2} are the same. In some embodiments, R^{N}₁ and R^{N2} are each hydrogen. In some embodiments, R^{N}₁ and R^{N2} are each C₂-C₄ alkenyl, e.g., —CH₂CH═CH₂. In some embodiments, R^{N}₁ and R^{N2} are each hydroxy-C₁-C₃ alkylene, e.g., —CH₂CH₂OH. In some embodiments, R^{N}₁ and R^{N2} are different. In some embodiments, one of R^{N1} and R^{N2} is hydrogen and the other one is C₃-C₄ cycloalkyl. In some embodiments, one of R^{N}₁ and R^{N2} is hydrogen and the other one is.

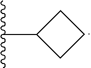 [SMILES: *C1CCC1]

In some embodiments, A is an optionally substituted 4-7-membered heterocyclyl ring containing at least one N. In some embodiments, A is an optionally substituted 4-7-membered heterocyclyl ring containing exactly one N. In some embodiments, A is an unsubstituted 4-7-membered heterocyclyl ring containing at least one N. In some embodiments, A is unsubstituted 4-7-membered heterocyclyl ring containing exactly one N. In some embodiments, A is an optionally substituted 5-6-membered heterocyclyl ring containing at least one N. In some embodiments, A is unsubstituted 5-6-membered heterocyclyl ring containing at least one N.

In some embodiments, A is an optionally substituted 4-7-membered heterocyclyl ring containing at least one N, and the N atom of A is a tertiary amine.

In some embodiments, A is an optionally substituted 4-7-membered heterocyclyl ring containing at least one N, further containing one or more S. In some embodiments, A is an optionally substituted 4-7-membered heterocyclyl ring containing at least one N, further containing exactly one S.

In some embodiments, A is selected from the group consisting of azetidine, pyrrolidine, piperidine, azepane, and thiomorpholine. In some embodiments, A is selected from the group consisting of pyrrolidine and piperidine.

In some embodiments, L¹ is selected from the group consisting of an optionally substituted C₁-C₂₀ alkylene chain and a bivalent optionally substituted C₁-C₂₀ alkenylene chain. In some embodiments, L² is selected from the group consisting of an optionally substituted C₁-C₂₀ alkylene chain and a bivalent optionally substituted C₁-C₂₀ alkenylene chain. In some embodiments, L¹ is an optionally substituted C₁-C₂₀ alkylene chain. In some embodiments, L² is an optionally substituted C₁-C₂₀ alkylene chain.

In some embodiments, L¹ and L² are the same. In some embodiments, L¹ and L² are different.

In some embodiments, L¹ is an optionally substituted C₁-C₁₀ alkylene chain. In some embodiments, L² is an optionally substituted C₁-C₁₀ alkylene chain. In some embodiments, L¹ is an optionally substituted C₁-C₅ alkylene chain. In some embodiments, L² is an optionally substituted C₁-C₅ alkylene chain.

In some embodiments, L¹ and L² are each —CH₂CH₂CH₂CH₂—. In some embodiments, L¹ and L² are each —CH₂CH₂CH₂—. In some embodiments, L¹ and L² are each —CH₂CH₂—.

In some embodiments, L³ is a bond, an optionally substituted C₁-C₆ alkylene chain, or a bivalent optionally substituted C₃-C₆ cycloalkylene. In some embodiments, L³ is a bond. In some embodiments, L³ is an optionally substituted C₁-C₆ alkylene chain. In some embodiments, L³ is an optionally substituted C₁-C₃ alkylene chain. In some embodiments, L³ is an unsubstituted C₁-C₃ alkylene chain. In some embodiments, L³ is —CH₂—. In some embodiments, L³ is —CH₂CH₂—. In some embodiments, L³ is —CH₂CH₂CH₂—. In some embodiments, L³ is a bivalent C₃-C₆ cyclcoalkylene. In some embodiments, L³ is.

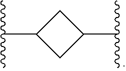 [SMILES: *C1CC(*)C1]

In some embodiments, the number of carbon atoms between the S of the thiolate of Formula (I) and the N of A is 2-10. In some embodiments, the number of carbon atoms between the S of the thiolate of Formula (I) and the N of A is 2-8. In some embodiments, the number of carbon atoms between the S of the thiolate of Formula (I) and the N of A is 2-5. In some embodiments, the number of carbon atoms between the S of the thiolate of Formula (I) and the N of A is 2-4. In some embodiments, the number of carbon atoms between the S of the thiolate of Formula (I) and the N of A is 2. In some embodiments, the number of carbon atoms between the S of the thiolate of Formula (I) and the N of A is 3. In some embodiments, the number of carbon atoms between the S of the thiolate of Formula (I) and the N of A is 4.

In some embodiments, R¹ is selected from the group consisting of optionally substituted C₁-C₃₁ aliphatic and optionally substituted steroidyl. In some embodiments, R² is selected from the group consisting of optionally substituted C₁-C₃₁ aliphatic and optionally substituted steroidyl. In some embodiments, R¹ is optionally substituted C₁-C₃₁ alkyl. In some embodiments, R² is optionally substituted C₁-C₃₁ alkyl. In some embodiments, R¹ is optionally substituted C₅-C₂₅ alkyl. In some embodiments, R² is optionally substituted C₅-C₂₅ alkyl. In some embodiments, R¹ is optionally substituted C₁₀-C₂₀ alkyl. In some embodiments, R² is optionally substituted C₁₀-C₂₀ alkyl. In some embodiments, R¹ is optionally substituted C₁₀-C₂₀ alkyl. In some embodiments, R² is optionally substituted C₁₀-C₂₀ alkyl. In some embodiments, R¹ is unsubstituted C₁₀-C₂₀ alkyl. In some embodiments, R² is unsubstituted C₁₀-C₂₀ alkyl.

In some embodiments, R¹ is optionally substituted C₁₄-C₁₆ alkyl. In some embodiments, R² is optionally substituted C₁₄-C₁₆ alkyl. In some embodiments, R¹ is unsubstituted C₁₄-C₁₆ alkyl. In some embodiments, R² is unsubstituted C₁₄-C₁₆ alkyl.

In some embodiments, R¹ is optionally substituted branched C₃-C₃₁ alkyl. In some embodiments, R² is optionally substituted branched C₃-C₃₁ alkyl. In some embodiments, R¹ is optionally substituted branched C₁₀-C₂₀ alkyl. In some embodiments, R² is optionally substituted branched C₁₀-C₂₀ alkyl. In some embodiments, R¹ is optionally substituted branched C₁₄-C₁₆ alkyl. In some embodiments, R² is optionally substituted branched C₁₄-C₁₆ alkyl. In some embodiments, R¹ is substituted branched C₃-C₃₁ alkyl. In some embodiments, R² is substituted branched C₃-C₃₁ alkyl. In some embodiments, R¹ is substituted branched C₁₀-C₂₀ alkyl. In some embodiments, R² is substituted branched C₁₀-C₂₀ alkyl. In some embodiments, R¹ is substituted branched C₁₄-C₁₆ alkyl. In some embodiments, R² is substituted branched C₁₄-C₁₆ alkyl.

In some embodiments, R¹ and R² are the same.

In some embodiments, R¹ and R² are different. In some embodiments, R¹ is optionally substituted C₆-C₂₀ alkenyl and R² is optionally substituted C₁₀-C₂₀ alkyl. In some embodiments, R¹ is C₆-C₂₀ alkenyl and R² is branched C₁₀-C₂₀ alkyl.

In some embodiments, A is 4-7-membered heterocyclyl ring containing at least one N and optionally substituted with 0-6 R³. In some embodiments, R³ is optionally substituted C₁-C₆ aliphatic. In some embodiments, R³ is optionally substituted C₁-C₃ aliphatic. In some embodiments, R³ is optionally substituted C₁-C₆ alkyl. In some embodiments, R³ is optionally substituted C₁-C₃ alkyl. In some embodiments, R³ is unsubstituted C₁-C₆ alkyl. In some embodiments, R³ is unsubstituted C₁-C₃ alkyl. In some embodiments, R³ is optionally substituted C₁-C₆ alkenyl. In some embodiments, R³ is optionally substituted C₁-C₃ alkenyl. In some embodiments, R³ is unsubstituted C₁-C₆ alkenyl. In some embodiments, R³ is unsubstituted C₁-C₃ alkenyl.

In some embodiments, R³ is substitute with 1-3 C₃-C₆ cycloalkyl. In some embodiments, R³ is substitute with 1 C₃-C₆ cycloalkyl. In some embodiments, R³ is substitute with a cyclopropanyl. In some embodiments, R³ is substitute with 1-3 —OH. In some embodiments, R³ is substitute with 1 —OH.

In some embodiments, m is 0, 1, 2, 3, 4, 5, or 6. In some embodiments m is 0 or 1. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is 0, 1, 2, or 3. In some embodiments n is 1 or 2. In some embodiments, n is 0. In some embodiments, n is 1. In some embodiments, n is 2. In some embodiments, n is 3.

In some embodiments, a compound of Formula (I) is a compound selected from Table 21, or a pharmaceutically acceptable salt or solvate thereof.

TABLE 21

| Compound | |
|---|---|
| No. | Structure |
| CAT1 | 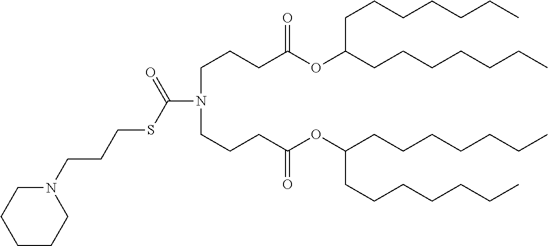 [SMILES: CCCCCCCC(CCCCCCC)OC(=O)CCCN(CCCC(=O)OC(CCCCCCC)CCCCCCC)C(=O)SCCCN1CCCCC1] |
| CAT2 | 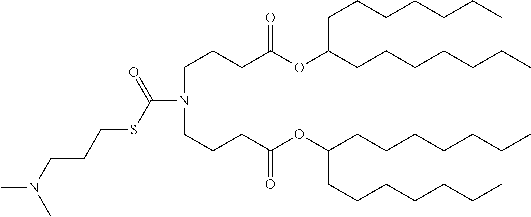 [SMILES: CCCCCCCC(CCCCCCC)OC(=O)CCCN(CCCC(=O)OC(CCCCCCC)CCCCCCC)C(=O)SCCCN(C)C] |
| CAT3 | 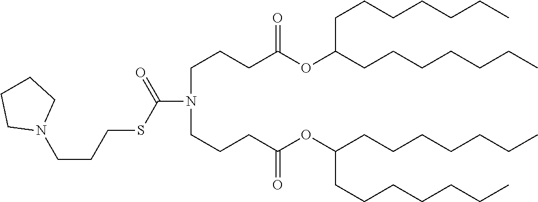 [SMILES: CCCCCCCC(CCCCCCC)OC(=O)CCCN(CCCC(=O)OC(CCCCCCC)CCCCCCC)C(=O)SCCCN1CCCC1] |
| CAT4 | 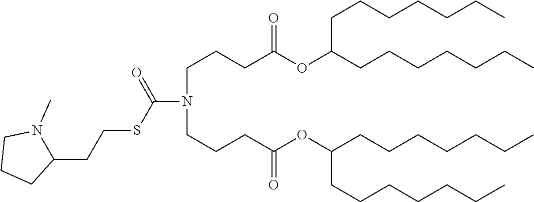 [SMILES: CCCCCCCC(CCCCCCC)OC(=O)CCCN(CCCC(=O)OC(CCCCCCC)CCCCCCC)C(=O)SCCC1CCCN1C] |
| CAT5 | 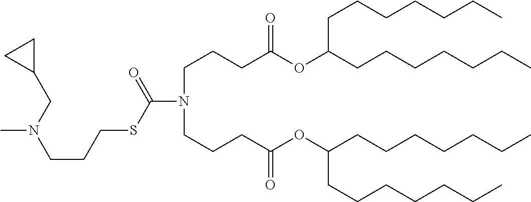 [SMILES: CCCCCCCC(CCCCCCC)OC(=O)CCCN(CCCC(=O)OC(CCCCCCC)CCCCCCC)C(=O)SCCCN(C)CC1CC1] |
| CAT6 | 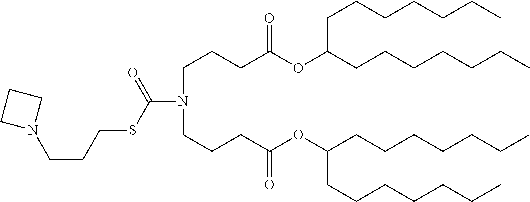 [SMILES: CCCCCCCC(CCCCCCC)OC(=O)CCCN(CCCC(=O)OC(CCCCCCC)CCCCCCC)C(=O)SCCCN1CCC1] |
| CAT7 | 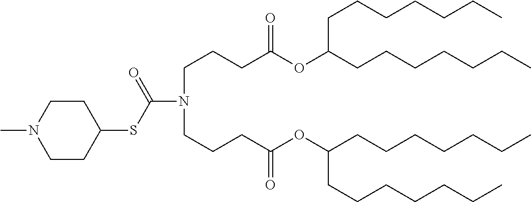 [SMILES: CCCCCCCC(CCCCCCC)OC(=O)CCCN(CCCC(=O)OC(CCCCCCC)CCCCCCC)C(=O)SC1CCN(C)CC1] |
| CAT8 | 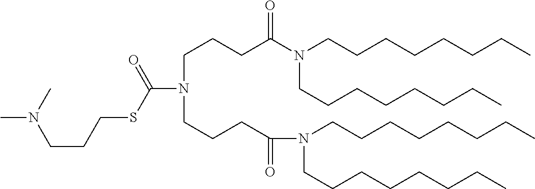 [SMILES: CCCCCCCCN(CCCCCCCC)C(=O)CCCN(CCCC(=O)N(CCCCCCCC)CCCCCCCC)C(=O)SCCCN(C)C] |
| CAT9 | 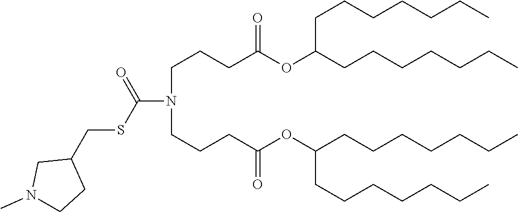 [SMILES: CCCCCCCC(CCCCCCC)OC(=O)CCCN(CCCC(=O)OC(CCCCCCC)CCCCCCC)C(=O)SCC1CCN(C)C1] |
| CAT10 | 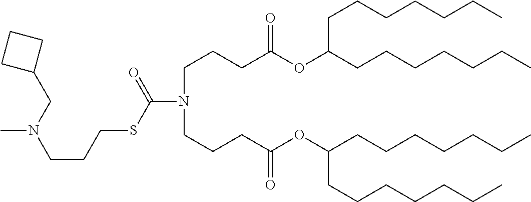 [SMILES: CCCCCCCC(CCCCCCC)OC(=O)CCCN(CCCC(=O)OC(CCCCCCC)CCCCCCC)C(=O)SCCCN(C)CC1CCC1] |
| CAT11 | 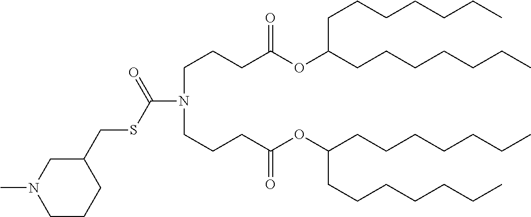 [SMILES: CCCCCCCC(CCCCCCC)OC(=O)CCCN(CCCC(=O)OC(CCCCCCC)CCCCCCC)C(=O)SCC1CCCN(C)C1] |
| CAT12 | 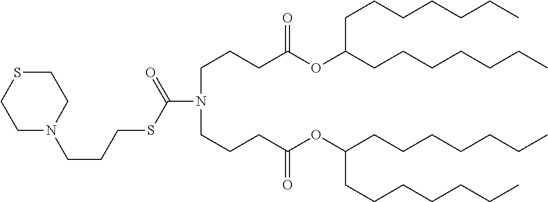 [SMILES: CCCCCCCC(CCCCCCC)OC(=O)CCCN(CCCC(=O)OC(CCCCCCC)CCCCCCC)C(=O)SCCCN1CCSCC1] |
| CAT13 | 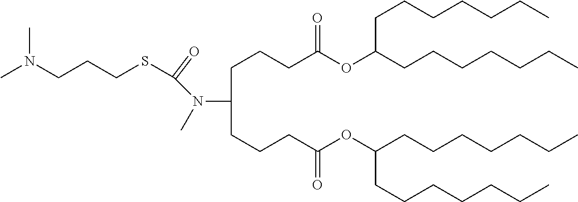 [SMILES: CCCCCCCC(CCCCCCC)OC(=O)CCCC(CCCC(=O)OC(CCCCCCC)CCCCCCC)N(C)C(=O)SCCCN(C)C] |
| CAT14 | 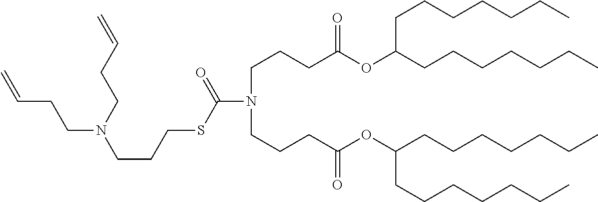 [SMILES: C=CCCN(CCC=C)CCCSC(=O)N(CCCC(=O)OC(CCCCCCC)CCCCCCC)CCCC(=O)OC(CCCCCCC)CCCCCCC] |
| CAT15 | 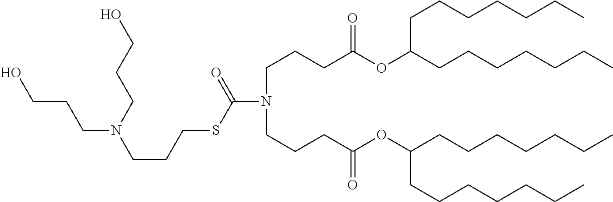 [SMILES: CCCCCCCC(CCCCCCC)OC(=O)CCCN(CCCC(=O)OC(CCCCCCC)CCCCCCC)C(=O)SCCCN(CCCO)CCCO] |
| CAT16 | 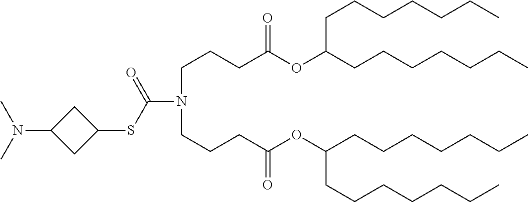 [SMILES: CCCCCCCC(CCCCCCC)OC(=O)CCCN(CCCC(=O)OC(CCCCCCC)CCCCCCC)C(=O)SC1CC(N(C)C)C1] |
| CAT17 | 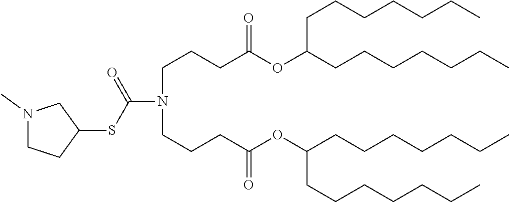 [SMILES: CCCCCCCC(CCCCCCC)OC(=O)CCCN(CCCC(=O)OC(CCCCCCC)CCCCCCC)C(=O)SC1CCN(C)C1] |
| CAT18 | 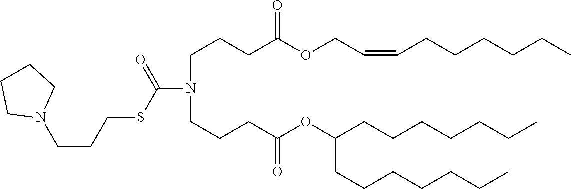 [SMILES: CCCCCC/C=C\COC(=O)CCCN(CCCC(=O)OC(CCCCCCC)CCCCCCC)C(=O)SCCCN1CCCC1] |
| CAT19 | 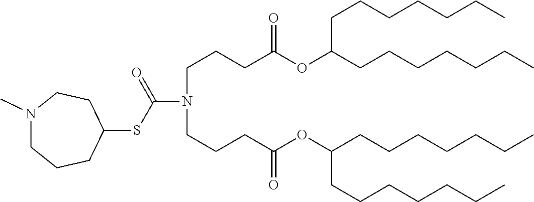 [SMILES: CCCCCCCC(CCCCCCC)OC(=O)CCCN(CCCC(=O)OC(CCCCCCC)CCCCCCC)C(=O)SC1CCCN(C)CC1] |
| CAT20 | 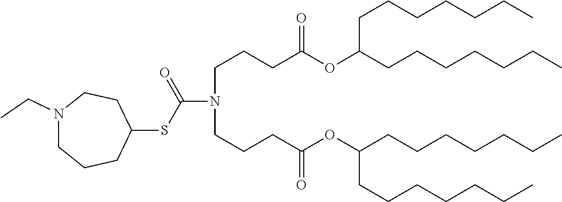 [SMILES: CCCCCCCC(CCCCCCC)OC(=O)CCCN(CCCC(=O)OC(CCCCCCC)CCCCCCC)C(=O)SC1CCCN(CC)CC1] |
| CAT21 | 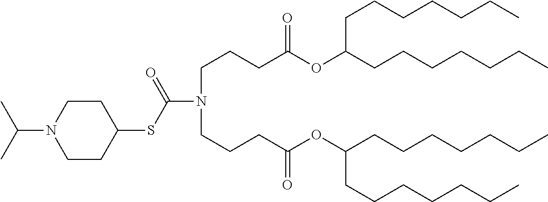 [SMILES: CCCCCCCC(CCCCCCC)OC(=O)CCCN(CCCC(=O)OC(CCCCCCC)CCCCCCC)C(=O)SC1CCN(C(C)C)CC1] |
| CAT22 | 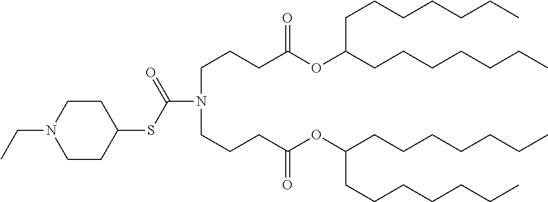 [SMILES: CCCCCCCC(CCCCCCC)OC(=O)CCCN(CCCC(=O)OC(CCCCCCC)CCCCCCC)C(=O)SC1CCN(CC)CC1] |
| CAT23 | 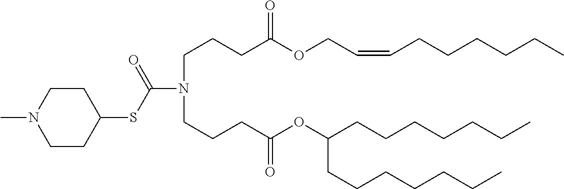 [SMILES: CCCCCC/C=C\COC(=O)CCCN(CCCC(=O)OC(CCCCCCC)CCCCCCC)C(=O)SC1CCN(C)CC1] |
| CAT24 | 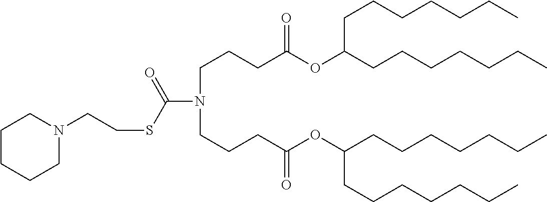 [SMILES: CCCCCCCC(CCCCCCC)OC(=O)CCCN(CCCC(=O)OC(CCCCCCC)CCCCCCC)C(=O)SCCN1CCCCC1] |
| CAT25 | 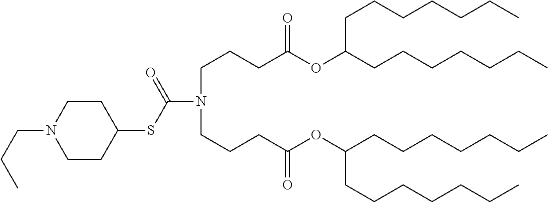 [SMILES: CCCCCCCC(CCCCCCC)OC(=O)CCCN(CCCC(=O)OC(CCCCCCC)CCCCCCC)C(=O)SC1CCN(CCC)CC1] |
| CAT26 | 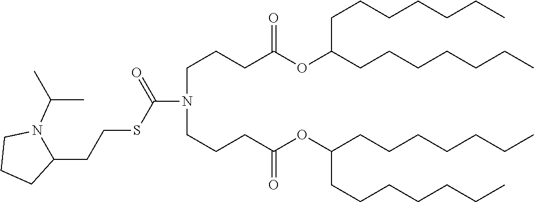 [SMILES: CCCCCCCC(CCCCCCC)OC(=O)CCCN(CCCC(=O)OC(CCCCCCC)CCCCCCC)C(=O)SCCC1CCCN1C(C)C] |
| CAT27 | 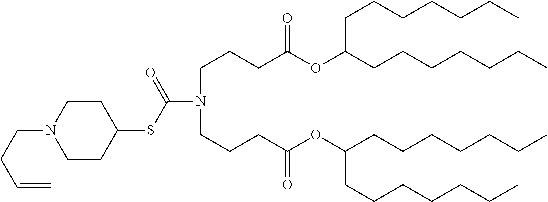 [SMILES: C=CCCN1CCC(SC(=O)N(CCCC(=O)OC(CCCCCCC)CCCCCCC)CCCC(=O)OC(CCCCCCC)CCCCCCC)CC1] |
| CAT28 | 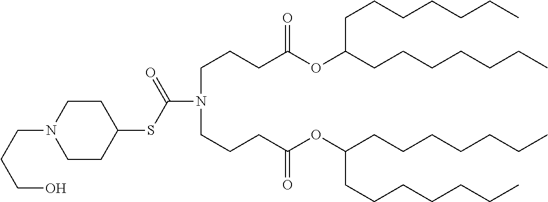 [SMILES: CCCCCCCC(CCCCCCC)OC(=O)CCCN(CCCC(=O)OC(CCCCCCC)CCCCCCC)C(=O)SC1CCN(CCCO)CC1] |
| CAT29 | 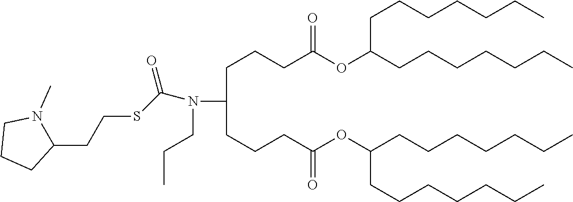 [SMILES: CCCCCCCC(CCCCCCC)OC(=O)CCCC(CCCC(=O)OC(CCCCCCC)CCCCCCC)N(CCC)C(=O)SCCC1CCCN1C] |
| CAT30 | 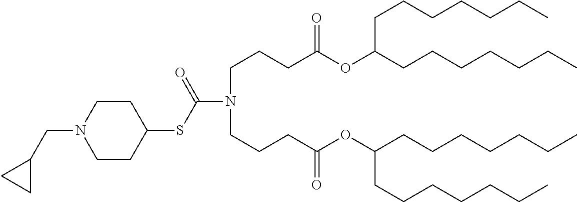 [SMILES: CCCCCCCC(CCCCCCC)OC(=O)CCCN(CCCC(=O)OC(CCCCCCC)CCCCCCC)C(=O)SC1CCN(CC2CC2)CC1] |
| CAT31 | 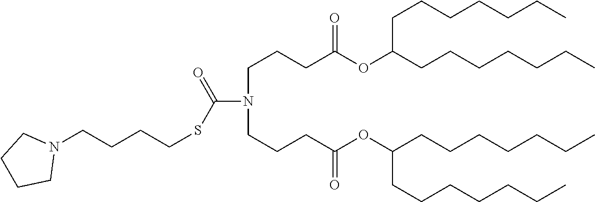 [SMILES: CCCCCCCC(CCCCCCC)OC(=O)CCCN(CCCC(=O)OC(CCCCCCC)CCCCCCC)C(=O)SCCCCN1CCCC1] |
| CAT32 | 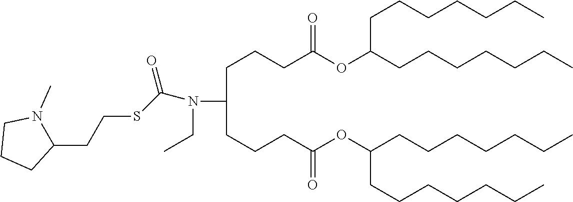 [SMILES: CCCCCCCC(CCCCCCC)OC(=O)CCCC(CCCC(=O)OC(CCCCCCC)CCCCCCC)N(CC)C(=O)SCCC1CCCN1C] |
| CAT33 | 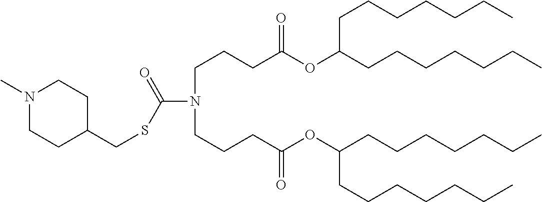 [SMILES: CCCCCCCC(CCCCCCC)OC(=O)CCCN(CCCC(=O)OC(CCCCCCC)CCCCCCC)C(=O)SCC1CCN(C)CC1] |
| CAT34 | 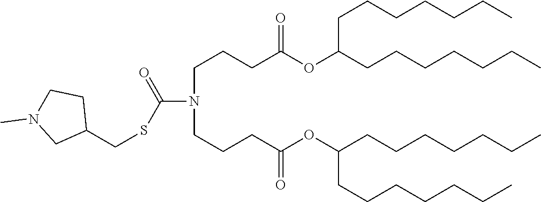 [SMILES: CCCCCCCC(CCCCCCC)OC(=O)CCCN(CCCC(=O)OC(CCCCCCC)CCCCCCC)C(=O)SCC1CCN(C)C1] |
| CAT35 | 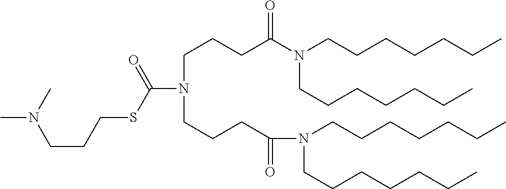 [SMILES: CCCCCCCN(CCCCCCC)C(=O)CCCN(CCCC(=O)N(CCCCCCC)CCCCCCC)C(=O)SCCCN(C)C] |

### Compounds of Formula (A)

In various embodiments, provided herein are compounds of Formula (A):

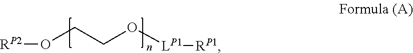

or a pharmaceutically acceptable salt thereof, wherein:
n is an integer between 10 to 200, inclusive of all endpoints;
L^{P1} is —[(CH₂)₀₋₃—C(O)O]₁₋₃—, —(CH₂)₀₋₃—C(O)O—(CH₂)₁₋₃-OC(O)—, or —C(O)N(H)—;
R^{P1} is C₅-C₂₅ alkyl or C₅-C₂₅ alkenyl; and
R^{P2} is hydrogen or —CH₃.

In some embodiments, Formula (A) is not HO—(CH₂CH₂O)ₙ—C(O)N(H)—(CH₂)₁₇CH₃.

In some embodiments, L^{P1} is —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, the PEG-lipid is a compound of Formula (A-a), Formula (A-b), Formula (A-c), Formula (A-d), or Formula (A-e):

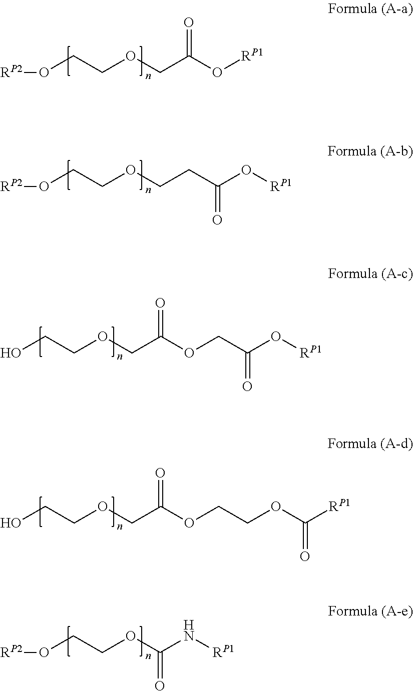

or a pharmaceutically acceptable salt thereof.

In some embodiments, R^{P1} is C₆-C₂₄, C₁₀-C₂₀, C₁₀-C₁₈, C₁₀-C₁₆, C₁₀-C₁₄, C₁₀-C₁₂, C₁₂-C₂₀, C₁₂-C₁₈, C₁₂-C₁₆, C₁₂-C₁₄, C₁₄-C₂₀, C₁₄-C₁₈, C₁₄-C₁₆, C₁₆-C₂₀, C₁₆-C₁₈, or C₁₈-C₂₀ alkyl. In some embodiments, R^{P1} is C₁₄-C₁₈ alkyl. In some embodiments, R^{P1} is C₁₄-C₁₆ alkyl. In some embodiments, R¹ is C₁₅-C₁₇ alkyl. In some embodiments, R¹ is C₁₆-C₁₈ alkyl. In some embodiments, R¹ is C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl. In some embodiments, R¹ is C₆-C₂₄, C₁₀-C₂₀, C₁₀-C₁₈, C₁₀-C₁₆, C₁₀-C₁₄, C₁₀-C₁₂, C₁₂-C₂₀, C₁₂-C₁₈, C₁₂-C₁₆, C₁₂-C₁₄, C₁₄-C₂₀, C₁₄-C₁₈, C₁₄-C₁₆, C₁₆-C₂₀, C₁₆-C₁₈, or C₁₈-C₂₀ alkenyl. In some embodiments, R¹ is C₁₄-C₁₈ alkenyl. In some embodiments, R¹ is C₁₄-16 alkenyl. In some embodiments, R^{P1} is C₁₅-C₁₇ alkenyl. In some embodiments, R¹ is C₁₆-C₁₈ alkenyl. In some embodiments, R¹ is C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkenyl.

In some embodiments, R^{P2} is hydrogen. In some embodiments, R^{P2} is —CH₃.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to 200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, a compound of Formula (A) is a compound selected from the group consisting of:
HO—(CH₂CH₂O)ₙ—CH₂C(O)O—(CH₂)₁₇CH₃, n is on average about 45;
H₃CO—(CH₂CH₂O)ₙ—CH₂C(O)O—(CH₂)₁₇CH₃, n is on average about 45;
HO—(CH₂CH₂O)ₙ—CH₂C(O)O—(CH₂)₁₅CH₃, n is on average about 45;
HO—(CH₂CH₂O)ₙ—CH₂C(O)O—(CH₂)₁₃CH₃, n is on average about 45; and
HO—(CH₂CH₂O)ₙ—C(O)N(H)—(CH₂)₁₇CH₃, n is on average about 45;
   or a pharmaceutically acceptable salt thereof.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Lipid Nanoparticles

In certain embodiments, the synthetic RNA viral genomes described herein are encapsulated in a lipid nanoparticle (LNP). In certain embodiments, the LNP comprises one or more lipids such as such as triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). In some embodiments, the LNP comprises one or more cationic lipids, one or more structural lipids, and one or more helper lipids. In some embodiments, the LNP comprises one or more cationic lipids, a cholesterol, and one or more neutral lipids.

In some embodiments, compounds of the present disclosure are used to form a nanoparticle. In some embodiments, the nanoparticle is a lipid nanoparticle (LNP). In some embodiments, an LNP comprises a PEG-lipid, an ionizable lipid, a helper lipid, and a structural lipid. In some embodiments, LNPs described herein are formulated for delivery of therapeutic agents to a subject in need thereof. In some embodiments, LNPs described herein are formulated for delivery of nucleic acid molecules to a subject in need thereof.

The formulation of lipids in an LNP significantly impacts the therapeutic use and efficacy of a particular LNP. For example, LNP formulations such as SS—OC/Cholesterol/DSPC/PEG2k-DPG typically display increased clearance rate upon repeat intravenous (IV) administration, e.g., in mice, non-human primates (NHPs), and/or humans and a much shorter circulation time in vivo post-second dose than post-first dose. The shortened circulation time can negatively impact the delivery efficiency of the LNPs, likely due to less exposure of the LNPs to the target. Therefore, while such formulations may be useful in delivering agents that do not require multiple administrations, their use for delivery of agents that require subsequent administration may be constrained by this shortened circulation time.

There remains a need for LNP formulations that demonstrate tunable circulation and exposure to target cells, e.g., sustained circulation and consistent exposure, in vivo upon repeat dosing. The present disclosure provides such LNP formulations by incorporating ionizable lipid and/or PEG-lipid of the disclosure into the lipid formulation of the LNP. The sustained circulation of the LNP of the present disclosure upon repeat administration consequently allows for sustained therapeutic effect of the synthetic RNA viral genomes encapsulated therein.

In some embodiments, in the absence of the ionizable lipid and/or PEG-lipid of the disclosure, rapid clearance of the LNP and components thereof upon repeated dosing reduces the delivery efficiency of the encapsulated synthetic RNA viral genome in subsequent doses as the body may clear the LNP prior to the release of the synthetic RNA viral genome. In some embodiments, ionizable lipid and/or PEG-lipid of the disclosure, when incorporated into an LNP, delays clearance of the LNP upon repeated dosing, allowing for the sustained release and therapeutic effect of the encapsulated synthetic RNA viral genome.

### Polyethyleneglycol (PEG)-Lipid

In some embodiments, the PEG-lipid of the disclosure comprises a hydrophilic head group and a hydrophobic lipid tail. In some embodiments, the hydrophilic head group is a PEG moiety. In some embodiments, PEG-lipid of the disclosure comprises a mono lipid tail. In some embodiments, PEG-lipid of the disclosure comprises a mono alkyl lipid tail, a mono alkenyl lipid tail, a mono alkynyl lipid tail, or a mono acyl lipid tail. In some embodiments, the mono lipid tail comprises an ether group, a carbonyl group, or an ester group. In some embodiments, the PEG-lipid of the disclosure may contain a polyoxyethylene alkyl ether, a polyoxyethylene alkenyl ether, or a polyoxyethylene alkynyl ether (such molecules are also known as BRIJ™ or Brij molecules). In some embodiments, the PEG-lipid of the disclosure may contain a polyoxyethylene alkyl ester, a polyoxyethylene alkenyl ester, or a polyoxyethylene alkynyl ester (such molecules are also known as MYRJ™ molecules).

In some embodiments, the PEG-lipid may contain di-acyl lipid tails.

In some embodiments, the PEG-lipid is a compound of Formula (A)

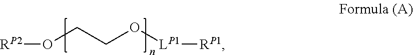

or a pharmaceutically acceptable salt or solvate thereof, wherein the variables are defined herein.

In some embodiments, the PEG-lipid is a compound of Formula (A′):

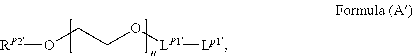

or a pharmaceutically acceptable salt thereof, wherein:
n is an integer between 10 to 200, inclusive of all endpoints;
L^{P1′} is a bond, —C(O)—, —[(CH₂)₀₋₃—C(O)O]₁₋₃—, —(CH₂)₀₋₃—C(O)O—(CH₂)₁₋₃—OC(O)—, or —C(O)N(H)—;
R^{P1′} is C₅-C₂₅ alkyl or C₅-C₂₅ alkenyl; and
R^{P2′} is hydrogen or —CH₃.

In some embodiments, L^{P1′} is a bond, —C(O)—, —CH₂C(O)O—, —CH₂CH₂C(O)O—, —CH₂C(O)OCH₂C(O)O—, —CH₂C(O)OCH₂CH₂OC(O)—, or —C(O)N(H)—. In some embodiments, R^{P1′} is R^{P1}. In some embodiments, R^{P2′} is R^{P2}.

In some embodiments, the PEG-lipid is a compound of Formula (A″):

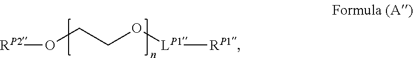

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃.

In some embodiments, L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

In some embodiments, the PEG-lipid is a compound of Formula (A″-a), Formula (A″-b), Formula (A″-c), Formula (A″-cd), Formula (A″-e), or Formula (A″-f):

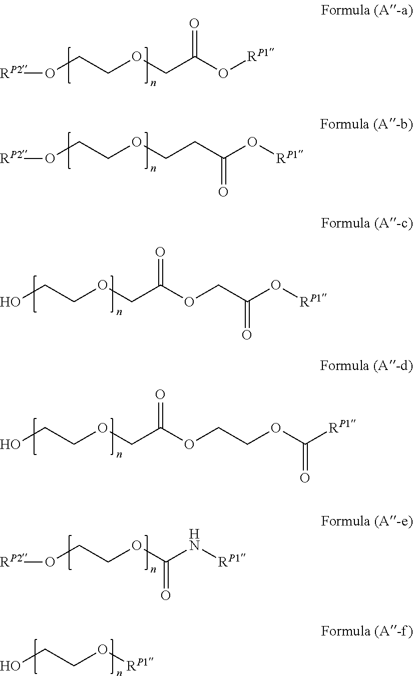

or a pharmaceutically acceptable salt thereof.

In some embodiments, R^{P1}″ is R¹. In some embodiments, R^{P2}″ is R^{P2}.

In some embodiments, the PEG-lipid is a compound of Formula (A″-f1):

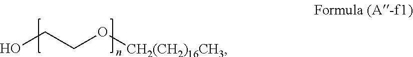

or a pharmaceutically acceptable salt thereof.

In some embodiments, the PEG-lipid is a compound of Formula (A″-f2):

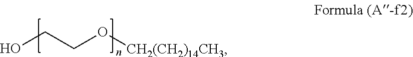

or a pharmaceutically acceptable salt thereof.

In some embodiments, the PEG-lipid is a compound of Formula (A″-f3):

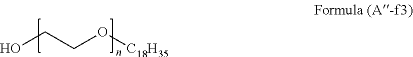

or a pharmaceutically acceptable salt thereof.

In some embodiments, a PEG-lipid of the disclosure is a compound of Formula (B):

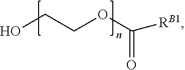

### Formula (B)

or a pharmaceutically acceptable salt thereof, wherein:
n is an integer between 10 to 200, inclusive of all endpoints; and
R^{B1} is C₅-C₂₅ alkyl or C₅-C₂₅ alkenyl.

In some embodiments, R^{B1} is R^{P1}.

In some embodiments, the PEG-lipid is a compound of Formula (B-a):

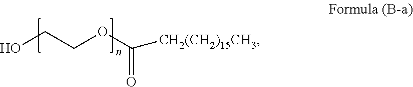

or a pharmaceutically acceptable salt thereof.

In some embodiments, the PEG-lipid is a compound of Formula (B-b):

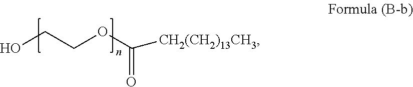

or a pharmaceutically acceptable salt thereof.

In some embodiments, n is, on average, 10 to 200, 10 to 180, 10 to 160, 10 to 140, 10 to 120, 10 to 100, 10 to 80, 10 to 60, 10 to 40, 10 to 20, 20 to 200, 20 to 180, 20 to 160, 20 to 140, 20 to 120, 20 to 100, 20 to 80, 20 to 60, 20 to 40, 40 to 200, 40 to 180, 40 to 160, 40 to 140, 40 to 120, 40 to 100, 40 to 80, 40 to 60, 60 to 200, 60 to 180, 60 to 160, 60 to 140, 60 to 120, 60 to 100, 60 to 80, 80 to 200, 80 to 180, 80 to 160, 80 to 140, 80 to 120, 80 to 100, 100 to 200, 100 to 180, 100 to 160, 100 to 140, 100 to 120, 120 to 200, 120 to 180, 120 to 160, 120 to 140, 140 to 200, 140 to 180, 140 to 160, 160 to 200, 160 to 180, or 180 to 200. In some embodiments, n is, on average, 10, 20, 30, 40, 50, 60, 70, 80, 90, 100, 110, 120, 130, 140, 150, 160, 170, 180, 190, or 200. In some embodiments, n is on average about 20. In some embodiments, n is on average about 40. In some embodiments, n is on average about 45. In some embodiments, n is on average about 50. In some embodiments, n is on average about 68. In some embodiments, n is on average about 75. In some embodiments, n is on average about 100.

In some embodiments, the PEG-lipid comprises a PEG moiety having an average molecular weight of about 500 to about 10,000 daltons. In some embodiments, the PEG-lipid comprises a PEG moiety having an average molecular weight of about 500 to about 5,000 daltons, about 500 to about 4,000 daltons, about 500 to about 3,000 daltons, about 500 to about 2,000 daltons, about 500 to about 1,000 daltons, about 500 to about 800 daltons, about 500 to about 600 daltons, about 600 to about 5,000 daltons, about 600 to about 4,000 daltons, about 600 to about 3,000 daltons, about 600 to about 2,000 daltons, about 600 to about 1,000 daltons, about 600 to about 800 daltons, about 800 to about 5,000 daltons, about 800 to about 4,000 daltons, about 800 to about 3,000 daltons, about 800 to about 2,000 daltons, about 800 to about 1,000 daltons, about 1,000 to about 5,000 daltons, about 1,000 to about 4,000 daltons, about 1,000 to about 3,000 daltons, about 1,000 to about 2,000 daltons, about 2,000 to about 5,000 daltons, about 2,000 to about 4,000 daltons, about 2,000 to about 3,000 daltons, about 3,000 to about 5,000 daltons, about 3,000 to about 4,000 daltons, about 5,000 to about 10,000 daltons, about 5,000 to about 7,500 daltons, or about 7,500 to about 10,000 daltons. In some embodiments, the PEG moiety of the PEG-lipid has an average molecular weight of about 1,500 to about 2,500 daltons. In some embodiments, the PEG moiety of the PEG-lipid has an average molecular weight of about 1,000 to about 5,000 daltons. In some embodiments, the PEG-lipid comprises a PEG moiety having an average molecular weight of about 500, about 600, about 800, about 1,000, about 1,500, about 2,000, about, 2500, about 3,000, about 3,500, about 4,000, about 4,500, about 5,000, about 6,000, about 7,000, about 8,000, about 9,000, or about 10,000 daltons. In some embodiments, the PEG-lipid comprises a PEG moiety having an average molecular weight of at least 500, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000, or at least 10,000 daltons. In some embodiments, the PEG-lipid comprises a PEG moiety having an average molecular weight of no more than 500, no more than 1,000, no more than 1,500, no more than 2,000, no more than 2,500, no more than 3,000, no more than 3,500, no more than 4,000, no more than 4,500, no more than 5,000, no more than 6,000, no more than 7,000, no more than 8,000, no more than 9,000, or no more than 10,000 daltons. All values are inclusive of all endpoints.

In some embodiments, the PEG-lipid is polyoxyethylene (100) stearyl ether, polyoxyethylene (20) cetyl ether, polyoxyethylene (20) oleyl ether, polyoxyethylene (20) stearyl ether, or a mixture thereof. In some embodiments, the PEG-lipid is polyoxyethylene (100) stearate, polyoxyethylene (50) stearate, polyoxyethylene (40) stearate, polyoxyethylene palmitate, or a mixture thereof.

In some embodiments of the disclosure, the PEG-lipid is

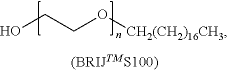

having a CAS number of 9005-00, a linear formula of C₁₈H₃₇(OCH₂CH₂)ₙOH wherein n is 100. BRIJ™ S100 is also known, generically, as polyoxyethylene (100) stearyl ether. Accordingly, in some embodiments, the PEG-lipid is HO-PEG100-CH₂(CH₂)₁₆CH₃.

In some embodiments of the disclosure, the PEG-lipid is

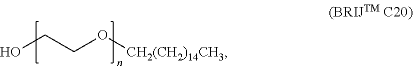

having a CAS number of 9004-95-9, a linear formula of C₁₆H₃₃(OCH₂CH₂)ₙOH wherein n is 20. BRIJ™ C₂₀ is also known as BRIJ™ 58, and, generically, as polyethylene glycol hexadecyl ether, polyoxyethylene (20) cetyl ether. Accordingly, in some embodiments, the PEG-lipid is HO-PEG20-CH₂(CH₂)₁₄CH₃.

In some embodiments of the disclosure, the PEG-lipid is

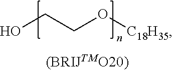

having a CAS number of 9004-98-2, a linear formula of C₁₈H₃₅(OCH₂CH₂)ₙOH wherein n is 20. BRIJ™ 020 is also known, generically, as polyoxyethylene (20) oleyl ether. Accordingly, in some embodiments, the PEG-lipid is HO-PEG20-C₁₈H₃₅.

In some embodiments of the disclosure, the PEG-lipid is

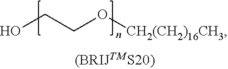

having a CAS number of 9005-00-9, a linear formula of C₁₈H₃₇(OCH₂CH₂)ₙOH wherein n is 20. BRIJ™ S20 is also known, generically, as polyethylene glycol octadecyl ether or polyoxyethylene (20) stearyl ether. Accordingly, in some embodiments, the PEG-lipid is HO-PEG20-CH₂(CH₂)₁₆CH₃.

In some embodiments of the disclosure, the PEG-lipid is

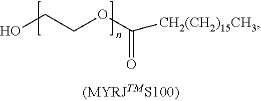

having a CAS number of 9004-99-3, a linear formula of C₁₇H₃₅C(O)(OCH₂CH₂)ₙOH wherein n is 100. MYRJ™ S100 is also known, generically, as polyoxyethylene (100) stearate. Accordingly, in some embodiments, the PEG-lipid is HO-PEG100-CH₂(CH₂)₁₅CH₃.

In some embodiments of the disclosure, the PEG-lipid is

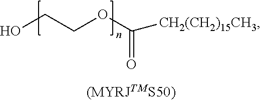

having a CAS number of 9004-99-3, a linear formula of C₁₇H₃₅C(O)(OCH₂CH₂)ₙOH wherein n is 50. MYRJ™ S50 is also known, generically, as polyoxyethylene (50) stearate. Accordingly, in some embodiments, the PEG-lipid is HO-PEG50-CH₂(CH₂)₁₅CH₃.

In some embodiments of the disclosure, the PEG-lipid is

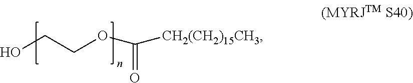

having a CAS number of 9004-99-3, a linear formula of C₁₇H₃₅C(O)(OCH₂CH₂)ₙOH wherein n is 40. MYRJ™ S40 is also known, generically, as polyoxyethylene (40) stearate. Accordingly, in some embodiments, the PEG-lipid is HO-PEG40-CH₂(CH₂)₁₅CH₃.

In some embodiments of the disclosure, the PEG-lipid is

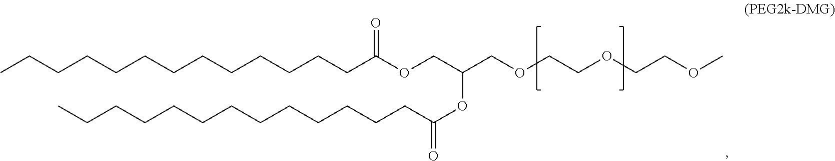

having a CAS number of 1607430-62-04, a linear formula of C₁₂₂H₂₄₂₀₅₀. PEG2k-DMG is also known as 1,2-dimyristoyl-rac-glycero-3-methoxypolyethylene glycol-2000.

In some embodiments of the disclosure, the PEG-lipid is:

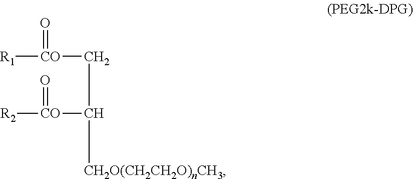

having an alkyl composition of R₁COO═C16:0, R₂COO═C16:0. PEG2k-DPG is also known, generically, as 1,2-Dipalmitoyl-rac-glycero-3-methylpolyoxyethylene.

In some embodiments of the disclosure, the PEG-lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE), 1,2-distearoyl-snglycerol, methoxypolyethylene glycol (PEG2k-DSG), poly(ethylene glycol)-2000-dimethacrylate (PEG2k-DMA), or 1,2-distearyloxypropyl-3-amine-N-[methoxy(polyethylene glycol)-2000] (PEG2k-DSA). In some embodiments, the PEG-lipid may be PEG2k-DMG. In some embodiments, the PEG-lipid may be PEG2k-DSG. In other embodiments, the PEG-lipid may be PEG2k-DSPE. In some embodiments, the PEG-lipid may be PEG2k-DMA. In yet other embodiments, the PEG-lipid may be PEG2k-C-DMA. In some embodiments, the PEG-lipid may be PEG2k-DSA. In other embodiments, the PEG-lipid may be PEG2k-C₁₁. In some embodiments, the PEG-lipid may be PEG2k-C₁₄. In some embodiments, the PEG-lipid may be PEG2k-C₁₆. In some embodiments, the PEG-lipid may be PEG2k-C₁₈.

In some embodiments, a PEG-lipid having single lipid tail of the disclosure (e.g., PEG-lipid of Formula (A), (A′), (A″), or (B)) may reduce accelerated blood clearance (ABC) upon administration and/or repeat administration of an LNP composition of the disclosure. In some embodiments, a PEG-lipid having single lipid tail of the disclosure may reduce or deplete PEG-specific antibodies (e.g., anti-PEG IgM) generated by a subject's immune system upon administration and/or repeat administration of an LNP composition of the disclosure.

In some embodiments, the PEG-lipid comprises a poly(ethylene)glycol chain of up to 5 kDa in length covalently attached to a lipid comprising one or more C₆-C₂₀ alkyls. In some embodiments, the PEG-lipid is 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K). In some embodiments, the PEG-lipid is DSPE-PEG5K. In some embodiments, the PEG-lipid is DPG-PEG2K. In some embodiments, the PEG-lipid is DSG-PEG2K. In some embodiments, the PEG-lipid is DMG-PEG2K. In some embodiments, the PEG-lipid is DSG-PEG5K. In some embodiments, the PEG-lipid is DMG-PEG5K.

In some embodiments, the PEG lipid is a cleavable PEG lipid. Examples of PEG derivatives with cleavable bonds include those modified with peptide bonds (Kulkarni et al. (2014). Mmp-9 responsive PEG cleavable nanovesicles for efficient delivery of chemotherapeutics to pancreatic cancer. Mol Pharmaceutics 11:2390-9; Lin et al. (2015). Drug/dye-loaded, multifunctional peg-chitosan-iron oxide nanocomposites for methotraxate synergistically self-targeted cancer therapy and dual model imaging. ACS Appl Mater Interfaces 7:11908-20.), disulfide keys (Yan et al (2014). A method to accelerate the gelation of disulfide-crosslinked hydrogels. Chin J Polym Sci 33:118-27; Wu & Yan (2015). Copper nanopowder catalyzed cross-coupling of diaryl disulfides with aryl iodides in PEG-400. Synlett 26:537-42), vinyl ether bonds, hydrazone bonds (Kelly et al. (2016). Polymeric prodrug combination to exploit the therapeutic potential of antimicrobial peptides against cancer cells. Org Biomol Chem 14:9278-86.), and ester bonds (Xu et al. (2008). Esterase-catalyzed dePEGylation of pH-sensitive vesicles modified with cleavable PEG-lipid derivatives. J Control Release 130:238-45). See also, Fang et al., (2017) Cleaveable PEGylation: a strategy for overcoming the “PEG dilemma” in efficient drug delivery. Drug Delivery 24:2, 22-32.

In some embodiments, the PEG lipid is an activated PEG lipid. Exemplary activated PEG lipids include PEG-NH₂, PEG-MAL, PEG-NHS, and PEG-ALD. Such functionalized PEG lipids are useful in the conjugation of targeting moieties to lipid nanoparticles to direct the particles to a particular target cell or tissue (e.g., by the attachment of antigen-binding molecules, peptides, glycans, etc.). In some embodiments, the functionalized moiety (e.g., —NH₂, _MAL, —NHS, -ALD) is added to the free end of the PEG moiety of the PEG-lipid of the disclosure (e.g., BRIJ™ or MYRJ™ family PEG lipid)

### Cationic Lipid

In some embodiments, the LNP provided herein comprises one or more cationic lipids. “Cationic lipid” and “ionizable lipid” are used interchangeably herein.

Cationic lipids refer to any of a number of lipid species that carry a net positive charge at a selected pH, such as physiological pH. Such lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N-(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C₁₈ alkyl chains, ether linkages between the head group and alkyl chains, and 0 to 3 double bonds. Such lipids include, e.g., DSDMA, DLinDMA, DLenDMA, and DODMA.

In some embodiments, the cationic lipids comprise a protonatable tertiary amine head group. Such lipids are referred to herein as ionizable lipids. Ionizable lipids refer to lipid species comprising an ionizable amine head group and typically comprising a pKa of less than about 7. Therefore, in environments with an acidic pH, the ionizable amine head group is protonated such that the ionizable lipid preferentially interacts with negatively charged molecules (e.g., nucleic acids such as the recombinant polynucleotides described herein) thus facilitating nanoparticle assembly and encapsulation. Therefore, in some embodiments, ionizable lipids can increase the loading of nucleic acids into lipid nanoparticles. In environments where the pH is greater than about 7 (e.g., physiologic pH of≈7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle. In some embodiments, the LNP comprises an ionizable lipid, e.g., a 7.SS-cleavable and pH-responsive Lipid Like Material (such as the COATSOME® SS-Series).

In some embodiments, the cationic lipid of the LNP is DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME@SS—OP, Di((Z)-non-2-en-1-yl)9-((4-dimethylamino)butanoyl)oxy)heptadecanedioate (L-319), N-(2,3-dioleoyloxy)propyl)-N,N,N-trimethylammonium chloride (DOTAP), or a mixture thereof.

In some embodiments the cationic lipid of the LNP is a compound of Formula (I):

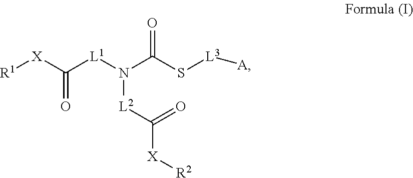

or a pharmaceutically acceptable salt or solvate thereof, wherein the variables are defined herein.

In some embodiments, cationic lipid of the disclosure is a compound selected from Table 21 or a pharmaceutically acceptable salt thereof.

In some embodiments, the cationic lipid of the LNP is a compound of Formula (II-1):

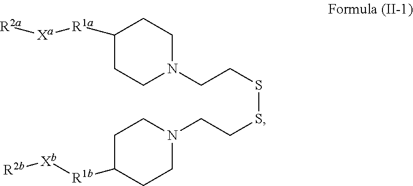

or a pharmaceutically acceptable salt or solvate thereof, wherein:
R^{1a} and R^{1b} are each independently C₁-C₈ aliphatic or —O(C₁-C₈ aliphatic)-, wherein the O atom, when present, is bonded to the piperidine ring;
X^{a} and X^{b} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, or —O—, wherein-* indicates the attachment point to
R₂ₐ or R^{2b}, respectively and wherein each occurrence of Rₓ¹ is independently selected from hydrogen and optionally substituted C₁-C₄ alkyl; and
R^{2a} and R^{2b} are each independently a sterol residue, a liposoluble vitamin residue, or an C₁₃-C₂₃ aliphatic.

In some embodiments, the cationic lipid of the LNP is a compound of Formula (II-2):

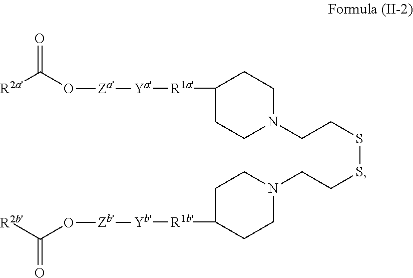

or a pharmaceutically acceptable salt or solvate thereof, wherein:
R^{1a′} and R^{1b′} are each independently C₁-C₈ alkylene or —O(C₁-C₈ alkylene), wherein the O atom, when present, is bonded to the piperidine ring;
Y^{a′} and Y^{b′} are each independently —C(O)O—*, —OC(O)—*, —C(O)N(Rₓ¹)—*, —N(Rₓ¹)C(O)—*, —O(C═O)N(Rₓ¹)—*, —N(Rₓ¹)(C═O)O—*, —N(Rₓ¹)C(O)N(Rₓ¹)—, or —O—, wherein-* indicates the attachment point to R^{2a} or R^{2b}, and wherein each occurrence of Rₓ¹ is independently selected from hydrogen and optionally substituted C₁-C₄ alkyl;
Z^{a′} and Z^{b′} are each independently optionally substituted arylene-C₀-C₈ alkylene or optionally substituted arylene-C₀-C₈ heteroalkylene, wherein the alkylene or heteroalkylene group is bonded to Y^{a′} and Y^{b′}, respectively;
R^{2a′} and R^{2b′} are each independently a sterol residue, a liposoluble vitamin residue, or an C₁₂-C₂₂ aliphatic.

In some embodiments, the cationic lipid of the LNP is a compound of Formula (II-1a) (COATSOME® SS—OC) or Formula (II-2a) (COATSOME® SS—OP):

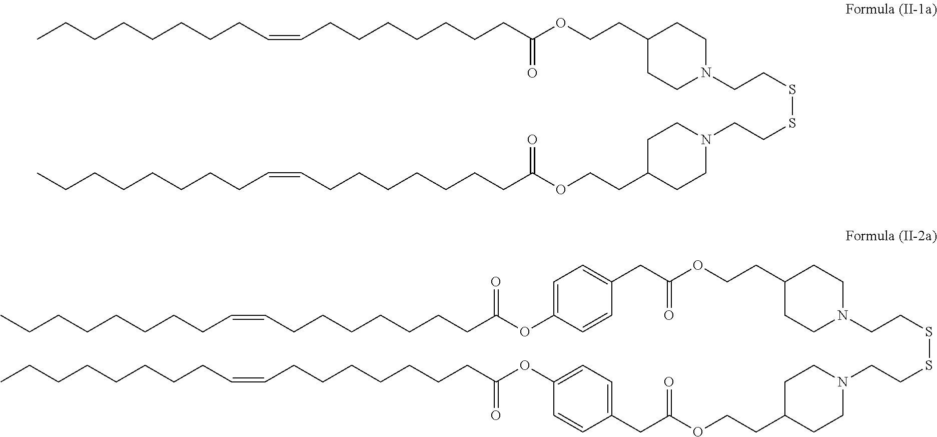 [SMILES: CCCCCCCC/C=C\CCCCCCCC(=O)OCCC1CCN(CCSSCCN2CCC(CCOC(=O)CCCCCCC/C=C\CCCCCCCC)CC2)CC1 ; CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In some embodiments, the cationic lipid of the LNP is a compound of Formula II-1a) (COATSOME® SS—OC). COATSOME® SS—OC is also known as SS-18/4PE-16.

In some embodiments, the cationic lipid of the LNP is a compound of Formula (II-2a) (COATSOME® SS—OP).

In some embodiments, the cationic lipid of the LNP is 1,2-dioleoyl-3-trimethylammonium-propane (DOTAP).

### Helper Lipid

In some embodiments, the LNP described herein comprises one or more helper lipids. The term “helper lipid” refers to a lipid capable of increasing the delivery of the LNP to a target, e.g., into a cell. Without wishing to be bound by any particular theory, it is contemplated that a helper lipid may enhance the stability and/or membrane fusogenicity of the lipid nanoparticle. In some embodiments, the helper lipid is a phospholipid. In some embodiments, the helper lipid is a phospholipid substitute or replacement. In some embodiments the helper lipid is an alkyl resorcinol.

In some embodiments, the helper lipid is a phosphatidyl choline (PC). In some embodiments, the helper lipid is not a phosphatidyl choline (PC). In some embodiments the helper lipid is a phospholipid or a phospholipid substitute. In some embodiments, the phospholipid or phospholipid substitute can be, for example, one or more saturated or (poly)unsaturated phospholipids, or phospholipid substitutes, or a combination thereof. In general, phospholipids comprise a phosphate head group and one or more fatty acid tails. In some embodiments, a phospholipid may include one or more multiple (e.g., double or triple) bonds (i.e. one or more unsaturations). In some embodiments, the helper lipid is non-cationic.

A phosphate head group can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, the non-cationic helper lipid is a DSPC analog, a DSPC substitute, oleic acid, or an oleic acid analog.

In some embodiments, a non-cationic helper lipid is a non-phosphatidyl choline (PC) zwitterionic lipid, a DSPC analog, oleic acid, an oleic acid analog, or a 1,2-distearoyl-sn-glycero-3-phosphocholine (DSPC) substitute.

In some embodiments, the phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

In some embodiments, a phosphate head group can be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid tail can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

In some embodiments, the phospholipid is a compound according to Formula (III):

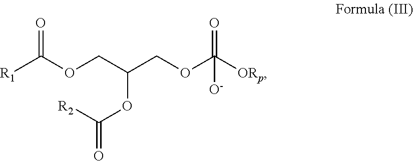

wherein: Rₚ represents a phosphate head group and R₁ and R² represent fatty acid tails with or without unsaturation that may be the same or different. A phosphate head group may be selected from the non-limiting group consisting of phosphatidylcholine, phosphatidyl ethanolamine, phosphatidyl glycerol, phosphatidyl serine, phosphatidic acid, 2-lysophosphatidyl choline, and a sphingomyelin. A fatty acid tail may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated. For example, a phospholipid may be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of an LNP to facilitate membrane permeation or cellular recognition or in conjugating an LNP to a useful component such as a targeting or imaging moiety (e.g., a dye).

In some embodiments, the LNPs comprise one or more non-cationic helper lipids (e.g., neutral lipids). Exemplary neutral helper lipids include (1,2-dilauroyl-sn-glycero-3-phosphoethanolamine) (DLPE), 1,2-diphytanoyl-sn-glycero-3-phosphoethanolamine (DiPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-dioleoyl-sn-glycero-3-phosphocholine (DOPC), 1,2-distearoyl-sn-glycero-3-phosphoethanolamine (DSPE), ceramides, and sphingomyelins. In some embodiments, the one or more helper lipids are selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE). In some embodiments, the helper lipid of the LNPs comprises, consists essentially of, or consist of 1,2-Dilauroyl-sn-glycero-3-phosphoethanolamine (DLPE) or 1,2-Dioleoyl-sn-glycero-3-phosphoethanolamine (DOPE). In some embodiments, the LNP comprises DSPC. In some embodiments, the LNP comprises DOPC. In some embodiments, the LNP comprises DLPE. In some embodiments, the LNP comprises DOPE.

In some embodiments, the phospholipid is selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine (18:3 (cis) PC), 1,2-diarachidonoyl-sn-glycero-3-phosphocholine (DAPC), 1,2-didocosahexaenoyl-sn-glycero-3-phosphocholine (22:6 (cis) PC) 1,2-diphytanoyl-sn-glycero-3-phosphoethanolamine (4ME 16.0 PE), 1,2-distearoyl-sn-glycero-3-phosphoethanolamine (DSPE), 1,2-dilinoleoyl-sn-glycero-3-phosphoethanolamine (PE(18:2/18:2), 1,2-dilinolenoyl-sn-glycero-3-phosphoethanol amine (PE 18:3 (9Z,12Z, 15Z), 1,2-diarachidonoyl-sn-glycero-3-phosphoethanolamine (DAPE 18:3 (9Z,12Z, 15Z), 1,2-didocosahexaenoyl-sn-glycero-3-phosphoethanolamine (22:6 (cis) PE), 1,2-dioleoyl-sn-glycero-3-phospho-rac-(1-glycerol) sodium salt (DOPG), and sphingomyelin.

In some embodiments, a helper lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoylphosphatidylethanolamine (POPE), dioleoylphosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine, dimethylphosphatidylethanolamine, 18-1-trans PE, 1-stearoyl-2-oleoylphosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidyl serine, phosphatidylinositol, sphingomyelin, egg sphingomyelin (ESM), cephalin, cardiolipin, phosphatidicacid, cerebrosides, dicetylphosphate, lysophosphatidylcholine, and dilinoleoylphosphatidylcholine.

In some embodiments, the helper lipid of the disclosure is DSPC.

In some embodiments, an LNP includes DSPC. In some embodiments, an LNP includes DOPE. In some embodiments, an LNP includes DMPE. In some embodiments, an LNP includes both DSPC and DOPE.

In some embodiments, a helper lipid is selected from the group consisting of DSPC, DMPE, and DOPC or combinations thereof.

In some embodiments of the disclosure, the helper lipid is

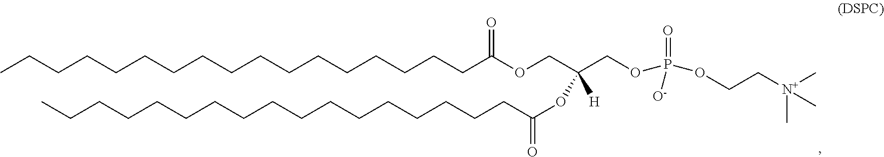 [SMILES: CCCCCCCCCCCCCCCCCC(=O)OC[C@H](COP(=O)([O-])OCC[N+](C)(C)C)OC(=O)CCCCCCCCCCCCCCCCC]

having a CAS number of 816-94-4, a linear formula of C₄₄H₈₈NO₈P. DSPC is also known as 1,2-distearoyl-sn-glycero-3-phosphocholine.

In some embodiments, a phospholipid of the disclosure comprises a modified tail. In some embodiments, the phospholipid is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, the helper lipid of the disclosure is an alternative lipid that is not a phospholipid.

In some embodiments, a phospholipid useful in the present disclosure comprises a modified tail. In some embodiments, a phospholipid useful in the present disclosure is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof.

In some embodiments, a phospholipid useful in the present disclosure comprises a modified phosphocholine moiety, wherein the alkyl chain linking the quaternary amine to the phosphoryl group is not ethylene (e.g., n is not 2).

In some embodiments, the LNP of the disclosure comprises an oleic acid or an oleic acid analog as the helper lipid. In some embodiments, an oleic acid analog comprises a modified oleic acid tail, a modified carboxylic acid moiety, or both. In some embodiments, an oleic acid analog is a compound wherein the carboxylic acid moiety of oleic acid is replaced by a different group.

In some embodiments, the LNP of the disclosure comprises a different zwitterionic group in place of a phospholipid as the helper lipid.

In some embodiments, the helper lipid of the disclosure is a naturally occurring membrane lipid. In some embodiments, the helper lipid of the disclosure is 1,2-Dipalmitoyl-sn-glycero-3-O-4′-(N,N,N-trimethyl)-homoserine (DGTS), Monogalactosyldiacylglycerol (MGDG), Digalactosyldiacylglycerol (DGDG), Sulfoquinovosyldiacylglycerol (SQDG), 1-Palmitoyl-2-cis-9,10-methylenehexadecanoyl-sn-glycero-3-phosphocholine (Cyclo PC), or a combination thereof. In some embodiments, the LNP of the disclosure comprises a combination of helper lipids. In some embodiments, the combination of helper lipids does not comprise DSPC. In some embodiments, the combination of helper lipid comprises DSPC. In some embodiments, the LNP comprising one or more naturally occurring membrane lipids (e.g., DGTS) has improved liver transfection/delivery of the target molecule encapsulated in the LNP as compared to the LNP comprising DSPC as the only helper lipid.

In some embodiments, the helper lipid of disclosure is 5-heptadecylresorcinol or a derivative thereof.

### Structural Lipid

In some embodiments, the LNP of the disclosure comprises one or more structural lipids. Incorporation of structural lipids in the lipid nanoparticle may help mitigate aggregation of other lipids in the particle. Structural lipids may be, but are not limited to, sterols or lipids containing sterol moieties.

In some embodiments, the structural lipid of the LNP is a sterol (e.g., phytosterols or zoosterols). In some embodiments, the sterol is cholesterol, or an analog or a derivative thereof. In some embodiments, the sterol is cholesterol. In some embodiments, the sterol is cholesterol, 0-sitosterol, fecosterol, ergosterol, sitosterol, campesterol, stigmasterol, brassicasterol, ergosterol, tomatidine, tomatine, ursolic acid, alpha-tocopherol, including analogs, salts or esters thereof, alone or in combination.

In some embodiments, the structural lipid of the LNP is a cholesterol, a corticosteroid (such as, for example, prednisolone, dexamethasone, prednisone, and hydrocortisone), or a combination thereof.

In some embodiments, the structural lipid of the LNP is a pytosterol. In some embodiments, the phytosterol is a sitosterol, a stigmasterol, a campesterol, a sitostanol, a campestanol, a brassicasterol, a fucosterol, beta-sitosterol, stigmastanol, beta-sitostanol, ergosterol, lupeol, cycloartenol, A5-avenaserol, A7-avenaserol or a A7-stigmasterol, including analogs, salts or esters thereof, alone or in combination.

In some embodiments, the LNP comprises one or more phytosterols. In some embodiments, the phytosterol component of the LNP is a single phytosterol. In some embodiments, the phytosterol component of the LNP of the disclosure is a mixture of different phytosterols (e.g. 2, 3, 4, 5 or 6 different phytosterols). In some embodiments, the phytosterol component of the LNP of the disclosure is a blend of one or more phytosterols and one or more zoosterols, such as a blend of a phytosterol (e.g., a sitosterol, such as beta-sitosterol) and cholesterol.

In some embodiments of the disclosure, the structural lipid of the LNP is cholesterol:

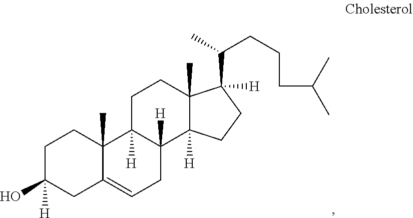

having a CAS number of 57-88-5, a linear formula of C₂₇H₄₆O.

In some embodiments, the LNP comprises a cationic lipid and one or more helper lipids, wherein the cationic lipid is DOTAP. In some embodiments, the LNP comprises a cationic lipid and one or more helper lipids, wherein the cationic lipid is DLin-MC3-DMA (MC3). In some embodiments, the LNP comprises a cationic lipid and one or more helper lipids, wherein the cationic lipid is COATSOME® SS-EC. In some embodiments, the LNP comprises a cationic lipid and one or more helper lipids, wherein the cationic lipid is COATSOME® SS-LC. In some embodiments, the LNP comprises a cationic lipid and one or more helper lipids, wherein the cationic lipid is COATSOME® SS—OC. In some embodiments, the LNP comprises a cationic lipid and one or more helper lipids, wherein the cationic lipid is COATSOME® SS—OP. In some embodiments, the LNP comprises a cationic lipid and one or more helper lipids, wherein the cationic lipid is L-319. In some embodiments, the LNP further comprises a structural lipid. In some embodiments, the structural lipid is cholesterol.

In some embodiments, the LNP comprises a cationic lipid and one or more helper lipids. In some embodiments, the LNP comprises a cationic lipid and one or more helper lipids, wherein the one or more helper lipids comprises DLPE. In some embodiments, the LNP comprises a cationic lipid and one or more helper lipids, wherein the one or more helper lipids comprises DSPC. In some embodiments, the LNP comprises a cationic lipid and one or more helper lipids, wherein the one or more helper lipids comprises DOPE. In some embodiments, the LNP comprises a cationic lipid and one or more helper lipids, wherein the one or more helper lipids comprises DOPC. In some embodiments, the LNP further comprises a structural lipid. In some embodiments, the structural lipid is cholesterol.

In some embodiments, the LNP comprises a cationic lipid, a helper lipid, and a structural lipid. In some embodiments, the structural lipid is cholesterol. In some embodiments, the cationic lipid is DOTAP, and the helper lipid is DLPE. In some embodiments, the cationic lipid is MC3, and the helper lipid is DSPC. In some embodiments, the helper lipid is DOPE. In some embodiments, the helper lipid is DSPC. In some embodiments, the LNP comprises a cationic lipid, a structural lipid, and at least two helper lipids, wherein the cationic lipid is DOTAP, and the at least two helper lipids comprise DLPE and DSPE. In some embodiments, the LNP comprises a cationic lipid, a structural lipid, and at least two helper lipids, wherein the cationic lipid is MC3, and the at least two helper lipids comprise DSPC and DMG. In some embodiments, the at least two helper lipids comprise DOPE and DSPE. In some embodiments, the at least two helper lipids comprise DSPC, and DMG. In some embodiments, the structural lipid is cholesterol. In some embodiments, the LNP comprises DOTAP, cholesterol, and DLPE. In some embodiments, the LNP comprises MC3, cholesterol, and DSPC. In some embodiments, the LNP comprises DOTAP, cholesterol, and DOPE. In some embodiments, the LNP comprises DOTAP, cholesterol, DLPE, and DSPE. In some embodiments, the LNP comprises MC3, cholesterol, DSPC, and DMG. In some embodiments, the LNP comprises DOTAP, cholesterol, DLPE, and DSPE-PEG. In some embodiments, the LNP comprises MC3, cholesterol, DSPC, and DMG-PEG. In some embodiments, the LNP comprises DOTAP, cholesterol, DOPE, and DSPE. In some embodiments, the LNP comprises DOTAP, cholesterol, DOPE, and DSPE-PEG. In some embodiments, the LNP comprises SS—OC, DSPC, cholesterol, and DPG-PEG (e.g., DPG-PEG2K). In some embodiments, the LNP comprises SS—OC, DSPC, cholesterol, and a PEG-lipid of formula (I) (e.g., BRIJ™ S100).

### Lipid Molar Ratio in the LNP Composition

In some embodiments, the LNP of the disclosure comprises between 40 mol % and 70 mol % of the cationic lipid, up to 50 mol % of the helper lipid, between 10 mol % and 50 mol % of the structural lipid, and between 0.001 mol % and 5 mol % of the PEG-lipid, inclusive of all endpoints. In some embodiments, the total mol % of the cationic lipid, the helper lipid, the structural lipid and the PEG-lipid is 100%.

In some embodiments, the mol % of the cationic lipid in the LNP is 40-70 mol %, 40-55 mol %, 40-50 mol %, 40-45 mol %, 44-54 mol %, 45-60 mol %, 45-55 mol %, 45-50 mol %, 50-60 mol %, 49-64 mol %, 50-55 mol %, or 55-60 mol %. In some embodiments, the mol % of the cationic lipid in the LNP is 44-54 mol %. In some embodiments, the mol % of the cationic lipid in the LNP is 40, 41, 42, 43, 44, 45, 46, 47, 48, 49, 50, 51, 52, 53, 54, 55, 56, 57, 58, 59, or 60 mol %. In some embodiments, the mol % of the cationic lipid in the LNP is about 40, about 41, about 42, about 43, about 44, about 45, about 46, about 47, about 48, about 49, about 50, about 51, about 52, about 53, about 54, about 55, about 56, about 57, about 58, about 59, or about 60 mol %. All values are inclusive of all endpoints.

In some embodiments, the mol % of the structural lipid in the LNP is 10-60 mol %, 10-30 mol %, 15-35 mol %, 20-40 mol %, 20-45 mol %, 25-33 mol %, 24-32 mol %, 25-45 mol %, 30-50 mol %, 35-43 mol %, 35-55 mol %, or 40-60 mol %. In some embodiments, the mol % of the structural lipid in the LNP is 20-45 mol %. In some embodiments, the mol % of the structural lipid in the LNP is 24-32 mol %. In some embodiments, the mol % of the structural lipid in the LNP is 25-33 mol %. In some embodiments, the mol % of the structural lipid in the LNP is 22-28 mol %. In some embodiments, the mol % of the structural lipid in the LNP is 35-45 mol %. In some embodiments, the mol % of the structural lipid in the LNP is 35-43 mol %. In some embodiments, the mol % of the structural lipid in the LNP is 10-60 mol %. In some embodiments, the mol % of the structural lipid in the LNP is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %. In some embodiments, the mol % of the structural lipid in the LNP is about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 mol %. In some embodiments, the structural lipid is cholesterol. All values are inclusive of all endpoints.

In some embodiments, the mol % of the helper lipid in the LNP is 1-50 mol %. In some embodiments, the mol % of the helper lipid in the LNP is up to 29 mol %. In some embodiments, the mol % of the helper lipid in the LNP is 1-10 mol %, 5-9 mol %, 5-15 mol %, 8-14 mol %, 18-22%, 19-25 mol %, 10-20 mol %, 10-25 mol %, 15-25 mol %, 20-30 mol %, 25-35 mol %, 30-40 mol %, or 35-50 mol %. In some embodiments, the mol % of the helper lipid in the LNP is 10-25 mol %. In some embodiments, the mol % of the helper lipid in the LNP is 5-9 mol %. In some embodiments, the mol % of the helper lipid in the LNP is 8-14 mol %. In some embodiments, the mol % of the helper lipid in the LNP is 18-22 mol %. In some embodiments, the mol % of the helper lipid in the LNP is 19-25 mol %. In some embodiments, the mol % of the helper lipid in the LNP is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 mol %. In some embodiments, the mol % of the helper lipid in the LNP is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 mol %. In some embodiments, the helper lipid is DSPC. All values are inclusive of all endpoints.

In some embodiments, the mol % of the PEG-lipid in the LNP is greater than 0 mol % and up to 5 mol % of the total lipid present in the LNP. In some embodiments, the mol % of the PEG-lipid is 0.1 mol %, 0.2 mol %, 0.25 mol %, 0.3 mol %, 0.4 mol %, 0.5 mol %, 0.6 mol %, 0.7 mol %, 0.8 mol %, 0.9 mol %, 1.0 mol %, 1.1 mol %, 1.2 mol %, 1.3 mol %, 1.4 mol %, 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, 2.5 mol %, 2.6 mol %, 2.7 mol %, 2.8 mol %, 2.9 mol %, 3.0 mol %, 3.1 mol %, 3.2 mol %, 3.3 mol %, 3.4 mol %, 3.5 mol %, 4.0 mol %, 4.5 mol %, or 5 mol % of the total lipid present in the LNP. In some embodiments, the mol % of the PEG-lipid is about 0.1 mol %, about 0.2 mol %, about 0.25 mol %, about 0.3 mol %, about 0.4 mol %, about 0.5 mol %, about 0.6 mol %, about 0.7 mol %, about 0.8 mol %, about 0.9 mol %, about 1.0 mol %, about 1.1 mol %, about 1.2 mol %, about 1.3 mol %, about 1.4 mol %, about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, about 3.5 mol %, about 4.0 mol %, about 4.5 mol %, or about 5 mol % of the total lipid present in the LNP. In some embodiments, the mol % of the PEG-lipid is at least 0.1 mol %, at least 0.2 mol %, at least 0.25 mol %, at least 0.3 mol %, at least 0.4 mol %, at least 0.5 mol %, at least 0.6 mol %, at least 0.7 mol %, at least 0.8 mol %, at least 0.9 mol %, at least 1.0 mol %, at least 1.1 mol %, at least 1.2 mol %, at least 1.3 mol %, at least 1.4 mol %, at least 1.5 mol %, at least 1.6 mol %, at least 1.7 mol %, at least 1.8 mol %, at least 1.9 mol %, at least 2.0 mol %, at least 2.1 mol %, at least 2.2 mol %, at least 2.3 mol %, at least 2.4 mol %, at least 2.5 mol %, at least 2.6 mol %, at least 2.7 mol %, at least 2.8 mol %, at least 2.9 mol %, at least 3.0 mol %, at least 3.1 mol %, at least 3.2 mol %, at least 3.3 mol %, at least 3.4 mol %, at least 3.5 mol %, at least 4.0 mol %, at least 4.5 mol %, or at least 5 mol % of the total lipid present in the LNP. In some embodiments, the mol % of the PEG-lipid is at most 0.1 mol %, at most 0.2 mol %, at most 0.25 mol %, at most 0.3 mol %, at most 0.4 mol %, at most 0.5 mol %, at most 0.6 mol %, at most 0.7 mol %, at most 0.8 mol %, at most 0.9 mol %, at most 1.0 mol %, at most 1.1 mol %, at most 1.2 mol %, at most 1.3 mol %, at most 1.4 mol %, at most 1.5 mol %, at most 1.6 mol %, at most 1.7 mol %, at most 1.8 mol %, at most 1.9 mol %, at most 2.0 mol %, at most 2.1 mol %, at most 2.2 mol %, at most 2.3 mol %, at most 2.4 mol %, at most 2.5 mol %, at most 2.6 mol %, at most 2.7 mol %, at most 2.8 mol %, at most 2.9 mol %, at most 3.0 mol %, at most 3.1 mol %, at most 3.2 mol %, at most 3.3 mol %, at most 3.4 mol %, at most 3.5 mol %, at most 4.0 mol %, at most 4.5 mol %, or at most 5 mol % of the total lipid present in the LNP. In some embodiments, the mol % of the PEG-lipid is between 0.1-4 mol % of the total lipid present in the LNP. In some embodiments, the mol % of the PEG-lipid is between 0.1-2 mol % of the total lipid present in the LNP. In some embodiments, the mol % of the PEG-lipid is between 0.2-0.8 mol %, 0.4-0.6 mol %, 0.7-1.3 mol %, 1.2-1.8 mol %, or 1-3.5 mol % of the total lipid present in the LNP. In some embodiments, the mol % of the PEG-lipid is 0.1-0.7 mol %, 0.2-0.8 mol %, 0.3-0.9 mol %, 0.4-0.8 mol %, 0.4-0.6 mol %, 0.4-1 mol %, 0.5-1.1 mol %, 0.6-1.2 mol %, 0.7-1.3 mol %, 0.8-1.4 mol %, 0.9-1.5 mol %, 1-3.5 mol % 1-1.6 mol %, 1.1-1.7 mol %, 1.2-1.8 mol %, 1.3-1.9 mol %, 1.4-2 mol %, 1.5-2.1 mol %, 1.6-2.2 mol %, 1.7-2.3 mol %, 1.8-2.4 mol %, 1.9-2.5 mol %, 2-2.6 mol %, 2.4-3.8 mol %, or 2.6-3.4 mol % of the total lipid present in the LNP. All values are inclusive of all endpoints.

In some embodiments, the LNP of the disclosure comprises 44-60 mol % of the cationic lipid, 19-25 mol % of the helper lipid, 25-33 mol % of the structural lipid, and 0.2-0.8 mol % of the PEG-lipid, inclusive of the endpoints. In some embodiments, the LNP of the disclosure comprises 44-54 mol % of the cationic lipid, 19-25 mol % of the helper lipid, 24-32 mol % of the structural lipid, and 1.2-1.8 mol % of the PEG-lipid, inclusive of the endpoints. In some embodiments, the LNP of the disclosure comprises 44-54 mol % of the cationic lipid, 8-14 mol % of the helper lipid, 35-43 mol % of the structural lipid, and 1.2-1.8 mol % of the PEG-lipid, inclusive of the endpoints. In some embodiments, the LNP of the disclosure comprises 45-55 mol % of the cationic lipid, 5-9 mol % of the helper lipid, 36-44 mol % of the structural lipid, and 2.5-3.5 mol % of the PEG-lipid, inclusive of the endpoints.

In some embodiments, the LNP of the disclosure comprises one or more of the cationic lipids of the disclosure, one or more helper lipids of the disclosure, one or more structural lipids of the disclosure, and one or more PEG-lipid of the disclosure at a mol % of total lipid (or the mol % range of total lipid) in the LNP according to Table 6 below. In some embodiments, the total mol % of these four lipid components equals 100%. In some embodiments, the total mol % of these four lipid components is less than 100%. In some embodiments, the cationic lipid is a compound of Formula (I) or a compound selected from Table 21. In some embodiments, the structural lipid is cholesterol. In some embodiments, the helper lipid is DSPC. In some embodiments, the PEG-lipid is of Formula (A), Formula (A′), or Formula (A″).

TABLE 6
Mol % of the Lipid Components in the LNP

| Cationic Lipid | Structural Lipid | Helper Lipid | PEG-lipid |
|---|---|---|---|
| (mol %) | (mol %) | (mol %) | (mol %) |
| 49 | 28.5 | 22 | 0.5 |
| 47-52 | 27-30 | 21-23 | 0.4-0.6 |
| 44-54 | 25-32 | 19-25 | 0.2-0.8 |
| 44-54 | 25-33 | 19-25 | 0.2-0.8 |
| 49 | 27.5 | 22 | 1.5 |
| 47-52 | 26-29 | 21-23 | 1.3-1.7 |
| 44-54 | 24-31 | 19-25 | 1.1-1.9 |
| 44-54 | 24-32 | 19-25 | 1.2-1.8 |
| 49 | 39.5 | 11 | 0.5 |
| 47-52 | 38-41 | 11-13 | 0.4-0.6 |
| 44-54 | 36-43 | 9-15 | 0.2-0.8 |
| 49 | 38.5 | 11 | 1.5 |
| 47-52 | 37-40 | 11-13 | 1.3-1.7 |
| 44-54 | 35-42 | 9-15 | 1.1-1.9 |
| 44-54 | 35-43 | 8-14 | 1.2-1.8 |
| 20-60 | 10-60 | ≥20 | 0.5 |
| 20-60 | 10-60 | ≥20 | 0.3-0.7 |
| 20-60 | 10-60 | ≥20 | 0.1-0.9 |
| 20-60 | 10-60 | 10 | 1.5 |
| 20-60 | 10-60 | 8-12 | 1.3-1.7 |
| 20-60 | 10-60 | 6-14 | 1.1-1.9 |
| 50 | 40 | 7 | 3 |
| 45-55 | 35-45 | 5-9 | 2.5-3.5 |
| 54.5 | 25 | 20 | 0.5 |
| 50-60 | 22-28 | 18-22 | 0.3-0.7 |
| 54.6 | 25.1 | 20.1 | 0.25 |
| 44.5 | 50 | 5 | 0.5 |
| 40 | 50 | 8.75 | 1.25 |
| 60 | 25 | 14.5 | 0.5 |
| 60 | 34.3 | 5 | 0.7 |
| 50 | 42.5 | 7 | 0.5 |
| 58 | 33.5 | 7 | 1.5 |
| 58 | 34.5 | 7 | 0.5 |
| 35-65 | 25-55 | 5-25 | 0.3-3 |
| 49 | 28.5 | 22 | 0.5 |
| 40 | 39.5 | 20 | 0.5 |
| 40 | 50 | 8.75 | 1.25 |
| 60 | 34.3 | 5 | 0.7 |
| 54.6 | 25.1 | 20.1 | 0.25 |
| 50.1 | 42.6 | 7 | 0.24 |

In some embodiments, the LNP of the disclosure comprises 44-54 mol 00 of the cationic lipid, 19-25 mol 00 of the helper lipid, 25-33 mol 00 of the structural lipid, and 0.2-0.8 mol % of the PEG-lipid, inclusive of the endpoints. In some embodiments, the LNP of the disclosure comprises 44-54 mol 00 of the compound of Formula (II-1a), 19-25 mol 00 of the DSPC, 25-33 mol 00 of the cholesterol, and 0.2-0.8 mol 00 of the PEG-lipid selected from HO-PEG100—CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG50-C(O)—CH₂(CH₂)₁₅CH₃, and HO-PEG40-C(O)—CH₂(CH₂)₁₅CH₃, inclusive of the endpoints.

In some embodiments, the LNP of the disclosure comprises 44-54 mol % of the cationic lipid, 19-25 mol % of the helper lipid, 24-32 mol % of the structural lipid, and 1.2-1.8 mol % of the PEG-lipid, inclusive of the endpoints. In some embodiments, the LNP of the disclosure comprises 44-54 mol % of the compound of Formula (II-1a), 19-25 mol % of the DSPC, 24-32 mol % of the cholesterol, and 1.2-1.8 mol % of the PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG50-C(O)—CH₂(CH₂)₁₅CH₃, and HO-PEG40-C(O)—CH₂(CH₂)₁₅CH₃, inclusive of the endpoints.

In some embodiments, the LNP of the disclosure comprises 44-54 mol % of the cationic lipid, 8-14 mol % of the helper lipid, 35-43 mol % of the structural lipid, and 1.2-1.8 mol % of the PEG-lipid, inclusive of the endpoints. In some embodiments, the LNP of the disclosure comprises 44-54 mol % of the compound of Formula (II-1a), 8-14 mol % of the DSPC, 35-43 mol % of the cholesterol, and 1.2-1.8 mol % of the PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG50-C(O)—CH₂(CH₂)₁₅CH₃, and HO-PEG40-C(O)—CH₂(CH₂)₁₅CH₃, inclusive of the endpoints.

In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about A:B:C:D, wherein A=40 mol %-60 mol %, B=10 mol %-25 mol %, C=20 mol %-30 mol %, and D=0 mol %-3 mol % and wherein A+B+C+D=100 mol %. In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about A:B:C:D, wherein A=45 mol %-50 mol %, B=20 mol %-25 mol %, C=25 mol %-30 mol %, and D=0 mol %-1 mol % and wherein A+B+C+D=100 mol %. In some embodiments, the LNP comprises SS—OC, DSPC, cholesterol (Chol), and a PEG-lipid, wherein the ratio of SS—OC:DSPC:Chol:PEG-lipid (as a percentage of total lipid content) is about 49:22:28.5:0.5. In some embodiments, the PEG-lipid is a compound of Formula (A), Formula (A′), or Formula (A″). In some embodiments, the PEG-lipid is selected from the group consisting of BRIJ™ S100, BRIJ™ S20, BRIJ™ 020 and BRIJ™ C₂₀. In some embodiments, the PEG-lipid is BRIJ™ S100.

In some embodiments, the LNP comprises DOTAP, cholesterol (Chol), and DLPE, wherein the ratio of DOTAP:Chol:DLPE (as a percentage of total lipid content) is about 50:35:15. In some embodiments, the LNP comprises DOTAP, cholesterol (Chol), and DLPE, wherein the ratio of DOTAP:Chol:DOPE (as a percentage of total lipid content) is about 50:35:15. In some embodiments, the LNP comprises DOTAP, cholesterol (Chol), DLPE, DSPE-PEG, wherein the ratio of DOTP:Chol:DLPE (as a percentage of total lipid content) is about 50:35:15 and wherein the particle comprises about 0.2 mol % DSPE-PEG. In some embodiments, the LNP comprises MC3, cholesterol (Chol), DSPC, and DMG-PEG, wherein the ratio of MC3:Chol:DSPC:DMG-PEG (as a percentage of total lipid content) is about 49:38.5:11:1.5.

In some embodiments, the LNP comprises SS—OC, DSPC, cholesterol (Chol), and DPG-PEG2K), wherein the ratio of SS—OC:DSPC:Chol:DPG-PEG2K (as a percentage of total lipid content) is about A:B:C:D, wherein A=40 mol %-60 mol %, B=10 mol %-25 mol %, C=20 mol %-30 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-50 mol %, B=20 mol %-25 mol %, C=25 mol %-30 mol %, and D=0 mol %-1 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about 49:22:28.5:0.5.

In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=40 mol %-60 mol %, B=10 mol %-30 mol %, C=20 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=40 mol %-60 mol %, B=10 mol %-30 mol %, C=25 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-55 mol %, B=10 mol %-20 mol %, C=30 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-50 mol %, B=10 mol %-15 mol %, C=35 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is 49:11:38.5:1.5.

In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=45 mol %-65 mol %, B=5 mol %-20 mol %, C=20 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=50 mol %-60 mol %, B=5 mol %-15 mol %, C=30 mol %-45 mol %, and D=0 mol %-3 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=55 mol %-60 mol %, B=5 mol %-15 mol %, C=30 mol %-40 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is about A:B:C:D, wherein A=55 mol %-60 mol %, B=5 mol %-10 mol %, C=30 mol %-35 mol %, and D=1 mol %-2 mol % and wherein A+B+C+D=100 mol %. In some embodiments, the LNP comprises SS—OC, DSPC, cholesterol (Chol), and DPG-PEG2K, wherein the ratio of SS—OC:DSPC:Chol:DPG-PEG2K (as a percentage of total lipid content) is 58:7:33.5:1.5.

In some embodiments, the nanoparticle is coated with a glycosaminoglycan (GAG) in order to modulate or facilitate uptake of the nanoparticle by target cells. The GAG may be heparin/heparin sulfate, chondroitin sulfate/dermatan sulfate, keratin sulfate, or hyaluronic acid (HA). In a particular embodiment, the surface of the nanoparticle is coated with HA and targets the particles for uptake by tumor cells. In some embodiments, the lipid nanoparticle is coated with an arginine-glycine-aspartate tri-peptide (RGD peptides) (See Ruoslahti, Advanced Materials, 24, 2012, 3747-3756; and Bellis et al., Biomaterials, 32(18), 2011, 4205-4210).

### Properties of LNP Composition

The disclosure provides compositions (e.g., pharmaceutical compositions) comprising a plurality of LNPs as described herein. Also provided herein are compositions comprising LNPs as described herein and encapsulated molecules.

In some embodiments, the LNP of the present disclosure may reduce immune response in vivo as compared to a control LNP. In some embodiments, the control LNP is an LNP comprising a PEG-lipid that is not of Formula (A), Formula (A′), or Formula (A″). In some embodiments, the PEG-lipid of the control LNP is PEG2k-DPG. In some embodiments, the PEG-lipid of the control LNP is PEG2k-DMG. In some embodiments, the control LNP has the same molar ratio of the PEG-lipid as the LNP of the present disclosure. In some embodiments, the control LNP is identical to an LNP of the present disclosure except that the control LNP comprises a PEG-lipid that is not of Formula (A), Formula (A′), or Formula (A″) (e.g., the control LNP may comprise PEG2k-DPG or PEG2k-DMG as PEG-lipid).

In some embodiments, the control LNP is an LNP comprising a cationic lipid that is not of Formula (I). In some embodiments, the cationic lipid of the control LNP is SS—OC. In some embodiments, the control LNP has the same molar ratio of the cationic lipid as the LNP of the present disclosure. In some embodiments, the control LNP is identical to an LNP of the present disclosure except that the control LNP comprises a cationic lipid that is not of Formula (I) (e.g., the control LNP may comprise SS—OC as cationic lipid).

In some embodiments, the reduced immune response may be a reduction in accelerated blood clearance (ABC). In some embodiments, the ABC is associated with the secretion of natural IgM and/or anti-PEG IgM. The term “natural IgM,” as used herein, refers to circulating IgM in the serum that exists independent of known immune exposure (e.g., the exposure to a LNP of the disclosure). The term “reduction of ABC” refers to any reduction in ABC in comparison to a control LNP. In some embodiments, a reduction in ABC may be a reduced clearance of the LNP upon a second or subsequent dose, relative to a control LNP. In some embodiments, the reduction may be at least 10%, 15%, 20%, 25%, 30%, 35%, 40%, 45%, 50%, 55%, 60%, 65%, 70%, 75%, 80%, 85%, 90%, 95%, 98%, or 100%. In some embodiments, the reduction is about 10% to about 100%, about 10 to about 50%, about 20 to about 100%, about 20 to about 50%, about 30 to about 100%, about 30 to about 50%, about 40% to about 100%, about 40 to about 80%, about 50 to about 90%, or about 50 to about 100%. In some embodiments, a reduction in ABC may be measured by an increase in or a sustained detectable level of an encapsulated synthetic RNA viral genome following a second or subsequent administration. In some embodiments, a reduction in ABC may result in an increase (e.g., a 2-fold, a 3-fold, a 4-fold, a 5-fold, or higher fold increase) in the level of the encapsulated synthetic RNA viral genome relative to the level of encapsulated synthetic RNA viral genome following administration of a control LNP. In some embodiments, the reduced ABC is associated with a lower serum level of anti-PEG IgM.

In some embodiments, the LNP of the present disclosure may delay clearance of the LNP and components thereof upon repeat dosing compared to a control LNP, which may be cleared prior to release of encapsulated molecule. Accordingly, the LNP of the present disclosure may increase the delivery efficiency of the encapsulated molecule (e.g., synthetic RNA viral genome) in subsequent doses.

In some embodiments, the LNPs have an average size (i.e., average outer diameter) of about 50 nm to about 500 nm. In some embodiments, the LNPs have an average size of about 50 nm to about 200 nm, about 100 nm to about 200 nm, about 150 nm to about 200 nm, about 50 nm to about 100 nm, about 50 nm to about 150 nm, about 100 nm to about 150 nm, about 200 nm to about 250 nm, about 250 nm to about 300 nm, about 300 nm to about 400 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the LNPs have an average size of about 50 nm, 60 nm, 70 nm, 80 nm, 90 nm, 100 nm, 110 nm, about 120, or about 125 nm. In some embodiments, the LNPs have an average size of about 100 nm. In some embodiments, the LNPs have an average size of 50 nm to 150 nm. In some embodiments, the LNPs have an average size (average outer diameter) of 50 nm to 150 nm, 50 nm to 125 nm, 50 nm to 100 nm, 50 nm to 75 nm, 75 nm to 150 nm, 75 nm to 125 nm, 75 nm to 100 nm, 100 nm to 150 nm, 100 nm to 125 nm, or 125 nm to 150 nm. In some embodiments, the LNPs have an average size of 70 nm to 90 nm, 80 nm to 100 nm, 90 nm to 110 nm, 100 nm to 120 nm, 110 nm to 130 nm, 120 nm to 140 nm, or 130 nm to 150 nm. In some embodiments, the LNPs have an average size of 90 nm to 110 nm. All values are inclusive of end points.

In some embodiments, the LNPs have an average size (i.e., average outer diameter) of about 50 nm to about 150 nm. In some embodiments, the disclosure provides a therapeutic composition comprising a plurality of lipid nanoparticles, wherein the plurality of LNPs have an average size of about 60 nm to about 130 nm. In some embodiments, the disclosure provides a therapeutic composition comprising a plurality of lipid nanoparticles, wherein the plurality of LNPs have an average size of about 70 nm to about 120 nm. In some embodiments, the disclosure provides a therapeutic composition comprising a plurality of lipid nanoparticles, wherein the plurality of LNPs have an average size of about 70 nm. In some embodiments, the disclosure provides a therapeutic composition comprising a plurality of lipid nanoparticles, wherein the plurality of LNPs have an average size of about 80 nm. In some embodiments, the disclosure provides a therapeutic composition comprising a plurality of lipid nanoparticles, wherein the plurality of LNPs have an average size of about 90 nm. In some embodiments, the disclosure provides a therapeutic composition comprising a plurality of lipid nanoparticles, wherein the plurality of LNPs have an average size of about 100 nm. In some embodiments, the disclosure provides a therapeutic composition comprising a plurality of lipid nanoparticles, wherein the plurality of LNPs have an average size of about 110 nm. All values are inclusive of end points.

In some embodiments, the encapsulation efficiency of the synthetic RNA viral genome by the LNP is about 70%, about 75%, about 80%, about 85%, about 90%, about 91%, about 92%, about 93%, about 94%, about 95%, about 96%, about 97%, about 98%, about 99%, or 100%. In some embodiments, about 70%, about 75%, about 80%, about 90%, about 95%, about 97%, about 98%, or about 99% of the plurality of LNPs comprises an encapsulated synthetic RNA viral genome. In some embodiments, the encapsulation efficiency of the synthetic RNA viral genome by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In some embodiments, at least 70%, at least 75%, at least 80%, at least 90%, at least 95%, at least 97%, at least 98%, or at least 99% of the plurality of LNPs comprises an encapsulated synthetic RNA viral genome. In some embodiments, about 70% to 100%, about 75% to 100%, about 80% to 100%, about 85% to 100%, about 90% to 100%, about 91% to 100%, about 92% to 100%, about 93% to 100%, about 94% to 100%, about 95% to 100%, about 96% to 100%, about 97% to 100%, about 98% to 100%, about 99% to 100% of the plurality of LNPs comprises an encapsulated synthetic RNA viral genome.

In some embodiments, the LNPs have a neutral charge (e.g., an average zeta-potential of between about 0 mV and 1 mV). In some embodiments, the LNPs have an average zeta-potential of between about 40 mV and about −40 mV. In some embodiments, the LNPs have an average zeta-potential of between about 40 mV and about 0 mV. In some embodiments, the LNPs have an average zeta-potential of between about 35 mV and about 0 mV, about 30 mV and about 0 mV, about 25 mV to about 0 mV, about 20 mV to about 0 mV, about 15 mV to about 0 mV, about 10 mV to about 0 mV, or about 5 mV to about 0 mV. In some embodiments, the LNPs have an average zeta-potential of between about 20 mV and about −40 mV. In some embodiments, the LNPs have an average zeta-potential of between about 20 mV and about −20 mV. In some embodiments, the LNPs have an average zeta-potential of between about 10 mV and about −20 mV. In some embodiments, the LNPs have an average zeta-potential of between about 10 mV and about −10 mV. In some embodiments, the LNPs have an average zeta-potential of about 10 mV, about 9 mV, about 8 mV, about 7 mV, about 6 mV, about 5 mV, about 4 mV, about 3 mV, about 2 mV, about 1 mV, about 0 mV, about −1 mV, about −2 mV, about −3 mV, about −4 mV, about −5 mV, about −6 mV, about −7 mV, about −8 mV, about −9 mV, about −9 mV or about −10 mV.

In some embodiments, the LNPs have an average zeta-potential of between about 0 mV and −20 mV. In some embodiments, the LNPs have an average zeta-potential of less than about −20 mV. For example in some embodiments, the LNPs have an average zeta-potential of less than about less than about −30 mV, less than about 35 mV, or less than about −40 mV. In some embodiments, the LNPs have an average zeta-potential of between about −50 mV to about −20 mV, about −40 mV to about −20 mV, or about −30 mV to about −20 mV. In some embodiments, the LNPs have an average zeta-potential of about 0 mV, about −1 mV, about −2 mV, about −3 mV, about −4 mV, about −5 mV, about −6 mV, about −7 mV, about −8 mV, about −9 mV, about −10 mV, about −11 mV, about −12 mV, about −13 mV, about −14 mV, about −15 mV, about −16 mV, about −17 mV, about −18 mV, about −19 mV, about −20 mV, about −21 mV, about −22 mV, about −23 mV, about −24 mV, about −25 mV, about −26 mV, about −27 mV, about −28 mV, about −29 mV, about −30 mV, about −31 mV, about −32 mV, about −33 mV, about −34 mV, about −35 mV, about −36 mV, about −37 mV, about −38 mV, about −39 mV, or about −40 mV. In some embodiments, the LNPs have an average zeta-potential of less than about −20 mV, less than about −30 mV, less than about 35 mV, or less than about −40 mV.

In some embodiments, the LNP comprises a recombinant nucleic acid molecule described herein and has a mass ratio of lipid (L) to nucleic acid (N) of about 10:1 to about 60:1. In some embodiments, the LNP comprises a recombinant nucleic acid molecule described herein and has a mass ratio of lipid (L) to nucleic acid (N) of about 20:1. In some embodiments, the LNP comprises a recombinant nucleic acid molecule described herein and has a mass ratio of lipid (L) to nucleic acid (N) of about 30:1. In some embodiments, the LNP comprises a recombinant nucleic acid molecule described herein and has a mass ratio of lipid (L) to nucleic acid (N) of about 40:1. In some embodiments, the LNP comprises a recombinant nucleic acid molecule described herein and has an L:N mass ratio of about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 237:1, about 28:1, about 39:1, about 40:1, about 41:1, about 42:1, about 43:1, about 44:1, or about 45:1.

In some embodiments, the LNP has a lipid (L) to nucleic acid molecule (N) mass ratio of between 10:1 and 60:1, between 20:1 and 60:1, between 30:1 and 60:1, between 40:1 and 60:1, between 50:1 and 60:1, between 10:1 and 50:1, between 20:1 and 50:1, between 30:1 and 50:1, between 40:1 and 50:1, between 10:1 and 40:1, between 20:1 and 40:1, between 30:1 and 40:1, between 10:1 and 30:1, between 20:1 and 30:1, or between 10:1 and 20:1, inclusive of all endpoints. In some embodiments, the LNP has a lipid:nucleic acid molecule mass ratio of between 30:1 and 40:1. In some embodiments, the LNP has a lipid:nucleic acid molecule mass ratio of between 30:1 and 36:1.

In some embodiments, the LNP comprises a recombinant nucleic acid molecule described herein and has a mass ratio of lipid (L) to nucleic acid (N) of about 10:1 to about 60:1. In some embodiments, the LNP comprises a recombinant nucleic acid molecule described herein and has a mass ratio of lipid (L) to nucleic acid (N) of about 20:1. In some embodiments, the LNP comprises a recombinant nucleic acid molecule described herein and has a mass ratio of lipid (L) to nucleic acid (N) of about 30:1 (L:N). In some embodiments, the LNP comprises a recombinant nucleic acid molecule described herein and has a mass ratio of lipid (L) to nucleic acid (N) of about 40:1 (L:N). In some embodiments, the LNP comprises a recombinant nucleic acid molecule described herein and has an L:N mass ratio of about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 31:1, about 32:1, about 33:1, about 34:1, about 35:1, about 36:1, about 237:1, about 28:1, about 39:1, about 40:1, about 41:1, about 42:1, about 43:1, about 44:1, or about 45:1.

In some embodiments, the LNP comprises a nucleic acid molecule and has a lipid-nitrogen-to-phosphate ratio (N:P) of between 1 to 25. In some embodiments, the N:P is 1, 2, 3, 4, 5, 6, 7, 8, 9, 10, 11, 12, 13, 14, 15, 16, 17, 18, 19, 20, 21, 22, 23, 24, or 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the LNP comprises a nucleic acid molecule and has a lipid-nitrogen-to-phosphate ratio (N:P) of 14.

In some embodiments, the LNP comprises a synthetic RNA viral genome encoding an oncolytic virus, wherein the encoded oncolytic virus is capable of reducing the size of a tumor that is remote from the site of LNP administration to a subject. For example, as demonstrated in the examples provided herein, intravenous administration of the LNPs described herein results in viral replication in tumor tissue and reduction of tumor size. These data indicate that the LNPs of the present disclosure are capable of localizing to tumors or cancerous tissues that are remote from the site of LNP administration. Such effects enable the use of the LNP-encapsulated oncolytic viruses described herein in the treatment of tumors that are not easily accessible and therefore not suitable for intratumoral delivery of treatment.

### Method of LNP Preparation

In some embodiments, the disclosure provides methods for preparing a composition of lipid nanoparticles (LNPs) containing a nucleic acid molecule, comprising the steps of:
(a) diluting the nucleic acid molecule to a desired concentration in an aqueous solution;
(b) mixing organic lipid phase comprising all lipid components of the LNPs with the aqueous phase containing the nucleic acid molecule using microfluidic flow to form the LNPs;
(c) dialyzing the LNPs against a buffer to remove the organic solvent;
(d) concentrating the LNPs to a target volume; and
(e) optionally, filtered through a sterile filter.

In some embodiments, the organic lipid phase and the aqueous phase are mixed at a ratio of between 1:1 (v:v) and 1:10 (v:v). In some embodiments, the organic lipid phase and the aqueous phase are mixed at a ratio of 1:1 (v:v), 1:2 (v:v), 1:3 (v:v), 1:4 (v:v), 1:5 (v:v), 1:6 (v:v), 1:7 (v:v), 1:8 (v:v), 1:9 (v:v), or 1:10 (v:v). In some embodiments, the organic lipid phase and the aqueous phase are mixed at a ratio of between 1:1 (v:v) and 1:3 (v:v), between 1:2 (v:v) and 1:4 (v:v), between 1:3 (v:v) and 1:5 (v:v), between 1:4 (v:v) and 1:6 (v:v), between 1:5 (v:v) and 1:7 (v:v), between 1:6 (v:v) and 1:8 (v:v), between 1:7 (v:v) and 1:9 (v:v), or between 1:8 (v:v) and 1:10 (v:v). In some embodiments, the organic lipid phase and the aqueous phase are mixed at a ratio of between 1:3 (v:v) and 1:5 (v:v). In some embodiments, the organic lipid phase and the aqueous phase are mixed at a ratio of 1:3 (v:v). In some embodiments, the organic lipid phase and the aqueous phase are mixed at a ratio of 1:5 (v:v).

In some embodiments, the total flow rate of the microfluidic flow is 5-20 mL/min. In some embodiments, the total flow rate of the microfluidic flow is 5, 6, 7, 8, 9, 10, 11, 12, 13, 14, 15, 16, 17, 18, 19, or 20 mL/min. In some embodiments, the total flow rate of the microfluidic flow is 9-20 mL/min. In some embodiments, the total flow rate of the microfluidic flow is 11-13 mL/min.

In some embodiments, the solvent in the organic lipid phase in step (b) is ethanol. In some embodiments, heat is applied to the organic lipid phase in step (b). In some embodiments, about 40, 45, 50, 55, 60, 65, 70, 75, or 80° C. is applied to the organic lipid phase in step (b). In some embodiments, 60° C. heat is applied to the organic lipid phase in step (b). In some embodiments, no heat is applied to the organic lipid phase in step (b).

In some embodiments, the aqueous solution in step (a) has a pH of between 1 and 7. In some embodiments, the aqueous solution in step (a) has a pH of between 1 and 3, between 2 and 4, between 3 and 5, between 4 and 6, or between 5 and 7. In some embodiments, the aqueous solution in step (a) has a pH of 1, 1.5, 2, 2.5, 3, 3.5, 4, 4.5, 5, 5.5, 6, 6.5, or 7. In some embodiments, the aqueous solution in step (a) has a pH of 3. In some embodiments, the aqueous solution in step (a) has a pH of 5.

In some embodiments, the total lipid concentration is between 5 mM and 80 mM. In some embodiments, the total lipid concentration is about 5, 10, 15, 20, 25, 30, 35, 40, 45, 50, 55, 60, 65, 70, 75, or 80 mM. In some embodiments, the total lipid concentration is about 20 mM. In some embodiments, the total lipid concentration is about 40 mM.

In some embodiments, the LNP generated by the method has a lipid-nitrogen-to-phosphate ratio (N:P) of between 1 to 25. In some embodiments, the N:P is 1, 2, 3, 4, 5, 6, 7, 8, 9, 10, 11, 12, 13, 14, 15, 16, 17, 18, 19, 20, 21, 22, 23, 24, or 25. In some embodiments, the N:P is between 1 to 25, between 1 to 20, between 1 to 15, between 1 to 10, between 1 to 5, between 5 to 25, between 5 to 20, between 5 to 15, between 5 to 10, between 10 to 25, between 10 to 20, between 10 to 15, between 15 to 25, between 15 to 20, or between 20 to 25. In some embodiments, the LNP comprises a nucleic acid molecule and has a lipid-nitrogen-to-phosphate ratio (N:P) of 14.

In some embodiments, the buffer in step (c) has a neutral pH (e.g., lx PBS, pH 7.2). In some embodiments, step (d) uses centrifugal filtration for concentrating.

In some embodiments, the encapsulation efficiency of the method of the disclosure is at least 70%, at least 75%, at least 75%, at least 80%, at least 90%, at least 95%, at least 97%, at least 98%, or at least 99%. In some embodiments, the encapsulation efficiency of the method of the disclosure is at least 90%. In some embodiments, the encapsulation efficiency of the method of the disclosure is at least 95%. In some embodiments, the encapsulation efficiency is determined by RiboGreen.

In some embodiments, the LNPs produced by the method of the disclosure have an average size (i.e., average outer diameter) of about 50 nm to about 500 nm. In some embodiments, the LNPs have an average size of about 50 nm to about 200 nm, about 100 nm to about 200 nm, about 150 nm to about 200 nm, about 50 nm to about 100 nm, about 50 nm to about 150 nm, about 100 nm to about 150 nm, about 200 nm to about 250 nm, about 250 nm to about 300 nm, about 300 nm to about 400 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm. In some embodiments, the plurality of LNPs have an average size of about 50 nm, 60 nm, 70 nm, 80 nm, 90 nm, 100 nm, 110 nm, about 120, or about 125 nm. In some embodiments, the plurality of LNPs have an average size of about 100 nm. In some embodiments, the plurality of LNPs have an average size of 50 nm to 150 nm. In some embodiments, the plurality of LNPs have an average size (average outer diameter) of 50 nm to 150 nm, 50 nm to 125 nm, 50 nm to 100 nm, 50 nm to 75 nm, 75 nm to 150 nm, 75 nm to 125 nm, 75 nm to 100 nm, 100 nm to 150 nm, 100 nm to 125 nm, or 125 nm to 150 nm. In some embodiments, the plurality of LNPs have an average size of 70 nm to 90 nm, 80 nm to 100 nm, 90 nm to 110 nm, 100 nm to 120 nm, 110 nm to 130 nm, 120 nm to 140 nm, or 130 nm to 150 nm. In some embodiments, the plurality of LNPs have an average size of 90 nm to 110 nm.

In some embodiments, the polydispersity index of the plurality of LNPs is between 0.01 and 0.3. In some embodiments, the polydispersity index of the plurality of LNPs is between 0.1 and 0.15. In some embodiments, the polydispersity index of the plurality of LNPs is about 0.01, about 0.02, about 0.03, about 0.04, about 0.05, about 0.06, about 0.07, about 0.08, about 0.09, about 0.10, about 0.11, about 0.12, about 0.13, about 0.14, about 0.15, about 016, about 0.17, about 0.18, about 0.19, about 0.20, about 0.21, about 0.22, about 0.23, about 0.24, about 0.25, about 0.26, about 0.27, about 0.28, about 0.29, or about 0.30. In some embodiments, the polydispersity index of the plurality of LNPs is about 0.10, about 0.11, about 0.12, about 0.13, about 0.14, or about 0.15. In some embodiments, the average diameter and/or the polydispersity is determined via dynamic light scattering.

### Payload Molecules

In some embodiments, the particles comprise a synthetic RNA viral genome and further comprise a recombinant RNA polynucleotide encoding a payload molecule. In some embodiments, the particles are lipid nanoparticles and comprise a synthetic RNA viral genome and further comprise a recombinant RNA polynucleotide encoding a payload molecule. In some embodiments, the synthetic RNA viral genome in the particle (e.g., LNP) comprises the recombinant RNA polynucleotide encoding the payload molecule. In some embodiments, the particle (e.g., LNP) comprises 1) the synthetic RNA viral genome (which may or may not encode a payload molecule) and 2) a second recombinant RNA polynucleotide encoding a payload molecule. In some embodiments, the synthetic RNA viral genome and the second recombinant RNA polynucleotide encoding the payload molecule are not linked in the particle (e.g., LNP). In some embodiments, the synthetic RNA viral genome and the second recombinant RNA polynucleotide encoding the payload molecule are non-covalently linked. In some embodiments, the synthetic RNA viral genome and the second recombinant RNA polynucleotide encoding the payload molecule are covalently linked via a covalent bond other than a regular 3′, 5′ phosphodiester linkage. In some embodiments, one or more miRNA target sequences are incorporated into the 3′ or 5′ UTR of the RNA polynucleotide encoding the payload molecule. In some embodiments, one or more miRNA target sequences are inserted into the polynucleotide encoding the payload molecule. In such embodiments, translation and subsequent expression of the payload does not occur, or is substantially reduced, in cells where the corresponding miRNA is expressed. In some embodiments, the recombinant RNA polynucleotide encoding a payload molecule is a replicon.

In some embodiments, the payload is a cytotoxic peptide. As used herein, a “cytotoxic peptide” refers to a protein capable of inducing cell death when expressed in a host cell and/or cell death of a neighboring cell when secreted by the host cell. In some embodiments, the cytotoxic peptide is a caspase, p53, diphtheria toxin (DT), Pseudomonas Exotoxin A (PEA), Type I ribozyme inactivating proteins (RIPs) (e.g., saporin and gelonin), Type II RIPs (e.g., ricin), Shiga-like toxin 1 (Slt1), photosensitive reactive oxygen species (e.g. killer-red). In certain embodiments, the cytotoxic peptide is encoded by a suicide gene resulting in cell death through apoptosis, such as a caspase gene.

In some embodiments, the payload is an immune modulatory peptide. As used herein, an “immune modulatory peptide” is a peptide capable of modulating (e.g., activating or inhibiting) a particular immune receptor and/or pathway. In some embodiments, the immune modulatory peptides can act on any mammalian cell including immune cells, tissue cells, and stromal cells. In a preferred embodiment, the immune modulatory peptide acts on an immune cell such as a T cell, an NK cell, an NKT T cell, a B cell, a dendritic cell, a macrophage, a basophil, a mast cell, or an eosinophil. Exemplary immune modulatory peptides include antigen-binding molecules such as antibodies or antigen binding fragments thereof, cytokines, chemokines, soluble receptors, cell-surface receptor ligands, bipartite peptides, and enzymes.

In some embodiments, the payload is a cytokine such as IL-1, IL-12, IL-15, IL-18, IL-36γ, TNFα, IFNα, IFNβ, IFNγ, or TNFSF14. In some embodiments, the payload is a chemokine such as CXCL10, CXCL9, CCL21, CCL4, or CCL5. In some embodiments, the payload is a ligand for a cell-surface receptor such as an NKG2D ligand, a neuropilin ligand, Flt3 ligand, a CD47 ligand (e.g., SIRP1α). In some embodiments, the payload is a soluble receptor, such as a soluble cytokine receptor (e.g., IL-13R, TGFβR1, TGFβR2, IL-35R, IL-15R, IL-2R, IL-12R, and interferon receptors) or a soluble innate immune receptor (e.g., Toll-like receptors, complement receptors, etc.). In some embodiments, the payload is a dominant agonist mutant of a protein involved in intracellular RNA and/or DNA sensing (e.g. a dominant agonist mutant of STING, RIG-1, or MDA-5).

In some embodiments, the payload is an antigen-binding molecule such as an antibody or antigen-binding fragments thereof (e.g., a single chain variable fragment (scFv), an F(ab), etc.). In some embodiments, the antigen-binding molecule specifically binds to a cell surface receptor, such as an immune checkpoint receptor (e.g., PD-1, PD-L1, and CTLA4) or additional cell surface receptors involved in cell growth and activation (e.g., OX40, CD200R, CD47, CSF1R, TREM2, 4-1BB, CD40, and NKG2D).

In some embodiments, the payload molecule is a scorpion polypeptide such as chlorotoxin, BrmKn-2, neopladine 1, neopladine 2, and mauriporin. In some embodiments, the payload molecule is a snake polypeptide such as contortrostatin, apoxin-I, bothropstoxin-I, BJcuL, OHAP-1, rhodostomin, drCT-I, CTX-III, B1L, and ACTX-6. In some embodiments, the payload molecule is a spider polypeptide such as a latarcin and hyaluronidase. In some embodiments, the payload molecule is a bee polypeptide such as melittin and apamin. In some embodiments, the payload molecule is a frog polypeptide such as PsT-1, PdT-1, and PdT-2.

In some embodiments, the payload molecule is an enzyme. In some embodiments, the enzyme is capable of modulating the tumor microenvironment by way of altering the extracellular matrix. In such embodiments, the enzyme may include, but is not limited to, a matrix metalloprotease (e.g., MMP9), a collagenase, a hyaluronidase, a gelatinase, or an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450. In some embodiments, the enzyme is capable of inducing or activating cell death pathways in the target cell (e.g., a caspase). In some embodiments, the enzyme is capable of degrading an extracellular metabolite or message (e.g. adenosine deaminase or arginase or 15-Hydroxyprostaglandin Dehydrogenase).

In some embodiments, the payload molecule is MLKL. In some embodiments, the MLKL polypeptide comprises or consists of an amino acid sequence having at least 70%, at least 75%, at least 80%, at least 85%, at least 90%, at least 95%, at least 97%, at least 98%, at least 99%, or 100% identity to SEQ ID NO: 104. In some embodiments, the payload molecule comprises or consists of a MLKL 4HB domain. In some embodiments, the MLKL 41113 domain comprises or consists of an amino acid sequence having at least 70%, at least 75%, at least 80%, at least 85%, at least 90%, at least 95%, at least 97%, at least 98%, at least 99%, or 100% identity to amino acids 1-120 of SEQ ID NO: 104.

In some embodiments, the payload molecule is a Gasdermin D (GSDMD). In some embodiments, the Gasdermin D (GSDMD) comprises or consists of an amino acid sequence having at least 70%, at least 75%, at least 80%, at least 85%, at least 90%, at least 95%, at least 97%, at least 98%, at least 99%, or 100% identity to SEQ ID NO: 105. In some embodiments, the payload molecule comprises or consists of a Gasdermin D N-terminal fragment. In some embodiments, the Gasdermin D N-terminal fragment comprises or consists of an amino acid sequence having at least 70%, at least 75%, at least 80%, at least 85%, at least 90%, at least 95%, at least 97%, at least 98%, at least 99%, or 100% identity to amino acids 1-233 of SEQ ID NO: 105. In some embodiments, the payload molecule comprises a mutation corresponding to L192A of SEQ ID NO: 105.

In some embodiments, the payload molecule is a Gasdermin E (GSDME). In some embodiments, the Gasdermin E (GSDME) comprises or consists of an amino acid sequence having at least 70%, at least 75%, at least 80%, at least 85%, at least 90%, at least 95%, at least 97%, at least 98%, at least 99%, or 100% identity to SEQ ID NO: 106. In some embodiments, the payload molecule comprises or consists of a Gasdermin E N-terminal fragment. In some embodiments, the Gasdermin E N-terminal fragment comprises or consists of an amino acid sequence having at least 70%, at least 75%, at least 80%, at least 85%, at least 90%, at least 95%, at least 97%, at least 98%, at least 99%, or 100% identity to amino acids 1-237 of SEQ ID NO: 106.

In some embodiments, the payload molecule is a HMGB1. In some embodiments, the HMGB1 polypeptide comprises or consists of an amino acid sequence having at least 70%, at least 75%, at least 80%, at least 85%, at least 90%, at least 95%, at least 97%, at least 98%, at least 99%, or 100% identity to SEQ ID NO: 107. In some embodiments, the payload molecule comprises or consists of a HMGB1 Box B domain. In some embodiments, the HMGB1 Box B domain comprises or consists of an amino acid sequence having at least 70%, at least 75%, at least 80%, at least 85%, at least 90%, at least 95%, at least 97%, at least 98%, at least 99%, or 100% identity to amino acids 96-162 of SEQ ID NO: 107.

In some embodiments, the payload molecule is a SMAC/Diablo. In some embodiments, the SMAC/Diablo comprises or consists of an amino acid sequence having at least 70%, at least 75%, at least 80%, at least 85%, at least 90%, at least 95%, at least 97%, at least 98%, at least 99%, or 100% identity to SEQ ID NO: 108. In some embodiments, the payload molecule comprises or consists of an amino acid sequence having at least 70%, at least 75%, at least 80%, at least 85%, at least 90%, at least 95%, at least 97%, at least 98%, at least 99%, or 100% identity to amino acids 56-239 of SEQ ID NO: 108.

In some embodiments, the payload molecule is a Melittin. In some embodiments, the Melittin comprises or consists of an amino acid sequence having at least 70%, at least 75%, at least 80%, at least 85%, at least 90%, at least 95%, at least 97%, at least 98%, at least 99%, or 100% identity to SEQ ID NO: 109.

In some embodiments, the payload molecule is a L-amino-acid oxidase (LAAO). In some embodiments, the L-amino-acid oxidase (LAAO) comprises or consists of an amino acid sequence having at least 70%, at least 75%, at least 80%, at least 85%, at least 90%, at least 95%, at least 97%, at least 98%, at least 99%, or 100% identity to SEQ ID NO: 110.

In some embodiments, the payload molecule is a disintegrin. In some embodiments, the disintegrin comprises or consists of an amino acid sequence having at least 70%, at least 75%, at least 80%, at least 85%, at least 90%, at least 95%, at least 97%, at least 98%, at least 99%, or 100% identity to SEQ ID NO: 111.

In some embodiments, the payload molecule is a TRAIL (TNFSF10). In some embodiments, the TRAIL (TNFSF10) comprises or consists of an amino acid sequence having at least 70%, at least 75%, at least 80%, at least 85%, at least 90%, at least 95%, at least 97%, at least 98%, at least 99%, or 100% identity to SEQ ID NO: 112.

In some embodiments, the payload molecule is a nitroreductase. In some embodiments, the nitroreductase is NfsB (e.g., from E. coli). In some embodiments, the NfsB comprises or consists of an amino acid sequence having at least 70%, at least 75%, at least 80%, at least 85%, at least 90%, at least 95%, at least 97%, at least 98%, at least 99%, or 100% identity to SEQ ID NO: 113. In some embodiments, the nitroreductase is NfsA (e.g., from E. coli). In some embodiments, the NfsA comprises or consists of an amino acid sequence having at least 70%, at least 75%, at least 80%, at least 85%, at least 90%, at least 95%, at least 97%, at least 98%, at least 99%, or 100% identity to SEQ ID NO: 114.

In some embodiments, the payload molecule is a reovirus FAST protein. In some embodiments, the reovirus FAST protein is an ARV p14, a BRV p15, or a p14-p15 hybrid. In some embodiments, the payload molecule is an ARV p14. In some embodiments, the ARV p14 comprises or consists of an amino acid sequence having at least 70%, at least 75%, at least 80%, at least 85%, at least 90%, at least 95%, at least 97%, at least 98%, at least 99%, or 100% identity to SEQ ID NO: 115. In some embodiments, the payload molecule is a BRV p15. In some embodiments, the BRV p15 comprises or consists of an amino acid sequence having at least 70%, at least 75%, at least 80%, at least 85%, at least 90%, at least 95%, at least 97%, at least 98%, at least 99%, or 100% identity to SEQ ID NO: 116. In some embodiments, the payload molecule is a p14-p15 hybrid. In some embodiments, the p14-p15 hybrid comprises or consists of an amino acid sequence having at least 70%, at least 75%, at least 80%, at least 85%, at least 90%, at least 95%, at least 97%, at least 98%, at least 99%, or 100% identity to SEQ ID NO: 117.

In some embodiments, the payload molecule is a Leptin/FOSL2. In some embodiments, the Leptin/FOSL2 comprises or consists of an amino acid sequence having at least 70%, at least 75%, at least 80%, at least 85%, at least 90%, at least 95%, at least 97%, at least 98%, at least 99%, or 100% identity to SEQ ID NO: 118.

In some embodiments, the payload molecule is an adenosine deaminase 2 (ADA2). In some embodiments, the adenosine deaminase (ADA2) comprises or consists of an amino acid sequence having at least 70%, at least 75%, at least 80%, at least 85%, at least 90%, at least 95%, at least 97%, at least 98%, at least 99%, or 100% identity to SEQ ID NO: 119.

In some embodiments, the payload molecule is an α-1,3-galactosyltransferase. In some embodiments, the α-1,3-galactosyltransferase comprises or consists of an amino acid sequence having at least 70%, at least 75%, at least 80%, at least 85%, at least 90%, at least 95%, at least 97%, at least 98%, at least 99%, or 100% identity to SEQ ID NO: 120.

In some embodiments, the payload molecule is IL-2. In some embodiments, the IL-2 comprises or consists of an amino acid sequence having at least 70%, at least 75%, at least 80%, at least 85%, at least 90%, at least 95%, at least 97%, at least 98%, at least 99%, or 100% identity to SEQ ID NO: 121.

In some embodiments, the payload molecule is IL-7. In some embodiments, the IL-7 comprises or consists of an amino acid sequence having at least 70%, at least 75%, at least 80%, at least 85%, at least 90%, at least 95%, at least 97%, at least 98%, at least 99%, or 100% identity to SEQ ID NO: 122.

In some embodiments, the payload molecule is IL12. In some embodiments, the payload molecule comprises an IL-12 beta subunit comprising or consisting of an amino acid sequence having at least 70%, at least 75%, at least 80%, at least 85%, at least 90%, at least 95%, at least 97%, at least 98%, at least 99%, or 100% identity to SEQ ID NO: 123. In some embodiments, the payload molecule comprises an IL-12 alpha subunit comprising or consisting of an amino acid sequence having at least 70%, at least 75%, at least 80%, at least 85%, at least 90%, at least 95%, at least 97%, at least 98%, at least 99%, or 100% identity to SEQ ID NO: 124.

In some embodiments, the payload molecule is IL18. In some embodiments, the IL18 comprises or consists of an amino acid sequence having at least 70%, at least 75%, at least 80%, at least 85%, at least 90%, at least 95%, at least 97%, at least 98%, at least 99%, or 100% identity to SEQ ID NO: 125.

In some embodiments, the payload molecule is IL-21. In some embodiments, the IL-21 comprises or consists of an amino acid sequence having at least 70%, at least 75%, at least 80%, at least 85%, at least 90%, at least 95%, at least 97%, at least 98%, at least 99%, or 100% identity to SEQ ID NO: 126.

In some embodiments, the payload molecule is IL-36γ. In some embodiments, the IL-367 comprises or consists of an amino acid sequence having at least 70%, at least 75%, at least 80%, at least 85%, at least 90%, at least 95%, at least 97%, at least 98%, at least 99%, or 100% identity to SEQ TD NO: 127.

In some embodiments, the payload molecule is IFNγ. In some embodiments, the IFNγ comprises or consists of an amino acid sequence having at least 70%, at least 7500 at least 80%, at least 85%, at least 90%, at least 9500 at least 9700 at least 98%, at least 9900 or 100% identity to SEQ TD NO: 128.

In some embodiments, the payload molecule is CCL21. In some embodiments, the CCL21 comprises or consists of an amino acid sequence having at least 70%, at least 75%, at least 80%, at least 85%, at least 90%, at least 9500 at least 9700 at least 98%, at least 9900 or 100% identity to SEQ TD NO: 129.

In some embodiments, the payload molecule is encoded by a polynucleotide molecule according to one of the embodiments provided in Table 20 below.

TABLE 20
Non-limiting Embodiments of Payload Configurations

| | | LNP comprising a | | LNP comprising a |
|---|---|---|---|---|
| | CVA (e.g., | CVA (e.g., CVA21) | | SVV viral genome |
| | CVA21) viral | viral genome and a | SVV viral | and a second RNA |
| | genome encoding | second RNA molecule | genome encoding | molecule encoding |
| Payload | the payload | encoding the payload | the payload | the payload |
| MLKL | x | x | x | x |
| MLKL 4HB domain | x | x | x | x |
| Gasdermin D | x | x | x | x |
| Gasdermin D with | x | x | x | x |
| L192A | | | | |
| Gasdermin D N- | x | x | x | x |
| terminal fragment | | | | |
| Gasdermin D N- | x | x | x | x |
| terminal fragment | | | | |
| with L192A | | | | |
| Gasdermin E | x | x | x | x |
| Gasdermin E N- | x | x | x | x |
| terminal fragment | | | | |
| HMGB1 | x | x | x | x |
| HMGB1 Box B domain | x | x | x | x |
| SMAC/Diablo | x | x | x | x |
| Melittin | x | x | x | x |
| L-amino-acid | x | x | x | x |
| oxidase (LAAO) | | | | |
| disintegrin | x | x | x | x |
| TRAIL (TNFSF10) | x | x | x | x |
| nitroreductase | x | x | x | x |
| NfsB | x | x | x | x |
| NfsA | x | x | x | x |
| reovirus FAST | x | x | x | x |
| protein | | | | |
| ARV p14 | x | x | x | x |
| BRV p15 | x | x | x | x |
| p14-p15 hybrid | x | x | x | x |
| Leptin/FOSL2 | x | x | x | x |
| Adenosine deaminase | x | x | x | x |
| 2 (ADA2) | | | | |
| alpha-1,3- | x | x | x | x |
| galactosyltransferase | | | | |
| IL-2 | x | x | x | x |
| IL-7 | x | x | x | x |
| IL-12 | x | x | x | x |
| IL-12 beta subunit | x | x | x | x |
| IL-12 alpha subunit | x | x | x | x |
| IL-18 | x | x | x | x |
| IL-21 | x | x | x | x |
| IL-36 gamma | x | x | x | x |
| IFN gamma | x | x | x | x |
| CCL21 | x | x | x | x |

*Each ‘x’ indicates an embodiment

In some embodiments, the payload molecule is a bipartite peptide. As used herein, a “bipartite peptide” refers to a multimeric protein comprised of a first domain capable of binding a cell surface antigen expressed on a non-cancerous effector cell and a second domain capable of binding a cell-surface antigen expressed by a target cell (e.g., a cancerous cell, a tumor cell, or an effector cell of a different type). In some embodiments, the individual polypeptide domains of a bipartite polypeptide may comprise an antibody or binding fragment thereof (e.g, a single chain variable frag ent (scFv) or n a nanobody, a diabody, a flexibody, a DOCK-AND-LOCK™ antibody, or a monoclonal anti-idiotypic antibody (mAb2). In some embodiments, the structure of the bipartite polypeptides may be a dual-variable domain antibody (DVD-Ig™) a Tandab® a bi-specific T cell engager (BiTE™ a DuoBody®, or a dual affinity retargeting (DART) polypeptide. In some embodiment, the bipartite polypeptide is a BiTE and comprises a domain that specifically binds to an antigen shown in Table 8 and/or 9. Exemplary BiTEs are shown below in Table 7.

TABLE 7
Validated BiTEs used in preclinical and clinical studies

| Target | Name | Target Disease | Clinical Status | References |
|---|---|---|---|---|
| CD19 | Blinatumomab/MT- | NHL, ALL | Phase I/II/III | 1, 2, 3, 4, 5, 6 |
| | 103/MEDI-538 | | | |
| EpCAM | MT110 | Solid tumors | Phase I | 7, 8, 9, 10 |
| CEA | MT111/MEDI-565 | GI adenocarcinoma | Phase I | 11, 12 |
| PSMA | BAY2010112/AMG112 | Prostate | Phase I | 13 |
| CD33 | AMG330 | AML | Preclinical | 14, 15 |
| EGFR | C-BiTE and P-BiTE | Colorectal cancer | Preclinical | 16 |
| | antibodies | | | |
| Her2 | FynomAb, COVA420, | Breast and gastric | Preclinical | 17, 18 |
| | HER2-BsAb | carcinoma | | |
| EphA2 | bscEphA2xCD3 | Multiple solid | Preclinical | 19 |
| | | tumors | | |
| MCSP | MCSP-BiTE | Melanoma | Preclinical | 20 |
| ADAM17 | A300E | Prostate cancer | Preclinical | 21 |
| PSCA | CD3-PSCA(MB1) | Prostate cancer | Preclinical | 22 |
| 17-A1 | CD3/17-1A-bispecific | Colorectal cancer | Preclinical | 23 |
| NKG2D | scFv-NKG2D, | Multiple solid and | Preclinical | 24, 25 |
| ligands | huNKG2D-OKT3 | liquid tumors | | |
| DLL3 | AMG757 | Small Cell Lung | Clinical | 26 |
| | | Cancer | | |

In some embodiments, the cell-surface antigen expressed on an effector cell is selected from Table 8 below. In some embodiments, the cell-surface antigen expressed on a tumor cell or effector cell is selected from Table 9 below. In some embodiments, the cell-surface antigen expressed on a tumor cell is a tumor antigen. In some embodiments, the tumor antigen is selected from CD19, EpCAM, CEA, PSMA, CD33, EGFR, Her2, EphA2, MCSP, ADAM17, PSCA, 17-A1, an NKGD2 ligand, CSF1R, FAP, GD2, DLL3, TROP2, Nectin 4, or neuropilin. In other embodiments, the antigen is a viral antigen associated with the development of cancer. In some embodiments, the viral antigen associated with the development of cancer is HBV-core (Hepatitis B core antigen), HBV-pol, HbS-Ag, HPV E6, HPV E7, Merkel cell polyoma large T antigen, or Epstein Barr virus antigen EBNA2 or BZLF1. In some embodiments, the tumor antigen is selected from those listed in Table 9.

TABLE 8
Exemplary effector cell target antigens

| T cell | | NKT cell | NK Cell | Other |
|---|---|---|---|---|
| CD3 | CD30 | CD3 | CD16 | CD48 |
| CD3γ | CD38 | CD3γ | CD94/NKG2 | LIGHT |
| | | | (e.g., NKG2D) | |
| CD3δ | CD40 | CD3δ | NKp30 | CD44 |
| CD3ε | CD57 | CD3ε | NKp44 | CD45 |
| CD3ζ | CD69 | CD3ζ | NKp46 | IL-1R2 |
| CD2 | CD70 | invariant TCR | KARs | IL-1Rα |
| CD4 | CD73 | | | IL-1Rα2 |
| CD5 | CD81 | | | IL-13Rα2 |
| CD6 | CD82 | | | IL-15Ra |
| CD7 | CD96 | | | CCR5 |
| CD8 | CD134 | | | CCR8 |
| CD16 | CD137 | | | |
| CD25 | CD152 | | | |
| CD27 | CD278 | | | |
| CD28 | | | | |

TABLE 9
Exemplary target cell antigens
Target Cell Antigens

| | | | |
|---|---|---|---|
| 8H9 | CRISP3 | Lewis-Y | Fas |
| GnT-V, β1,6-N | DC-SIGN | LIV-1 (SLC39A6) | SOX2 |
| AFP | DHFR | Livin | STEAP1 |
| ART1 | EGP40 | LAMP1 | SLITRK6 |
| ART4 | EZH2 | MAGEA3 | NaPi2a |
| ABCG2 | EpCAM | MAGEA4 | SOX1 |
| B7-H3 | EphA2 | MAGEB6 | SOX11 |
| B7-H4 | EphA2/Eck | MAGEA1 | SPANXA1 |
| B7-H6 | EGFRvIII | MART-1 | SART-1 |
| BCMA | E-cadherin | MCSP | SSX4 |
| B-cyclin | EGP2 | MME | SSX5 |
| BMI1 | ETA | mesothelin (MSLN) | Survivin |
| CA-125 | ERBB3 | MAPK1 | SSX2 |
| cadherin | ERBB3/4 | MUC16 | TAG72 |
| CABYR | ERBB4 | MUC1 | TEM1 |
| CTAG2 | EPO | MRP-3 | TEM8 |
| CA6 | F3 | MyoD-1 | TSGA10 |
| CAIX | FAR | NCAM | TSSK6 |
| CEA | FBP | nectin 4 | thyroglobulin |
| CEACAM5 | FTHL17 | Nestin | transferrin receptor |
| CEACAM6 | fetal AchR | NEP | TACSTD2 (TROP2) |
| Cav-1 | FAP | NY-ESO-1 | TMEM97 |
| CD10 | FGFR3 | hHLA-A | TRP-2 |
| CD117 | FR-a | H60 | TULP2 |
| CD123 | Fra-l/Fosl 1 | OLIG2 | TROP2 |
| CD133 | GAGE1 | 5T4 | tyrosinase |
| CD138 | GD2 | p53 | TRP1 |
| CD15 | GD3 | P-Cadherin | UPAR |
| CD171 | Glil | PB | VEGF |
| CD19 | GP100 | P-glycoprotein | VEGF receptors |
| CD20 | GPA33 | PMCT (SLC13A5) | VEGRR2 |
| CD21 | | PRAME | BRAF |
| CD22 | Glypican-3 | PROXl | WT-1 |
| CD30 | HIV gp120 | PSA | XAGE2 |
| CD33 | HLA-A | PSCA | ZNF165 |
| CD37 | HLA-A2 | PSMA | αᵥβ₆ integrin |
| CD38 | HLA-AI | PSC1 | β-catenin |
| CD44v6 | HLA-B | PVRL4 | cathepsin B |
| CD44v7/8 | HLA-C | Ras | CSAG2 |
| CD74 | HMW-MAA | ROR1 | CTAG2 |
| Cd79b | Her2/Neu | SART2 | EGFR |
| CD124 (IL-4R) | Her3 | SART3 | EGP40 |
| CDH3 | u70/80 | oncofetal variants | EZH2 |
| | | of fibronectin | |
| Ki-67 | LICAM | tenascin | HIV sp120 |
| CSPG4 | ULBP1 | LICAM | kappa light chain |
| CALLA | ULBP2 | Rae-1α | LDHC |
| CSAG2 | ULBP3 | Rae-1β | TRP-1 |
| COX-2 | ULBP6 | Rae-1δ | Fas-L |
| Lambda | MICA | Rae-1γ | DLL3 |
| LAYN | MICB | PDGF | |
| LeuM-1 | Her3 | TROP2 | |
| KDR | EGF | | |
| CD47 | SIRP1α | | |

### Pharmaceutical Compositions and Methods of Use

One aspect of the disclosure relates to pharmaceutical compositions comprising the recombinant RNA molecules described herein, or particles comprising a recombinant RNA molecule described herein, and methods for the treatment of cancer. In some embodiments, the present disclosure provides methods of treating cancer in a subject in need thereof comprising administering an effective amount of a CVA21-EF, a CVA21-KY, or an SVV virus or the corresponding RNA viral genome to the subject. Compositions described herein can be formulated in any manner suitable for a desired delivery route. Typically, formulations include all physiologically acceptable compositions including derivatives or prodrugs, solvates, stereoisomers, racemates, or tautomers thereof with any pharmaceutically acceptable carriers, diluents, and/or excipients.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-O-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline, and caffeine.

The present disclosure provides methods of killing a cancerous cell or a target cell comprising exposing the cell to an RNA polynucleotide or particle described herein, or composition thereof, under conditions sufficient for the intracellular delivery of the composition to the cancerous cell. As used herein, a “cancerous cell” or a “target cell” refers to a mammalian cell selected for treatment or administration with a polynucleotide or particle described herein, or composition thereof described herein. As used herein “killing a cancerous cell” refer specifically to the death of a cancerous cell by means of apoptosis or necrosis. Killing of a cancerous cell may be determined by methods known in the art including but not limited to, tumor size measurements, cell counts, and flow cytometry for the detection of cell death markers such as Annexin V and incorporation of propidium iodide.

The present disclosure further provides for a method of treating or preventing cancer in a subject in need thereof wherein an effective amount of the pharmaceutical compositions described herein is administered to the subject. The route of administration will vary, naturally, with the location and nature of the disease being treated, and may include, for example intradermal, transdermal, subdermal, parenteral, nasal, intravenous, intramuscular, intranasal, subcutaneous, percutaneous, intratracheal, intraperitoneal, intratumoral, perfusion, lavage, direct injection, and oral administration. The encapsulated polynucleotide compositions described herein are particularly useful in the treatment of metastatic cancers, wherein systemic administration may be necessary to deliver the compositions to multiple organs and/or cell types. Therefore, in a particular embodiment, the compositions described herein are administered systemically.

An “effective amount” or an “effective dose,” used interchangeably herein, refers to an amount and or dose of the compositions described herein that results in an improvement or remediation of the symptoms of the disease or condition. The improvement is any improvement or remediation of the disease or condition, or symptom of the disease or condition. The improvement is an observable or measurable improvement or may be an improvement in the general feeling of well-being of the subject. Thus, one of skill in the art realizes that a treatment may improve the disease condition but may not be a complete cure for the disease. Improvements in subjects may include, but are not limited to, decreased tumor burden, decreased tumor cell proliferation, increased tumor cell death, activation of immune pathways, increased time to tumor progression, decreased cancer pain, increased survival, or improvements in the quality of life.

In some embodiments, administration of an effective dose may be achieved with administration a single dose of a composition described herein. As used herein, “dose” refers to the amount of a composition delivered at one time. In some embodiments, the dose of the recombinant RNA molecules is measured as the 50% Tissue culture Infective Dose (TCID₅₀). In some embodiments, the TCID₅₀ is at least about 10³-10⁹ TCID₅₀/mL, for example, at least about 10³ TCID₅₀/mL, about 10⁴ TCID₅₀/mL, about 10⁵ TCID₅₀/mL, about 10⁶ TCID₅₀/mL, about 10⁷ TCID₅₀/mL, about 10⁸ TCID₅₀/mL, or about 10⁹ TCID₅₀/mL. In some embodiments, a dose may be measured by the number of particles in a given volume (e.g., particles/mL). In some embodiments, a dose may be further refined by the genome copy number of the RNA polynucleotides described herein present in each particle (e.g., # of particles/mL, wherein each particle comprises at least one genome copy of the polynucleotide). In some embodiments, delivery of an effective dose may require administration of multiple doses of a composition described herein. As such, administration of an effective dose may require the administration of at least 2, 3, 4, 5, 6, 7, 8, 9, 10, 20, 30, 40, 50, or more doses of a composition described herein.

In embodiments wherein multiple doses of a composition described herein are administered, each dose need not be administered by the same actor and/or in the same geographical location. Further, the dosing may be administered according to a predetermined schedule. For example, the predetermined dosing schedule may comprise administering a dose of a composition described herein daily, every other day, weekly, bi-weekly, monthly, bi-monthly, annually, semi-annually, or the like. The predetermined dosing schedule may be adjusted as necessary for a given patient (e.g., the amount of the composition administered may be increased or decreased and/or the frequency of doses may be increased or decreased, and/or the total number of doses to be administered may be increased or decreased).

As used herein “prevention” or “prophylaxis” can mean complete prevention of the symptoms of a disease, a delay in onset of the symptoms of a disease, or a lessening in the severity of subsequently developed disease symptoms.

The term “subject” or “patient” as used herein, is taken to mean any mammalian subject to which a composition described herein is administered according to the methods described herein. In a specific embodiment, the methods of the present disclosure are employed to treat a human subject. The methods of the present disclosure may also be employed to treat non-human primates (e.g., monkeys, baboons, and chimpanzees), mice, rats, bovines, horses, cats, dogs, pigs, rabbits, goats, deer, sheep, ferrets, gerbils, guinea pigs, hamsters, bats, birds (e.g., chickens, turkeys, and ducks), fish, and reptiles.

In some embodiments, the present disclosure provides a method of treating a cancer in a subject in need thereof, comprising administering a therapeutically effective amount of an oncolytic Coxsackievirus, wherein the Coxsackievirus is a CVA21 strain, or a polynucleotide encoding the CVA21 to the subject, wherein the cancer is classified as sensitive to CVA21 infection based on the expression level of ICAM-1 and/or the percentage of ICAM-1 positive cancer cells in the cancer. In some embodiments, the CVA21 strain is CVA21-KY.

Intracellular adhesion molecule 1 (ICAM-1, also known as BB2, CD54, P3.58) is a protein (UniProt Ref: P03562) encoded by the ICAM1 gene (NCBI Gene ID: 3383) and is important in stabilizing cell-cell interactions and facilitating leukocyte endothelial transmigration. In some embodiments, treatment decisions for a particular cancer are made based on ICAM-1 expression, wherein the expression of ICAM-1 is determined in the cancer and the cancer is identified as sensitive or resistant to CVA21 expression based on the level of ICAM-1 expression. In general, higher (% of positive tumor cells or intensity or both) expression of ICAM-1 indicates greater sensitivity to CVA21 infection (See Example 8). ICAM-1 expression can be determined by means known in the art for mRNA and/or protein expression. mRNA expression can be determined by northern blots, ribonuclease protection assays, PCR-based methods, sequencing methods, and the like. Protein expression can be determined by immunoblotting (e.g., western blot), immunohistochemistry, immunofluorescence, enzyme-linked immunosorbent assay (ELISA), flow cytometry, cytometric bead array, mass spectroscopy, proteomics-based methods, and the like.

In some embodiments, the present disclosure provides a method of treating a cancer in a subject in need thereof, comprising: (a) determining the expression level of ICAM-1 and/or the percentage of ICAM-1 positive cancer cells in the cancer; (b) classifying the cancer as sensitive to Coxsackievirus 21 (CVA21) infection based on the expression level of ICAM-1 and/or the percentage of ICAM-1 positive cancer cells determined in (a); and (c) administering a therapeutically effective amount of CVA21 or a polynucleotide encoding the CVA21 to the subject if the cancer is classified as sensitive to CVA21 infection in step (b). In some embodiments, the CVA21 strain is CVA21-KY.

In some embodiments, the present disclosure provides a method of selecting a subject suffering from a cancer for treatment with a Coxsackievirus 21 (CVA21) or a polynucleotide encoding the CVA21, comprising: (a) determining the expression level of ICAM-1 and/or the percentage of ICAM-1 positive cancer cells in the cancer; (b) classifying the cancer as sensitive to CVA21 infection based on the expression level of ICAM-1 and/or the percentage of ICAM-1 positive cancer cells as determined in (a); (c) selecting the subject for treatment with the CVA21 or the polynucleotide encoding the CVA21 if the cancer is classified as sensitive to CVA21 infection in (b); and (d) administering the CVA21 or the polynucleotide encoding the CVA21 to the selected subject. In some embodiments, the CVA21 strain is CVA21-KY.

### Lipid Nanoparticle Composition and Methods of Use

In some embodiments, the disclosure provides methods of treating a disease or disorder in a subject in need thereof, comprising administering to the subject a therapeutically effective amount of a composition (e.g., pharmaceutical composition) of the disclosure. In some embodiments, the disease or disorder comprises a cancer. In some embodiments, the composition comprises a PEG-lipid of the disclosure. In some embodiments, the composition comprises an LNP of the disclosure comprising a PEG-lipid. In some embodiments, the composition comprises an LNP of the disclosure comprising a PEG-lipid and an encapsulated molecule of the disclosure (e.g., synthetic RNA viral genome).

The method may be a method of treating a subject having or at risk of having a condition that benefits from the encapsulated molecule, particularly if the encapsulated molecule is a therapeutic agent. Alternatively, the method may be a method of diagnosing a subject, in which case the encapsulated molecule may be is a diagnostic agent.

In prophylactic applications, pharmaceutical compositions comprising an LNP of the disclosure are administered to a subject susceptible to, or otherwise at risk of, a particular disorder in an amount sufficient to eliminate or reduce the risk or delay the onset of the disorder. In therapeutic applications, compositions comprising an LNP of the disclosure are administered to a subject suspected of, or already suffering from such a disorder in an amount sufficient to cure, or at least partially arrest, the symptoms of the disorder and its complications. An amount adequate to accomplish this is referred to as a therapeutically effective dose or amount. In both prophylactic and therapeutic regimes, the pharmaceutical composition can be administered in several dosages until a sufficient response has been achieved. Typically, the response is monitored and repeated dosages are given if the desired response starts to fade.

For administration, the LNP of the disclosure may be formulated as a pharmaceutical composition. In some embodiments, the LNP comprises an encapsulated molecule. A pharmaceutical composition may comprise: (i) an LNP of the disclosure; and (ii) a pharmaceutically acceptable carrier, diluent or excipient. A pharmaceutical composition can be formulated according to known methods to prepare pharmaceutically useful compositions, whereby the therapeutic molecule is combined in a mixture with a pharmaceutically acceptable carrier, diluent, or excipient. A carrier is said to be a “pharmaceutically acceptable carrier” if its administration can be tolerated by a recipient subject. Sterile phosphate-buffered saline is one example of a pharmaceutically acceptable carrier. Other suitable carriers, diluents, or excipients are well-known to those in the art. (See, e.g., Gennaro (ed.), Remington's Pharmaceutical Sciences (Mack Publishing Company, 19th ed. 1995).) Formulations can further include one or more excipients, preservatives, solubilizers, buffering agents, albumin to prevent protein loss on vial surfaces, etc.

A pharmaceutical composition comprising LNPs of the disclosure may be formulated in a dosage form selected from the group consisting of: an oral unit dosage form, an intravenous unit dosage form, an intranasal unit dosage form, a suppository unit dosage form, an intradermal unit dosage form, an intramuscular unit dosage form, an intraperitoneal unit dosage form, a subcutaneous unit dosage form, an epidural unit dosage form, a sublingual unit dosage form, and an intracerebral unit dosage form. The oral unit dosage form may be selected from the group consisting of: tablets, pills, pellets, capsules, powders, lozenges, granules, solutions, suspensions, emulsions, syrups, elixirs, sustained-release formulations, aerosols, and sprays.

A pharmaceutical composition may be administered to a subject in a therapeutically effective amount. According to the methods of the disclosure, a composition can be administered to subjects by a variety of administration modes, including, for example, by intramuscular, subcutaneous, intravenous, intra-atrial, intra-articular, parenteral, intranasal, intrapulmonary, transdermal, intrapleural, intrathecal, intratumoral, and oral routes of administration. For prevention and treatment purposes, a composition can be administered to a subject in a single bolus delivery, via continuous delivery (e.g., continuous transdermal delivery) over an extended time period, or in a repeated administration protocol (e.g., on an hourly, daily, weekly, or monthly basis).

Administration can occur by injection, irrigation, inhalation, consumption, electro-osmosis, hemodialysis, iontophoresis, and other methods known in the art. The route of administration will vary, naturally, with the location and nature of the disease being treated, and may include, for example auricular, buccal, conjunctival, cutaneous, dental, endocervical, endosinusial, endotracheal, enteral, epidural, interstitial, intra-articular, intra-arterial, intra-abdominal, intraauricular, intrabiliary, intrabronchial, intrabursal, intracavernous, intracerebral, intracisternal, intracorneal, intracronal, intracoronary, intracranial, intradermal, intradiscal, intraductal, intraduodenal, intraduodenal, intradural, intraepicardial, intraepidermal, intraesophageal, intragastric, intragingival, intrahepatic, intraileal, intralesional, intralingual, intraluminal, intralymphatic, intramammary, intramedulleray, intrameningeal, instramuscular, intranasal, intranodal, intraocular, intraomentum, intraovarian, intraperitoneal, intrapericardial, intrapleural, intraprostatic, intrapulmonary, intraruminal, intrasinal, intraspinal, intrasynovial, intratendinous, intratesticular, intratracheal, intrathecal, intrathoracic, intratubular, intratumoral, intratympanic, intrauterine, intraperitoneal, intravascular, intraventricular, intravesical, intravestibular, intravenous, intravitreal, larangeal, nasal, nasogastric, oral, ophthalmic, oropharyngeal, parenteral, percutaneous, periarticular, peridural, perineural, periodontal, respiratory, retrotubular, rectal, spinal, subarachnoid, subconjunctival, subcutaneous, subdermal, subgingival, sublingual, submucosal, subretinal, topical, transdermal, transendocardial, transmucosal, transplacental, trantracheal, transtympanic, ureteral, urethral, and/or vaginal perfusion, lavage, direct injection, and oral administration.

In some embodiments, the pharmaceutical composition is formulated for systemic administration. In some embodiments, the systemic administration comprises intravenous administration, intra-arterial administration, intraperitoneal administration, intramuscular administration, intradermal administration, subcutaneous administration, intranasal administration, oral administration, or a combination thereof. In some embodiments, the pharmaceutical composition is formulated for intravenous administration. In some embodiments, the pharmaceutical composition is formulated for local administration. In some embodiments, the pharmaceutical composition is formulated for intratumoral administration.

Effective doses of the compositions of the disclosure vary depending upon many different factors, including means of administration, target site, physiological state of the subject, whether the subject is human or an animal, other medications administered, whether treatment is prophylactic or therapeutic, as well as the specific activity of the composition itself and its ability to elicit the desired response in the individual. In some embodiments, the subject is a human. In some embodiments, the subject can be a nonhuman mammal. Typically, dosage regimens are adjusted to provide an optimum therapeutic response, i.e., to optimize safety and efficacy.

Determination of effective dosages in this context is typically based on animal model studies followed up by human clinical trials and is guided by determining effective dosages and administration protocols that significantly reduce the occurrence or severity of the subject disorder in model subjects. Compositions of the disclosure may be suitably administered to the subject at one time or over a series of treatments and may be administered to the subject at any time from diagnosis onwards. Compositions of the disclosure may be administered as the sole treatment, as a monotherapy, or in conjunction with other drugs or therapies, as a combinatorial therapy, useful in treating the condition in question.

In some embodiments, the therapeutically effective amount of a composition of the disclosure is between about 1 ng/kg body weight to about 100 mg/kg body weight. In some embodiments, the range of a composition of the disclosure administered is from about 1 ng/kg body weight to about 1 μg/kg body weight, about 1 ng/kg body weight to about 100 ng/kg body weight, about 1 ng/kg body weight to about 10 ng/kg body weight, about 10 ng/kg body weight to about 1 μg/kg body weight, about 10 ng/kg body weight to about 100 ng/kg body weight, about 100 ng/kg body weight to about 1 μg/kg body weight, about 100 ng/kg body weight to about 10 pg/kg body weight, about 1 μg/kg body weight to about 10 pg/kg body weight, about 1 μg/kg body weight to about 100 pg/kg body weight, about 10 pg/kg body weight to about 100 pg/kg body weight, about 10 pg/kg body weight to about 1 mg/kg body weight, about 100 μg/kg body weight to about 10 mg/kg body weight, about 1 mg/kg body weight to about 100 mg/kg body weight, or about 10 mg/kg body weight to about 100 mg/kg body weight. Dosages within this range can be achieved by single or multiple administrations, including, e.g., multiple administrations per day or daily, weekly, bi-weekly, or monthly administrations. Compositions of the disclosure may be administered, as appropriate or indicated, as a single dose by bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. In some embodiments, a composition of the disclosure is administered weekly. In some embodiments, a composition of the disclosure is administered biweekly. In some embodiments, a composition of the disclosure is administered every three weeks. However, other dosage regimens may be useful. The progress of this therapy is easily monitored by conventional techniques.

For administration to a human adult subject, the therapeutically effective amount may be administered in doses in the range of 0.0006 mg to 1000 mg per dose, including but not limited to 0.0006 mg per dose, 0.001 mg per dose, 0.003 mg per dose, 0.006 mg per dose, 0.01 mg per dose, 0.03 mg per dose, 0.06 mg per dose, 0.1 mg per dose, 0.3 mg per dose, 0.6 mg per dose, 1 mg per dose, 3 mg per dose, 6 mg per dose, 10 mg per dose, 30 mg per dose, 60 mg per dose, 100 mg per dose, 300 mg per dose, 600 mg per dose and 1000 mg per dose, and multiple, usually consecutive daily doses may be administered in a course of treatment. In some embodiments, a composition of the disclosure is administered at a dose level of about 0.001 mg/kg/dose to about 10 mg/kg/dose, about 0.001 mg/kg/dose to about 6 mg/kg/dose, about 0.001 mg/kg/dose to about 3 mg/kg/dose, about 0.001 mg/kg/dose to about 1 mg/kg/dose, about 0.001 mg/kg/dose to about 0.6 mg/kg/dose, about 0.001 mg/kg/dose to about 0.3 mg/kg/dose, about 0.001 mg/kg/dose to about 0.1 mg/kg/dose, about 0.001 mg/kg/dose to about 0.06 mg/kg/dose, about 0.001 mg/kg/dose to about 0.03 mg/kg/dose, about 0.001 mg/kg/dose to about 0.01 mg/kg/dose, about 0.001 mg/kg/dose to about 0.006 mg/kg/dose, about 0.001 mg/kg/dose to about 0.003 mg/kg/dose, about 0.003 mg/kg/dose to about 10 mg/kg/dose, about 0.003 mg/kg/dose to about 6 mg/kg/dose, about 0.003 mg/kg/dose to about 3 mg/kg/dose, about 0.003 mg/kg/dose to about 1 mg/kg/dose, about 0.003 mg/kg/dose to about 0.6 mg/kg/dose, about 0.003 mg/kg/dose to about 0.3 mg/kg/dose, about 0.003 mg/kg/dose to about 0.1 mg/kg/dose, about 0.003 mg/kg/dose to about 0.06 mg/kg/dose, about 0.003 mg/kg/dose to about 0.03 mg/kg/dose, about 0.003 mg/kg/dose to about 0.01 mg/kg/dose, about 0.003 mg/kg/dose to about 0.006 mg/kg/dose, about 0.006 mg/kg/dose to about 10 mg/kg/dose, about 0.006 mg/kg/dose to about 6 mg/kg/dose, about 0.006 mg/kg/dose to about 3 mg/kg/dose, about 0.006 mg/kg/dose to about 1 mg/kg/dose, about 0.006 mg/kg/dose to about 0.6 mg/kg/dose, about 0.006 mg/kg/dose to about 0.3 mg/kg/dose, about 0.006 mg/kg/dose to about 0.1 mg/kg/dose, about 0.006 mg/kg/dose to about 0.06 mg/kg/dose, about 0.006 mg/kg/dose to about 0.03 mg/kg/dose, about 0.006 mg/kg/dose to about 0.01 mg/kg/dose, about 0.01 mg/kg/dose to about 10 mg/kg/dose, about 0.01 mg/kg/dose to about 6 mg/kg/dose, about 0.01 mg/kg/dose to about 3 mg/kg/dose, about 0.01 mg/kg/dose to about 1 mg/kg/dose, about 0.01 mg/kg/dose to about 0.6 mg/kg/dose, about 0.01 mg/kg/dose to about 0.3 mg/kg/dose, about 0.01 mg/kg/dose to about 0.1 mg/kg/dose, about 0.01 mg/kg/dose to about 0.06 mg/kg/dose, about 0.01 mg/kg/dose to about 0.03 mg/kg/dose, about 0.03 mg/kg/dose to about 10 mg/kg/dose, about 0.03 mg/kg/dose to about 6 mg/kg/dose, about 0.03 mg/kg/dose to about 3 mg/kg/dose, about 0.03 mg/kg/dose to about 1 mg/kg/dose, about 0.03 mg/kg/dose to about 0.6 mg/kg/dose, about 0.03 mg/kg/dose to about 0.3 mg/kg/dose, about 0.03 mg/kg/dose to about 0.1 mg/kg/dose, about 0.03 mg/kg/dose to about 0.06 mg/kg/dose, about 0.06 mg/kg/dose to about 10 mg/kg/dose, about 0.06 mg/kg/dose to about 6 mg/kg/dose, about 0.06 mg/kg/dose to about 3 mg/kg/dose, about 0.06 mg/kg/dose to about 1 mg/kg/dose, about 0.06 mg/kg/dose to about 0.6 mg/kg/dose, about 0.06 mg/kg/dose to about 0.3 mg/kg/dose, about 0.06 mg/kg/dose to about 0.1 mg/kg/dose, about 0.1 mg/kg/dose to about 10 mg/kg/dose, about 0.1 mg/kg/dose to about 6 mg/kg/dose, about 0.1 mg/kg/dose to about 3 mg/kg/dose, about 0.1 mg/kg/dose to about 1 mg/kg/dose, about 0.1 mg/kg/dose to about 0.6 mg/kg/dose, about 0.1 mg/kg/dose to about 0.3 mg/kg/dose, about 0.3 mg/kg/dose to about 10 mg/kg/dose, about 0.3 mg/kg/dose to about 6 mg/kg/dose, about 0.3 mg/kg/dose to about 3 mg/kg/dose, about 0.3 mg/kg/dose to about 1 mg/kg/dose, about 0.3 mg/kg/dose to about 0.6 mg/kg/dose, about 0.6 mg/kg/dose to about 10 mg/kg/dose, about 0.6 mg/kg/dose to about 6 mg/kg/dose, about 0.6 mg/kg/dose to about 3 mg/kg/dose, about 0.6 mg/kg/dose to about 1 mg/kg/dose, about 1 mg/kg/dose to about 10 mg/kg/dose, about 1 mg/kg/dose to about 6 mg/kg/dose, about 1 mg/kg/dose to about 3 mg/kg/dose, about 3 mg/kg/dose to about 10 mg/kg/dose, about 3 mg/kg/dose to about 6 mg/kg/dose, or about 6 mg/kg/dose to about 10 mg/kg/dose. In some embodiments, a composition of the disclosure is administered at a dose level of about 0.001 mg/kg/dose, about 0.003 mg/kg/dose, about 0.006 mg/kg/dose, about 0.01 mg/kg/dose, about 0.03 mg/kg/dose, about 0.06 mg/kg/dose, about 0.1 mg/kg/dose, about 0.3 mg/kg/dose, about 0.6 mg/kg/dose, about 1 mg/kg/dose, about 3 mg/kg/dose, about 6 mg/kg/dose, or about 10 mg/kg/dose. Compositions of the disclosure can be administered at different times of the day. In one embodiment the optimal therapeutic dose can be administered in the evening. In another embodiment the optimal therapeutic dose can be administered in the morning. As expected, the dosage will be dependent on the condition, size, age, and condition of the subject.

Dosage of the pharmaceutical composition can be varied by the attending clinician to maintain a desired concentration at a target site. Higher or lower concentrations can be selected based on the mode of delivery. Dosage should also be adjusted based on the release rate of the administered formulation.

In some embodiments, the pharmaceutical composition of the disclosure is administered to a subject for multiple times (e.g., multiple doses). In some embodiments, the pharmaceutical composition is administered two or more times, three or more times, four or more times, etc. In some embodiments, administration of the pharmaceutical composition may be repeated once, twice, 3, 4, 5, 6, 7, 8, 9, 10, or more times. The pharmaceutical composition may be administered chronically or acutely, depending on its intended purpose.

In some embodiments, the interval between two consecutive doses of the pharmaceutical composition is less than 4, less than 3, less than 2, or less than 1 weeks. In some embodiments, the interval between two consecutive doses is less than 3 weeks. In some embodiments, the interval between two consecutive doses is less than 2 weeks. In some embodiments, the interval between two consecutive doses is less than 1 week. In some embodiments, the interval between two consecutive doses is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive doses of the pharmaceutical composition is at least 4, at least 3, at least 2, or at least 1 weeks. In some embodiments, the interval between two consecutive doses of the pharmaceutical composition of the disclosure is at least 3 weeks. In some embodiments, the interval between two consecutive doses of the pharmaceutical composition of the disclosure is at least 2 weeks. In some embodiments, the interval between two consecutive doses of the pharmaceutical composition of the disclosure is at least 1 week. In some embodiments, the interval between two consecutive doses of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the subject is administered a dose of the pharmaceutical composition of the disclosure once daily, every 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the subject is administered a dose of the pharmaceutical composition of the disclosure once every 4, 5, 6, 7, 8, 9, 10, 11, or 12 weeks. In some embodiments, the subject is administered a dose of the pharmaceutical composition of the disclosure once every 1, 2, 3, 4, 5, 6, 7, 8, 9, 10, 11, or 12 months.

In some embodiments, the pharmaceutical composition of the disclosure is administered multiple times, wherein the serum half-life of the LNP in the subject following the second and/or subsequent administration is at least 40%, 50%, 60%, 70%, 80%, 85%, 90%, or 95% of the serum half-life of the LNP following the first administration.

In some embodiments, the second and subsequent doses of the pharmaceutical composition comprising an encapsulated molecule (e.g., encapsulated in an LNP) may maintain an activity of the encapsulated molecule of at least 50% of the activity of the first dose, or at least 60% of the first dose, or at least 70% of the first dose, or at least 75% of the first dose, or at least 80% of the first dose, or at least 85% of the first dose, or at least 90% of the first dose, or at least 95% of the first dose, or more, for at least 1 day, 2 days, 3 days, 4 days, 5 days, 6 days, or 7 days after second administration or subsequent administration.

In some embodiments, the pharmaceutical composition of the disclosure has an duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer. In some embodiments, the pharmaceutical composition of the disclosure has an duration of therapeutic effect in vivo of at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1.5 days, 2 days, 3 days, 4 days, 5 days, 6 days, 7 days, 8 days, 9 days, or 10 days.

In some embodiments, the pharmaceutical composition of the disclosure has a half-life in vivo comparable to that of a pre-determined threshold value. In some embodiments, the pharmaceutical composition of the disclosure has a half-life in vivo greater than that of a pre-determined threshold value. In some embodiments, the pharmaceutical composition of the disclosure has a half-life in vivo shorter than that of a pre-determined threshold value. In some embodiments, the pre-determined threshold value is the half-life of a control composition comprising the same payload molecule and LNP except that the LNP comprises (i) a PEG-lipid that is not of Formula (A), (A′), or (A″) (for example, the PEG-lipid of the LNP in the control composition may be PEG2k-DPG); or (ii) a cationic lipid that is not of Formula (I).

In some embodiments, the pharmaceutical composition of the disclosure has an AUC (area under the blood concentration-time curve) following a repeat dose that is at least 30%, at least 40%, at least 50%, at least 60%, at least 70%, at least 80%, at least 90%, or at least 100% of the AUC following the previous dose. In some embodiments, the pharmaceutical composition has an AUC that is at least 60% of the AUC following the previous dose. In some embodiments, following a repeat dose, AUC of the pharmaceutical composition decreases less than 70%, less than 60%, less than 60%, less than 40%, less than 30%, less than 20%, less than 10%, or less than 5% compared to the AUC following the previous dose. In some embodiments, following a repeat dose, AUC of the pharmaceutical composition decreases less than 40% compared to the AUC following the previous dose.

In some embodiments, the pharmaceutical composition of the disclosure comprises a nucleic acid molecule encoding viral genome of an oncolytic virus, and wherein administration of the pharmaceutical composition to a subject bearing a tumor delivers the nucleic acid molecule into tumor cells. In some embodiments, the nucleic acid molecule is a RNA molecule. In some embodiments, administration of the pharmaceutical composition results in replication of the oncolytic virus in tumor cells. In some embodiments, administration of the pharmaceutical composition to a subject bearing a tumor results in selective replication of the oncolytic virus in tumor cells as compared to normal cells.

In some embodiments, administration of the pharmaceutical composition of the disclosure to a subject bearing a tumor inhibits growth of the tumor. In some embodiments, administration of the pharmaceutical composition inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, administration of the pharmaceutical composition to a subject bearing a tumor leads to tumor shrinkage or elimination. In some embodiments, administration of the pharmaceutical composition leads to tumor shrinkage or elimination for at least 1 week, at least 1 month, at least 2 months, at least 3 months, at least 4 months, at least 6 months, at least 9 months, at least 12 months, at least 2 years, or longer. In some embodiments, administration of the pharmaceutical composition leads to tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

Pharmaceutical compositions can be supplied as a kit comprising a container that comprises the pharmaceutical composition as described herein. A pharmaceutical composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a pharmaceutical composition. Such a kit can further comprise written information on indications and usage of the pharmaceutical composition

The disclosure relates to a method of treating cancer in a subject in need thereof, comprising administering a therapeutically effective amount of a composition as described herein to the subject.

In some embodiments, the disclosure provides methods of delivering a encapsulated molecule to a cell, the method comprising contacting the cell with the LNP or pharmaceutical composition thereof, wherein the LNP comprises the encapsulated molecule. In some embodiments, the encapsulated molecule is a nucleic acid molecule encoding a virus, and wherein contacting the cell with the LNP results in production of viral particles by the cell, and wherein the viral particles are infectious and lytic.

In some embodiments, the disclosure provides methods of delivering an LNP to a subject, comprising administering the LNP or the pharmaceutical composition thereof of the disclosure to the subject. In some embodiments, the method comprises multiple administrations. In some embodiments, the interval between two consecutive administrations of the pharmaceutical composition is less than 4, less than 3, less than 2, or less than 1 weeks. In some embodiments, the interval between two consecutive administrations is less than 2 weeks. In some embodiments, the interval between two consecutive administrations is less than 1 week. In some embodiments, the interval between two consecutive administrations is less than 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the interval between two consecutive administrations of the pharmaceutical composition is at least 4, at least 3, at least 2, or at least 1 weeks. In some embodiments, the interval between two consecutive administrations of the pharmaceutical composition of the disclosure is at least 2 weeks. In some embodiments, the interval between two consecutive administrations of the pharmaceutical composition of the disclosure is at least 1 week. In some embodiments, the interval between two consecutive administrations of the pharmaceutical composition of the disclosure is at least 28, 27, 26, 25, 24, 23, 22, 21, 20, 19, 18, 17, 16, 15, 14, 13, 12, 11, 10, 9, 8, 7, 6, 5, 4, 3, 2 or 1 days. In some embodiments, the method comprises administering to a subject the pharmaceutical composition of the disclosure every 1, 2, 3, 4, 5, 6, 7, 8, 9, 10, 11, 12, 13, 14, 15, 16, 17, 18, 19, 20, 21, 22, 23, 24, 25, 26, 27, or 28 days. In some embodiments, the method comprises administering to a subject the pharmaceutical composition of the disclosure once every 4, 5, 6, 7, 8, 9, 10, 11, or 12 weeks. In some embodiments, the method comprises administering to a subject the pharmaceutical composition of the disclosure once every 1, 2, 3, 4, 5, 6, 7, 8, 9, 10, 11, or 12 months.

In some embodiments, the disclosure provides methods of delivering an LNP to a subject, comprising administering the LNP or the pharmaceutical composition thereof of the disclosure to the subject, wherein the method comprises multiple administrations. In some embodiments, serum half-life of the LNP in the subject following the second and/or subsequent administration of the method is at least 40%, 50%, 60%, 70%, 80%, 85%, 90%, or 95% of the serum half-life of the LNP following the first administration.

In some embodiments, the LNP has an AUC following a repeat dose that is at least 30%, at least 40%, at least 50%, at least 60%, at least 70%, at least 80%, at least 90%, or at least 100% of the AUC following the previous dose. In some embodiments, the LNP has an AUC that is at least 60% of the AUC following the previous dose. In some embodiments, following a repeat dose, AUC of the LNP decreases less than 70%, less than 60%, less than 60%, less than 40%, less than 30%, less than 20%, less than 10%, or less than 5% compared to the AUC following the previous dose. In some embodiments, following a repeat dose, AUC of the LNP decreases less than 40% compared to the AUC following the previous dose.

In some embodiments, the disclosure provides methods of delivering an LNP to a subject, comprising administering the LNP or the pharmaceutical composition thereof of the disclosure to the subject, wherein the LNP comprises a nucleic acid molecule encoding a viral genome of an oncolytic virus, wherein the subject has a tumor, and wherein administration of the LNP delivers the nucleic acid molecule into tumor cells. In some embodiments, administration of the LNP results in replication of the oncolytic virus in tumor cells. In some embodiments, administration of the LNP results in selective replication of the oncolytic virus in tumor cells as compared to normal cells.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits growth of the tumor. In some embodiments, the method inhibits growth of the tumor for at least 1 week, at least 1 month, at least 2 months, at least 3 months, at least 4 months, at least 6 months, at least 9 months, at least 12 months, at least 2 years, or longer. In some embodiments, inhibiting growth of the tumor means controlling the size of the tumor within 100% of the size of the tumor just before administration of the pharmaceutical composition for a specified time period. In some embodiments, inhibiting growth of the tumor means controlling the size of the tumor within 110%, within 120%, within 130%, within 140%, or within 150%, of the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor leads to tumor shrinkage or elimination. In some embodiments, the method results in tumor shrinkage or elimination for at least 1 week, at least 1 month, at least 2 months, at least 3 months, at least 4 months, at least 6 months, at least 9 months, at least 12 months, at least 2 years, or longer. In some embodiments, the method results in tumor shrinkage or elimination within 1 week, within 2 weeks, within 3 weeks, within 4 weeks, within 1 month, within 2 months, within 3 months, within 4 months, within 6 months, within 9 months, within 12 months, or within 2 years. In some embodiments, tumor shrinkage means reducing the size of the tumor by at least 10%, at least 20%, at least 30%, at least 40%, at least 50%, at least 60%, at least 70%, at least 80%, at least 90%, or at least 95%, compared to the size of the tumor just before administration of the pharmaceutical composition. In some embodiments, tumor shrinkage means reducing the size of the tumor at least 30% compared to the size of the tumor just before administration of the pharmaceutical composition.

In some embodiments, the disclosure provides methods of delivering an LNP to a subject, comprising administering the LNP or the pharmaceutical composition thereof of the disclosure to the subject, wherein administration of the LNP to a subject bearing a tumor inhibits the metastasis of the cancer.

In some embodiments, the subject is a mammal. In some embodiments, the subject is a human. In some embodiments, the subject has a cancer, and wherein the method inhibits or slows the growth and/or metastasis of the cancer.

In some embodiments, the disclosure provides methods of delivering an LNP to a subject, comprising systemically administering the LNP or pharmaceutical composition thereof. In some embodiments, the administration is intravenous, intra-arterial, intraperitoneal, intramuscular, intradermal, subcutaneous, intranasal, oral, or a combination thereof.

In some embodiments, the disclosure provides methods of delivering an LNP to a subject, comprising locally administering the LNP or pharmaceutical composition thereof. In some embodiments, the administration is intratumoral.

### Cancer

In some embodiments, the disclosure provides methods of killing a cancerous cell comprising exposing the cancerous cell to the lipid nanoparticles, the recombinant RNA molecules, or compositions thereof of the disclosure. In some embodiments, the cancerous cells are exposed under conditions sufficient for the intracellular delivery of the particles/recombinant RNA molecules/compositions to said cancerous cell, wherein the replication-competent virus produced by the encapsulated polynucleotide results in killing of the cancerous cell.

In some embodiments, the disclosure provides methods of treating a cancer in a subject comprising administering to a subject suffering from the cancer an effective amount of the particles, the recombinant RNA molecules, or compositions thereof of the disclosure.

“Cancer” herein refers to or describes the physiological condition in mammals that is typically characterized by unregulated cell growth. Examples of cancer include but are not limited to carcinoma, lymphoma, blastoma, sarcoma (including liposarcoma, osteogenic sarcoma, angiosarcoma, endotheliosarcoma, leiomyosarcoma, chordoma, lymphangiosarcoma, lymphangioendotheliosarcoma, rhabdomyosarcoma, fibrosarcoma, myxosarcoma, and chondrosarcoma), neuroendocrine tumors, mesothelioma, synovioma, schwannoma, meningioma, adenocarcinoma, melanoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, small cell lung carcinoma, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulvar cancer, thyroid cancer, hepatic carcinoma, anal carcinoma, penile carcinoma, testicular cancer, esophageal cancer, tumors of the biliary tract, Ewing's tumor, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, testicular tumor, lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstrom's macroglobulinemia, myelodysplastic disease, heavy chain disease, neuroendocrine tumors, Schwannoma, and other carcinomas, as well as head and neck cancer. In some embodiments, the cancer is a neuroendocrine cancer. Furthermore, benign (i.e., noncancerous) hyperproliferative diseases, disorders and conditions, including benign prostatic hypertrophy (BPH), meningioma, schwannoma, neurofibromatosis, keloids, myoma and uterine fibroids and others may also be treated using the disclosure disclosed herein. In some embodiments, the cancer is selected from small cell lung cancer (SCLC), small cell bladder cancer, large cell neuroendocrine carcinoma (LCNEC), castration-resistant small cell neuroendocrine prostate cancer (CRPC-NE), carcinoid (e.g., pulmonary carcinoid), and glioblastoma multiforme-IDH mutant (GBM-IDH mutant).

In some embodiments, the cancer is a metastatic cancer. In some embodiments, the cancer has metastasized. In some embodiments, the cancer is a non-metastatic cancer.

In some embodiments, the cancer is selected from the group consisting of lung cancer, breast cancer, colon cancer, pancreatic cancer, bladder cancer, renal cell carcinoma, ovarian cancer, gastric cancer and liver cancer. In some embodiments, the cancer is renal cell carcinoma, lung cancer, or liver cancer. In some embodiments, the lung cancer is NSCLC (non-small cell lung cancer). In some embodiments, the liver cancer is HCC (hepatocellular carcinoma). In some embodiments, the liver cancer is metastatic. In some embodiments, the breast cancer is TNBC (triple-negative breast cancer). In some embodiments, the bladder cancer is urothelial carcinoma. In some embodiments, the cancer is selected from the group consisting of breast cancer, esophageal cancer, stomach cancer, lung cancer, kidney cancer and skin cancer, and wherein the cancer has metastasized into liver. In some embodiments, the cancer is a metastasized cancer in the liver, wherein the cancer is originated from the group consisting of breast cancer, esophageal cancer, stomach cancer, lung cancer, kidney cancer and skin cancer. In some embodiments, the cancer is a hematologic cancer. In some embodiments, the hematologic cancer is multiple myeloma (see, e.g., Bradley, et al., Oncolytic Virotherapy, 2014:3 47-55, the content of which is incorporated by reference in its entirety). In some embodiments, the hematologic cancer is a leukemia or a lymphoma.

In some embodiments, the particles, the recombinant RNA molecules, or compositions thereof comprises a polynucleotide sequence derived from a CVA21-KY strain for treating cancer or killing cancer cells of lung cancer (e.g., NSCLC), breast cancer, colon cancer, or pancreatic cancer. In some embodiments, the cancer is lung cancer (e.g., NSCLC).

In some embodiments, the particles, the recombinant RNA molecules, or compositions thereof comprises a polynucleotide sequence derived from a CVA21-EF strain for treating cancer or killing cancer cells of bladder cancer, renal cell carcinoma, ovarian cancer, gastric cancer, or liver cancer (e.g., HCC). In some embodiments, the cancer is renal cell carcinoma. In some embodiments, the cancer is liver cancer (e.g., HCC). In some embodiments, the liver cancer is metastatic.

In some embodiments, the particles, the recombinant RNA molecules, or compositions thereof comprises a polynucleotide sequence derived from an SVV (e.g., a SVV-IRES2 chimeric virus) for treating cancer or killing cancer cells of lung cancer, liver cancer, prostate cancer, bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma (MCC), or melanoma. In some embodiments, the cancer is small cell lung cancer (SCLC). In some embodiments, the cancer is neuroblastoma. In some embodiments, the cancer is neuroendocrine cancer. In some embodiments, the cancer is rhabdomyosarcoma. In some embodiments, the cancer is castration-resistant prostate cancer with neuroendocrine phenotype (CRPC-NE). In some embodiments, the cancer is Merkel cell carcinoma (MCC).

In some embodiments, the disclosure provides methods of treating a cancer in a subject comprising administering to a subject suffering from the cancer (i) an effective amount of a particle (e.g., LNPs), a recombinant RNA molecule, or compositions thereof of the disclosure, and (ii) an effective amount of an immune checkpoint inhibitor. In some embodiments, the immune checkpoint inhibitor is an antibody or an antigen binding fragment thereof. In some embodiments, the immune checkpoint inhibitor binds to PD-1 (e.g., the inhibitor is an anti-PD-1 antibody). Anti-PD1 antibodies are known in the art, for example, Nivolumab, Pembrolizumab, Lambrolizumab, Pidilzumab, Cemiplimab, and AMP-224 (AstraZeneca/MedImmune and GlaxoSmithKline), JTX-4014 by Jounce Therapeutics, Spartalizumab (PDR001, Novartis), Camrelizumab (SHR1210, Jiangsu HengRui Medicine Co., Ltd), Sintilimab (IBI308, Innovent and Eli Lilly), Tislelizumab (BGB-A317), Toripalimab (JS 001), Dostarlimab (TSR-042, WBP-285, GlaxoSmithKline), INCMGA00012 (MGA012, Incyte and MacroGenics), and AMP-514 (MEDI0680, AstraZeneca). In some embodiments, the immune checkpoint inhibitor binds to PD-L1 (e.g., the inhibitor is an anti-PD-L1 antibody). Anti-PDL1 antibodies are known in the art, for example, MEDI-4736, MPDL3280A, Atezolizumab (Tecentriq, Roche Genentech), Avelumab (Bavencio, Merck Serono and Pfizer), and Durvalumab (Imfinzi, AstraZeneca). In some embodiments, the immune checkpoint inhibitor binds to CTLA4 (e.g., the inhibitor is an anti-CTLA4 antibody). Anti-CTLA4 antibodies are known in the art, for example, ipilumumab, tremelimumab, or any of the antibodies disclosed in WO2014/207063. In some embodiments, the immune checkpoint inhibitor is an anti-TIGIT antibody or fragment thereof. Anti-TIGIT antibodies are known in the art, for example tiragolumab (Roche), EOS-448 (iTeos Therapeutics), Vibostolimab (Merck), Domvanalimab (Arcus, Gilead), BMS-986207 (BMS), Etigilimab (Mereo), COM902 (Compugen), ASP8374 (Astellas), SEA-TGT (Seattle Genetics) BGB-A1217 (BeiGene), IBI-939 (Innovent), and M6223 (EMD Serono).

In some embodiments, both of 1) the particles, the recombinant RNA molecules, or compositions thereof and 2) the immune checkpoint inhibitor are concurrently administered. In some embodiments, these two therapeutic components are administered sequentially. In some embodiments, one or both therapeutic components are administered multiple times. In some embodiments, the particles, the recombinant RNA molecules, or compositions thereof comprises a polynucleotide sequence derived from an SVV (e.g., a SVV-IRES2 chimeric virus), and the immune checkpoint inhibitor binds to PD-1.

In some embodiments, the disclosure provides methods of treating a cancer in a subject comprising administering to a subject suffering from the cancer (i) an effective amount of a particle (e.g., LNPs), a recombinant RNA molecule, or compositions thereof of the disclosure, and (ii) an effective amount of an engineered immune cell comprising an “engineered antigen receptor”. Engineered antigen receptors refer to non-naturally occurring antigen-specific receptors such as a chimeric antigen receptors (CARs) or a recombinant T cell receptor (TCRs). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHIC-independent manner leading to activation and proliferation of the engineered immune cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. Antigen binding domains that are useful in the present disclosure include, for example, scFvs; antibodies; antigen binding regions of antibodies; variable regions of the heavy/light chains; and single chain antibodies.

In some embodiments, the intracellular signaling domain of a CAR may be derived from the TCR complex zeta chain (such as CD3ζ signaling domains), FcγRIII, FcεRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-α-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Rα2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta©) and Tisagenlecleucel (Kymriah©). See also, Li et al., J Hematol and Oncol (2018) 11(22), reviewing clinical trials of tumor-specific CARs.

In some embodiments, the engineered antigen receptor is an engineered TCR. Engineered TCRs comprise TCRα and/or TCRβ chains that have been isolated and cloned from T cell populations recognizing a particular target antigen. For example, TCRα and/or TCRβ genes (i.e., TRAC and TRBC) can be cloned from T cell populations isolated from individuals with particular malignancies or T cell populations that have been isolated from humanized mice immunized with specific tumor antigens or tumor cells. Engineered TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Engineered TCRs specific for tumor antigens are known in the art, for example WT1-specific TCRs (JTCR016, Juno Therapeutics; WT1-TCRc4, described in US Patent Application Publication No. 20160083449), MART-1 specific TCRs (including the DMF4T clone, described in Morgan et al., Science 314 (2006) 126-129); the DMF5T clone, described in Johnson et al., Blood 114 (2009) 535-546); and the ID3T clone, described in van den Berg et al., Mol. Ther. 23 (2015) 1541-1550), gp100-specific TCRs (Johnson et al., Blood 114 (2009) 535-546), CEA-specific TCRs (Parkhurst et al., Mol Ther. 19 (2011) 620-626), NY-ESO and LAGE-1 specific TCRs (1G4T clone, described in Robbins et al., J Clin Oncol 26 (2011) 917-924; Robbins et al., Clin Cancer Res 21 (2015) 1019-1027; and Rapoport et al., Nature Medicine 21 (2015) 914-921), and MAGE-A3-specific TCRs (Morgan et al., J Immunother 36 (2013) 133-151) and Linette et al., Blood 122 (2013) 227-242). (See also, Debets et al., Seminars in Immunology 23 (2016) 10-21).

In some embodiments, the engineered antigen receptor is directed against a target antigen selected from a cluster of differentiation molecule, such as CD3, CD4, CD8, CD16, CD24, CD25, CD33, CD34, CD45, CD64, CD71, CD78, CD80 (also known as B7-1), CD86 (also known as B7-2), CD96, CD116, CD117, CD123, CD133, and CD138, CD371 (also known as CLL1); a tumor-associated surface antigen, such as 5T4, BCMA (also known as CD269 and TNFRSF17, UniProt #Q02223), carcinoembryonic antigen (CEA), carbonic anhydrase 9 (CAIX or MN/CAIX), CD19, CD20, CD22, CD30, CD40, disialogangliosides such as GD2, ELF2M, ductal-epithelial mucin, ephrin B2, epithelial cell adhesion molecule (EpCAM), ErbB2 (HER2/neu), FCRL5 (UniProt #Q68SN8), FKBP11 (UniProt #Q9NYL4), glioma-associated antigen, glycosphingolipids, gp36, GPRC5D (UniProt #Q9NZD1), mut hsp70-2, intestinal carboxyl esterase, IGF-I receptor, ITGA8 (UniProt #P53708), KAMP3, LAGE-1a, MAGE, mesothelin, neutrophil elastase, NKG2D, Nkp30, NY-ESO-1, PAP, prostase, prostate-carcinoma tumor antigen-1 (PCTA-1), prostate specific antigen (PSA), PSMA, prostein, RAGE-1, ROR1, RU1 (SFMBTI), RU2 (DCDC2), SLAMF7 (UniProt #Q9NQ25), survivin, TAG-72, and telomerase; a major histocompatibility complex (MHC) molecule presenting a tumor-specific peptide epitope; tumor stromal antigens, such as the extra domain A (EDA) and extra domain B (EDB) of fibronectin; the A1 domain of tenascin-C(TnC A1) and fibroblast associated protein (FAP); cytokine receptors, such as epidermal growth factor receptor (EGFR), EGFR variant III (EGFRvIII), TFGβ-R or components thereof such as endoglin; a major histocompatibility complex (MHC) molecule; a virus-specific surface antigen such as an HIV-specific antigen (such as HIV gp120); an EBV-specific antigen, a CMV-specific antigen, a HPV-specific antigen, a Lassa virus-specific antigen, an Influenza virus-specific antigen as well as any derivate or variant of these surface antigens.

### FURTHER NUMBER EMBODIMENTS

Further numbered embodiments of the invention are provided as follows:

Embodiment 1. A lipid nanoparticle (LNP) comprising a synthetic RNA viral genome encoding an oncolytic Coxsackievirus virus, wherein the Coxsackievirus is a CVA21 strain selected from the EF strain and the KY strain.

Embodiment 1.1. A lipid nanoparticle (LNP) comprising a synthetic RNA viral genome encoding an oncolytic Coxsackievirus virus, wherein the Coxsackievirus is a CVA21 Kuykendall strain.

Embodiment 2. The LNP of Embodiment 1, wherein the Coxsackievirus is the CVA21-KY strain, and wherein the CVA21-KY strain comprises a polynucleotide sequence having at least 80%, at least 85%, at least 90%, at least 95%, or 100% sequence identity to SEQ ID NO: 5.

Embodiment 3. The LNP of Embodiment 1, wherein the Coxsackievirus is the CVA21-EF strain, and wherein the CVA21-EF strain comprises a polynucleotide sequence having at least 80%, at least 85%, at least 90%, at least 95%, or 100% sequence identity to SEQ ID NO: 9.

Embodiment 4. The LNP of Embodiment 1, wherein the Coxsackievirus comprises a 5′ UTR (IRES) sequence having at least 80%, at least 85%, at least 90%, at least 95%, or 100% sequence identity to SEQ ID NO: 6 or 10.

Embodiment 5. The LNP of Embodiment 1, wherein the Coxsackievirus comprises a P1 sequence having at least 80%, at least 85%, at least 90%, at least 95%, or 100% sequence identity to SEQ ID NO: 7 or 11.

Embodiment 6. The LNP of Embodiment 1, wherein the Coxsackievirus comprises a 3D sequence having at least 80%, at least 85%, at least 90%, at least 95%, or 100% sequence identity to SEQ ID NO: 8 or 12.

Embodiment 7. The LNP of any one of Embodiments 1-6, wherein the synthetic RNA viral genome does not comprise a polynucleotide sequence having more than 95%, more than 90%, more than 85%, or more than 80% sequence identity to SEQ ID NO: 1.

Embodiment 8. A lipid nanoparticle (LNP) comprising a synthetic RNA viral genome encoding an oncolytic Seneca Valley Virus (SVV), wherein the synthetic RNA viral genome comprises a polynucleotide sequence having at least 80%, at least 85%, at least 90%, at least 95%, or 100% sequence identity to SEQ ID NO: 68.

Embodiment 9. The LNP of Embodiment 8, wherein the synthetic RNA viral genome comprises a 5′ UTR (IRES) sequence having at least 80%, at least 85%, at least 90%, at least 95%, or 100% sequence identity to nucleic acids 1-670 of SEQ ID NO: 68.

Embodiment 10. The LNP of Embodiment 8 or 9, wherein the synthetic RNA viral genome encodes a SVV VP2 protein comprising a S177A mutation.

Embodiment 11. The LNP of any one of Embodiments 1-10, wherein delivery of the LNP to a cell results in production of viral particles by the cell, and wherein the viral particles are infectious and lytic.

Embodiment 12. The LNP of any one of Embodiments 1-11, wherein the synthetic RNA viral genome further comprises a heterologous polynucleotide encoding an exogenous payload protein.

Embodiment 13. The LNP of any one of Embodiments 1-11, further comprising a second recombinant RNA molecule encoding an exogenous payload protein.

Embodiment 14. The LNP of Embodiment 12 or 13, wherein the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2).

Embodiment 15. The LNP of Embodiment 14, wherein the nitroreductase is NfsB or NfsA.

Embodiment 16. The LNP of Embodiment 14, wherein the reovirus FAST protein is ARV p14, BRV p15, or a p14-p15 hybrid.

Embodiment 17. The LNP of Embodiment 12 or 13, wherein the exogenous payload protein is a fluorescent protein, an enzymatic protein, a cytokine, a chemokine, an antigen-binding molecule capable of binding to a cell surface receptor, or a ligand for a cell-surface receptor.

Embodiment 18. The LNP of Embodiment 17, wherein:
a) the cytokine is selected from GM-CSF, IFNγ, IL-2, IL-7, IL-12, IL-18, IL-21, and IL-367;
b) the ligand for a cell-surface receptor is Flt3 ligand or TNFSF14; or
c) the chemokine is selected from CXCL10, CCL4, CCL21, and CCL5.

Embodiment 19. The LNP of Embodiment 17, wherein the antigen-binding molecule is capable of binding to and inhibiting an immune checkpoint receptor.

Embodiment 20. The LNP of Embodiment 19, wherein the immune checkpoint receptor is PD-1.

Embodiment 21. The LNP of Embodiment 17, wherein the antigen-binding molecule is capable of binding to a tumor antigen.

Embodiment 22. The LNP of Embodiment 21, wherein the antigen binding molecule is a bispecific T cell engager molecule (BiTE) or a bispecific light T cell engager molecule (LiTE).

Embodiment 23. The LNP of Embodiment 21 or 22, wherein the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1.

Embodiment 24. The LNP of Embodiment 21 or 22, wherein the tumor antigen is DLL3 or EpCAM.

Embodiment 25. The LNP of any one of Embodiments 1-24, wherein the synthetic RNA viral genome and/or the recombinant RNA molecule comprises a microRNA (miRNA) target sequence (miR-TS) cassette, wherein the miR-TS cassette comprises one or more miRNA target sequences.

Embodiment 26. The LNP of Embodiment 25, wherein the one or more miRNAs are selected from miR-124, miR-1, miR-143, miR-128, miR-219, miR-219a, miR-122, miR-204, miR-217, miR-137, miR-142, and miR-126.

Embodiment 27. The LNP of Embodiment 26, wherein the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

Embodiment 28. The LNP of any one of Embodiments 1-27, wherein the LNP comprises a cationic lipid, a helper lipid, a structural lipid, and a PEG-lipid.

Embodiment 29. The LNP of Embodiment 28, wherein the cationic lipid is a compound of Formula (I):

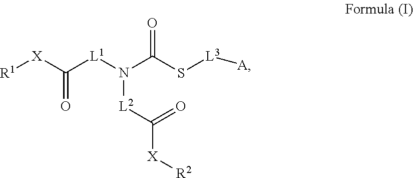

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(RI)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

Embodiment 30. The LNP of Embodiment 29, wherein the number of carbon atoms between the S of the thiolate and the closest N comprised in A is 2-4.

Embodiment 31. The LNP of Embodiment 29 or 30, wherein the cationic lipid is a compound of Formula (I-a):

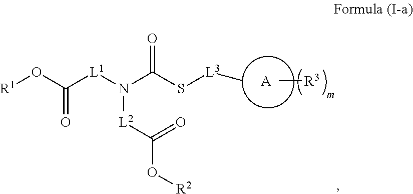

or a pharmaceutically acceptable salt or solvate thereof, wherein:
m is 0, 1, 2, 3, 4, 5, or 6.

Embodiment 32. The LNP of any one of Embodiments 29-31, wherein A is an optionally substituted 5-6-membered heterocyclyl ring.

Embodiment 33. The LNP of Embodiment 29, wherein the cationic lipid is

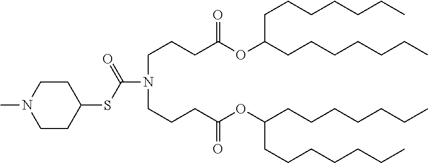 [SMILES: CCCCCCCC(CCCCCCC)OC(=O)CCCN(CCCC(=O)OC(CCCCCCC)CCCCCCC)C(=O)SC1CCN(C)CC1]

or a pharmaceutically acceptable salt or solvate thereof.

Embodiment 34. The LNP of Embodiment 28, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

Embodiment 35. The LNP of any one of Embodiments 28-34, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE).

Embodiment 36. The LNP of Embodiment 28, wherein the cationic lipid is 1,2-dioleoyl-3-trimethylammonium-propane (DOTAP), and wherein the helper lipid is 1,2-Dilauroyl-sn-glycero-3-phosphoethanolamine (DLPE) or 1,2-Dioleoyl-sn-glycero-3-phosphoethanolamine (DOPE).

Embodiment 37. The LNP of any one of Embodiments 28-36, wherein the structural lipid is cholesterol.

Embodiment 38. The LNP of any one of Embodiments 28-37, wherein the PEG-lipid is a compound of Formula (A″):

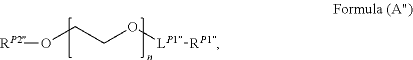

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃,
and wherein the LNP has a molar ratio of about 0.001% to about 5% PEG-lipid.

Embodiment 39. The LNP of Embodiment 38, wherein L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

Embodiment 40. The LNP of Embodiment 38, wherein L^{P1}″ is a bond.

Embodiment 41. The LNP of any one of Embodiments 38-40, wherein R^{P2}″ is hydrogen.

Embodiment 42. The LNP of any one of Embodiments 28-37, wherein the PEG-lipid is a compound of Formula (B):

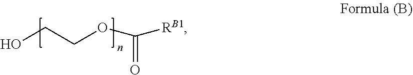

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

Embodiment 43. The LNP of any one of Embodiments 28-37, wherein the PEG-lipid is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

Embodiment 44. The LNP of any one of Embodiments 28-37, wherein the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

Embodiment 45. The LNP of Embodiment 28, wherein the cationic lipid comprises COATSOME® SS—OC, wherein the helper lipid comprises DSPC, the structural lipid comprises cholesterol (Chol) and wherein the PEG-lipid comprises DPG-PEG2000.

Embodiment 46. The LNP of Embodiment 28, wherein the cationic lipid comprises COATSOME® SS—OC, wherein the helper lipid comprises DSPC, the structural lipid comprises cholesterol (Chol) and wherein the PEG-lipid is a compound of Formula (A″):

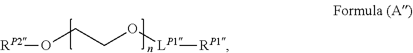

or a pharmaceutically acceptable salt thereof, wherein:
n is an integer between 10 to 200, inclusive of all endpoints;
L^{P1}″ is a bond;
R^{P1}″ is C₅-C₂₅ alkyl or C₅-C₂₅ alkenyl; and
R^{P2}″ is hydrogen, and
wherein the LNP has a molar ratio of about 0.001% to about 5% PEG-lipid.

Embodiment 47. The LNP of any one of Embodiments 28-37 and 46, wherein the PEG-lipid is selected from the group consisting of BRIJ™ S100, BRIJ™ S20, BRIJ™ 020 and BRIJ™ C₂₀.

Embodiment 48. The LNP of any one of Embodiments 28-37 and 46, wherein the PEG-lipid is BRIJ™ S100.

Embodiment 49. The LNP of any one of Embodiments 45-48, wherein the ratio of SS—OC:DSPC:Chol:PEG-lipid (as a percentage of total lipid content) is A:B:C:D, wherein A+B+C+D=100%, and wherein
a. A=40%-60%, B=10%-25%, C=20%-30%, and D=0.01%-3%;
b. A=45%-50%, B=20%-25%, C=25%-30%, and D=0.01%-1%; or
c. A=about 49%, B=about 22%, C=about 28%, and D=about 0.5%

Embodiment 50. The LNP of any one of Embodiments 45-48, wherein the ratio of SS—OC:DSPC:Chol:PEG-lipid (as a percentage of total lipid content) is A:B:C:D, wherein A+B+C+D=100%, and wherein
a. A=40%-60%, B=10%-30%, C=20%-45%, and D=0%-3%;
b. A=40%-60%, B=10%-30%, C=25%-45%, and D=0.01%-3%;
c. A=45%-55%, B=10%-20%, C=30%-40%, and D=1%-2%;
d. A=45%-50%, B=10%-15%, C=35%-40%, and D=1%-2%; or
e. A=about 49%, B=about 11%, C=about 38%, and D=about 1.5%.

Embodiment 51. The LNP of any one of Embodiments 45-48, wherein the ratio of SS—OC:DSPC:Chol:PEG-lipid (as a percentage of total lipid content) is about A:B:C:D, wherein A+B+C+D=100%, and wherein
a. A=45%-65%, B=5%-20%, C=20%-45%, and D=0%-3%;
b. A=50%-60%, B=5%-15%, C=30%-45%, and D=0.01%-3%;
c. A=55%-60%, B=5%-15%, C=30%-40%, and D=1%-2%;
d. A=55%-60%, B=5%-10%, C=30%-35%, and D=1%-2%; or
e. A=about 58%, B=about 7%, C=about 33%, and D=about 1.5%.

Embodiment 52. A lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (A″):

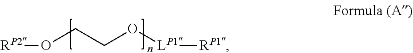

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

Embodiment 53. A lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (A″):

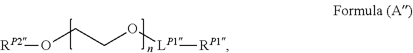

or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

Embodiment 54. The LNP of Embodiment 52 or 53, wherein R¹ is C₁₆-C₁₈ alkyl or C₁₆-C₁₈ alkenyl.

Embodiment 55. The LNP of any one of Embodiments 52-54, wherein L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

Embodiment 56. The LNP of any one of Embodiments 52-54, wherein L^{P1}″ is a bond.

Embodiment 57. The LNP of any one of Embodiments 52-56, wherein R^{P2}″ is hydrogen.

Embodiment 58. The LNP of Embodiment 52 or 53, wherein the PEG-lipid is a compound of Formula (A″-f1), Formula (A″-f2), or Formula (A″-f3):

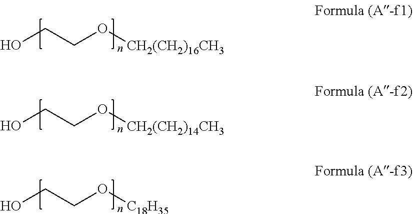

or a pharmaceutically acceptable salt thereof.

Embodiment 59. A lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (B):

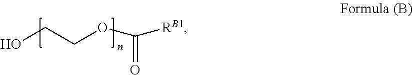

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

Embodiment 60. A lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (B):

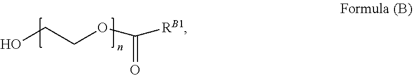

or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

Embodiment 61. The LNP of Embodiment 59 or 60, wherein R¹ is C₁₅-C₁₇ alkyl or C₁₅-C₁₇ alkenyl.

Embodiment 62. The LNP of Embodiment 59 or 60, wherein the PEG-lipid is a compound of Formula (B-a) or Formula (B-b):

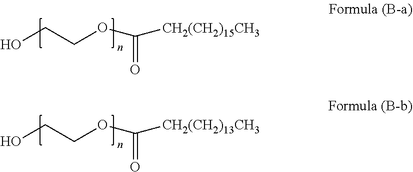

or a pharmaceutically acceptable salt thereof.

Embodiment 63. The LNP of any one of Embodiments 52-62, wherein n is on average about 20, about 40, about 50, or about 100.

Embodiment 64. The LNP of any one of Embodiments 52-62, wherein n is on average about 100.

Embodiment 65. The LNP of any one of Embodiments 52-64, wherein the PEG-lipid comprise a PEG moiety having an average molecular weight of of about 200 daltons to about 10,000 daltons, about 500 daltons to about 7,000 daltons, or about 800 daltons to about 6,000 daltons.

Embodiment 66. The LNP of any one of Embodiments 52-65, wherein the PEG-lipid is selected from the group consisting of HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃.

Embodiment 67. The LNP of any one of Embodiments 52-66, wherein the LNP induces a reduced immune response in vivo as compared to a control LNP lacking the PEG-lipid of Formula (A″) and/or a ionizable lipid of Formula (I), optionally wherein a PEG-lipid in the control LNP is PEG2K-DPG or PEG2K-DMG.

Embodiment 68. The LNP of Embodiment 67, wherein the immune response is accelerated blood clearance (ABC) of the LNP and/or an anti-PEG IgM response.

Embodiment 69. The LNP of any one of Embodiments 52-68, wherein the cationic lipid is a compound of Formula (I):

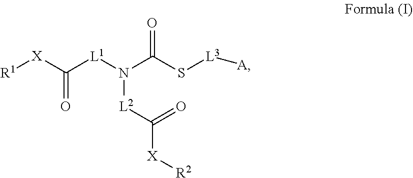

or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic; R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

Embodiment 70. The LNP of Embodiment 69, wherein the number of carbon atoms between the S of the thiolate and the closest N comprised in A is 2-4.

Embodiment 71. The LNP of Embodiment 69 or 70, wherein the cationic lipid is a compound of Formula (I-a):

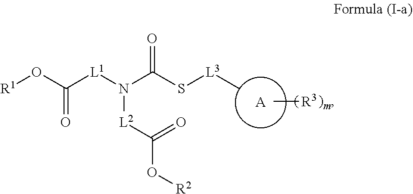

or a pharmaceutically acceptable salt or solvate thereof, wherein:
m is 0, 1, 2, 3, 4, 5, or 6.

Embodiment 72. The LNP of any one of Embodiments 69-71, wherein A is an optionally substituted 5-6-membered heterocyclyl ring.

Embodiment 73. The LNP of Embodiment 69, wherein the cationic lipid is

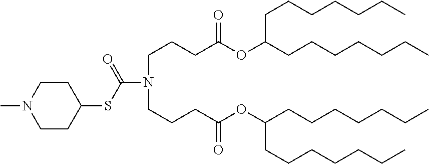 [SMILES: CCCCCCCC(CCCCCCC)OC(=O)CCCN(CCCC(=O)OC(CCCCCCC)CCCCCCC)C(=O)SC1CCN(C)CC1]

or a pharmaceutically acceptable salt or solvate thereof.

Embodiment 74. The LNP of any one of Embodiments 52-68, wherein the cationic lipid is selected from DLinDMA, DLin-KC2-DMA, DLin-MC3-DMA (MC3), COATSOME@SS-LC (former name: SS-18/4PE-13), COATSOME® SS-EC (former name: SS-33/4PE-15), COATSOME® SS—OC, COATSOME® SS—OP, Di((Z)-non-2-en-1-yl)9-((4-dimethylamino)butanoyl)oxy)heptadecanedioate (L-319), N-(2,3-dioleoyloxy)propyl)-N,N,N-trimethylammonium chloride (DOTAP), or a mixture thereof.

Embodiment 75. The LNP of any one of Embodiments 52-68, wherein the cationic lipid is a compound of Formula (II-1a):

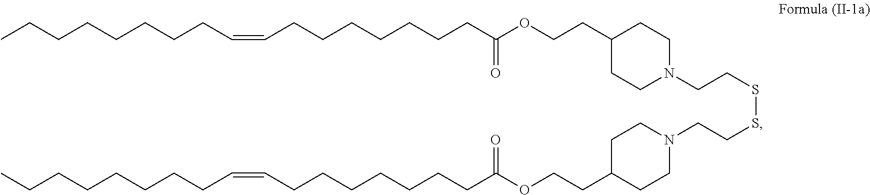 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

a compound of Formula (II-2a):

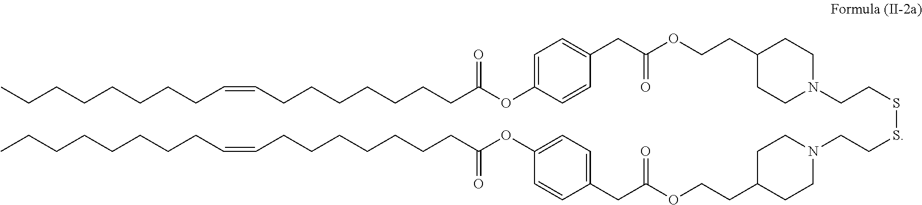 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

Embodiment 76. The LNP of any one of Embodiments 52-75, wherein the cationic lipid is a compound of Formula (II-1a), the structural lipid is cholesterol, the helper lipid is DSPC, and the PEG-lipid is BRIJ™ S100.

Embodiment 77. The LNP of any one of Embodiments 52-75, wherein the cationic lipid is a compound of Formula (II-1a), the structural lipid is cholesterol, the helper lipid is DSPC, and the PEG-lipid is MYRJ™ S100, MYRJ™ S50, or MYRJ™ 540.

Embodiment 78. The LNP of any one of Embodiments 52-77, wherein the LNP comprises a molar ratio of about 0.1% to about 2% PEG-lipid, such as about 0.2% to about 0.8 mol %, about 0.4% to about 0.6 mol %, about 0.7% to about 1.3%, or about 1.2% to about 1.8% PEG-lipid.

Embodiment 79. The LNP of any one of Embodiments 52-78, wherein the LNP comprises a molar ratio of about 0.2% to about 0.8%, or about 0.5% PEG-lipid.

Embodiment 80. The LNP of any one of Embodiments 52-78, wherein the LNP comprises a molar ratio of about 1.2% to about 1.8%, or about 1.5% PEG-lipid.

Embodiment 81. The LNP of any one of Embodiments 52-80, wherein the LNP has a molar ratio of about 44% to about 54% cationic lipid, about 19% to about 25% helper lipid, about 24% to about 33% structural lipid, and about 0.2% to about 0.8% PEG-lipid.

Embodiment 82. The LNP of any one of Embodiments 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC:PEG-lipid is 49:28.5:22:0.5.

Embodiment 83. The LNP of any one of Embodiments 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC:PEG-lipid is 49:27.5:22:1.5.

Embodiment 84. The LNP of any one of Embodiments 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol:DSPC:PEG-lipid is 49:38.5:11:1.5.

Embodiment 85. The LNP of any one of Embodiments 52-84, wherein the LNP has a lipid-nitrogen-to-phosphate (N:P) ratio of about 1 to about 25.

Embodiment 86. The LNP of any one of Embodiments 52-85, wherein the LNP has a N:P ratio of about 14.

Embodiment 87. The LNP of any one of Embodiments 1-86, wherein hyaluronan is conjugated to the surface of the LNP.

Embodiment 88. A pharmaceutical composition comprising a plurality of lipid nanoparticles according to any one of Embodiments 1-87.

Embodiment 89. The pharmaceutical composition of Embodiment 88, wherein the plurality of LNPs have an average diameter of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

Embodiment 90. The pharmaceutical composition of Embodiment 88, wherein the plurality of LNPs have an average diameter of about 50 nm to about 120 nm.

Embodiment 91. The pharmaceutical composition of Embodiment 88, wherein the plurality of LNPs have an average diameter of about 50 nm, 60 nm, 70 nm, 80 nm, 90 nm, 100 nm, 110 nm, or about 120 nm.

Embodiment 92. The pharmaceutical composition of Embodiment 88, wherein the plurality of LNPs have an average diameter of about 100 nm.

Embodiment 93. The pharmaceutical composition of any one of Embodiments 88-92, wherein the plurality of LNPs have an average zeta-potential of between about 40 mV to about −40 mV, about 20 mV to about −20 mV, about 10 mV to about −10 mV, about 5 mV to about −5 mV, or about 20 mV to about −40 mV.

Embodiment 94. The pharmaceutical composition of any one of Embodiments 88-92, wherein the plurality of LNPs have an average zeta-potential of less than about 5 mV, less than about 0 mV, less than about −5 mV, less than about −10 mV, less than about −20 mV, less than about −30 mV, less than about −35 mV, or less than about −40 mV.

Embodiment 95. The pharmaceutical composition of any one of Embodiments 88-92, wherein the plurality of LNPs have an average zeta-potential of between about −50 mV to about
20 mV, about −40 mV to about −20 mV, about −30 mV to about −10 mV, about −20 mV to about 0 mV, about −15 mV to about 5 mV, or about −10 mV to about 10 mV.

Embodiment 96. The pharmaceutical composition of Embodiment 94 or 95, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

Embodiment 97. The pharmaceutical composition of any one of Embodiments 88-96, wherein administering the pharmaceutical composition to a subject delivers the recombinant RNA polynucleotide to a target cell of the subject, and wherein the recombinant RNA polynucleotide produces an infectious oncolytic virus capable of lysing the target cell of the subject.

Embodiment 98. The pharmaceutical composition of Embodiment 97, wherein the target cell is a cancerous cell.

Embodiment 99. The pharmaceutical composition of any one of Embodiments 88-98, wherein the composition is formulated for intravenous and/or intratumoral delivery.

Embodiment 100. The pharmaceutical composition of any one of Embodiments 88-99, wherein the composition has a duration of therapeutic effect in vivo greater than that of a composition lacking the PEG-lipid of Formula (A″) and/or a ionizable lipid of Formula (I).

Embodiment 101. The pharmaceutical composition of Embodiment 99 or 100, wherein the composition has a duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer.

Embodiment 102. The pharmaceutical composition of Embodiment 99 or 100, wherein the composition has a half-life and/or an AUC in vivo greater than or equal to that of a pre-determined threshold value.

Embodiment 103. The pharmaceutical composition of any one of Embodiments 88-102, wherein the encapsulation efficiency of the synthetic RNA viral genome by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

Embodiment 104. The pharmaceutical composition of any one of Embodiments 88 to 103, wherein the composition has a total lipid concentration of about 10 mM, about 20 mM, about 30 mM, about 40 mM, or about 50 mM.

Embodiment 105. The pharmaceutical composition of any one of Embodiments 88-104, wherein the composition is formulated at a pH of about 2.5, about 3, about 3.5, about 4, about 4.5, about 5, about 5.5, or about 6.

Embodiment 106. The pharmaceutical composition of any one of Embodiments 88 to 105, wherein the composition is formulated for multiple administrations.

Embodiment 107. The pharmaceutical composition of Embodiment 106, wherein a subsequent administration is administered at least 3 days, at least 5 days, at least 7 days, at least 9 days, at least 11 days, at least 14 days, or at least 21 days after a first administration.

Embodiment 108. The pharmaceutical composition of any one of Embodiments 88 to 107, further comprising a pharmaceutically acceptable carrier.

Embodiment 109. A recombinant RNA molecule comprising a synthetic RNA viral genome encoding an oncolytic Coxsackievirus virus, wherein the Coxsackievirus is a CVA21 strain selected from the EF strain and the KY strain.

Embodiment 109.1. A recombinant RNA molecule comprising a synthetic RNA viral genome encoding an oncolytic Coxsackievirus virus, wherein the Coxsackievirus is a CVA21 Kuykendall strain.

Embodiment 110. The recombinant RNA molecule of Embodiment 109, wherein the Coxsackievirus is the CVA21-KY strain, and wherein the CVA21-KY strain comprises a polynucleotide sequence having at least 80%, at least 85%, at least 90%, at least 95%, or 100% sequence identity according to SEQ ID NO: 5.

Embodiment 111. The recombinant RNA molecule of Embodiment 109, wherein the Coxsackievirus is the CVA21-EF strain, and wherein the CVA21-EF strain comprises a polynucleotide sequence having at least 80%, at least 85%, at least 90%, at least 95%, or 100% sequence identity according to SEQ ID NO: 9.

Embodiment 112. The recombinant RNA molecule of Embodiment 109, wherein the Coxsackievirus comprises a 5′ UTR (IRES) sequence having at least 80%, at least 85%, at least 90%, at least 95%, or 100% sequence identity according to SEQ ID NO: 6 or 10.

Embodiment 113. The recombinant RNA molecule of Embodiment 109, wherein the Coxsackievirus comprises a P1 sequence having at least 80%, at least 85%, at least 90%, at least 95%, or 100% sequence identity according to SEQ ID NO: 7 or 11.

Embodiment 114. The recombinant RNA molecule of Embodiment 109, wherein the Coxsackievirus comprises a 3D sequence having at least 80%, at least 85%, at least 90%, at least 95%, or 100% sequence identity according to SEQ ID NO: 8 or 12.

Embodiment 115. The recombinant RNA molecule of any one of Embodiments 109-114, wherein the synthetic RNA viral genome does not comprise a polynucleotide sequence having more than 95%, more than 90%, more than 85%, or more than 80% sequence identity according to SEQ ID NO: 1.

Embodiment 116. The recombinant RNA molecule of any one of Embodiments 109-115, wherein the recombinant RNA molecule does not comprise an RNA viral genome having 100% sequence identity to that of a wildtype Coxsackievirus virus.

Embodiment 117. A recombinant RNA molecule comprising a synthetic RNA viral genome encoding a Seneca Valley virus (SVV), wherein the SVV comprises is a chimeric SVV, and wherein the synthetic RNA viral genome comprises a polynucleotide sequence having at least 80%, at least 85%, at least 90%, at least 95%, or 100% sequence identity to SEQ ID NO: 68.

Embodiment 118. The recombinant RNA molecule of any one of Embodiments 109-117, further comprising a microRNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell.

Embodiment 119. The recombinant RNA molecule of Embodiment 118, wherein the one or more miRNAs are selected from miR-124, miR-1, miR-143, miR-128, miR-219, miR-219a, miR-122, miR-204, miR-217, miR-137, miR-142, and miR-126.

Embodiment 120. The recombinant RNA molecule of Embodiment 119, wherein the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

Embodiment 121. The recombinant RNA molecule of any one of Embodiments 109-120, wherein the recombinant RNA molecule is capable of producing a replication-competent oncolytic virus when introduced into a cell by a non-viral delivery vehicle.

Embodiment 122. The recombinant RNA molecule of Embodiment 121, wherein the cell is a mammalian cell.

Embodiment 123. The recombinant RNA molecule of Embodiment 122, wherein the cell is a mammalian cell present in a mammalian subject.

Embodiment 124. The recombinant RNA molecule of any one of Embodiments 118-123, wherein the one or more miR-TS cassettes is incorporated into the 5′ untranslated region (UTR) or 3′ UTR of one or more viral genes.

Embodiment 125. The recombinant RNA molecule of any one of Embodiments 118-123, wherein the one or more miR-TS cassettes is incorporated into the open reading frame (ORF), the 5′ untranslated region (UTR), or the 3′ UTR of one or more viral genes.

Embodiment 126. The recombinant RNA molecule of any of Embodiments 109-125, wherein the recombinant RNA molecule is inserted into a nucleic acid vector.

Embodiment 127. The recombinant RNA molecule of Embodiment 126, wherein the nucleic acid vector is a replicon.

Embodiment 128. The recombinant RNA molecule of Embodiments 109-127, wherein the synthetic RNA viral genome further comprises a heterologous polynucleotide encoding an exogenous payload protein.

Embodiment 129. The recombinant RNA molecule of Embodiment 128, wherein the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2).

Embodiment 130. The LNP of Embodiment 129, wherein the nitroreductase is NfsB or NfsA.

Embodiment 131. The LNP of Embodiment 129, wherein the reovirus FAST protein is ARV p14, BRV p15, or a p14-p15 hybrid.

Embodiment 132. The recombinant RNA molecule of Embodiment 128, wherein the exogenous payload protein is a fluorescent protein, an enzymatic protein, a cytokine, a chemokine, an antigen-binding molecule capable of binding to a cell surface receptor, or a ligand capable of binding to a cell surface receptor.

Embodiment 133. The recombinant RNA molecule of Embodiment 132, wherein
a) the cytokine is selected from GM-CSF, IFNγ, IL-2, IL-7, IL-12, IL-18, IL-21, and IL-367;
b) the ligand for a cell-surface receptor is Flt3 ligand or TNFSF14;
c) the chemokine is selected from CXCL10, CCL4, CCL21, and CCL5.

Embodiment 134. The recombinant RNA molecule of Embodiment 132, wherein the antigen-binding molecule is capable of binding to and inhibiting an immune checkpoint receptor.

Embodiment 135. The recombinant RNA molecule of Embodiment 134, wherein the immune checkpoint receptor is PD-1.

Embodiment 136. The recombinant RNA molecule of Embodiment 132, wherein the antigen-binding molecule is capable of binding to a tumor antigen.

Embodiment 137. The recombinant RNA molecule of Embodiment 136, wherein the antigen binding molecule is a bispecific T cell engager molecule (BiTE) or a bispecific light T cell engager molecule (LiTE).

Embodiment 138. The recombinant RNA molecule of Embodiment 136 or 137, wherein the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1.

Embodiment 139. The recombinant RNA molecule of Embodiment 136 or 137, wherein the tumor antigen is DLL3 or EpCAM.

Embodiment 140. A recombinant DNA template comprising from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence.

Embodiment 141. A recombinant DNA molecule comprising from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence, wherein the RNA molecule is selected from any one of Embodiments 109-139.

Embodiment 142. The recombinant DNA molecule of Embodiment 140 or 141, comprising a leader sequence between the promoter sequence and the 5′ junctional cleavage sequence.

Embodiment 143. A recombinant DNA molecule comprising from 5′ to 3′, a promoter sequence, a leader sequence, a 5′ junctional cleavage sequence, a polynucleotide sequence encoding a recombinant RNA molecule comprising a synthetic RNA viral genome, a poly-A tail, and a 3′ junctional cleavage sequence.

Embodiment 144. The recombinant DNA molecule of Embodiment 142 or 143, wherein the leader sequence is less than 100 bp in length.

Embodiment 145. The recombinant DNA molecule of any one of Embodiments 140-144, wherein the promoter sequence is a T7 promoter sequence.

Embodiment 146. The recombinant DNA molecule of any one of Embodiments 140-145, wherein the poly-A tail is about 50-90 bp in length or about 65-75 bp in length.

Embodiment 147. The recombinant DNA molecule of Embodiment 145, wherein the poly-A tail is about 70 bp in length.

Embodiment 148. The recombinant DNA molecule of any one of Embodiments 140-145, wherein the poly-A tail is about 10-50 bp, or 25-35 bp in length.

Embodiment 149. The recombinant DNA molecule of any one of Embodiments 140-148, wherein the 5′ junctional cleavage sequence comprises or consists of a ribozyme sequence and the 3′ junctional cleavage sequence comprises or consists of a ribozyme sequence.

Embodiment 150. The recombinant DNA molecule of Embodiment 149, wherein the 5′ ribozyme sequence is a hammerhead ribozyme sequence and wherein the 3′ ribozyme sequence is a hepatitis delta virus ribozyme sequence.

Embodiment 151. The recombinant DNA molecule of any one of Embodiments 140-148, wherein the 5′ junctional cleavage sequence comprises or consists of an RNAseH primer binding sequence and the 3′ junctional cleavage sequence comprises or consists of a restriction enzyme recognition sequence.

Embodiment 152. The recombinant DNA molecule of any one of Embodiments 140-148, wherein the 5′ junctional cleavage sequence comprises or consists of a ribozyme sequence and the 3′ junctional cleavage sequence comprises or consists of a restriction enzyme recognition sequence.

Embodiment 153. The recombinant DNA molecule of Embodiment 152, wherein the 5′ ribozyme sequence comprises or consists of a hammerhead ribozyme sequence, a Pistol ribozyme sequence, or a modified Pistol ribozyme sequence.

Embodiment 154. The recombinant DNA molecule of any one of Embodiments 140-153, wherein the 3′ junctional cleavage sequence comprises or consists of a Type IIS restriction enzyme recognition sequence.

Embodiment 155. The recombinant DNA molecule of any one of Embodiments 140-154, wherein the RNA molecule encodes the RNA viral genome of a Coxsackievirus (CVA).

Embodiment 156. The recombinant DNA molecule of Embodiment 155, wherein the Coxsackievirus is a CVA21 strain.

Embodiment 157. The recombinant DNA molecule of any one of Embodiments 155-156, wherein the leader sequence comprises or consists of a polynucleotide sequence having at least 70%, at least 80%, at least 90%, at least 95%, or 100% sequence identity according to SEQ ID NO: 14 or 15.

Embodiment 158. The recombinant DNA molecule of any one of Embodiments 155-157, wherein the 5′ junctional cleavage sequence comprises or consists of a Pistol ribozyme sequence having at least 80%, at least 90%, or 100% sequence identity to SEQ ID NO: 18, and wherein the P2 motif of the 5′ ribozyme sequence has the polynucleotide sequence of “TTTT”.

Embodiment 159. The recombinant DNA molecule of any one of Embodiments 155-157, wherein the 5′ junctional cleavage sequence comprises or consists of a Pistol ribozyme sequence having at least 80%, at least 90%, or 100% sequence identity to SEQ ID NO: 17, and wherein the P2 motif of the 5′ ribozyme sequence has the polynucleotide sequence of “TTTA”.

Embodiment 160. The recombinant DNA molecule of any one of Embodiments 155-159, wherein the 3′ junctional cleavage sequence comprises or consists of a BsmBI recognition sequence.

Embodiment 161. The recombinant DNA molecule of any one of Embodiments 155-159, wherein the 3′ junctional cleavage sequence comprises or consists of a BsaI recognition sequence.

Embodiment 162. The recombinant DNA molecule of Embodiment 156, wherein the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsmBI recognition sequence.

Embodiment 163. The recombinant DNA molecule of Embodiment 156, wherein the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsaI recognition sequence.

Embodiment 164. The recombinant DNA molecule of any one of Embodiments 140-154, wherein the RNA molecule encodes the RNA viral genome of a Seneca Valley virus (SVV).

Embodiment 165. The recombinant DNA molecule of Embodiment 164, wherein the leader sequence comprises or consists of a polynucleotide sequence having at least 70%, at least 80%, at least 90%, at least 95%, or 100% sequence identity according to any one of SEQ ID NO: 53-63.

Embodiment 166. The recombinant DNA molecule of Embodiment 164, wherein the leader sequence comprises or consists of a polynucleotide sequence having at least 70%, at least 80%, at least 90%, at least 95%, or 100% sequence identity according to SEQ ID NO: 58.

Embodiment 167. The recombinant DNA molecule of any one of Embodiments 164 to 166, wherein the 5′ ribozyme sequence is a Pistol ribozyme sequence having at least 80%, at least 85%, at least 90%, at least 95%, or 100% sequence identity to SEQ ID NO: 64 or 65, and wherein the P2 motif of the 5′ ribozyme sequence has the polynucleotide sequence of “TCAA” or “TTAA”.

Embodiment 168. The recombinant DNA molecule of any one of Embodiments 164 to 167, wherein the RNA viral genome comprises a 5′ UTR (IRES) sequence having at least 80%, at least 85%, at least 90%, at least 95%, or 100% sequence identity to nucleic acids 1-670 of SEQ ID NO: 68.

Embodiment 169. The recombinant DNA molecule of any one of Embodiments 164 to 168, wherein the 3′ junctional cleavage sequence comprises or consists of a SapI recognition sequence.

Embodiment 170. The recombinant DNA molecule of Embodiment 164, wherein the promoter sequence is a T7 promoter sequence, wherein the leader sequence consists of a polynucleotide sequence according to SEQ ID NO: 53, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence.

Embodiment 171. The recombinant DNA molecule of Embodiment 164, wherein the promoter sequence is a T7 promoter sequence, wherein the leader sequence consists of a polynucleotide sequence according to SEQ ID NO: 58, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence.

Embodiment 172. The recombinant DNA molecule of any one of Embodiments 140-171, wherein the recombinant DNA molecule does not comprise additional nucleic acid within the region spanning the promoter sequence and the 3′ junctional cleavage sequence.

Embodiment 173. A method of producing a recombinant RNA molecule, comprising in vitro transcription of the DNA molecule of any one of Embodiments 140-172 and purification of the resulting recombinant RNA molecule.

Embodiment 174. The method of Embodiment 173, wherein the recombinant RNA molecule comprises 5′ and 3′ ends that are native to the oncolytic virus encoded by the synthetic RNA viral genome.

Embodiment 175. A composition comprising an effective amount of the recombinant RNA molecule of any one of Embodiments 109-139, and a carrier suitable for administration to a mammalian subject.

Embodiment 176. A particle comprising the recombinant RNA molecule of any one of Embodiments 109-139.

Embodiment 177. The particle of Embodiment 176, wherein the particle is biodegradable.

Embodiment 178. The particle of Embodiment 177, wherein the particle is selected from the group consisting of a nanoparticle, an exosome, a liposome, and a lipoplex.

Embodiment 179. The particle of Embodiment 178, wherein the exosome is a modified exosome derived from an intact exosome or an empty exosome.

Embodiment 180. A pharmaceutical composition comprising a plurality of particles according to any one of Embodiments 176-179.

Embodiment 181. The pharmaceutical composition of Embodiment 180, wherein the plurality of particles have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

Embodiment 182. The pharmaceutical composition of Embodiment 180 wherein the plurality of particles have an average size of about 50 nm to about 120 nm.

Embodiment 183. The pharmaceutical composition of Embodiment 180 wherein the plurality of particles have an average size of about 50 nm, 60 nm, 70 nm, 80 nm, 90 nm, 100 nm, 110 nm, or about 120 nm.

Embodiment 184. The pharmaceutical composition of Embodiment 180 wherein the plurality of particles have an average size of about 100 nm.

Embodiment 185. The pharmaceutical composition of any one of Embodiments 180-184, wherein the plurality of particles have an average zeta-potential of between about 40 mV to about −40 mV, about 20 mV to about −20 mV, about 10 mV to about −10 mV, about 5 mV to about −5 mV, or about 20 mV to about −40 mV.

Embodiment 186. The pharmaceutical composition of any one of Embodiments 180-184, wherein the plurality of particles have an average zeta-potential of less than about 5 mV, less than about 0 mV, less than about −5 mV, less than about −10 mV, less than about −20 mV, less than about −30 mV, less than about −35 mV, or less than about −40 mV.

Embodiment 187. The pharmaceutical composition of any one of Embodiments 180-186, wherein the plurality of particles have an average zeta-potential of between about −50 mV to about −20 mV, about −40 mV to about −20 mV, about −30 mV to about −10 mV, about −20 mV to about 0 mV, about −15 mV to about 5 mV, or about −10 mV to about 10 mV.

Embodiment 188. The pharmaceutical composition of any one of Embodiments 180-186, wherein the plurality of particles have an average zeta-potential of about −30 mV, about −31 mV, about −32 mV, about −33 mV, about −34 mV, about −35 mV, about −36 mV, about −37 mV, about −38 mV, about −39 mV, or about −40 mV.

Embodiment 189. The pharmaceutical composition of any one of Embodiments 180-188, wherein delivery of the composition to a subject delivers the encapsulated recombinant RNA molecule to a target cell, and wherein the encapsulated recombinant RNA molecule produces an infectious virus capable of lysing the target cell.

Embodiment 190. An inorganic particle comprising the recombinant RNA molecule of any one of Embodiments 109-139.

Embodiment 191. The inorganic particle of Embodiment 190, wherein the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

Embodiment 192. A composition comprising the inorganic particle of any one of Embodiments 190-191, wherein the average diameter of the particles is less than about 500 nm, is between about 50 nm and 500 nm, is between about 250 nm and about 500 nm, or is about 350 nm.

Embodiment 193. The LNP of any one of Embodiments 1-87, the particle of any one of Embodiments 176-179, or the inorganic particle of any one of Embodiments 190-191, further comprising a second recombinant RNA molecule encoding a payload molecule.

Embodiment 194. The LNP, particle, or inorganic particle of Embodiment 193, wherein the second recombinant RNA molecule is a replicon.

Embodiment 195. A pharmaceutical composition comprising the LNP of any one of Embodiments 1-87, the particle of any one of Embodiments 176-179, or the inorganic particle of any one of Embodiments 190-191, wherein the composition is formulated for intravenous and/or intratumoral delivery.

Embodiment 196. The pharmaceutical composition of Embodiment 195, wherein the target cell of the LNP, the particle, or the inorganic particle is a cancerous cell.

Embodiment 197. A method of killing a cancerous cell comprising exposing the cancerous cell to the particle of any one of Embodiments 1-87, 176-179, or 190-191, the recombinant RNA molecule of any one of Embodiments 109-139, or compositions thereof, under conditions sufficient for the intracellular delivery of the particle to said cancerous cell, wherein the replication-competent virus produced by the encapsulated polynucleotide results in killing of the cancerous cell.

Embodiment 198. The method of Embodiment 197, wherein the replication-competent virus is not produced in non-cancerous cells.

Embodiment 199. The method of Embodiment 197 or 198, wherein the method is performed in vivo, in vitro, or ex vivo.

Embodiment 200. A method of treating a cancer in a subject comprising administering to a subject suffering from the cancer an effective amount of the particle of any one of Embodiments 1-87, 176-179, or 190-191, the recombinant RNA molecule of any one of Embodiments 109-139, or compositions thereof.

Embodiment 201. The method of Embodiment 200, wherein the particle or composition thereof is administered intravenously, intranasally, intratumorally, intraperitoneally, or as an inhalant.

Embodiment 202. The method of Embodiment 200, wherein the particle or composition thereof is administered intratumorally and/or intravenously.

Embodiment 203. The method of any one of Embodiments 200-202, wherein the particle or composition thereof is administered to the subject repeatedly.

Embodiment 204. The method of any one of Embodiments 200-203, wherein the subject is a mouse, a rat, a rabbit, a cat, a dog, a horse, a non-human primate, or a human.

Embodiment 205. The method of any of Embodiments 200-204, wherein the cancer is lung cancer, breast cancer, colon cancer, or pancreatic cancer, and wherein the synthetic RNA viral genome comprises a polynucleotide sequence derived from the KY strain.

Embodiment 206. The method of any of Embodiments 200-204, wherein the cancer is bladder cancer, renal cell carcinoma, ovarian cancer, gastric cancer or liver cancer, and wherein the synthetic RNA viral genome comprises a polynucleotide sequence derived from the EF strain.

Embodiment 207. The method of any one of Embodiments 197-204, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

Embodiment 208. The method of any of Embodiments 197-204, wherein the cancer is selected from the groups consisting of lung cancer, breast cancer, colon cancer, pancreatic cancer, bladder cancer, renal cell carcinoma, ovarian cancer, gastric cancer and liver cancer.

Embodiment 209. The method of any of Embodiments 197-204, wherein the cancer is renal cell carcinoma, lung cancer, or liver cancer.

Embodiment 210. The method of Embodiment 205, 207, or 208, wherein the lung cancer is small cell lung cancer or non-small cell lung cancer (e.g., squamous cell lung cancer or lung adenocarcinoma).

Embodiment 211. The method of any of Embodiments 206, 207, and 208, wherein the liver cancer is hepatocellular carcinoma (HCC) (e.g., Hepatitis B virus associated HCC).

Embodiment 212. The method of Embodiment 207, wherein the prostate cancer is treatment-emergent neuroendocrine prostate cancer.

Embodiment 213. The method of any one of Embodiments 197-204, wherein the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma.

Embodiment 214. The method of any one of Embodiments 197-204, wherein the cancer is small cell lung cancer (SCLC) or neuroblastoma.

Embodiment 215. A method of treating a cancer in a subject in need thereof comprising administering an effective amount of a CVA21-EF virus to the subject.

Embodiment 216. A method of treating a cancer in a subject in need thereof comprising administering an effective amount of a CVA21-KY virus to the subject

Embodiment 217. A method of treating a cancer in a subject in need thereof comprising administering an effective amount of a CVA21-Kuykendall virus to the subject.

Embodiment 218. The method of any one of Embodiments 215-217, wherein the virus is administered intratumorally and/or intravenously.

Embodiment 219. The method of any one of Embodiments 197-218, further comprising administering an immune checkpoint inhibitor to the subject.

Embodiment 220. The method of Embodiment 219, wherein the immune checkpoint inhibitor is an inhibitor of PD-1.

Embodiment 221. The method of any one of Embodiments 197-218, further comprising administering an engineered immune cell comprising an engineered antigen receptor.

Embodiment 222. A method of treating a cancer in a subject in need thereof, comprising administering a therapeutically effective amount of an oncolytic Coxsackievirus, wherein the Coxsackievirus is a CVA21 strain, or a polynucleotide encoding the CVA21 to the subject, wherein the cancer is classified as sensitive to CVA21 infection based on the expression of ICAM-1 and/or the percentage of ICAM-1 positive cancer cells.

Embodiment 223. A method of treating a cancer in a subject in need thereof, comprising:
(a) determining the expression level of ICAM1 and/or the percentage of ICAM-1 positive cancer cells in the cancer;
(b) classifying the cancer as sensitive to Coxsackievirus 21 (CVA21) infection based on the expression of ICAM-1 and/or the percentage of ICAM-1 positive cancer cells determined in (a); and
(c) administering a therapeutically effective amount of CVA21 or a polynucleotide encoding the CVA21 to the subject if the cancer is classified as sensitive to CVA21 infection in step (b).

Embodiment 224. A method of selecting a subject suffering from a cancer for treatment with a Coxsackievirus 21 (CVA21) or a polynucleotide encoding the CVA21, comprising:
(a) determining the expression level of ICAM-1 and/or the percentage of ICAM-1 positive cancer cells in the cancer;
(b) classifying the cancer as sensitive to CVA21 infection based on the expression level of ICAM-1 and/or the percentage of ICAM1 positive cancer cells as determined in (a);
(c) selecting the subject for treatment with the CVA21 or the polynucleotide encoding the CVA21 if the cancer is classified as sensitive to CVA21 infection in (b); and
(d) administering the CVA21 or the polynucleotide encoding the CVA21 to the selected subjects

Embodiment 225. The method of any one of Embodiments 222-224, wherein the CVA21 strain is CVA21-KY.

### EXAMPLES

The following examples are given for the purpose of illustrating various embodiments of the disclosure and are not meant to limit the present disclosure in any fashion. The present examples; along with the methods described herein are presently representative of preferred embodiments; are exemplary; and are not intended as limitations on the scope of the disclosure. Changes therein and other uses which are encompassed within the spirit of the disclosure as defined by the scope of the claims will occur to those skilled in the art.

### Example 1: Production of Infectious Picornavirus Virus from Recombinant RNA Molecules

Experiments were performed to assess the ability to produce infectious CVA21 virus from recombinant RNA molecules. Briefly, RNA polynucleotides comprising CVA21 viral genomes were generated by T7 transcription in vitro and 293T cells were transfected with 1 μg of the CVA21-RNA constructs in Lipofectamine RNAiMax for 4 hours, cells were washed, and complete media was added to each well. Supernatants from transfected 293T were collected after 72 hours, syringe filtered with 0.45 μM filter and serially diluted onto NCI-H1299 cells. After 48 hours, supernatants were removed from the NCI-H1299 cultures and cells were stained with crystal violet to assess viral infectivity. RNA molecules comprising CVA21 viral genomes produced active lytic virus (data not shown).

In addition, supernatants of NCI-H1299 cells treated with 1 μg of CVA21-RNA lipid, CVA21 plasmid DNA, or CVA21-Negative pDNA control were collected after 72 hours and serially diluted onto uninfected NCI-H1299 cells. Cell viability assays were performed according to standard protocols. CVA21-RNA/LNP are capable of producing infectious virus that results in tumor cell lysis in vitro (data not shown).

### Example 2: Formulation of Lipid Nanoparticles for Intravenous Delivery of CVA21-Encoding RNA

Recombinant RNA molecules comprising CVA21 genomes were formulated in lipid nanoparticles for delivery of the RNA in vivo.

### Lipid Nanoparticle Production:

The following lipids were used in formulation of lipid nanoparticles:
(a) D-Lin-MC3-DMA (MC3);
(b)N-(2,3-dioleoyloxy)propyl)-N,N,N-trimethylammonium chloride (DOTAP)
(c) COATSOME® SS-LC (former name: SS-18/4PE-13);
(d) COATSOME® SS-EC (former name: SS-33/4PE-15);
(e) COATSOME® SS—OC;
(f) COATSOME® SS—OP;
(g) Di((Z)-non-2-en-1-yl)9-((4-dimethylamino)butanoyl)oxy)heptadecanedioate (L-319)
(h) cholesterol;
(i) 1,2-distearoyl-sn-glycero-3-phosphocholine (DSPC);
(j) 1,2-dilauroyl-sn-glycero-3-phosphoethanolamine (DLPE);
(k) 1,2-dioleoyl-sn-glycero-3-phosphocholine (DOPC);
(l) 1,2-dioleoyl-sn-glycero-3-phosphoethanolamine (DOPE);
(m) 1,2-distearoyl-sn-glycero-3-phosphoethanolamine-N-[amino(polyethyleneglycol)-5000] (DSPE-PEG5K);
(n) 1,2-dipalmitoyl-rac-glycerol methoxypolyethylene glycol-2000 (DPG-PEG2K);
(o) 1,2-distearoyl-rac-glycero-3-methylpolyoxyethylene-2000 (DSG-PEG2K);
(p) 1,2-dimyristoyl-rac-glycero-3-methylpolyoxyethylene-2000 (DMG-PEG2K)
(q) polyoxyethylene (100) stearyl ether (BRIJ™ S100; CAS number: 9005-00-9);
(r) polyoxyethylene (20) stearyl ether (BRIJ™ 520; CAS number: 9005-00-9);
(s) polyoxyethylene (20) oleyl ether (BRIJ™ O20; CAS number: 9004-98-2);
(t) polyoxyethylene (20) cetyl ether (BRIJ™ C₂₀, CAS number: 9004-95-9);
(u) Polyoxyethylene (40) stearate (MYRJ™ S40, CAS number: 9004-99-3).

Lipids were prepared in ethanol at various ratios. RNA lipid nanoparticles were then generated using microfluidic micromixture (Precision NanoSystems, Vancouver, BC) at a combined flow rate of 2 mL/min (0.5 mL/min for ethanol, lipid mix and 1.5 mL/min for aqueous buffer, RNA). The resulting particles were washed by tangential flow filtration with PBS containing Ca and Mg.

### Analysis of Physical Characteristics of Lipid Nanoparticles:

Physical characteristics of lipid nanoparticles were evaluated before and after tangential flow filtration. Particle size distribution and zeta potential measurements were determined by light scattering using a Malvern Nano-ZS Zetasizer (Malvern Instruments Ltd, Worcestershire, UK). Size measurements were performed in HBS at pH 7.4 and zeta potential measurements were performed in 0.01 M HBS at pH 7.4. Percentage of RNA entrapment was measured by Ribogreen assay. Lipid nanoparticles that showed greater than 80 percent RNA entrapment were tested in vivo.

### Example 3: In Vivo Efficacy of CVA21-Encoding RNA Lipid Nanoparticles in Melanoma

Experiments were performed to determine the ability of lipid nanoparticles comprising CVA21-encoding RNA molecules to produce infectious virus and inhibit melanoma tumor growth in vivo. CVA21 RNA lipid nanoparticle production, formulation, and analysis are described in Example 2.

The ability of CVA21 RNA lipid nanoparticles to inhibit tumor growth was evaluated using the SK-MEL28 xenograft model. Briefly, SK-MEL28 cells (1×10⁶ cells/0.1 mL in a 1:1 mixture of serum-free PBS and Matrigel®) were subcutaneously inoculated in the right flank of 8-week-old female athymic nude mice (Charles River Laboratories). When median tumor size reached approximately 150 mm³ (120-180 mm³ range), mice were intratumorally administered either PBS or CVA21-encoding RNA formulated with Lipofectamine RNAiMAx (1 μg), or intravenously administered CVA21-encoding RNA lipid nanoparticles (formulation ID: 70032-6C, 5 μg). Mice received intratumoral treatments on days 1 and 5, or intravenous treatment on days 1, 6, 11, and 16. Tumor volume was measured 3 times per week using electronic calipers.

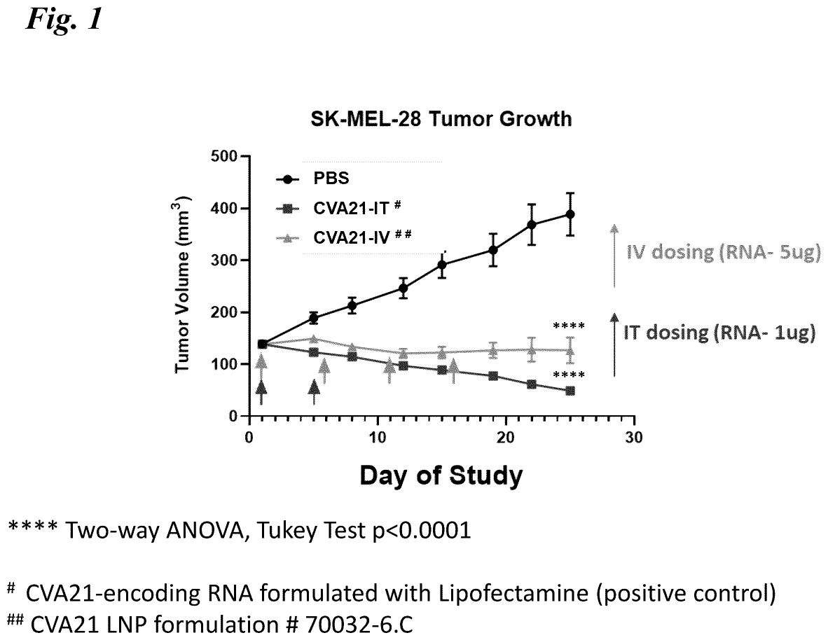

As shown in FIG. 1, intravenous treatment with LNPs comprising CVA21 Kuykendall strain RNA molecules (formulation ID: 70032-6C; MC3:Chol:DSPC:DPG-PEG5K is about 49:39.8:11:0.2 mol %) or intratumoral treatment of CVA21-Kuykendall strain RNA molecules formulated with Lipofectamine prevented tumor growth in tumor-bearing mice compared to mice treated with PBS (two-way ANOVA, p<0.0.001). Collectively, these results suggest that lipid nanoparticles comprising CVA21 RNA molecules are an effective therapeutic strategy for the treatment of melanoma.

### Example 4: Strategies for Generation of Discrete 3′ Termini of CVA21

As described above, the synthetic genomes described herein require discrete 3′ and 5′ ends native to the virus in order to produce a replication-competent and infective virus from the synthetic genome. The RNA transcripts produced by T7 RNA polymerase in vitro mammalian 5′ and 3′ UTRs and therefore do not contain the discrete, native ends required for production of an infectious ssRNA virus.

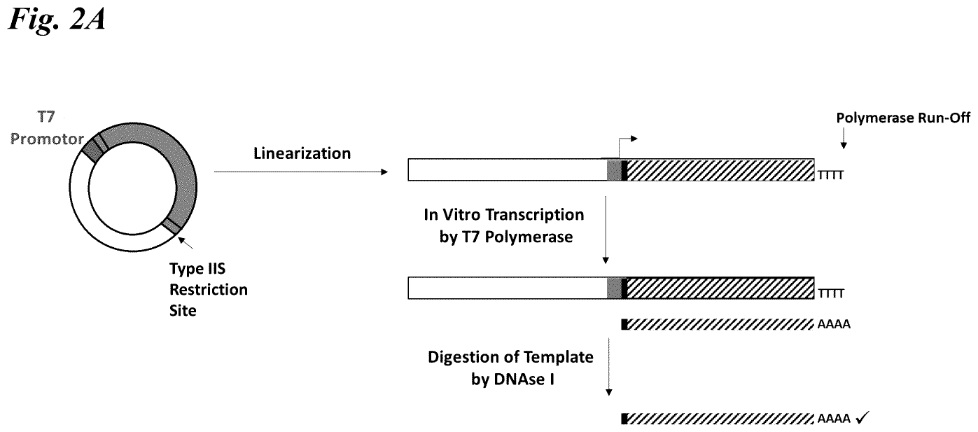

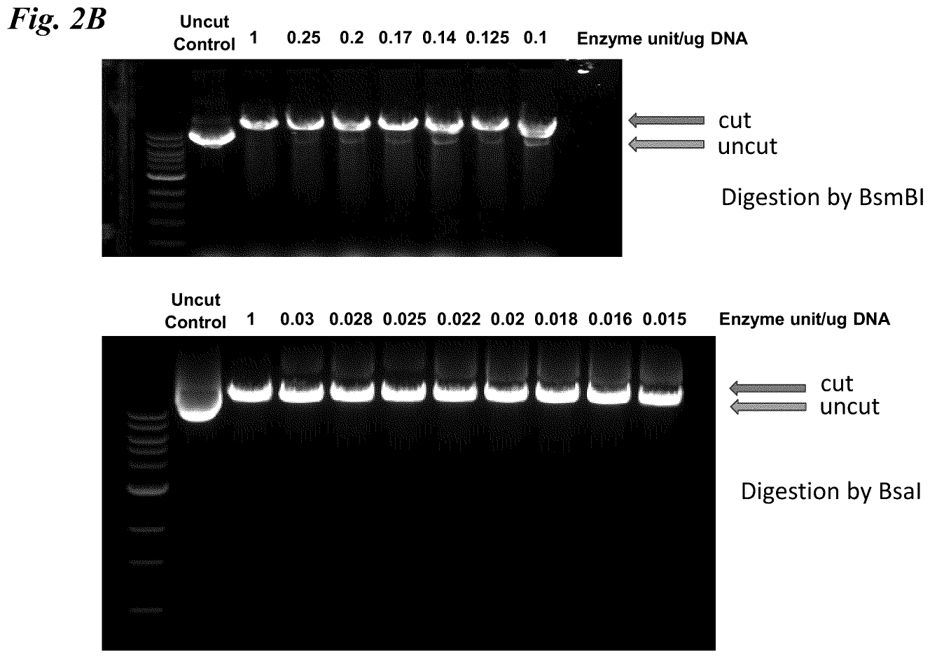

A strategy using 3′ restriction enzyme recognition sequences was employed to generate the discrete 3′ ends required for infectious CVA21. The Type IIS restriction recognition sequence (e.g., BsmBI, BsaI, or SapI recognition sequence) was inserted at the 3′ end of the DNA template. The corresponding restriction enzyme (e.g., BsmBI, BsaI, or SapI) cleaves 5′ of its recognition site to generate a polythymidine run of the appropriate length to generate the discrete virus polyadenylation site native to the virus. This process is illustrated in FIG. 2A.

Both BsaI-HF®v2 and BsmBI-v2 (New England Biolabs) were tested for their efficiency of generating discrete 3′ ends of DNA templates. DNA templates containing either a BsmBI recognition sequence or a BsaI recognition sequence were constructed and subjected to digestion by the corresponding enzyme at the appropriate condition provided in the product manuals. As shown in FIG. 2B (upper gel image), BsmBI achieved complete digestion of the corresponding DNA construct at 1 enzyme unit/μg DNA concentration, but lower concentrations of BsmBI led to incomplete digestion. On the other hand, as shown in FIG. 2B (lower gel image), BsaI achieved completed digestion of the corresponding DNA construct at a lower concentration of 0.015 enzyme unit/μg DNA, suggesting that incorporating a BsaI recognition sequence after the poly-A tail region and the use of corresponding BsaI restriction enzyme can efficiently generate the discrete 3′ end of the in vitro DNA template for the RNA viral genome.

### Example 5: An RNaseH Strategy for Generation of Discrete 5′ Termini of CVA21

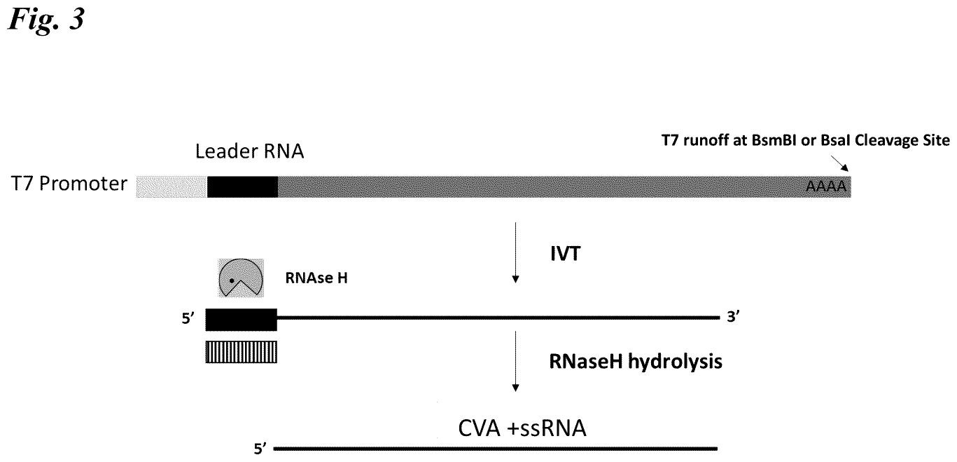

An RNAseH strategy was employed to generate the discrete 5′ termini native to CVA21. The T7 leader must be removed to generate an authentic terminus for the virus. Depicted in FIG. 3 is a diagram of the in vitro transcription (IVT) and 5′ leader processing approach. The IVT template is depicted at the top and the resulting RNA transcript is illustrated in the middle. This CVA21+ssRNA transcript is then annealed to a complementary dsDNA oligo (dashed box) and that portion is hydrolyzed with RNaseH. The final viral ssRNA product, with the correct 5′ terminus, is shown at the bottom.

This strategy, in combination with the 3′ restriction enzyme strategy, produces a final synthetic CVA21 genome with the discrete 5′ and 3′ termini required for production of infectious CVA21.

### Example 6: A Ribozyme Strategy for Generation of Discrete 5′ Termini of CVA21

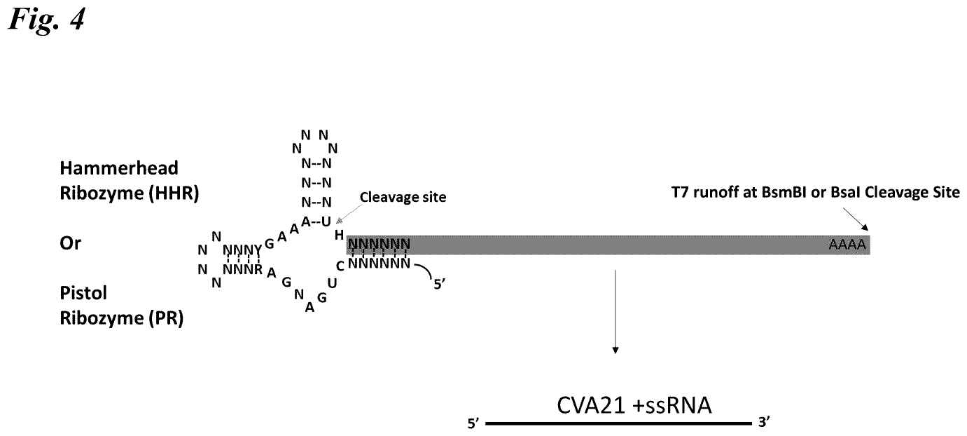

A ribozyme strategy was employed to generate the discrete 5′ termini native to CVA21. A schematic of this approach is illustrated in FIG. 4, showing the design of ribozymes to cleave at the 5′ terminus of a picornavirus. The two ribozymes depicted are hammerhead and pistol ribozymes, however multiple other ribozymes could be adapted to cleave specifically in this context.

Modifications of the hammerhead and pistol ribozymes for implementation in this strategy are shown in FIG. 5 and FIG. 6, respectively. A structural model of a minimal hammerhead ribozyme (HHR) that anneals and cleaves the 5′ end of CVA21 is shown in FIG. 5A (this ribozyme cleaves the 5′ end at the site indicated by the arrow). A structural model of hammerhead ribozyme with a stabilized stem I for cleavage of the CVA21 5′ terminus (STBL) is shown in FIG. 5B (this ribozyme cleaves the 5′ end at the site indicated by the arrow). FIG. 6A shows a schematic of Pistol ribozyme characteristics found in the wild (Pistol WT). FIG. 6B shows a Pistol ribozyme from P. Polymyxa modeled by mFOLD with a tetraloop added to fuse the P3 strands. The nucleic acids in the dashed box were mutagenized to retain the fold of the ribozyme in the context of the viral sequence. The WT “GUC” sequence shown in the dashed box was mutated to “UCA” to generate Pistol 1 and the “GUC” sequence was mutated to “TTA” to generate Pistol 2.

### Example 7: Optimization of Coxsackievirus-Encoding RNA Molecules

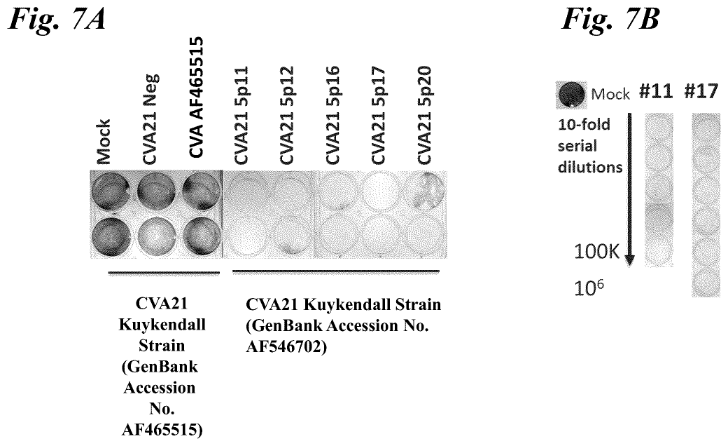

Experiments were performed to assess the ability to produce infectious Coxsackie Virus A21 (CVA21) from recombinant RNA molecules. Briefly, RNA polynucleotides comprising CVA21 viral genomes were generated by T7 transcription in vitro based on previously described CVA21 genome sequences (See Newcombe et al., Cellular receptor interactions of C-cluster human group A coxsackieviruses Journal of General Virology (2003), 84, 3041-3050. GenBank Accession No. AF465515). SK-MEL-28 cells were transfected with 1 μg of the CVA21 Kuykendall strain RNA constructs in Lipofectamine RNAiMax for 4 hours, at which point wells were washed and complete media was added to each well. After 48 hours, supernatants were removed from the SK-MEL-28 cultures and cells were stained with crystal violet to assess viral infectivity. As shown in FIG. 7A (left panel), RNA molecules comprising the Newcombe CVA21 sequences (CVA21 Kuykendall strain with viral genome sequence according to GenBank Accession No. AF465515) did not produce active lytic virus (indicated by crystal violate staining of un-lysed SK-MEL-28 cells).

Surprisingly, alterations to the 5′ UTR were required for the production of infectious CVA21 from recombinant RNA molecule. As shown in FIG. 7A (right panel), incorporation of the 5′ UTR sequence described by Brown et al. (Journal of Virology, (2003)77:16, p. 8973-8984. GenBank Accession No. AF546702) into the CVA21 genome sequence described by Newcombe (CVA21 Kuykendall strain with viral genome sequence according to GenBank Accession No. AF546702; hereafter CVA21-Brown) resulted in the production of infectious CVA21 virus and viral cell lysis, indicated by the lack of crystal violet staining across multiple independent clones. Supernatants from SK-MEL-28 cells transfected with two different CVA21-Brown clones were collected after 72 hours, and syringe filtered with 0.45 μM filter and serially diluted onto fresh SK-MEL-28 cells. After 48 hours, supernatants were removed from the SK-MEL-28 cultures and cells were stained with crystal violet to assess viral infectivity. As shown in FIG. 7B, CVA21 encoding RNA molecules comprising the Brown 5′ UTR sequence (UTR sequence—SEQ ID NO: 2, modified CVA21 Kuykendall strain sequence—SEQ ID NO: 1) resulted in the production and release of infectious CVA21 into the supernatant of transfected cells, indicated by the ability of the supernatants alone to mediate cell lysis.

### Example 8: CVA21-RNA In Vivo Efficacy

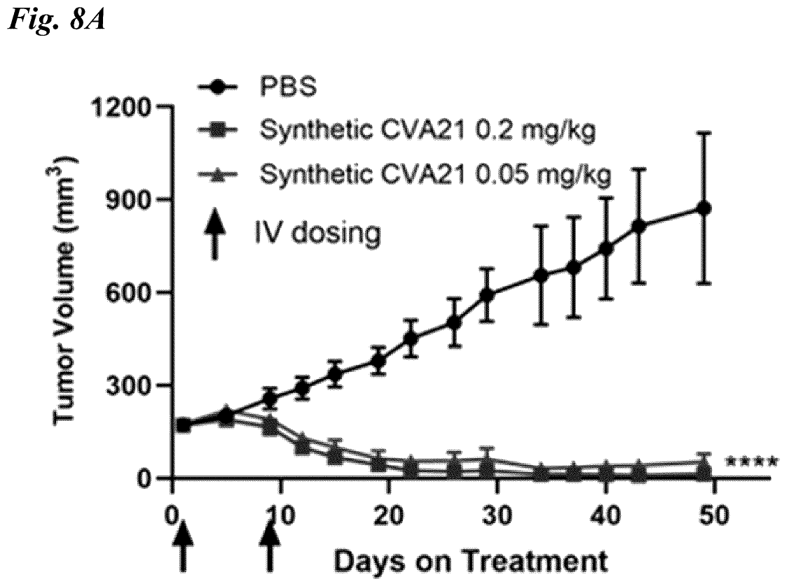

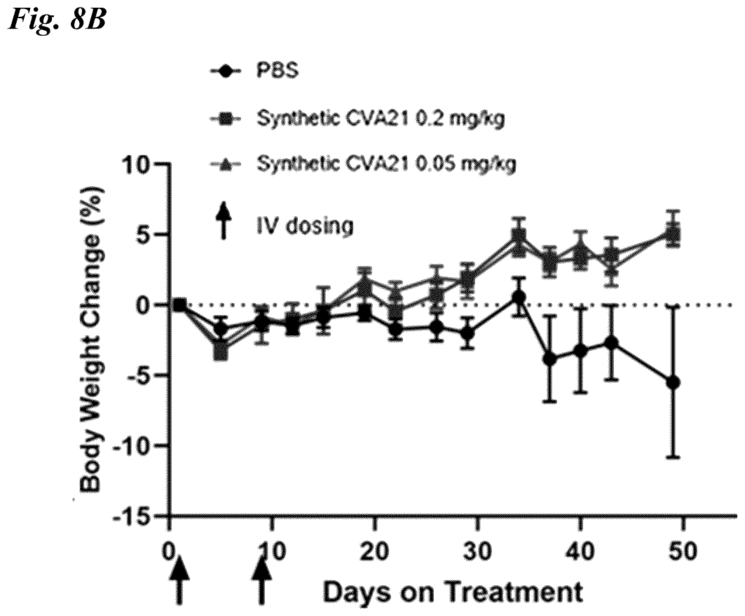

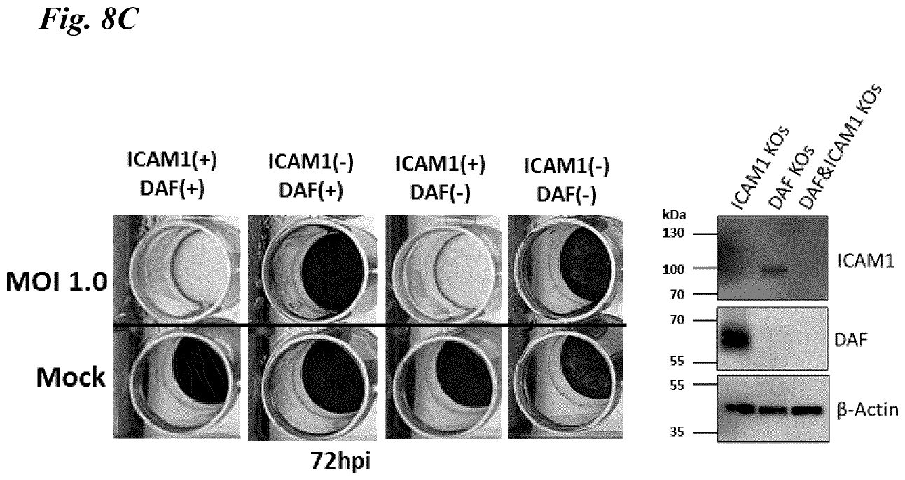

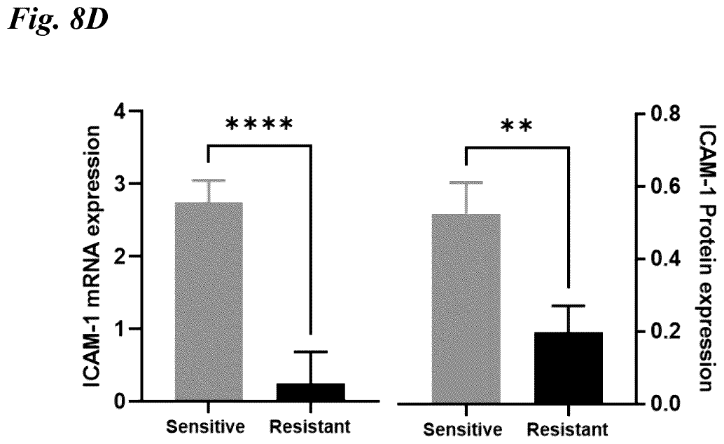

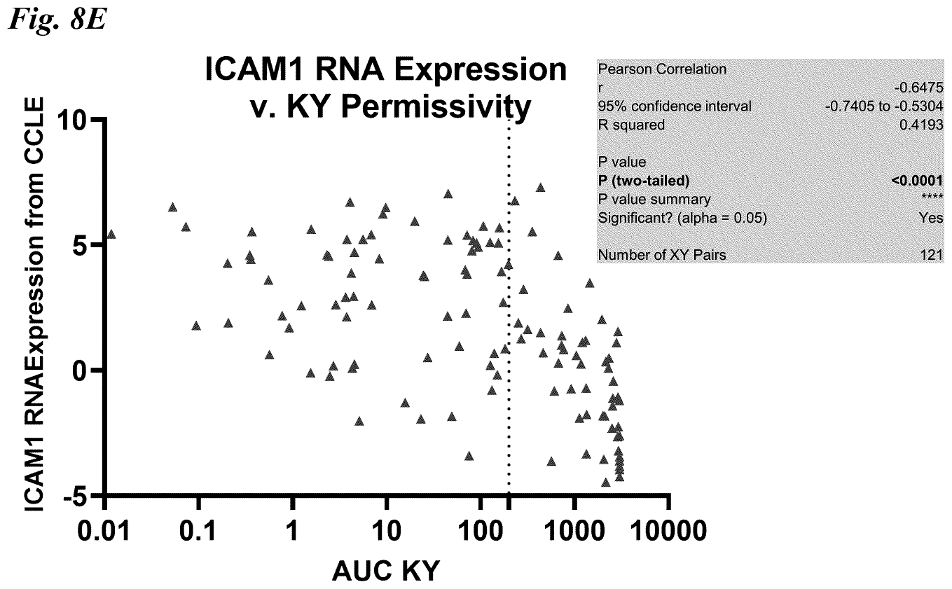

Experiments were performed to assess the in vivo efficacy of LNP-encapsulated CVA21 Kuykendall genomes. Briefly, an SK-MEL-28 model of melanoma was used. 8-12 week old NU/NU nude female mice were subcutaneously injected with 5×10⁶-1×10⁷ viable tumor cells in the right flank in 100 μL of Matrigel. Treatment was initiated when tumors reached the pre-determined volume of 150±30 mm³. Synthetic CVA21-LNPs were administered intravenously at 0.2 mg/kg or 0.05 mg/kg on days 1 and 8. Complete tumor regression at a dose level as low as 0.05 mg/kg was observed (FIG. 8A). Both doses were well tolerated, as indicated by stable body weight (FIG. 8B) and no adverse clinical signs.

### CVA21 Viral Entry and ICAM-1 Expression:

Experiments were performed to assess the requirement for ICAM-1 and decay accelerating factor (DAF) expression on cells for CVA21 viral entry. Briefly, H1299 cells were modified to knock-out ICAM-1 and/or DAF expression. The modified cells were infected with CVA21-KY for 72 hours. As shown in FIG. 8C, ICAM-1 expression was required for CVA21-mediated cell lysis (evidenced by the absence of the cell monolayer in ICAM-1+conditions).

130 human cell lines were then analyzed for ICAM-1 expression by mRNA and protein expression. The ICAM-1 expression level was then correlated with the sensitivity of the cell lines to CVA infection. A cell line was classified as sensitive to CVA21-KY infection if the TCID50 was <0.5. A cell line was classified as resistant to CVA21-KY infection if the TCID50 was >0.5. As shown in FIG. 8D, sensitive cell lines showed significantly increased ICAM-1 expression compared to resistant cell lines and the ICAM-1 expression correlated with CVA21-KY infection (FIG. 8E). Human tumor microarray tissues from 7 different disease indications including NSCLC, RCC, HCC, Melanoma, HNSCC, TNBC, and bladder cancer were further evaluated for human ICAM-1 expression via immunohistochemistry for potential disease indication selection (Table 19 below). The results indicated that most of these tumor tissue would be permissive for CVA21 infection.

TABLE 19
Analysis of human ICAM-1 expression using tumor
microarrays and IHC on 7 disease indications

| | | | Tumor Area | | | |
|---|---|---|---|---|---|---|
| | | Prevalence | positivity | | | |
| | | Rate | Mean % of tumor | | H-score* | |
| | No. of core | % of cores | cells that are | | Mean H-score for | |
| | tumor samples | positive: | ICAM1 + ≥1% | | ICAM1 + ≥1% | |
| Disease Indications | tested (N) | ICAM-1 + ≥1% | cores (median) | | cores (median) | |
| NSCLC | 97 | 72% | 39% | (30%) | 92 | (60) |
| RCC (Clear Cell) | 67 | 79% | 40% | (40%) | 83 | (50) |
| HCC | 69 | 72% | 40% | (25%) | 86 | (48) |
| Melanoma | 26 | 77% | 31% | (23%) | 67 | (60) |
| TNBC | 46 | 52% | 8% | (3%) | 21 | (4) |
| Head & Neck Cancer | 60 | 70% | 7% | (2%) | 14 | (4) |
| Bladder (Urothelial | 37 | 24% | 24% | (3%) | 50 | (9) |
| Carcinoma) | | | | | | |

*H-score is a measure of expression intensity (+1, +2, +3) and % tumor area = [1 × (% cells 1+) + 2 × (% cells 2+) + 3 × (% cells 3+)]

### Example 9: CVA21 Strain Selection

Experiments were performed to determine the differences in cell tropism and therapeutic efficacy of synthetic oncolytic RNAs derived from one of three CVA21 strains: Kuykendall strain, the EF strain, and the KY strain. FIG. 32 shows the domain organization schematics of the RNA viral genomes of three CVA21 strains (EF, KY, and Kuykendall) and the nucleic acid starting/ending positions of selective region.

Evaluation criteria included:
(a) tropism breadth and potency of cancer cell killing ability in vitro and in vivo;
(b) virus sensitivity to cellular anti-viral responses (e.g., interferon sensitivity);
(c) strain stability (e.g., replication fidelity, recombination frequency);
(d) safety and efficacy in vivo.

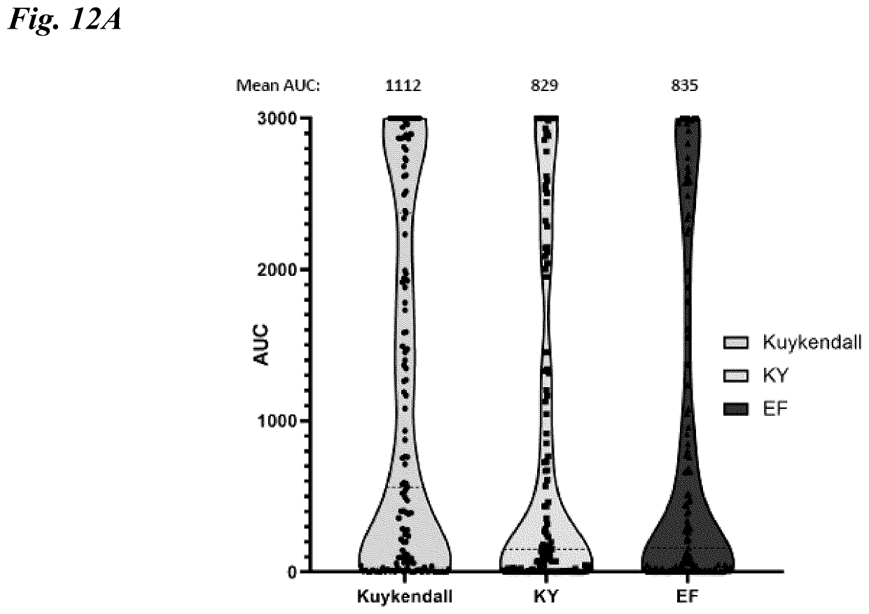

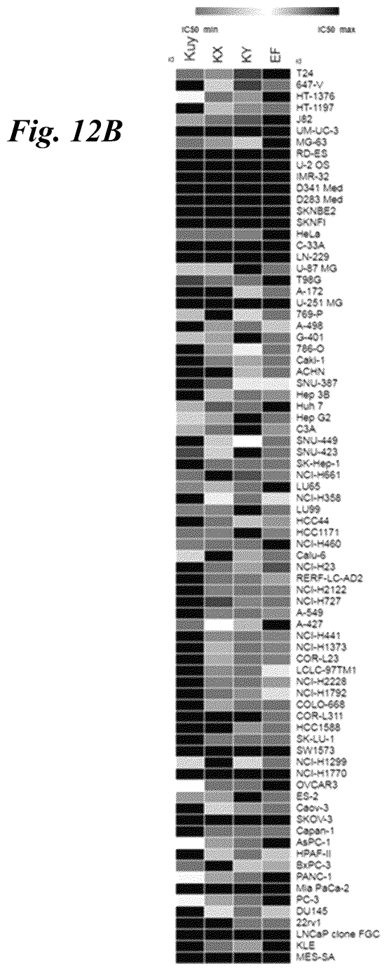

Overall, the results show that both KY and EF strains clearly outperform the lab strain Kuykendall (KuyK). Results of these experiments are provided in FIGS. 12A-28B and are summarized as follows:

### In Vitro Tropism and Potency:

Cytotoxicity screen and quantitative Western blotting were performed to compare the tropism and potency of the three CVA21 strains. As shown in FIG. 12A and FIG. 12B, the EF and KY outperformed the KX (not shown) and Kuykendall strains and had both a greater breadth of cell lines killed (i.e., broader tropism) when comparing individual strains within cell lines of interest. Using AUC values <200 as a cutoff for sensitivity, 82 of the 131 cell lines tested were resistant to KuyKendall lab strain, but only 59 of the 131 cell lines were resistant to KY strain and only 65 of the 131 cell lines were resistant to EF strain (FIG. 12A).

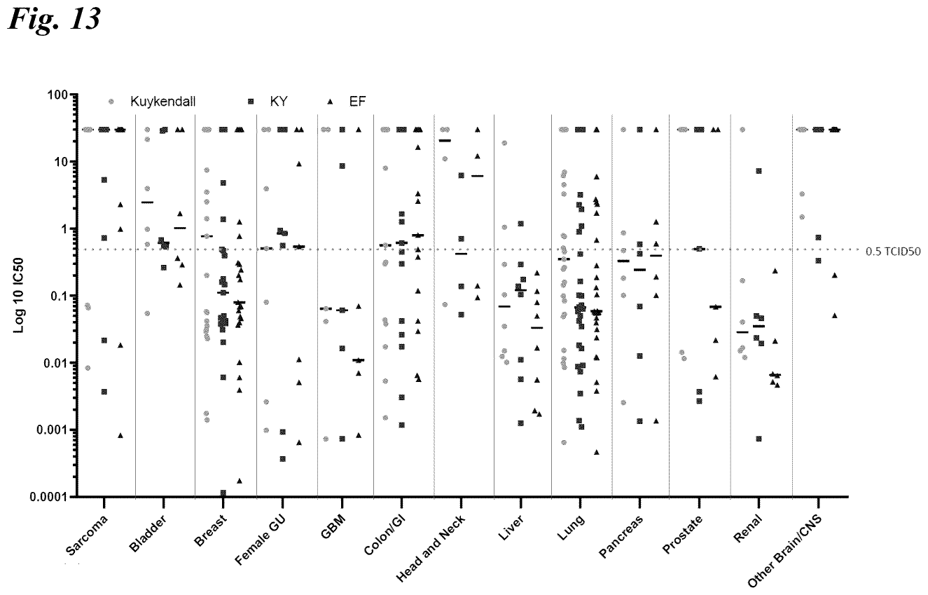

As shown in FIG. 13, the KY and EF strains exhibit broader tropism and/or increased potency across multiple indications compared to the Kuykendall lab strain.

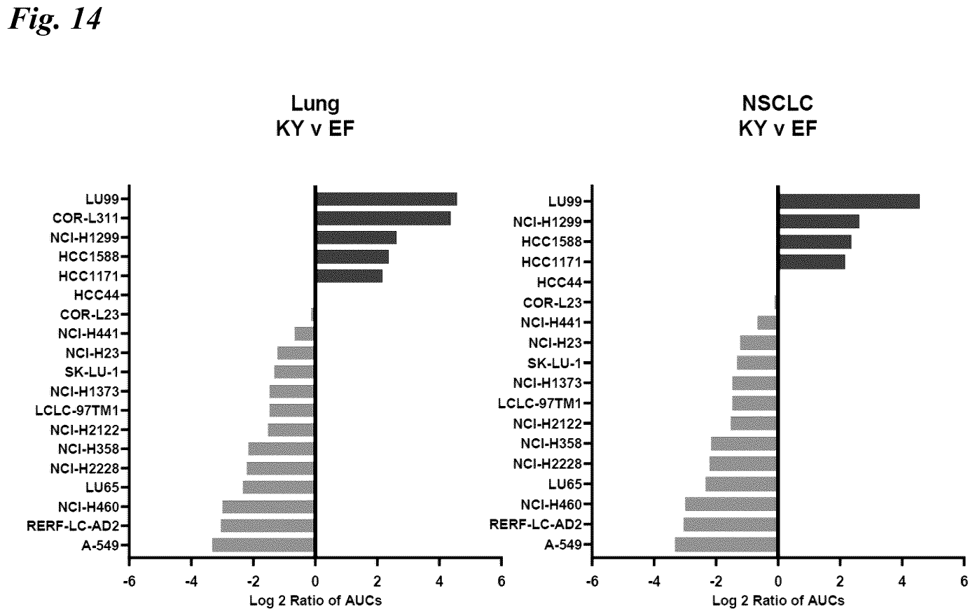

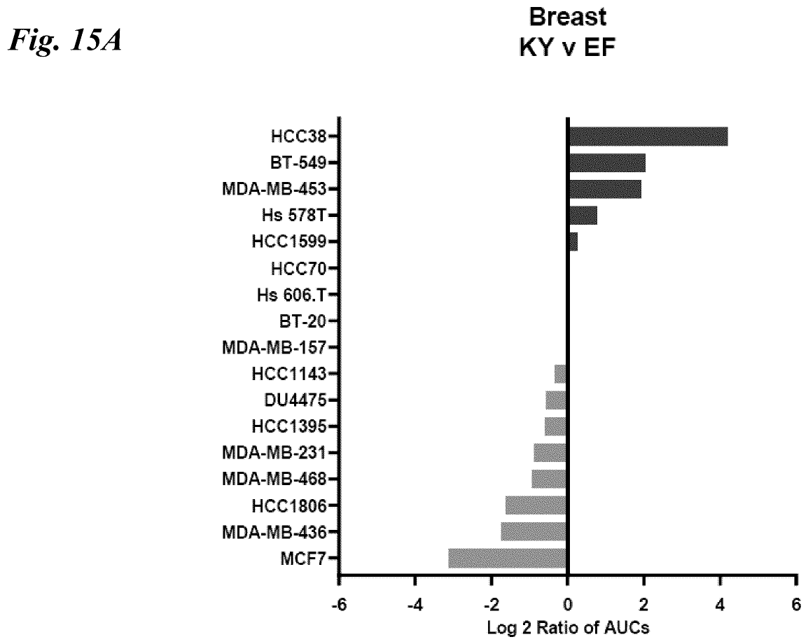

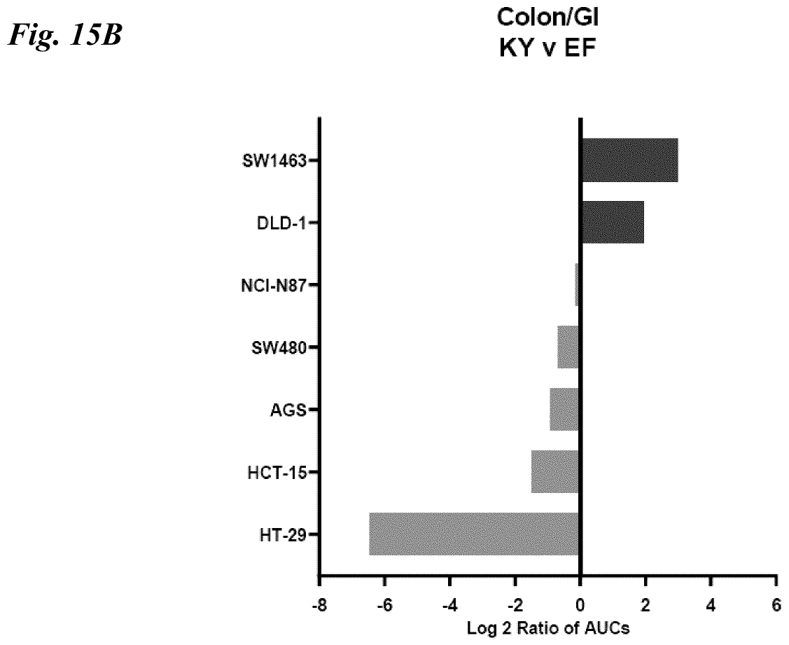

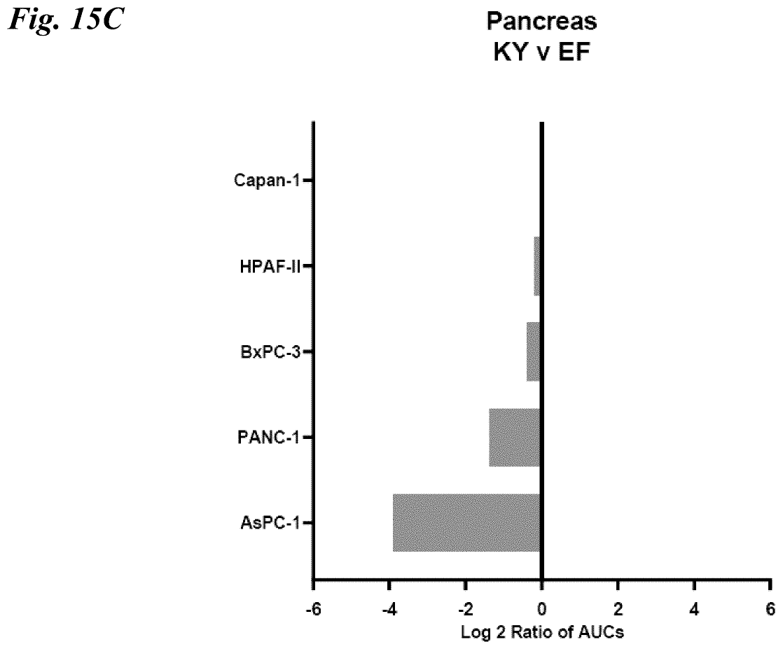

As shown in FIG. 14, the KY strain demonstrated a more favorable tropism in lung cancer cell lines, including NSCLC cell lines. Results of the in vitro cytotoxicity screen were plotted as a ratio of the AUC values for EF and KY strains. Cell lines with values >1 favor EF (dark grey bars), while cells lines with values <1 favor KY (light grey bars). Additional indications in which KY demonstrated favorable trophism are shown in FIG. 15 (breast cell lines FIG. 15A, colon/GI lines FIG. 15B, and pancreatic cell lines FIG. 15C). In FIG. 15, dark grey represents EF favored trophism and light grey represents KY favored trophism.

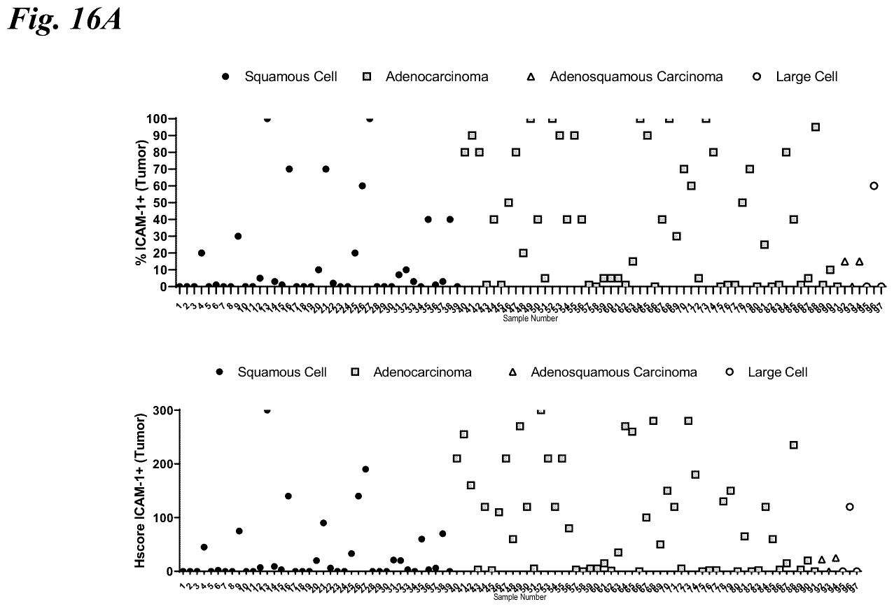

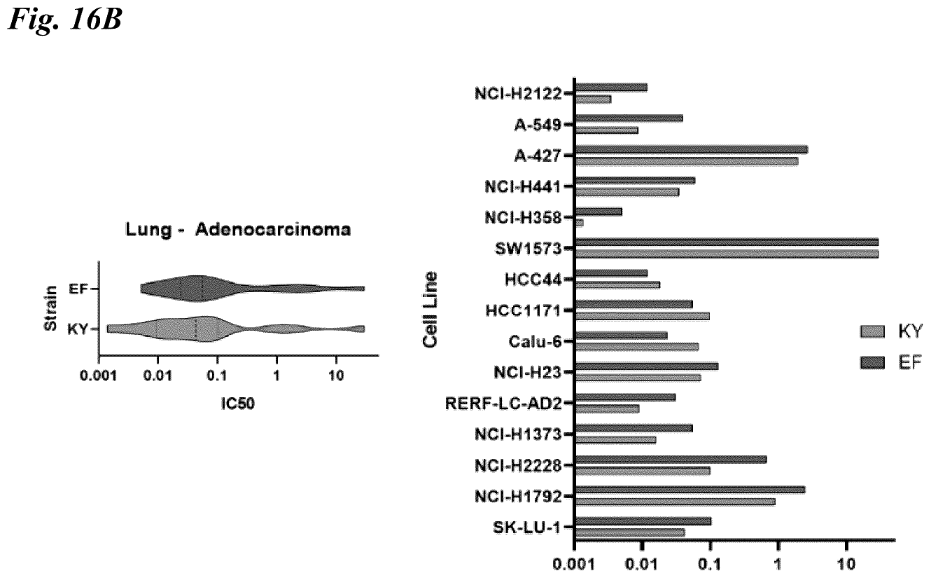

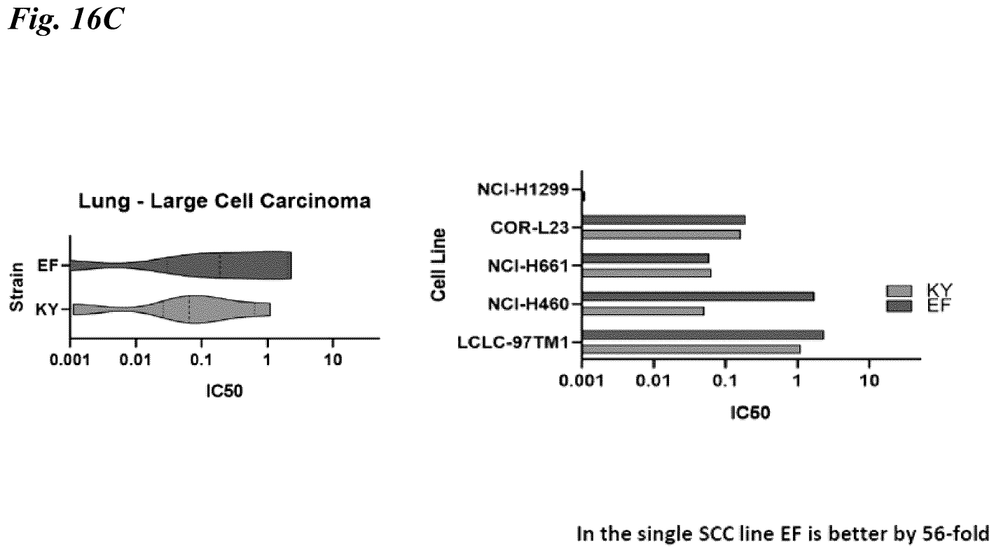

As shown in FIG. 16, the KY strain is more oncolytic in NSCLC/Adenocarcinoma in vitro models. Human NSCLC tumor microarray (n=97) was tested for ICAM-1 expression via immunohistochemistry (see FIG. 16A). The oncolytic efficacy of EF and KY strains was also compared across these cell line models. As shown in FIG. 16B, the KY strain demonstrated tumor cell lysis at a lower IC50 across multiple NSCLC/adenocarcinoma cell lines compared to the EF strain (see FIG. 16B, wherein the IC50 for the KY strain is lower in 11/15 NSCLC/adenocarcinoma cell lines). FIG. 16C also shows that the KY strain demonstrated a lower IC50 in large cell carcinoma cell lines (left panel).

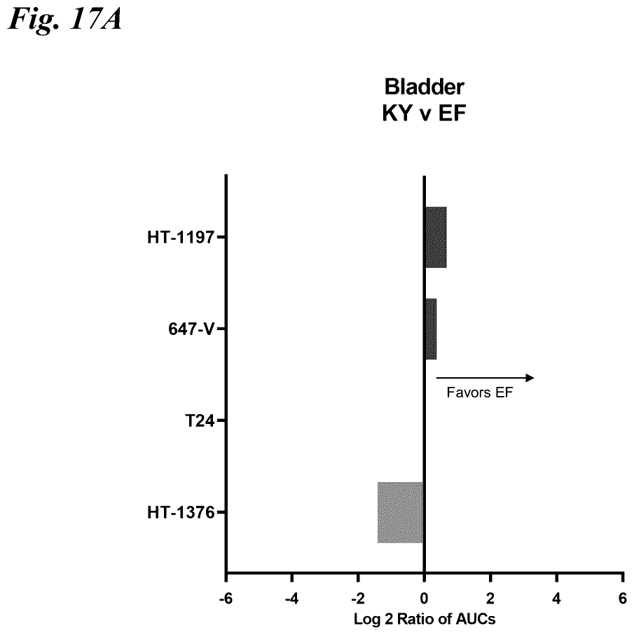

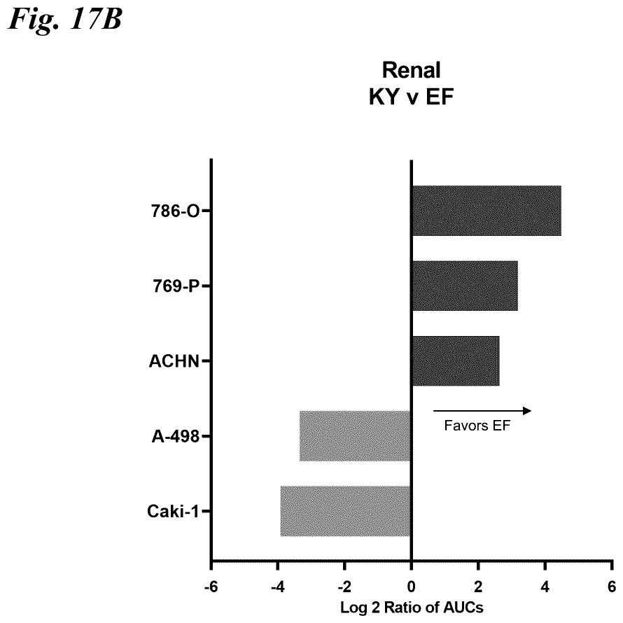

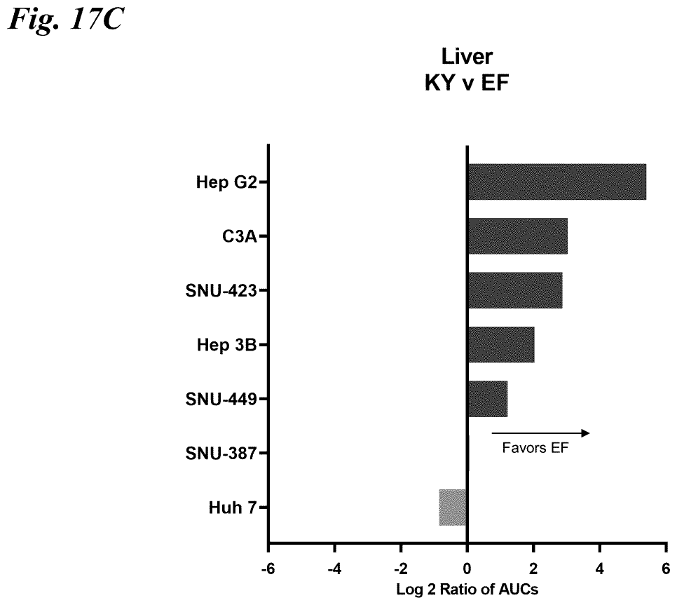

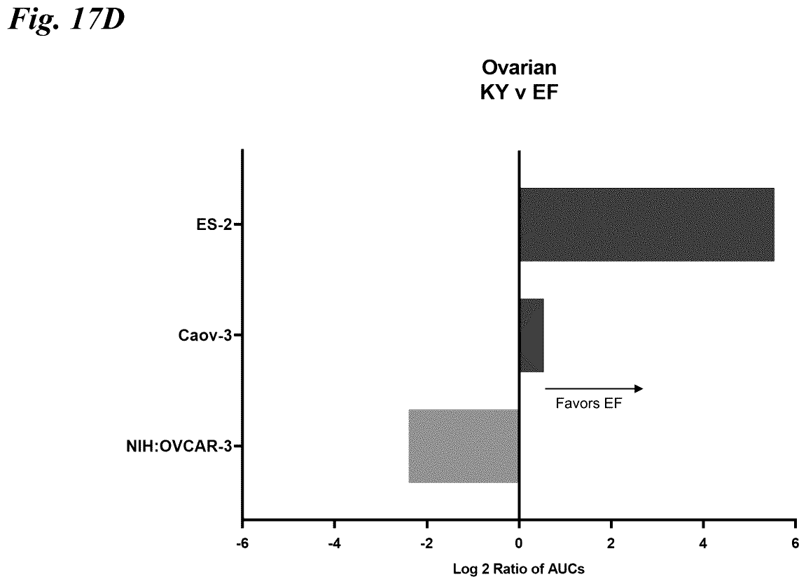

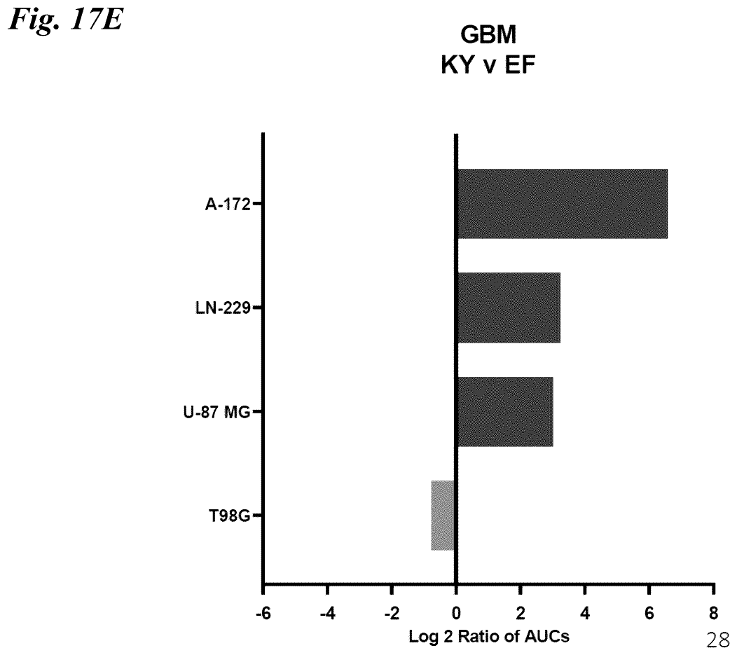

The EF strain demonstrated more favorable tropism in bladder (FIG. 17A), renal (FIG. 17B), liver (FIG. 17C), ovarian (FIG. 17D), and GBM (FIG. 17E) cell lines.

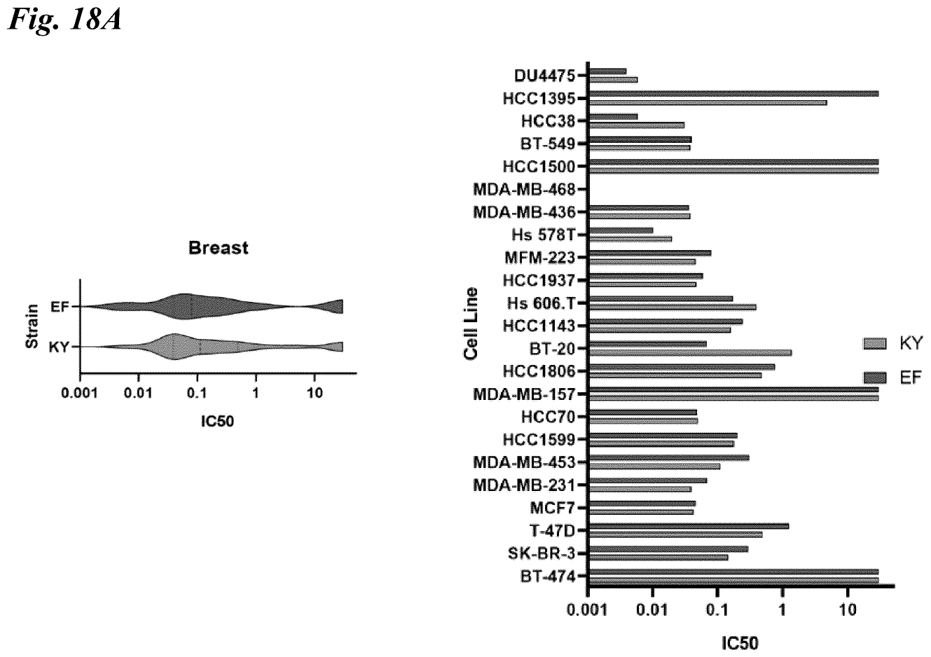

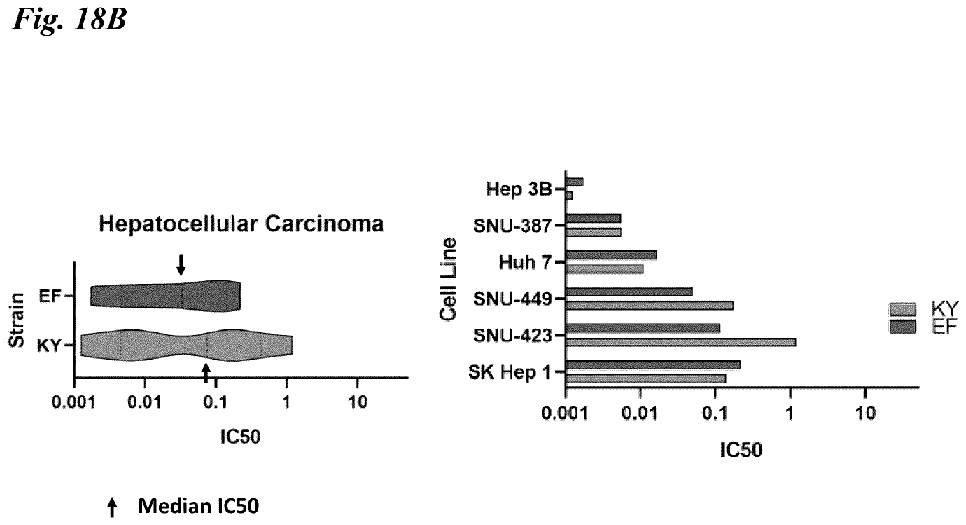

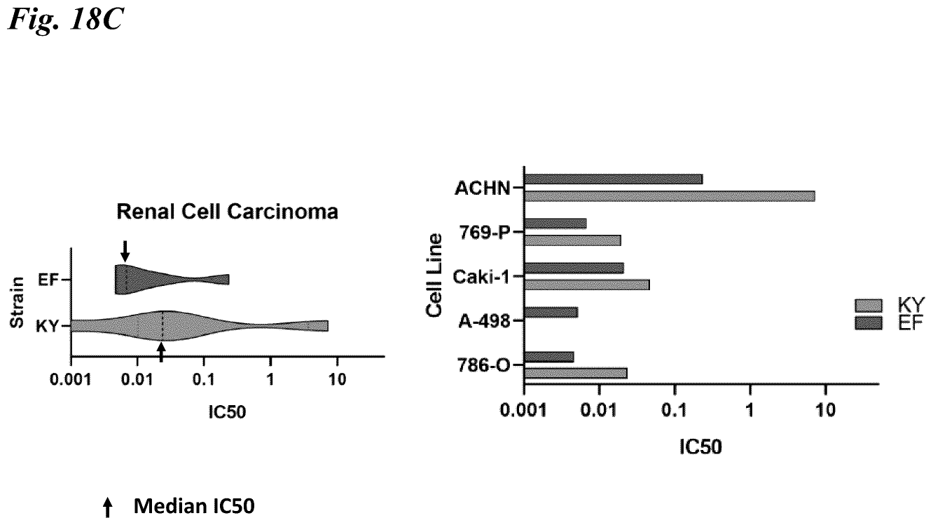

The KY and EF strains demonstrated similar oncolytic efficacy in breast cancer in vitro models (FIG. 18A). In renal cell carcinoma (RCC) and hepatocellular Carcinoma (HCC) in vitro models, the EF strain has a lower mean IC50 value than the KY strain, indicating that the EF strain has better oncolytic efficacy in HCC and RCC. See FIG. 17B, FIG. 17C, FIG. 18B and FIG. 18C.

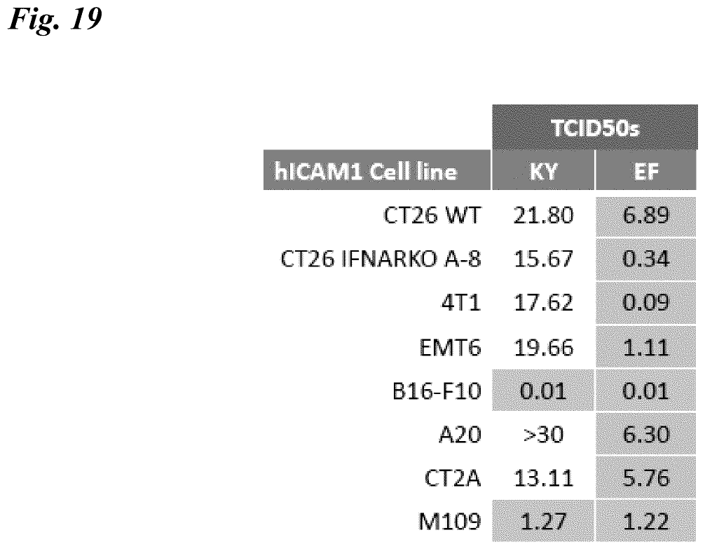

The EF strain is more potent in hICAM-1 syngeneic tumor models: Cell lines were transduced and selected for expression of hICAM-1. Cell lines were analyzed for sensitivity to infection by KY and EF strains of CVA21 by TCID50. As shown in FIG. 19, the EF strain outperformed the KY strain in all mouse lines tested with the only KY permissive BALB/c line being M109.

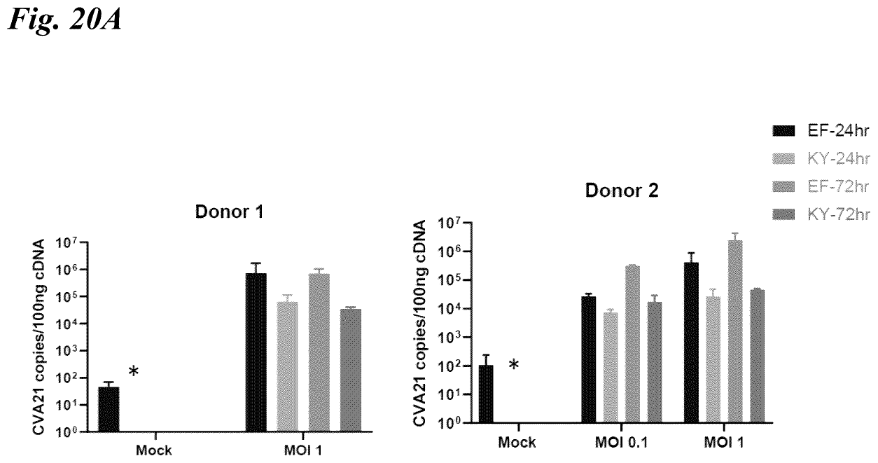

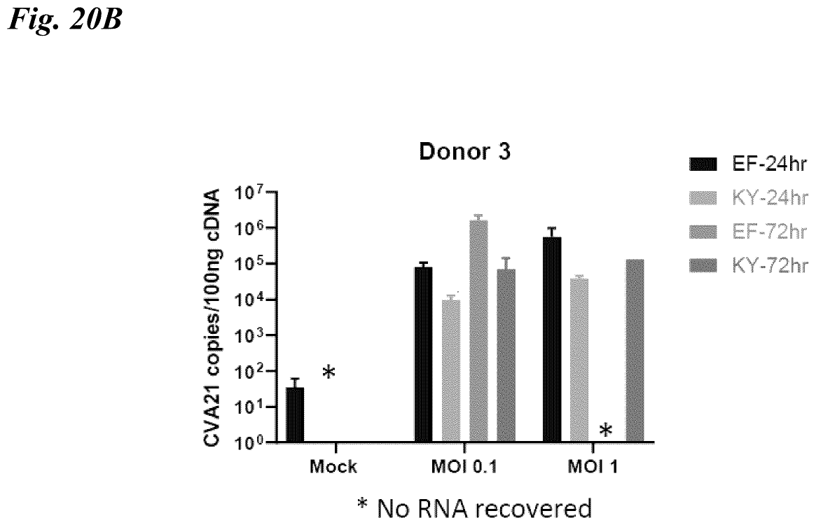

Further, the EF and KY strains replicate comparably in human dissociated tumor cells. NSCLC frozen dissociated tumor cells (DTC) were infected with KY and EF at 0.1 MOI. RNA was isolated from infected cells and was used for minus strand CVA21 qPCR to compare virus replication at 24 and 72 hours. Results from 3 donors are shown in FIG. 20A and FIG. 20B.

### In Vivo Efficacy:

The anti-tumor efficacy of these CVA21 strains (EF, KY, and Kuykendall (Ku)) were evaluated in vivo using mouse xenograft models. For these experiments, the RNA viral genomes of each strain were encapsulated in LNPs comprising a molar ratio of SS—OC:DSPC:Chol:BRIJ™ S100 of 49:22:28.5:0.5 mol %. For control groups, PBS buffer as well as LNPs encapsulating an SVV-neg viral genome were used. Tumor Growth Inhibition (TGI) percentage was calculated by comparing the % of tumor volume change in treatment arms relative to the control. The % TGI is defined as (1−(mean volume of treated tumors)/(mean volume of control tumors))×100%.

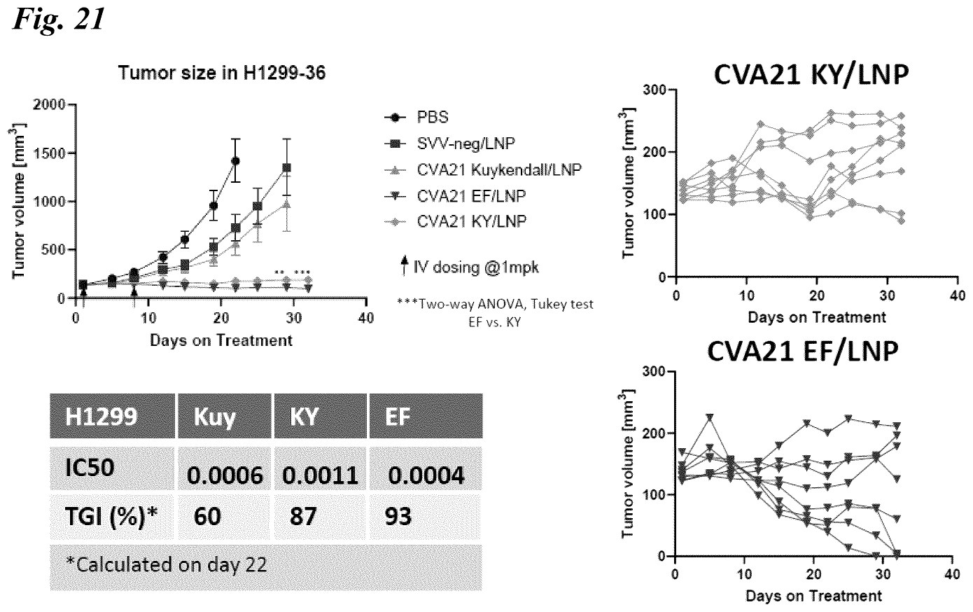

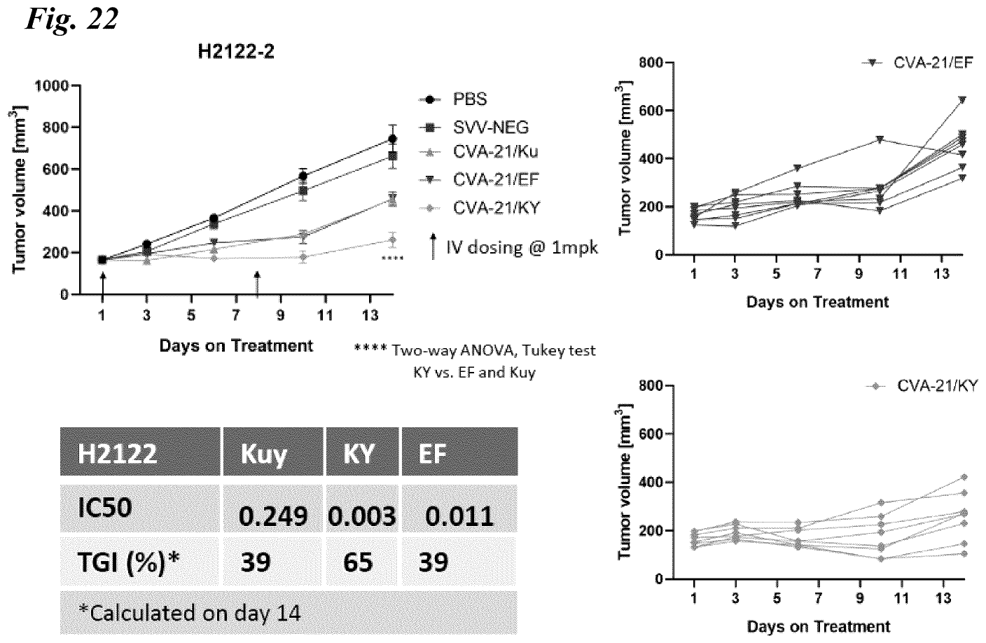

The EF and KY strain efficacy in an NCI-H1299 xenograft model of NSCLC is illustrated in FIG. 21. Both strains resulted in a significant reduction in tumor size compared to the Kuykendall strain and an SVV-neg synthetic RNA lipid nanoparticle. The EF strain appeared to work better than the KY strain in the NCI-H1299 xenograft model. The EF strain resulted in complete responses in 3 out of 8 animals, as compared to complete response in 1 out of 8 animals for the KY strain. The EF and KY strain efficacy in an NCI-H2122 xenograft model of NSCLC is illustrated in FIG. 22. The KY strain resulted in a significant reduction in tumor size compared to the Kuykendall strain, the EF strain, and an SVV-neg synthetic RNA lipid nanoparticle.

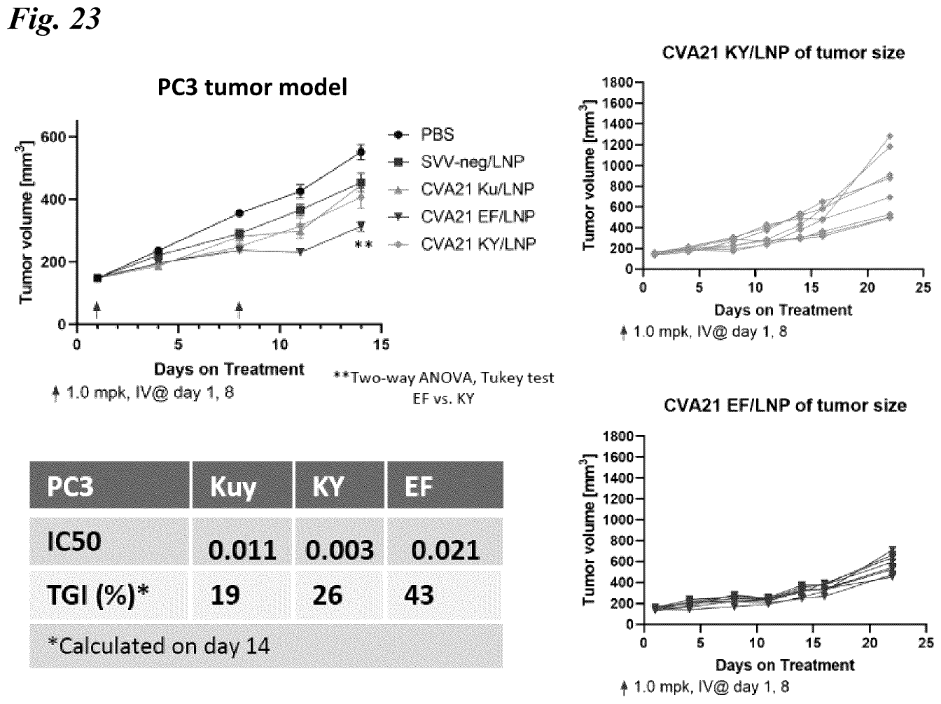

The EF and KY strain efficacy in a PC3 xenograft model of prostate cancer is illustrated in FIG. 23. The EF strain resulted in a significant reduction in tumor size compared to the Kuykendall strain, the KY strain, and an SVV synthetic RNA lipid nanoparticle.

### Strain Tolerability:

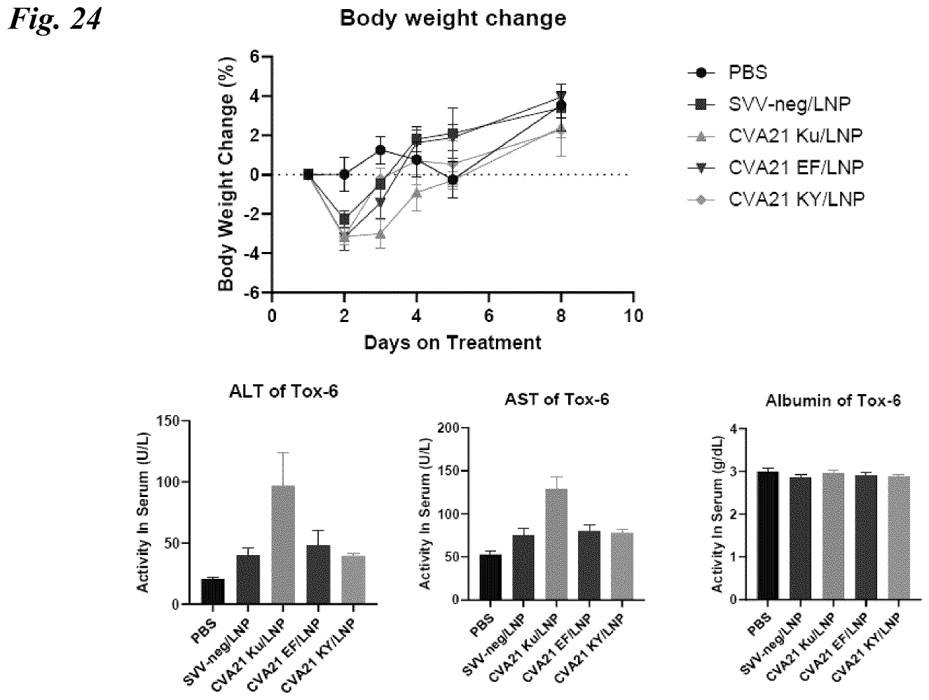

Tolerability of these CVA21 strains (EF, KY, and Kuykendall (Ku)) were also evaluated in vivo using a hICAM-1 transgenic mouse model. A tolerability study was conducted by dosing the mice with LNPs comprising the RNA viral genomes as indicated in FIG. 24 at a single intravenous dosage of 1 mg/kg. Necropsy was examined at 48 hours and 7 days post dosing. No in-life clinical toxicity signs were observed across the tested agents other than transient body weight loss (FIG. 24, upper panel). Live chemistry changes were minimal (FIG. 24, lower panels).

No CVA21 treatment-related macroscopic or microscopic pathological findings were noted in the brain, spinal cord, liver, kidney, lung, heart, and muscle with the Kuy, KY, and EF CVA21 strains (data not shown). Mild and sporadic microscopic findings such as increased mitosis of Kupffer cells were observed primarily in the liver, which is attributed to the LNP since the same finding was also observed with LNP vRNA negative control (SVV-neg/LNP) (data not shown). Other minor sporadic findings were all considered secondary to peri- or post-mortem procedures or not associated with the administration of LNP-RNA viral genome (data not shown). Overall, these results indicate that Synthetic EF, KY, and Kuykendall CVA21 were equally well tolerated in sensitive hICAM-1 transgenic mice.

### Virus Clearance:

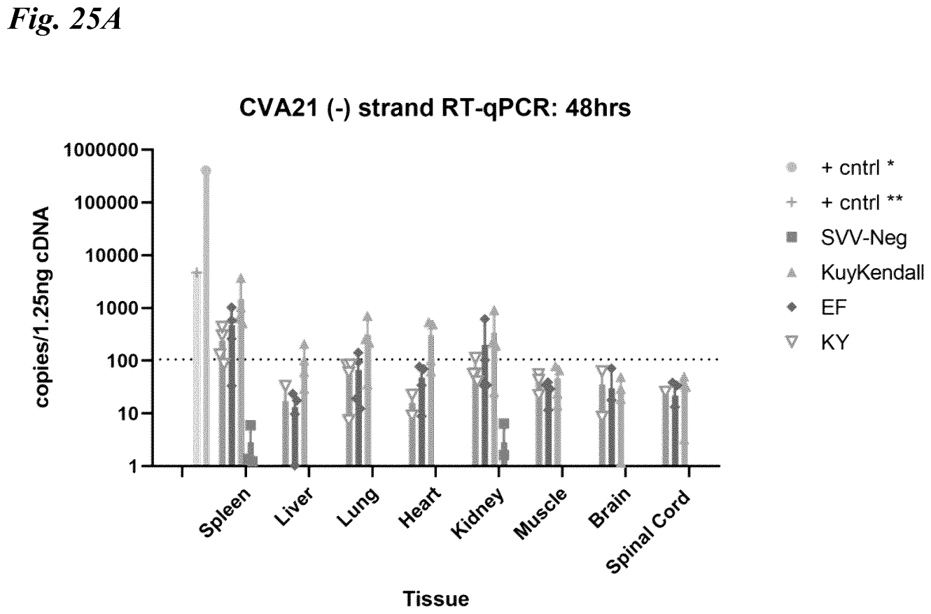

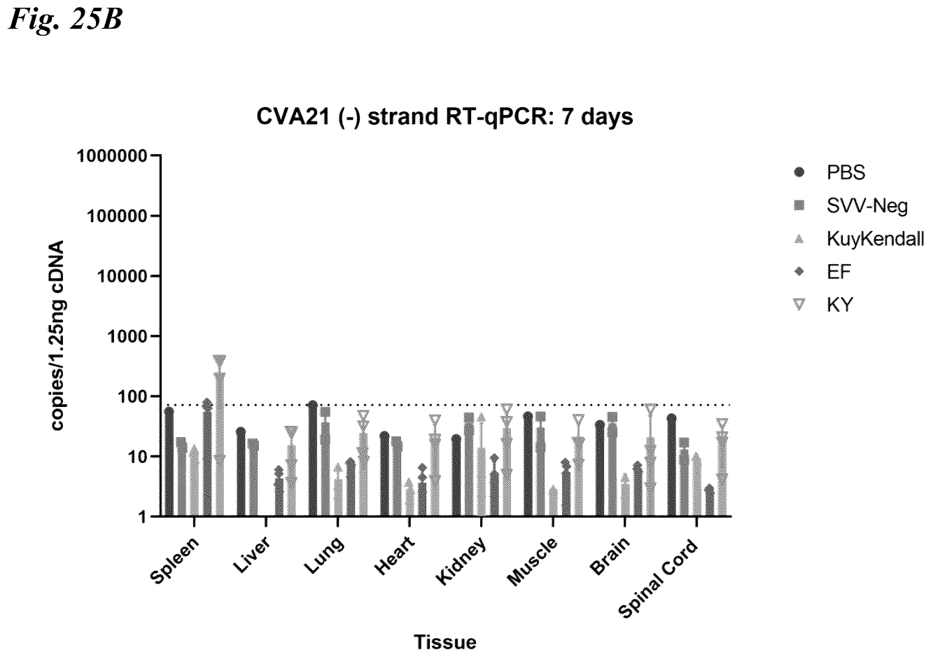

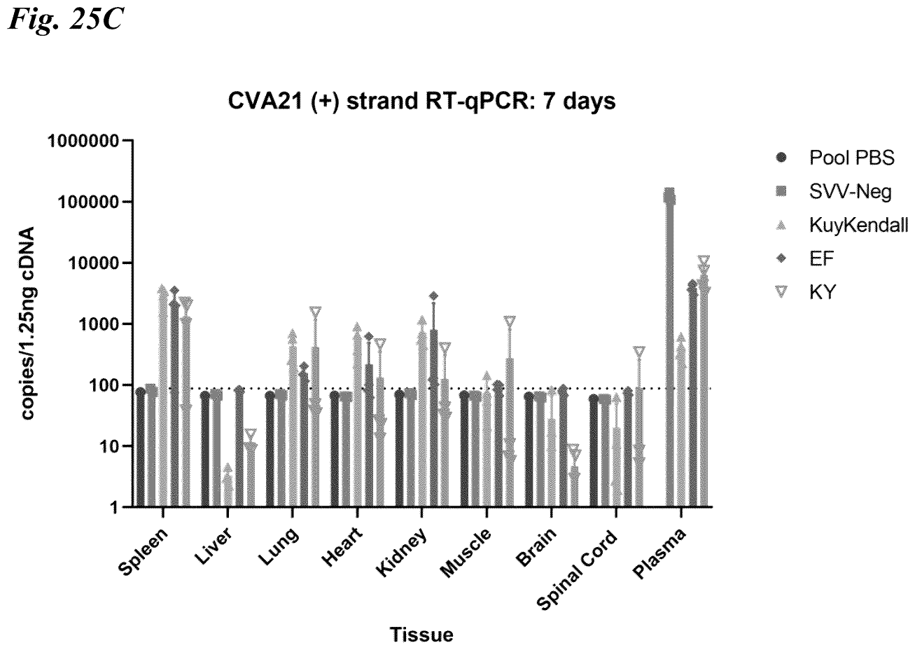

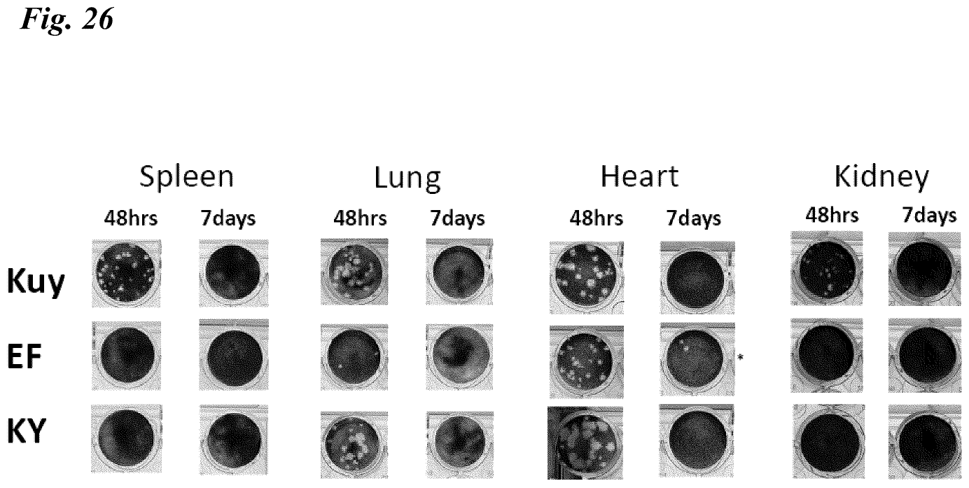

Viral replication was studied by RT-qPCR of the minus and positive strands of the viral genome, as well as viral plaque assay, at 48 hours and 7 day time points (FIG. 25A-FIG. 26). At 48 hrs post infection, the Kuykendall strain replicated to the highest minus strand RNA titer in spleen, liver, heart, and kidney, and CVA21 virus was detected in all strains (FIG. 25A). In contrast, all these viruses are mostly cleared in tissues by day 7 post infection as shown in FIG. 25B, in which the lack of minus strand detection indicates the lack of viral replication. Results of viral plaque assays support this conclusion—only the EF strain remained detectable by plaque titer at the 7-day time-point in the heart in 2 out of 4 animals (FIG. 26). On the other hand, the presence of (+) strand viral RNA detected in plasma at 7-day time point (FIG. 25C) suggests LNP circulation in blood after 7 days.

### Interferon (IFN) Resistance:

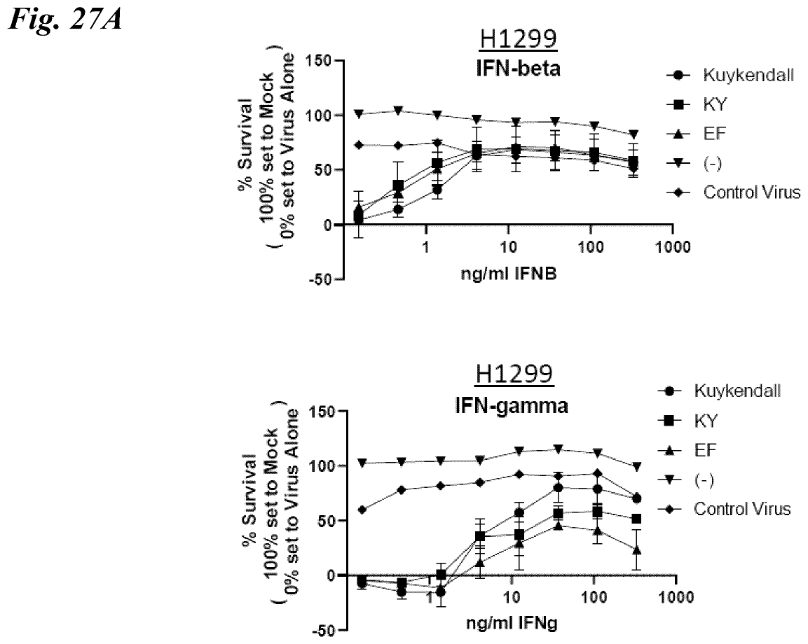

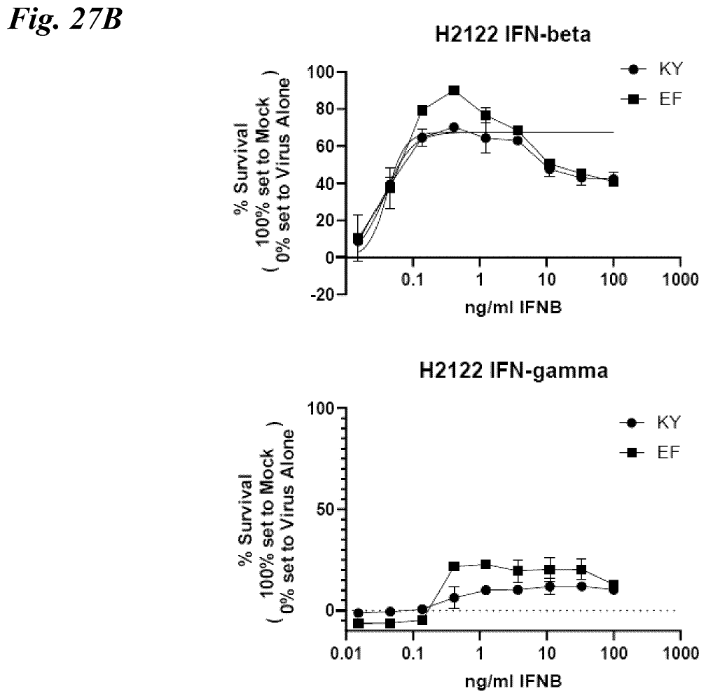

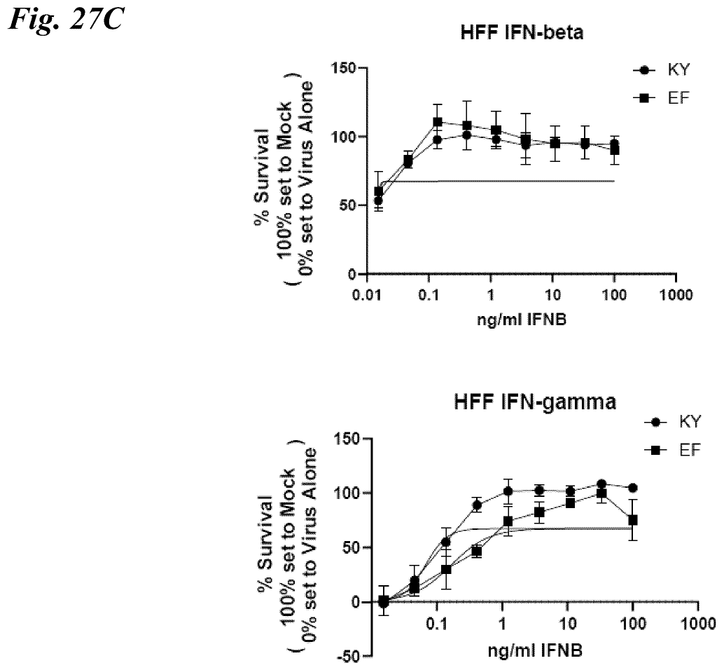

NSCLC cell lines NCI-H1299 (FIG. 27A) and NCI-H2122 (FIG. 27B) and HFF (Human foreskin fibroblasts, FIG. 27C) were pretreated with IFNβ or IFNγ before infection with CVA21 EF, KY, or Kuykendall strains, and cell survival was plotted against concentration of IFN pretreatment. The results are summarized in FIG. 27A-FIG. 27C and show that the EF and KY strains demonstrate similar interferon (IFN) resistance.

### Fidelity and Recombination Rate:

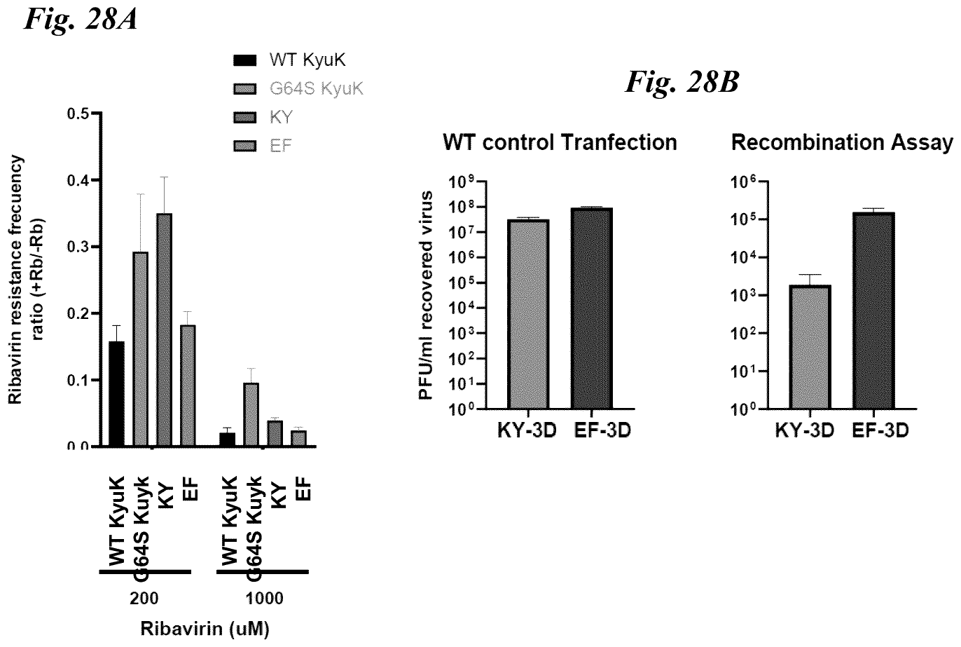

Fidelity of strains was tested by pretreating NCI-H1299 cells for 4 hours with ribavirin before 0.1 MOI infection of CVA21 strains including KYI, EF, Kuykendall, and Kuykendall G64S (a high fidelity Kuyk variant). Cells were harvested 24 hours after treatment and titered. The results are shown in FIG. 28A and KY strain displayed similar resistance to 200 μM of ribavirin as the high-fidelity Kuykendall G64S strain. To test recombination rate, the CVA21 strain KuyK was made with the 3D pol of the EF strain or the KY strain as the complete virus (WT), lacking capsid proteins (Δcap) and with a dead polymerase (ΔGDD). The two inactive forms (Δcap and ΔGDD) of KuyK chimeric virus comprising 3D pol of the KY or EF strain were transfected together into KuyK non-permissive 293TME. Recombined viable virus recovered after 72 h was then titered. The results are summarized in FIG. 28B. 3D pol of the KY strain displayed much lower recombination rate than the corresponding 3D pol of the EF strain, as KY-3D condition had ˜80 fold less recovered virus in the recombination assay. Overall, the KY strain exhibits higher baseline fidelity and lower recombination than the EF strain.

### Example 10: Leader Sequence Selection for CVA21

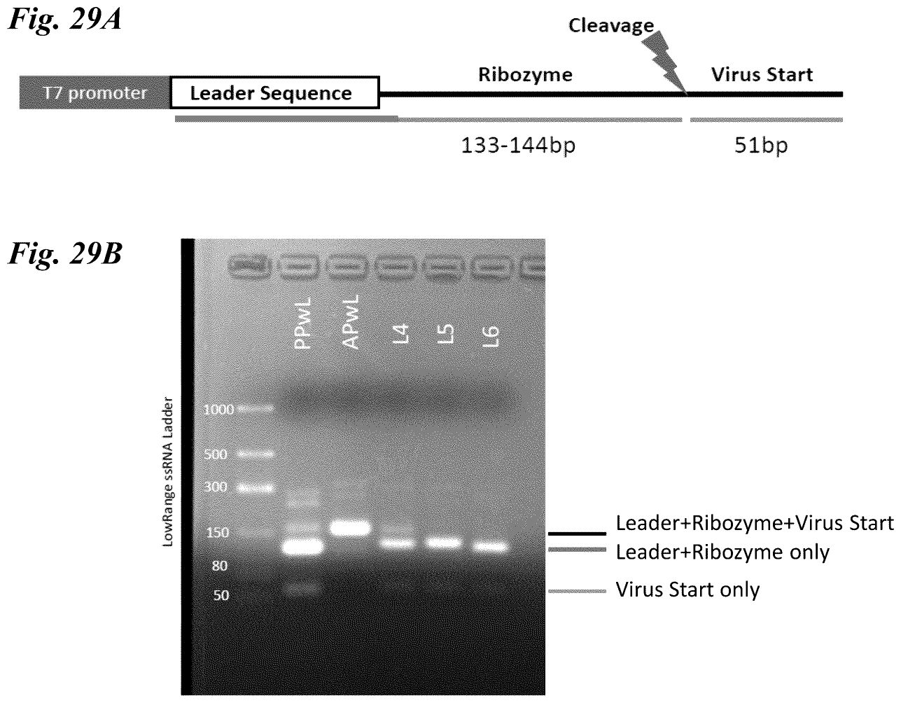

Experiments were performed to determine the leader sequence that promotes precise and complete cleavage of the ribozyme sequence at the 5′ junctional cleavage sequence region of the in vitro transcription template for CVA21. Viennafold program was used to generate a library of leader sequences that were predicted to form no secondary structure that may compete with ribozyme folding. A DNA library was generated comprising the leader sequences inserted into a template construct as shown in FIG. 29A. The DNA library was then subject to in vitro transcription, and the RNA product of each construct was purified and loaded onto a 2.5% TBE agarose gel to examine the result of ribozyme cleavage. FIG. 29B shows the result of three new leader sequences including CVA21-L4 (SEQ ID NO: 13), CVA21-L5 (SEQ ID NO: 14), and CVA21-L6 (SEQ ID NO: 15), along with a negative control (ApwL). Both CVA21-L5 and CVA21-L6 leader sequences display precise and complete cleavage of the ribozyme sequence, as only bands of cleavage products (leader+ribozyme fragment and virus start fragment) were visible in the corresponding lanes.

### Example 11: Optimization of 5′ Ribozyme Sequence for CVA21

Experiments were performed to optimize the ribozyme sequence at the 5′ junctional cleavage sequence region of the in vitro transcription template for CVA21. A library of ribozyme variant sequences based on Pistol ribozyme from P. polymyxa were screened; these variant sequences differed at the nucleic acid positions corresponding to nucleic acid positions 27-30 of SEQ ID NO: 17. In vitro transcription (IVT) templates were generated with random sequence complementary to the initiation of viral sequence in the P2 stem. IVT was performed and cleaved segments were isolated and small RNA-seq was performed to determine the sequence composition of the elements that were cleaved at high frequency.

Initial screening results showed that, compared to SEQ ID NO: 17 which comprises the nucleotides “TTTA” at the positions 27-30 of SEQ ID NO: 17, two variant ribozyme sequences are represented at a higher frequency. One is SEQ ID NO: 18, which comprises “TTTT” at the corresponding positions; the other is SEQ ID NO: 19, which comprises “TTGT” at the corresponding positions.

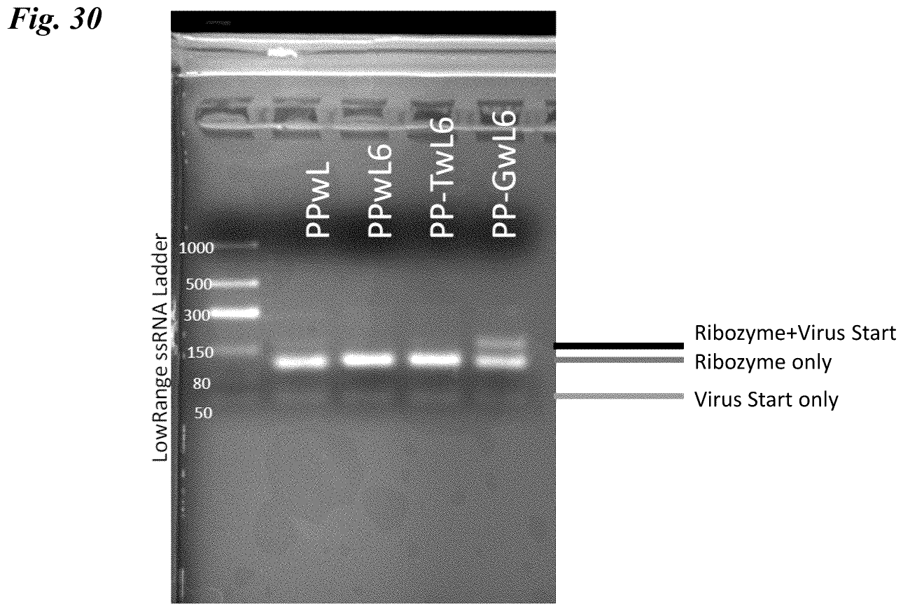

The junctional cleavage efficiency of these sequences was tested using a template construct design and in vitro transcription experimental setup similar to those in Example 10 above. FIG. 30 shows the result of four designs: (1) PPwL, comprising a ribozyme sequence according to SEQ ID NO: 17 and an alternative leader sequence; (2) PPwL6, comprising leader sequence L6 and a ribozyme sequence according to SEQ ID NO: 17; (3) PP-TwL6, comprising leader sequence L6 and a ribozyme sequence according to SEQ ID NO: 18; and (4) PP-GwL6, comprising leader sequence L6 and a ribozyme sequence according to SEQ ID NO: 19. The results demonstrated that the PP-TwL6 design has a cleavage efficiency close to 100%, which is similar or better than the PPwL6 design and much higher than the PP-GwL6 design, and therefore SEQ ID NO: 18 is superior to SEQ ID NO: 19 in this experimental setup. These results were recapitulated when the same designs were tested in the context of recombinant DNA templates encoding full CVA21-KY strain viral genomes.

A general schematic of a non-limiting example of the CVA21 expression construct design and corresponding in vitro transcription process to generate synthetic RNA viral genomes with precise ends at 5′ and 3′ is provided in FIG. 33.

### Example 12: Optimization of Poly-A Tail Sequence

Experiments were performed to optimize the length of the poly-A tail attached to the CVA21 viral genome or SVV viral genome.

Four different lengths of poly-A tails (30 pA, 50 pA, 70 pA, and 90 pA) were assessed by cloning into the corresponding region of the recombinant DNA molecule encoding the CVA21 or SVV viral genome. Purification assays were performed to assess the purification efficiency and recovery rate of the resulting RNA viral genomes on a monolith Oligo-dT chromatography at the following conditions: flow rate: 1 mL/min; loading concentration: 0.1 mg/mL; binding condition: 500 mM NaCl.

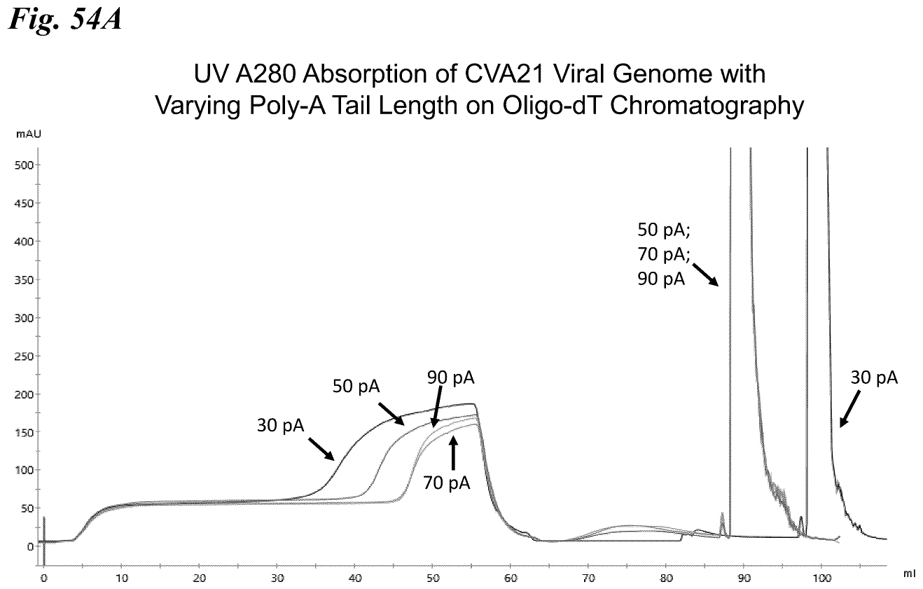

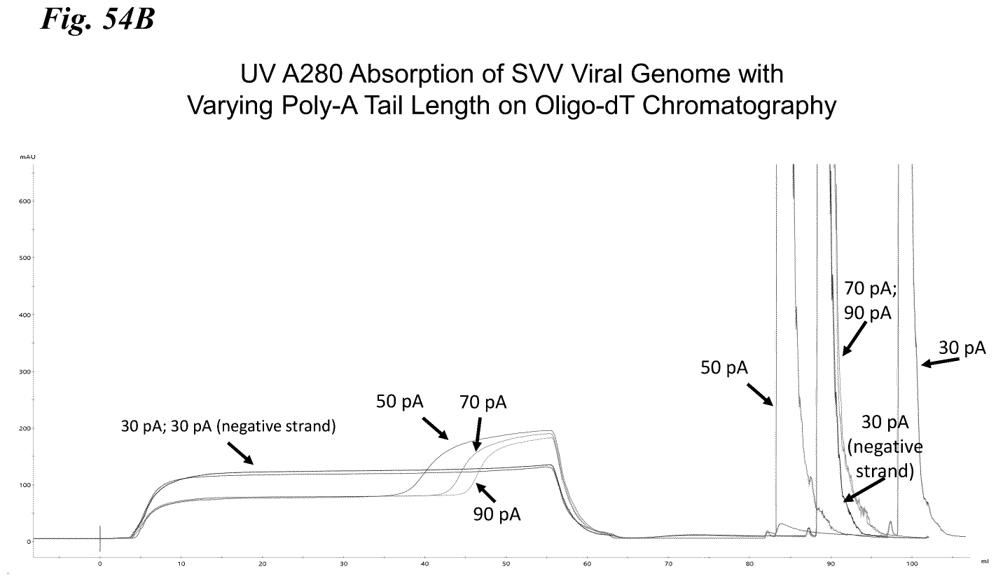

As shown in Table 15A below, a longer poly-A tail (>30 pA) resulted in a higher binding capacity and higher recovery rate of CVA21 RNA viral genome molecules after elution, but extending the length of poly-A tail beyond 70 pA provided minimal further improvement of purification efficiency. A representative chromatography profile is shown in FIG. 54A.

TABLE 15A
Oligo-dT Chromatography of CVA21-RNA
with Varying Poly-A Tail Lengths

| Poly-A Tail | Recovery % | Binding Capacity |
|---|---|---|
| Length | after Elution | (mg/mL) |
| 30 pA | 44.5 | 3 |
| 50 pA | 47.3 | 3.77 |
| 70 pA | 49.5 | 4.24 |
| 90 pA | 50.3 | 4.24 |

Similar experiments were performed to assess the purification efficiency and recovery rate of SVV RNA viral genome molecules with varying poly-A tail length, at the following conditions: flow rate: 1 mL/min; loading concentration: 0.1 mg/mL; binding condition: 500 mM NaCl. A negative strand control of SVV-RNA molecule with 30 pA tail was also included as an additional control.

As shown in Table 15B below, a longer poly-A tail resulted in higher binding capacity and higher recovery rate of SVV RNA viral genome molecules after elution but extending the length of poly-A tail beyond 70 pA provided minimal further improvement of purification efficiency. A representative chromatography profile is shown in FIG. 54B.

TABLE 15B
Oligo-dT Chromatography of SVV-RNA
with Varying Poly-A Tail Length

| Poly-A Tail | Recovery % | Binding Capacity |
|---|---|---|
| Length | after Elution | (mg/mL) |
| 30 pA | 33.1 | N/A |
| 50 pA | 52 | 3.41 |
| 70 pA | 56.3 | 3.9 |
| 90 pA | 57.7 | 4.12 |
| 30 pA | 32.4 | N/A |
| (negative strand) | | |

The synthetic RNA viral genomes with the 70 pA length poly-A tails showed increased binding capacity and recovery on oligo-dT chromatography column.

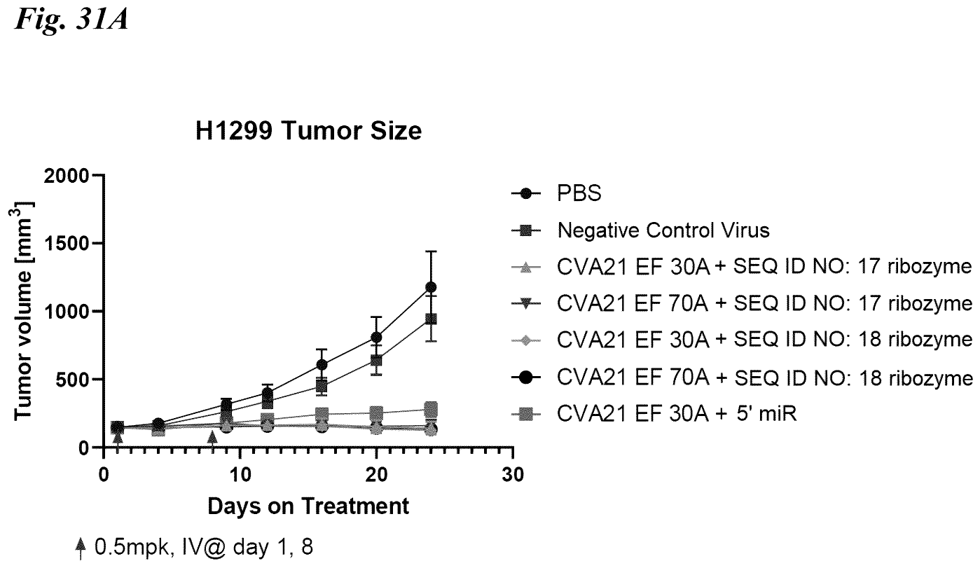

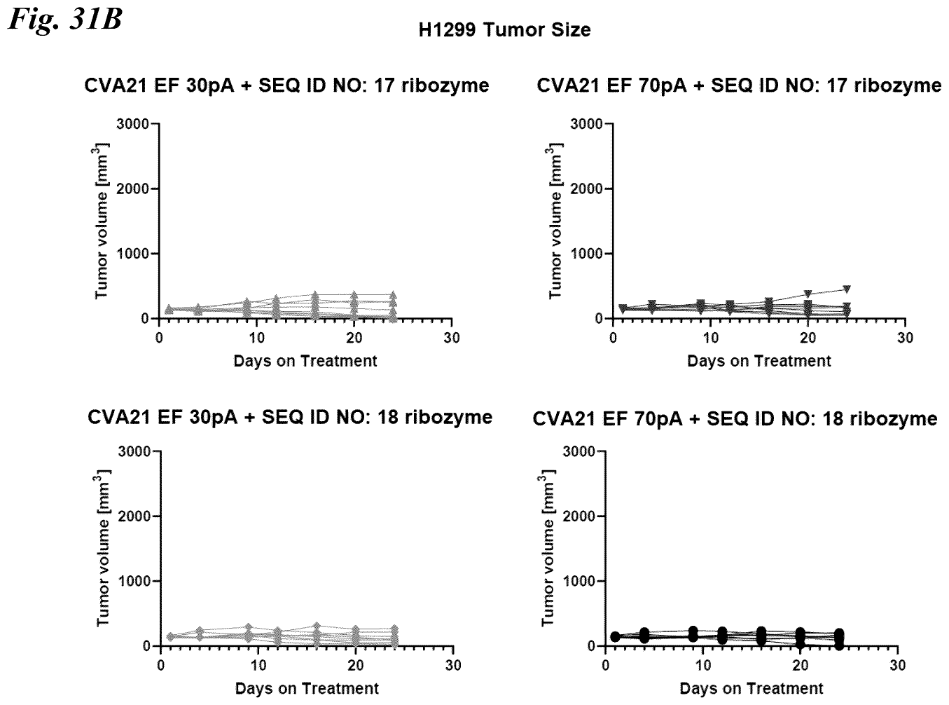

The anti-tumor efficacy of the synthetic CVA21-EF strain viral genomes produced with the 70 pA poly-A tail versus the 30 pA poly-A tail, and the ribozyme sequence SEQ ID NO: 18 versus SEQ ID NO: 17, were then compared. A mouse lung cancer model based on NCI-H1299 cells was used. As shown in FIG. 31A and FIG. 31B, the CVA21-EF strain viral genome produced with the 70 pA poly-A tail and the 5′ ribozyme sequence according to SEQ ID NO: 18 displayed similar or even better anti-tumor efficacy as compared to the other viral genome designs.

### Example 13: Construction of Chimeric SVVs and Test of their Oncolytic Potency

SVV virus comprising recombinant SVV RNA viral genomes were produced following similar procedures as those described in Example 1 above for CVA21. The discrete 5′ termini of SVV RNA vial genomes were generated using a 5′ Hammerhead ribozyme sequence or a 5′ Pistol ribozyme sequence following similar procedures as those described in Example 6 above. The discrete 3′ termini of SVV RNA viral genomes were generated using a 3′ hepatitis delta virus ribozyme sequence (via co-transcriptional cleavage), or a 3′ restriction enzyme recognition sequence (e.g., SapI, via template cleavage prior to in vitro transcription) following similar procedures as those described in Example 4 above.

### Generation of SVV Chimeric Viruses:

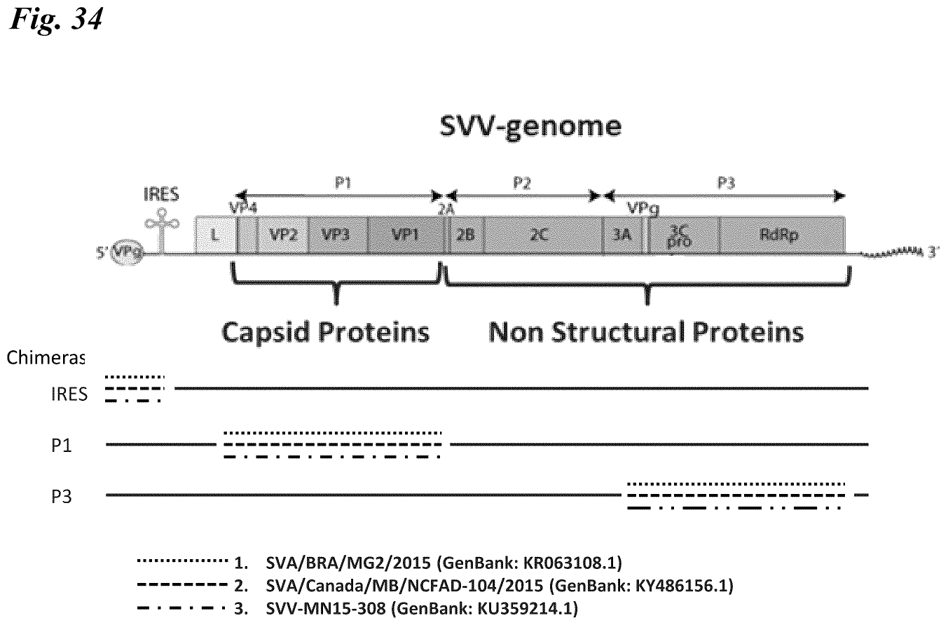

The SVV-001 virus (parental, SEQ ID NO: 25) was utilized to build chimeric viruses by swapping the IRES, P1 or P3 regions with the corresponding region of the different SVV strains as described in FIG. 34, including:
(a) SVV virus #1. SVA/BRA/MG2/2015 (GenBank: KR063108.1)
(b) SVV virus #2. SVA/Canada/MB/NCFAD-104/2015 (GenBank: KY486156.1)
(c) SVV virus #3. SVV-MN15-308 (GenBank: KU359214.1)

New SVV-GFP-pDNA constructs (SEQ ID NOs: 41-50) were transfected individually into H1299 cells to evaluate oncolytic activity based on GFP expression. Virions were collected from supernatants and used for further characterization. The chimeric viruses were named based on “region swapped—virus # of replaced sequence”. For example, “IRES2” indicates an SVV-001 parental virus with the IRES region replaced with the corresponding IRES region of the SVV virus #2 (SVA/Canada/MB/NCFAD-104/2015), “P1-1” indicates an SVV-001 parental virus with the P1 region replaced with the corresponding P1 region of SVV virus #1 (SVA/BRA/MG2/2015).

### Oncolytic Potency of SVV-IRES Chimeric Viruses:

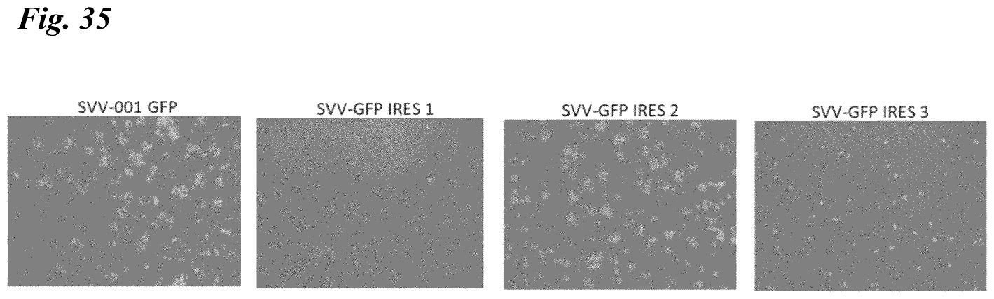

Clarified virions were used to infect NCI-H69AR cells. The intensity of GFP was measured 12 hours post infection at MOI 0.1. As shown in FIG. 35, the SVV-IRES1 chimeric virus (SEQ ID NO: 28) is oncolytic but does not express GFP. The SVV-IRES2 chimeric virus (SEQ ID NO: 29, containing IRES sequence from SVA/Canada/MB/NCFAD-104 swapped into the IRES region of SVV-001 parental genome) displays more robust oncolysis and stronger GFP expression. The SVV-IRES3 chimeric virus (SEQ ID NO: 30) showed less GFP expression that SVV-001 parental (SEQ ID NO: 27). In summary, the results indicate that SVV-IRES2 is an improved oncolytic virus with more robust oncolytic activity than the SVV-001 parental virus.

The SVV-IRES2 chimeric virus was further tested for its sensitivity to interferon alpha (IFNα). Viral replication and resistance to IFNα were tested in both sensitive (NCI-H69AR) and partially resistant (NCI-H69) SCLC cell lines. Cells were pretreated with INFa at 100 U/mL for 2 hours before SVV infection at MOI 0.1. SVV viruses were added to cells and GFP intensity was calculated. As shown in FIG. 36, the SVV-IRES2 chimeric virus demonstrated increase in viral replication and resistance to IFNα in both cell lines. Therefore, this chimeric virus is more robust and IFNα-resistant than the SVV-001 parental virus.

### Oncolytic Potency of SVV-P1 Chimeric Viruses.

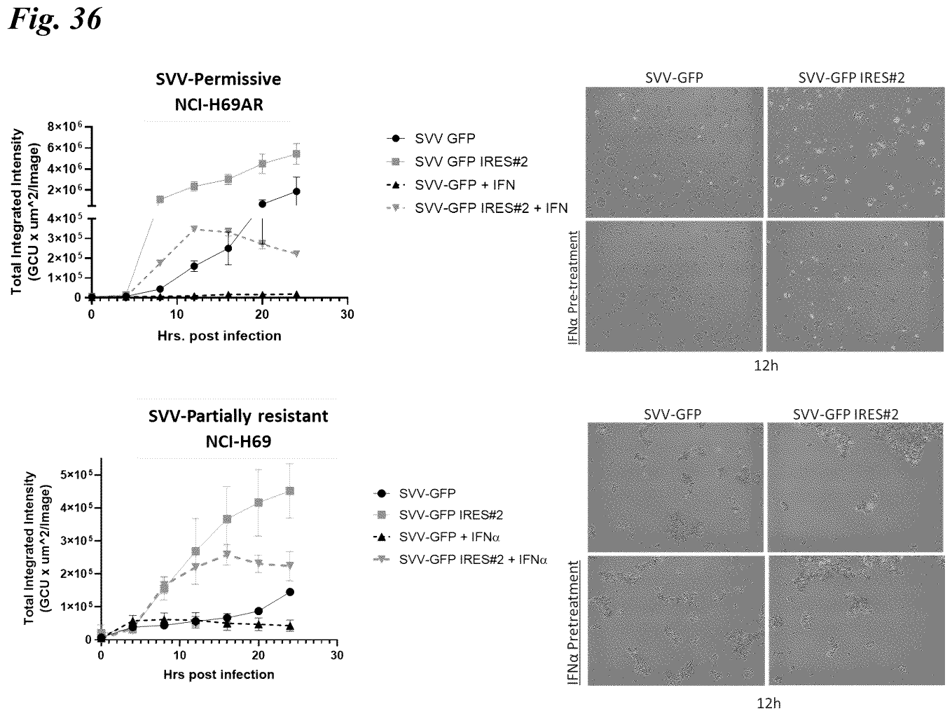

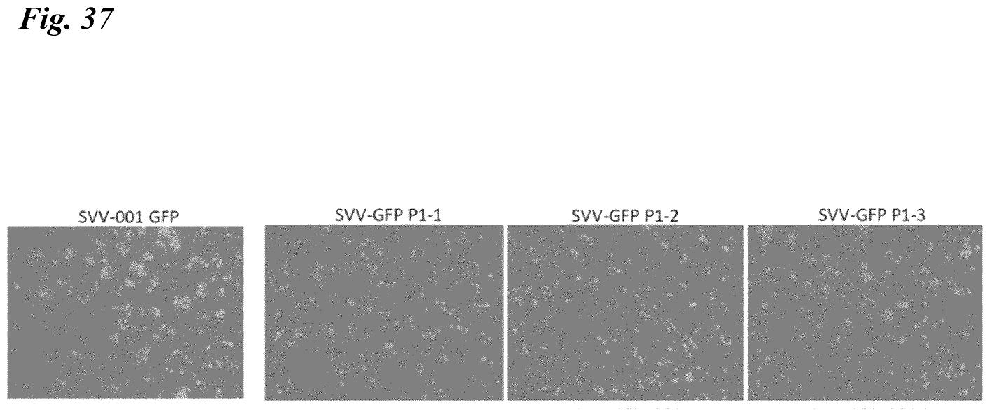

Clarified virions were used to infect NCI-H69AR cells. GFP intensity was measured 12 hours post infection at MOI 0.1. The chimeric virus tested were SVV-P1-1 (SEQ ID NO: 31), SVV-P1-2 (SEQ ID NO: 32) and SVV-P1-3 (SEQ ID NO: 33). As shown in FIG. 37, none of the new SVV-P1 chimeric viruses displayed increased oncolytic potency compared to SVV-001 parental.

### Oncolytic Potency of SVV-P3 Chimeric Viruses.

In FIG. 38A, clarified virions were used to infect NCI-H69AR cells. GFP intensity was measured 48 hours post infection at MOI 0.1. The P3-1 chimeric virus (SEQ ID NO: 34) showed lower GFP expression and the P3-3 chimeric virus (SEQ ID NO: 36) showed similar GFP expression as the SVV-001 parental virus. In FIG. 38B, clarified virions were used to infect NCI-H₆₉AR cells. GFP intensity was measured 12 hours post infection at MOI 0.1. SVV-P3-2 chimeric virus (SEQ ID NO: 35) displayed decreased oncolytic potency compared to SVV-001 parental. Therefore, none of these SVV-P3 chimeric viruses display increased oncolytic potency compared to the SVV-001 parental virus.

### Example 14: Leader Sequence Selection for SVV

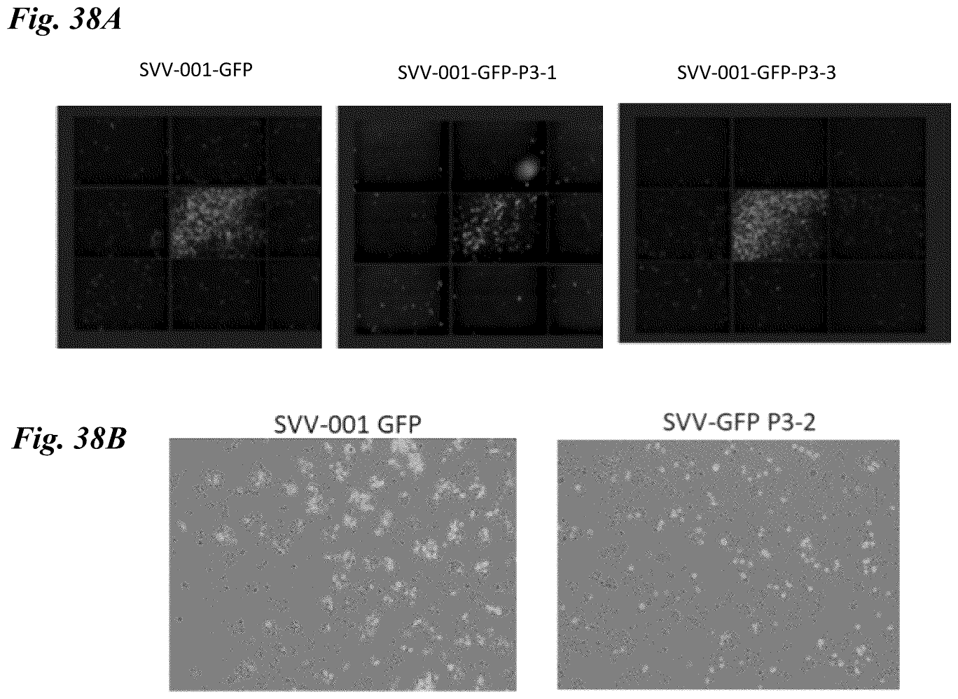

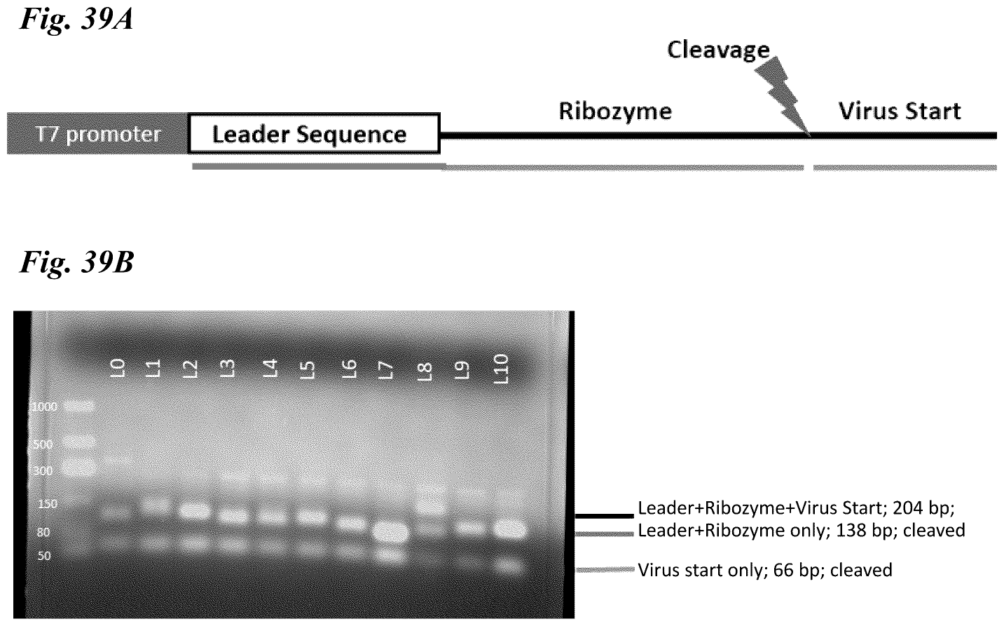

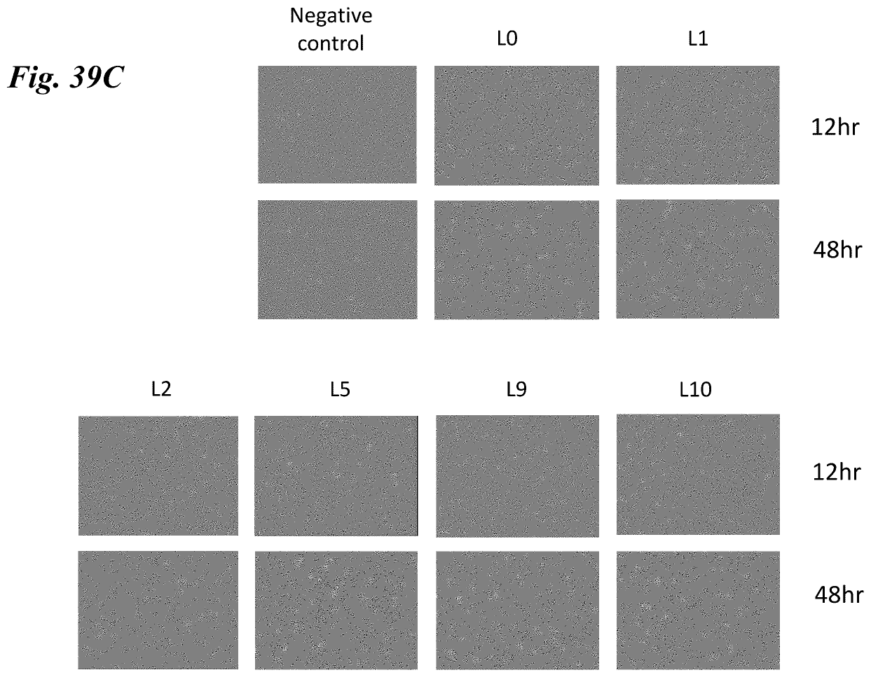

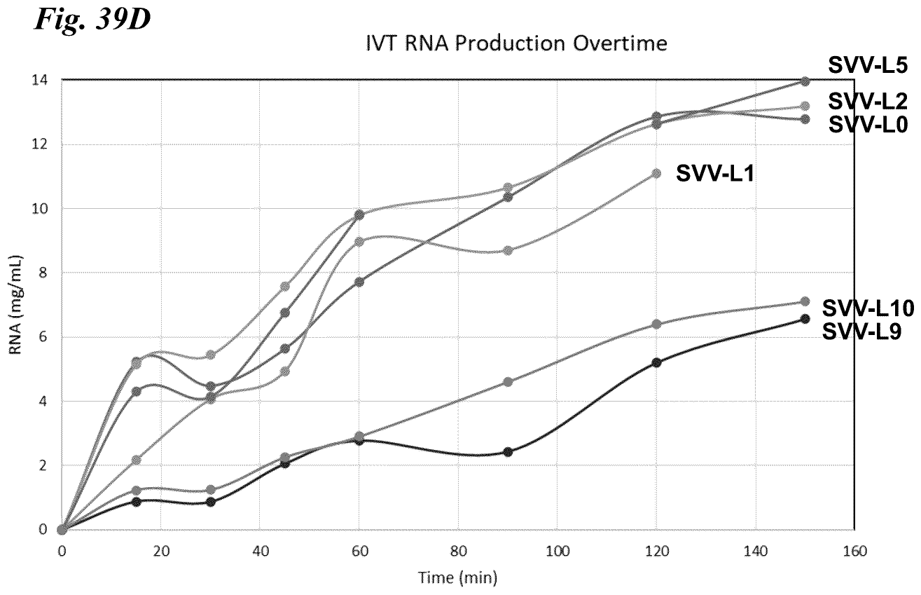

Experiments were performed to determine the leader sequence that promotes precise and complete cleavage of Pistol 1 ribozyme sequence (SEQ ID NO: 64) at the 5′ junctional cleavage sequence region of the in vitro transcription template for SVV. Viennafold program was used to generate a library of leader sequences that were predicted to form no secondary structure that may compete with ribozyme folding. A number of constructs was generated comprising the lead sequences inserted into a template construct as shown in FIG. 39A. The constructs were then subject to in vitro transcription, and the RNA product of each construct was purified and loaded onto a 2.5% TBE agarose gel to examine the result of ribozyme cleavage. FIG. 39B shows the result of 11 leader sequences tested, including SVV-L0 (SEQ ID NO: 53), SVV-L1 (SEQ ID NO: 54), SVV-L2 (SEQ ID NO: 55), SVV-L3 (SEQ ID NO: 56), SVV-L4 (SEQ ID NO: 57), SVV-L5 (SEQ ID NO: 58), SVV-L6 (SEQ ID NO: 59), SVV-L7 (SEQ ID NO: 60), SVV-L8 (SEQ ID NO: 61), SVV-L9 (SEQ ID NO: 62), and SVV-L10 (SEQ ID NO: 63). With the exception of SVV-L8, all other leader sequences tested promote precise and complete cleavage of the 5′ ribozyme sequence, since only bands of cleavage products (leader+ribozyme fragment and virus start fragment) are visible in those corresponding lanes.

SVV DNA templates comprising the leader sequence of SVV-L0, SVV-L1, SVV-L2, SVV-L5, SVV-L9 or SVV-L10 were used for in vitro transcription and the resultant SVV RNA viral molecules from each DNA template were tested for their ability to generate infectious viral particles in an in vitro viral kick-off assay. H1299 cells were transfected with 1 μg of each transcribed viral RNA and the plaque titers were analyzed 12 hrs and 48 hrs post-transfection. As shown in FIG. 39C and Table 17, the viral titers were similar for SVV RNA transcripts generated from each of the DNA templates comprising different leader sequences.

Each of the above DNA templates was tested for in vitro transcription efficiency.

For each of these DNA templates, an in vitro transcription reaction was carried out in 3 mL volume at 37° C. for 2.5 hours followed by 30 minutes of DNaseI treatment. The in vitro transcription products were analyzed using RP-HPLC as shown in FIG. 39D. The results showed that SVV-L1, SVV-L2, and SVV-L5 leader sequences provide similar in vitro transcription yield as the original SVV-L0 leader sequence, whereas SVV-L9 and SVV-L10 leader sequences only provide about half of the yield.

Previously, over 1000 candidate leader sequences were generated and the transcription efficiency of the corresponding DNA templates was predicted and ranked according to Conrad, T. et al. Commun Biol 3, 439 (2020) (as shown in Table 16 below). Notably, leader sequence SVV-L5 had a low rank in the prediction but showed unexpectedly robust yield in actual in vitro transcription experiment.

TABLE 16
Predicted Transcription Efficiency of
SVV DNA Template with Leader Sequence

| Leader | Rank of T7 Promoter according to |
|---|---|
| Sequence | the Prediction of Conrad, T. et al |
| SVV-L1 | 7 |
| SVV-L2 | 20 |
| SVV-L10 | 99 |
| SVV-L9 | 658 |
| SVV-L0 | 659 |
| SVV-L5 | 817 |

After 5′ ribozyme cleavage, the leader sequence-ribozyme fragment may non-specifically interact with the transcribed SVV RNA viral genomes, lowering the purification efficiency of the RNA viral genomes on the Oligo-dT column. An optimized leader sequence should ideally minimize such non-specific interaction during the purification. For each of these DNA templates comprising specific leader sequence, the transcription product was subjected to Oligo-dT chromatography purification at a denaturing temperature of 80° C., and the RNA products eluted from the Oligo-dT chromatography were analyzed for the presence of ribozyme fragment and purity of RNA viral genome. As shown in Table 17 below, cleaved 5′ ribozyme fragment comprising SVV-L5, SVV-L9, or SVV-L10 leader sequences were much more effectively removed by Oligo-dT chromatography than the cleaved 5′ ribozyme fragment comprising SVV-L0, SVV-L1, or SVV-L2 leader sequences.

TABLE 17
Purity of SVV RNA Viral Genomes after
Denaturing Oligo-dT Chromatography

| Leader Sequence in | Ribozyme Fragment | Purity of RNA |
|---|---|---|
| the DNA Template | [%] | viral genome [%] |
| SVV-L0 | 2.31 | 86.9 |
| SVV-L1 | 2.42 | 84.6 |
| SVV-L2 | 0.49 | 87 |
| SVV-L5 | 0.15 | 85.1 |
| SVV-L9 | 0.12 | 83.5 |
| SVV-L10 | 0.07 | 84.5 |

Accordingly, compared to other leader sequences tested, SVV-L5 leader sequence shows superior in vitro transcription yield when incorporated into the DNA template. In addition, SVV-L5 leader sequence also minimizes the non-specific interaction between the corresponding 5′ ribozyme cleavage product and the RNA viral genome, resulting in effective removal of the 5′ ribozyme cleavage product during chromatography purification.

### Example 15: LNPs Comprising Brij and SVV Viral RNA Demonstrate High Anti-Tumor Efficacy in Animal Models

The anti-tumor efficacy of lipid nanoparticles (LNPs) comprising Brij molecule (as PEG lipid) and SVV RNA viral genome was tested using various animal models. Such SVV/LNPs were produced following similar procedures as those described in Example 2 above.

The LNP formulations used in this example are provided in Table 10.

TABLE 10
SVV-RNA LNP Formulations

| Formulation ID | Cationic Lipid | Cholesterol | DSPC | PEG-Lipid |
|---|---|---|---|---|
| 96062-1 | SS-OC - 49% | 28.5% | 22% | BRIJ ™ S100 - 0.5% |
| 96062-2 | SS-OP - 49% | 28.5% | 22% | BRIJ ™ S100 - 0.5% |
| 96062-3 | SS-OP - 49% | 27.5% | 22% | BRIJ ™ S100 - 1.5% |
| 80097-2 | SS-OC - 49% | 28.5% | 22% | PEG2k-DPG - 0.5% |

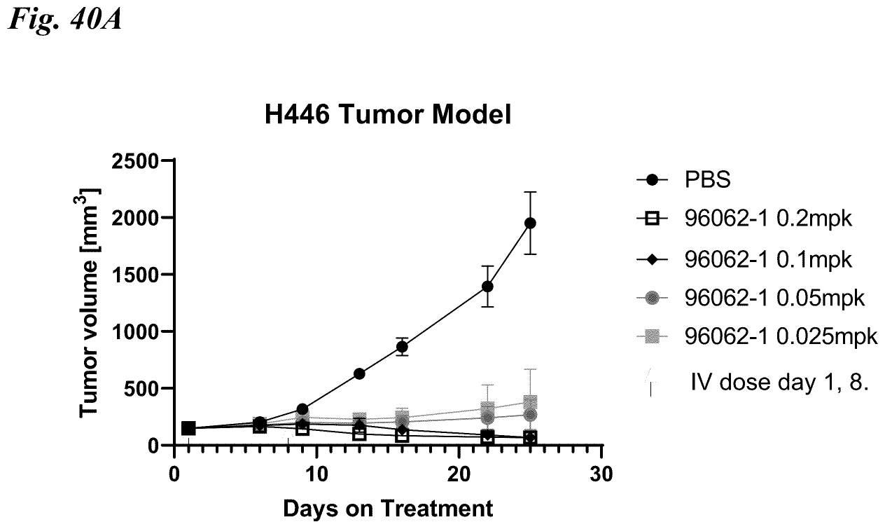

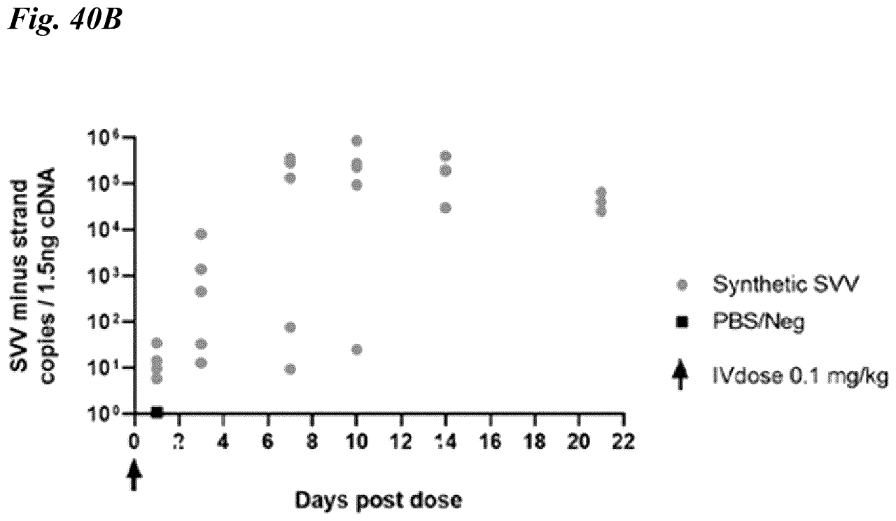

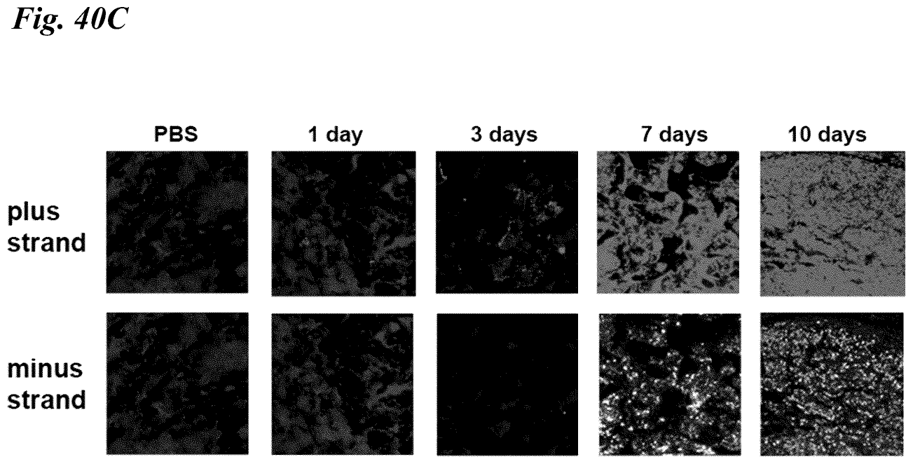

The anti-tumor efficacy of SVV/LNP was tested in an NCI-H₄₆₆ SCLC xenograft model. Athymic nude female mice were injected with NCI-H446 cells (5×10⁶ cells/0.1 mL in a 1:1 mixture of serum-free PBS and Matrigel®) were implanted subcutaneously in the right flank. When median tumor size was approximately 150 mm³ (120-180 mm³ range), mice were cohorted in groups of 7 mice per treatment arm. Mice were treated with the LNPs containing SVV RNA twice on day 1 and day 8. FIG. 40A shows dose titration of SVV/LNP comprising SS—OC as cationic lipid and 0.5 mol % BRIJ™ S100 as PEG-lipid (formulation #96062-1). The doses tested ranged from 0.025 mg/kg to 0.2 mg/kg. All doses tested displayed significant tumor growth inhibition as compared to the PBS negative control. (Two-way ANOVA, Tukey Test, p<0.0001; “mpd” stands for “mg/kg” in the figure.) The 0.1 mg/kg dose was used to characterize the kinetics of viral replication after a single IV administration of SVV-RNA in NCI-H446 tumor-bearing mice. Tumors were harvested at multiple time points and analyzed by RT-qPCR and FISH. Viral minus-strand was detected by RT-qPCR as early as 1 day post treatment and reached a plateau in most tumors at 7 days post treatment. Sustained SVV replication was detected up to 21 days after administering a single low dose of SVV-RNA (FIG. 40B). These findings were largely recapitulated with FISH detection of SVV minus and positive strands, with the FISH signal increasing to the maximum by day 10 indicative of virus spreading throughout the tumor bed at this timepoint (FIG. 40C)

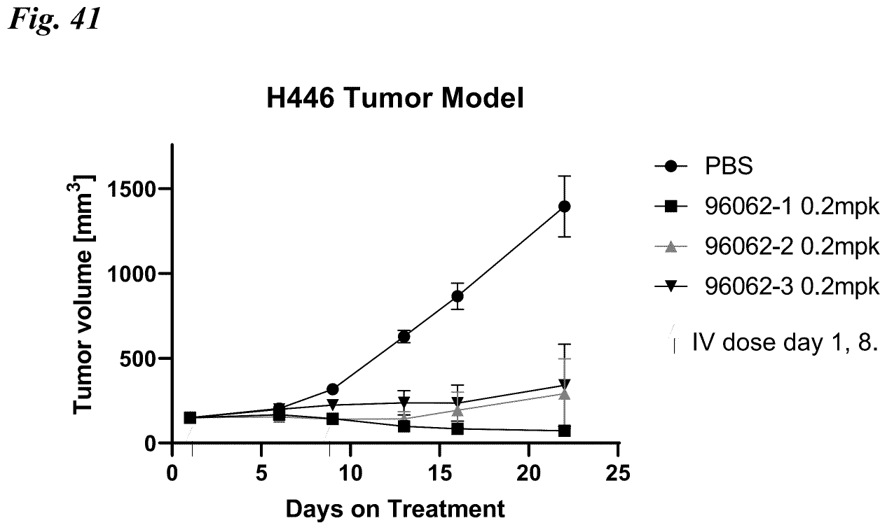

FIG. 41 shows the anti-tumor effector of SVV/LNP comprising different cationic lipid and varying amounts of BRIJ™ S100 molecule. Three different combinations of cationic lipid and BRIJ™ S100 amounts in the LNP formulations were tested as described in Table 10.

All doses tested displayed significant tumor growth inhibition as compared to the PBS negative control (Two-way ANOVA, Tukey Test, p<0.0001; “mpd” stands for “mg/kg” in the figure). Taken together, the results suggest that either SS—OC or SS—OP can be used as cationic lipid in formulating LNPs that effectively deliver the encapsulated synthetic RNA viral genomes to the target cells and achieve tumor growth inhibition over a range of doses.

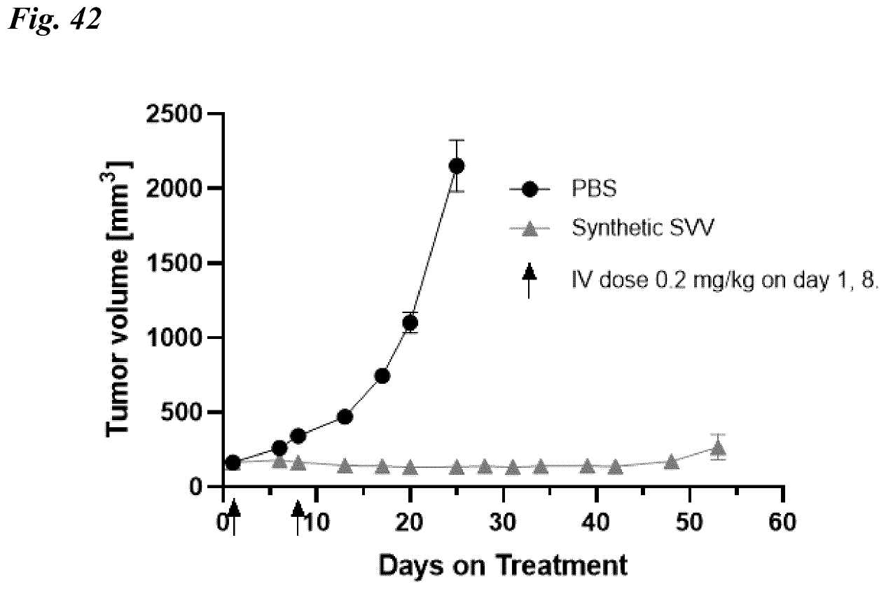

FIG. 42 shows the prolonged anti-tumor efficacy of SVV/LNP formulation comprising Brij in SCLC model with NCI-H₄₆₆. LNPs used herein comprises SS—OC as cationic lipid and 0.5 mol % BRIJ™ S100 as PEG-lipid (Formulation 96062-1). Athymic nude female mice were implanted subcutaneously with NCI-H446 cells (5×10⁶ cells/0.1 mL in a 1:1 mixture of serum-free PBS and Matrigel®) in the right flank. When median tumor size was approximately 150 mm³ (120-180 mm³ range), mice were cohorted in groups of 7 mice per treatment arm. Mice were dosed twice on day 1 and day 8 with SVV/LNP at 0.2 mg/kg. Compared to the negative control (PBS), the SVV/LNP composition demonstrated significant tumor inhibitory effect for a duration of more than 50 days (Two-way ANOVA, Tukey Test p<0.0001).

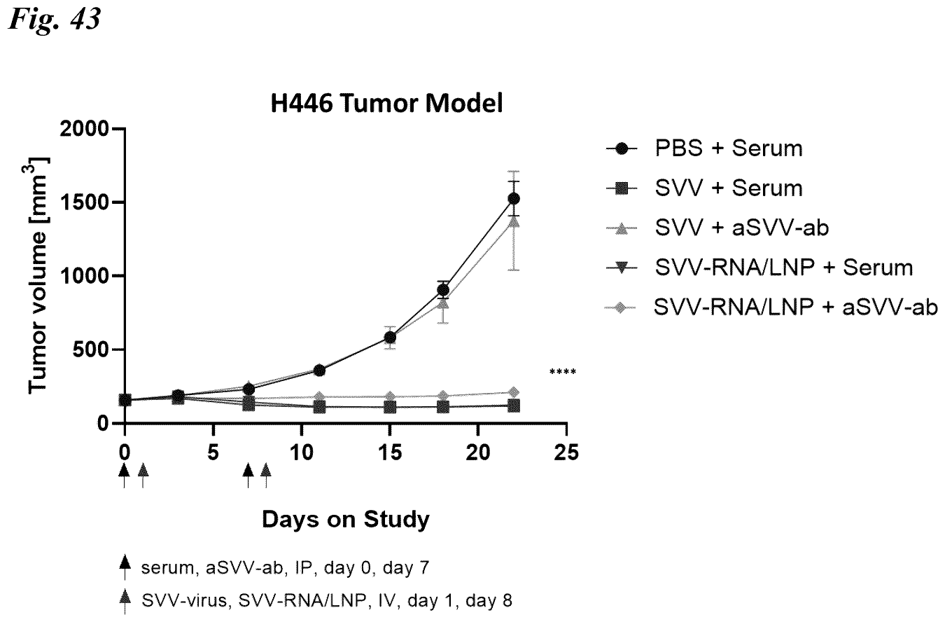

FIG. 43 shows that the presence of anti-SVV neutralizing antibodies does not inhibit SVV/LNP efficacy in a SCLC tumor model (NCI-H446). Athymic nude female mice were implanted with NCI-H446 cells (5×10⁶ cells/0.1 mL in a 1:1 mixture of serum-free PBS and Matrigel®) subcutaneously in the right flank. When median tumor size was approximately 150 mm³ (120-180 mm³ range), mice were cohorted in groups of 10 mice per treatment arm. Mice were intraperitoneal administered (passively immunized) with a rabbit serum containing anti-SVV neutralizing antibody (μSVV-ab) or a rabbit control serum (Serum alone) on day 0 and day 7, and treated with the SVV/LNP or SVV virions on day 1 and day 8. LNPs used herein comprises SS—OC as cationic lipid and 0.5 mol % BRIJ™ S100 as PEG-lipid (Formulation 96062-1). Tumor volumes in H446 tumor-bearing mice following intravenous administration of PBS, SVV virions, or SVV/LNPs were monitored. The tumor inhibitory effect of the SVV/LNP composition was not affected by the presence of anti-SVV neutralizing antibodies, whereas the tumor inhibitory effect of the SVV virions was completely negated by the presence of anti-SVV neutralizing antibodies. ****TGI significant against PBS (Two-way ANOVA, Tukey Test p<0.0001).

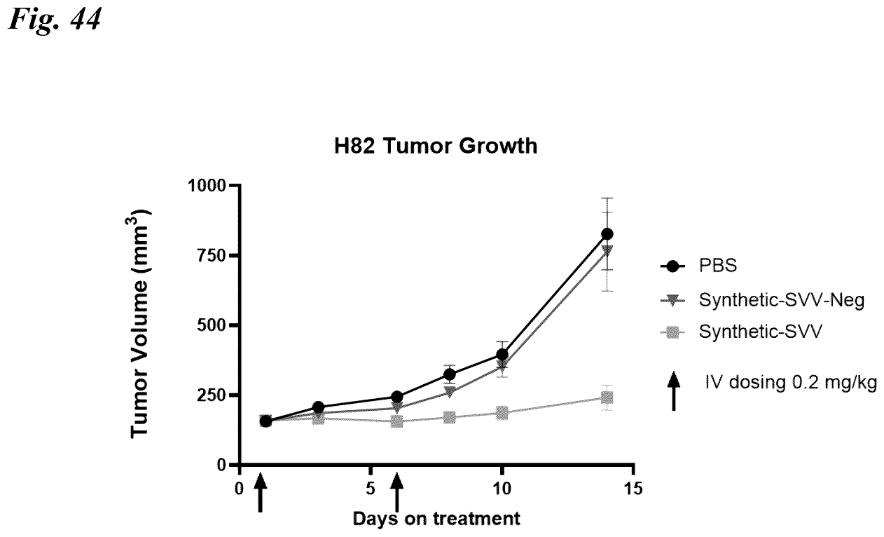

FIG. 44 shows the anti-tumor efficacy of SVV/LNP in SCLC model using NCI-H82 cells. LNPs used herein comprises SS—OC as cationic lipid and 0.5 mol % PEG2k-DPG as PEG-lipid (80097-2). Athymic nude female mice were injected with NCI-H82 cells (5×10⁶ cells/0.1 mL in a 1:1 mixture of serum-free PBS and Matrigel®) were implanted subcutaneously in the right flank. When median tumor size was approximately 160 mm³ (130-180 mm³ range), mice were cohorted in groups of 7 mice per treatment arm. Mice were dosed twice on day 1 and day 6 with SVV/LNP at 0.2 mg/kg. Compared to the negative controls (SVV-Neg/LNP or PBS), the SVV/LNP composition demonstrated significant tumor growth inhibition (Two-way ANOVA, Tukey Test p<0.0001).

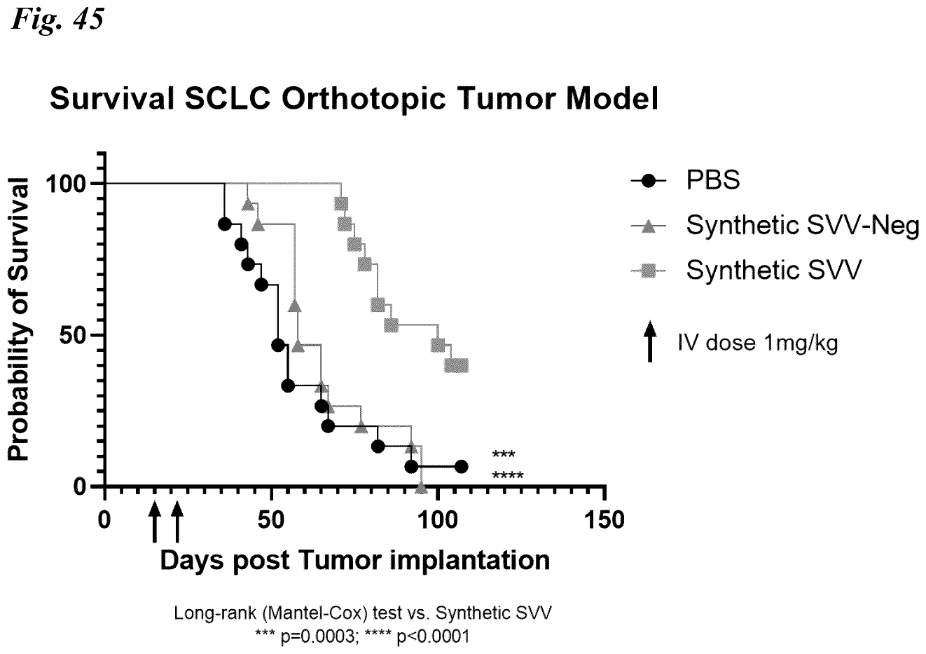

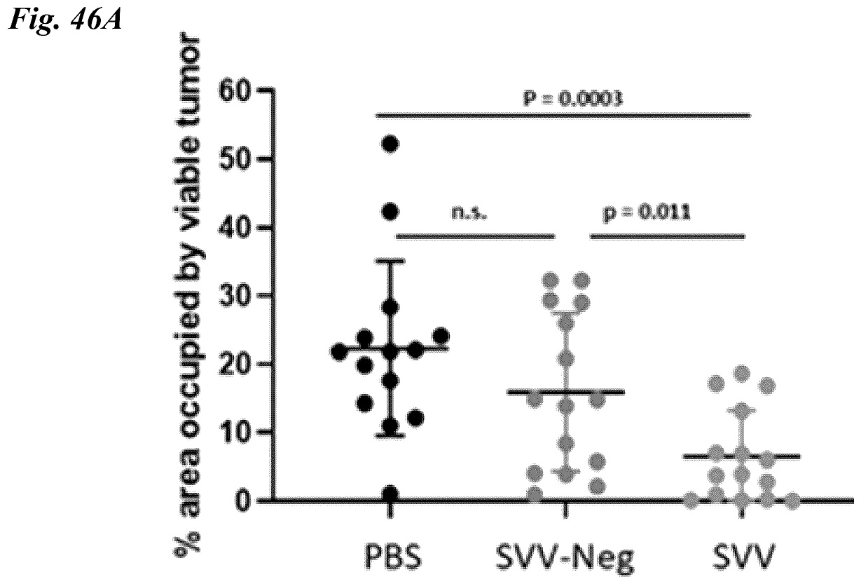

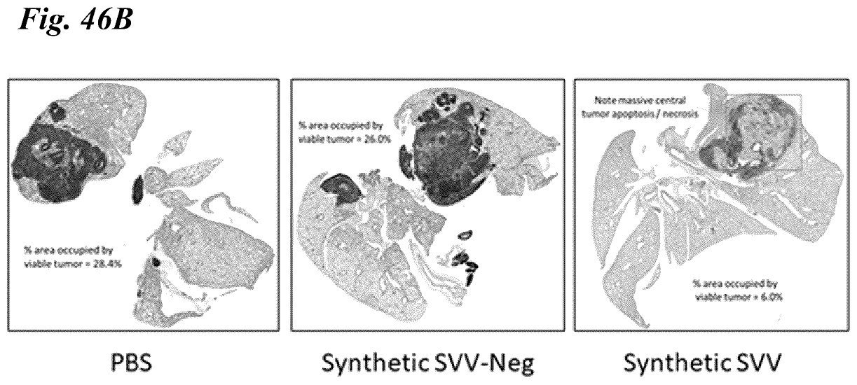

FIG. 45 shows effect of systemic administration of SVV/LNP at prolonging survival in a SCLC orthotopic tumor model (NCI-H82). Athymic nude mice were inoculated into the lung with the single-cell suspension of more than 90% viable H82 tumor cells (5×10⁶ cells injected on the right lung) in 0.05 ml in the mixture of serum-free DPBS and Matrigel (1:1 v/v), for tumor development. Mice were dosed with PBS or 1 mpk SVV-vRNA/LNP comprising SS—OC as cationic lipid and 0.5 mol % BRIJ™ S100 as PEG-lipid (Formulation 96076-2 (same lipid composition as 96062-1) intravenously (IV) on day 15 and 22 post tumor implantation. Compared to the negative controls (SVV-Neg/LNP or PBS), the SVV/LNP composition significantly extended survival of mice in this SCLC orthotopic tumor model. SVV-RNA administered in day 15 and 22 after tumor implantation yielded a significant therapeutic benefit vs. the control arm with mice surviving a median of 100 days, nearly doubling survival in this model. A cohort of mice were treated as described and lungs of SVV-RNA treated mice were harvested 10 days after the second treatment for IHC. hDLL3 IHC was used as a marker for tumor burden, and IHC quantification demonstrated a significant reduction of tumor burden relative to the SVV-Neg or PBS controls (FIG. 46A and FIG. 46B). SVV-RNA treated mice demonstrated extensive central tumor necrosis (FIG. 46B).

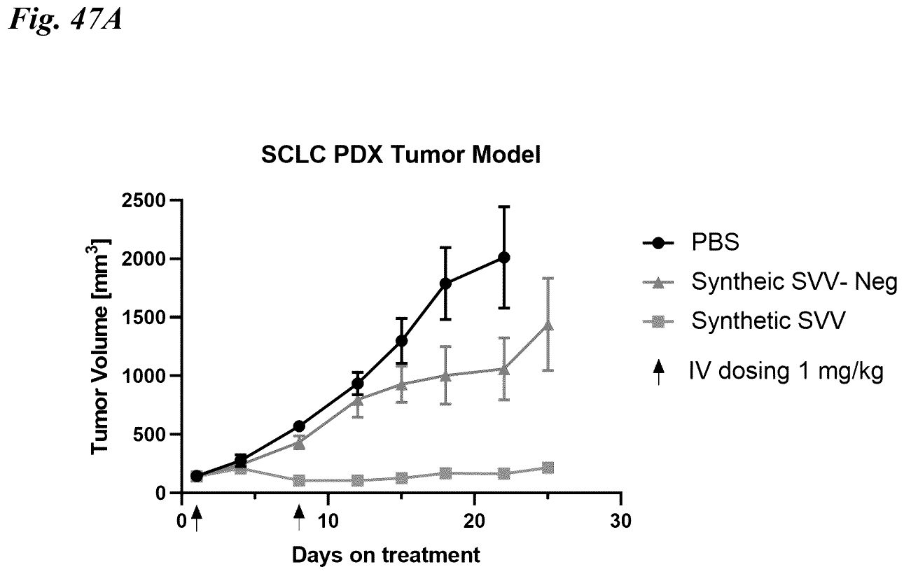

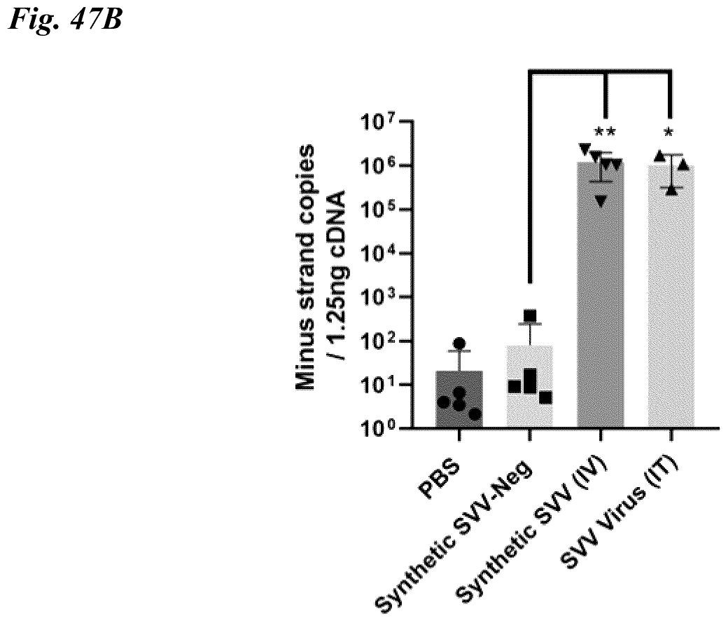

FIG. 47A shows the anti-tumor efficacy of SVV/LNP in SCLC PDX model. LNPs used herein comprises SS—OC as cationic lipid and 0.5 mol % BRIJ™ S100 as PEG-lipid (Formulation 96062-1). NOD-SCID female mice were injected with SCLC PDX cells (fragments 12×2 in 100 ul of serum-free media) implanted subcutaneously in the right flank. When median tumor size was approximately 150 mm³ (120-180 mm³ range), mice were cohorted in groups of 8 mice per treatment arm. Mice were dosed twice on day 1 and day 8 with SVV/LNP, or the negative control SVV-Neg/LNP or PBS, at 1 mg/kg. SVV/LNP demonstrated significant tumor growth inhibition as compared to the negative controls (Two-way ANOVA, Tukey Test p<0.0001). SVV replication was also measured by RT-qPCR and showed that SVV-RNA dosed IV achieved similar levels of viral replication as SVV virions IT (FIG. 47B).

Anti-Tumor Effect of SVV RNA Viral Genome with Varying Length of Poly-A Tail:

The anti-tumor efficacy of SVV/LNP comprising an SVV RNA viral genome with different lengths of poly-A tail was also tested using an SCLC model. Two different lengths of poly-A tail were tested—one is 30 nucleotides in length (pA30), and the other is 70 nucleotides in length (pA70). The corresponding synthetic RNA viral genomes with a poly-A tail are shown in SEQ ID NO: 37 and 38, which are derived from SVV-IRES2 chimeric virus and contains the VP2 protein S177A mutation. Both RNA molecules were in vitro transcribed based on DNA template constructs comprising SEQ ID NO: 51 and 52, respectively. Each of these DNA template constructs comprise a T7 promoter sequence, followed by a leader sequence according to SEQ ID NO: 53, followed by a Pistol ribozyme sequence according to SEQ ID NO: 64, followed by the SVV-IRES2 chimeric viral genome with the poly-A tail of the indicated length, followed by a SapI restriction enzyme recognition site, with no additional nucleotide inserted in between the adjacent components.

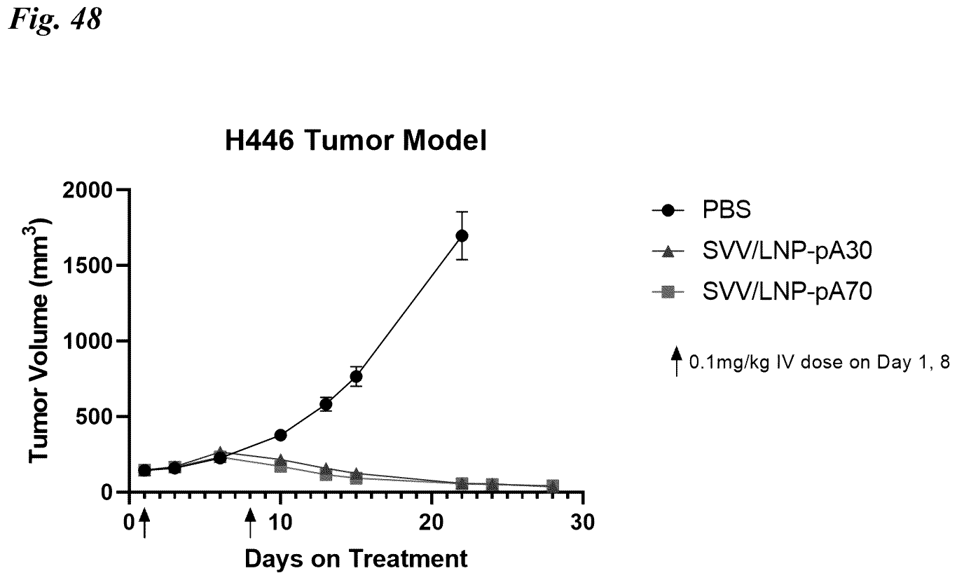

Both SVV RNAs were formulated with LNP comprising SS—OC as cationic lipid and 0.5 mol % BRIJ™ S100 as PEG-lipid. Athymic nude female mice were injected with NCI-H446 cells (5×10⁶ cells/0.1 mL in a 1:1 mixture of serum-free PBS and Matrigel®) were implanted subcutaneously in the right flank. When median tumor size was approximately 150 mm³ (120-180 mm³ range), mice were cohorted in groups of 8 mice per treatment arm. Mice were dosed twice on day 1 and day 8 with 0.1 mg/kg using SVV-RNA-pA30/LNP or SVV-RNA-pA70/LNP. As shown in FIG. 48, both SVV-RNA-pA30 and SVV-RNA-pA70 demonstrated similar efficacy at inhibiting tumor growth, which are significantly better than the PBS control (Two-way ANOVA, Tukey Test p<0.0001).

### Example 16: Combination Therapy of SVV-LNP and Anti-PD-1

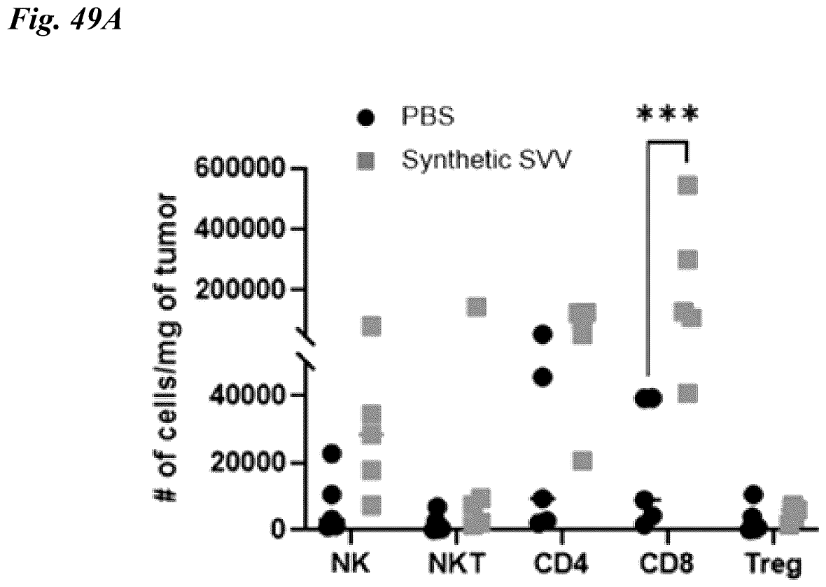

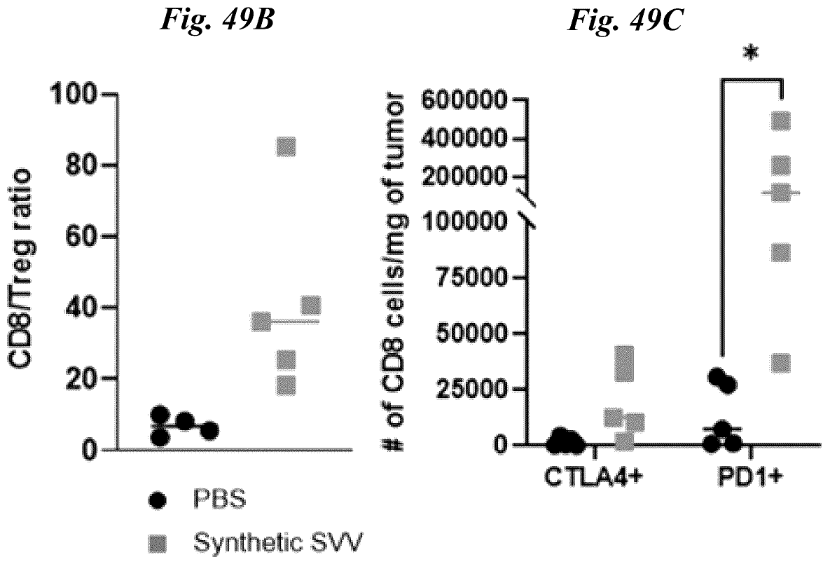

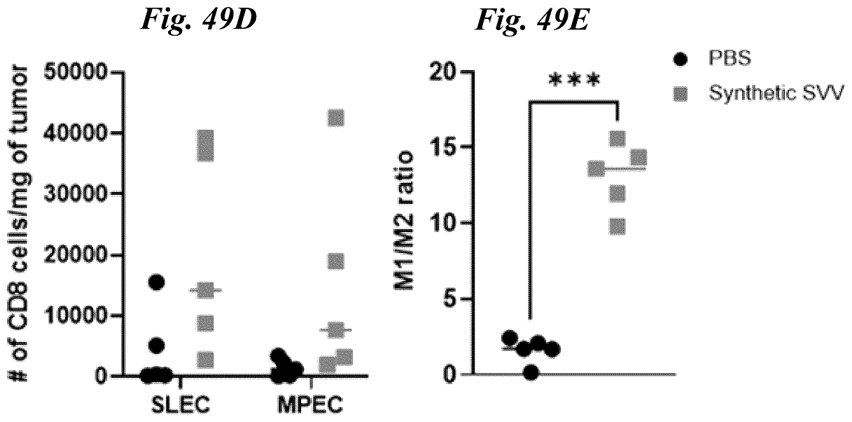

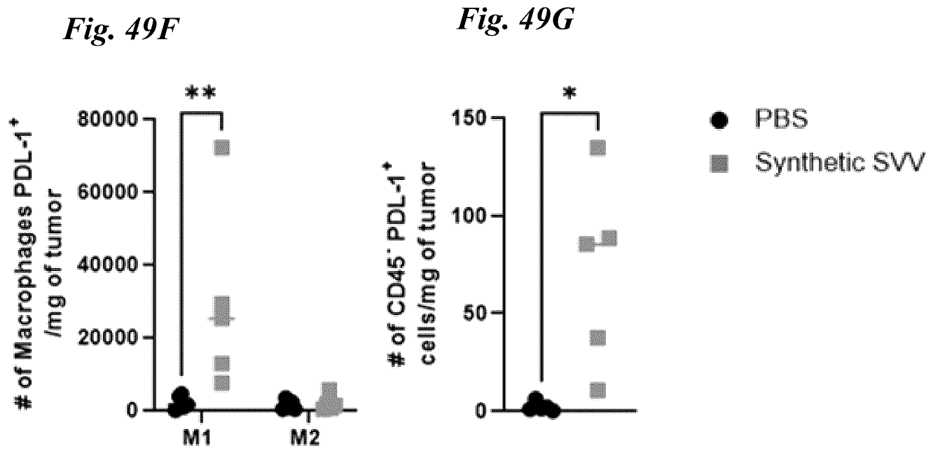

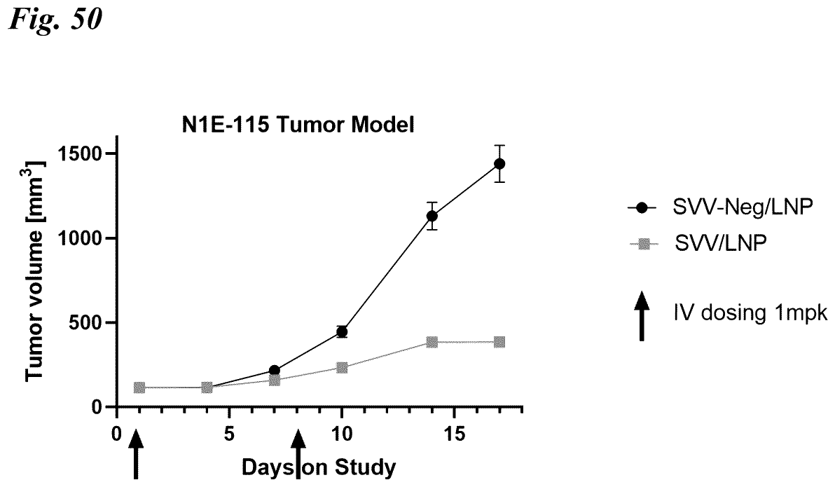

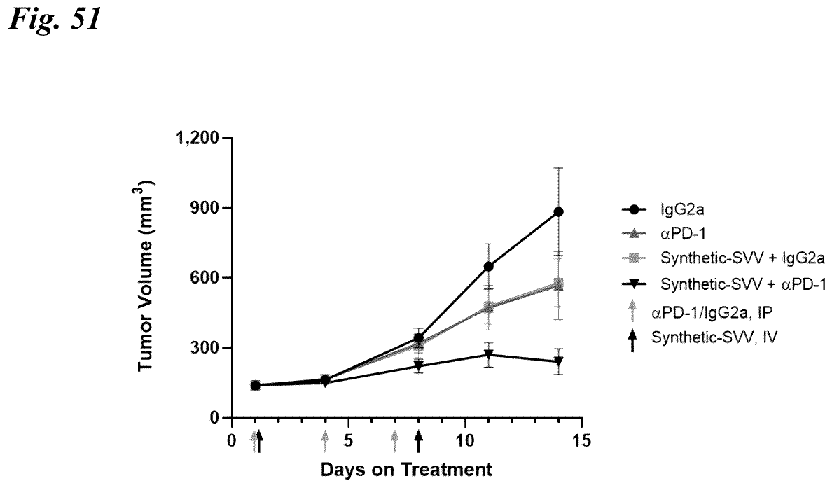

SVV/LNP comprising SS—OC as cationic lipid and 0.5 mol % BRIJ™ S100 as PEG-lipid in were tested in a syngeneic neuroblastoma model, N₁E-115. A/J female mice were injected with N₁E-115 cells (5×10⁵ cells/0.1 mL in a 1:1 mixture of serum-free PBS and Matrigel®) were implanted subcutaneously in the right flank. When median tumor size became approximately 100 mm³, mice were cohorted in groups of 10 mice per treatment arm. Mice were dosed twice on day 1 and day 8 with SVV/LNP, or the negative control SVV-Neg/LNP, at 1 mg/kg. In this tumor model, administration of Synthetic SVV led to a significant increase in the recruitment of CD8 T cells and a trend for CD4 T cells and NK cells in tumors (FIG. 49A). Regulatory T cells (Treg) numbers were not increased in tumors, leading to an overall elevated CD8/Treg ratio that has been associated with improved clinical benefit to anti-PD-1 (FIG. 49B). The CD8 T cells showed an activated phenotype, with an upregulation of CTLA4 and PD-1 (FIG. 49C). Both short-lived effector cells (SLEC) and memory precursor effector cells (MPEC) were increased in the Synthetic SVV treated group compared to control (FIG. 49D). Tumor-associated macrophages were also profiled and an increased M1 (phagocytic)/M2 (proinflammatory) ratio was observed (FIG. 49E). The number of M1 macrophages (FIG. 49F) and tumor cells expressing PD-1 ligand, PD-L1, was also significantly increased (FIG. 49G). These data indicate that Synthetic SVV promotes a change within the TME conducive to anti-tumor immunity. Mice bearing N₁E-155 tumors were treated with SVV/LNP or SVV-Neg-LNP demonstrated significant growth inhibition of this syngeneic model (FIG. 50). Notably, due to the increase in both PD-1 and PD-L1 expression, the efficacy of the combination of synthetic SVV with anti-PD-1 antibody in the N₁E-115 model was evaluated. Both agents administered as monotherapy led to modest significant anti-tumor activity that was significantly enhanced when administered as combination therapy with anti-PD-1 antibody compared to each single-agent arm (FIG. 51).

### Example 17: Test of SVV/LNP Formulation Using Different Brij Molecules

Lipid nanoparticles comprising SVV RNA viral genome were prepared and characterized according to Table 11 and Table 12. All LNPs were formulated with SS—OC:Cholesterol:DSPC:PEG-lipid, wherein the PEG-lipid was PEG2k-DPG, BRIJ™ S100, BRIJ™ C₂₀ or BRIJ™ S20, respectively.

TABLE 11
Formulation parameters for SVV-RNA LNPs

| Lot # | Buffer | PEG-lipid | SS-OC:Chol:DSPC:PEG-lipid |
|---|---|---|---|
| 96047-1 | 20 mM Malic, pH 3.0 | DPG-PEG2K | 49:28.5:22:0.5 |
| 96047-2 | 25 mM acetate pH 5.0 | BRIJ ™ S100 | 49:28.5:22:0.5 |
| 96047-3 | 25 mM acetate pH 5.0 | BRIJ ™ S100 | 49:27.5:22:1.5 |
| 96047-4 | 25 mM acetate pH 5.0 | BRIJ ™ S100 | 49:38.5:11:1.5 |
| 96047-5 | 25 mM acetate pH 5.0 | BRIJ ™ C20 | 49:28.5:22:0.5 |
| 96047-6 | 25 mM acetate pH 5.0 | BRIJ ™ S20 | 49:28.5:22:0.5 |

TABLE 12
SVV-RNA LNP characteristics

| Lot # | Z-Average (d · nm) | PDI | ZP (mV) | EE % |
|---|---|---|---|---|
| 96047-1 | 88.7 | 0.10 | −3.7 | 100% |
| 96047-2 | 107.8 | 0.12 | −0.6 | 96% |
| 96047-3 | 91.8 | 0.14 | −2.0 | 93% |
| 96047-4 | 81.4 | 0.14 | −0.9 | 98% |
| 96047-5 | 108.7 | 0.14 | −8.8 | 98% |
| 96047-6 | 100.6 | 0.11 | −6.6 | 99% |

Z-Average = average diameter;
PDI = polydispersity index;
% EE = Encapsulation Efficiency;
ZP = zeta potential

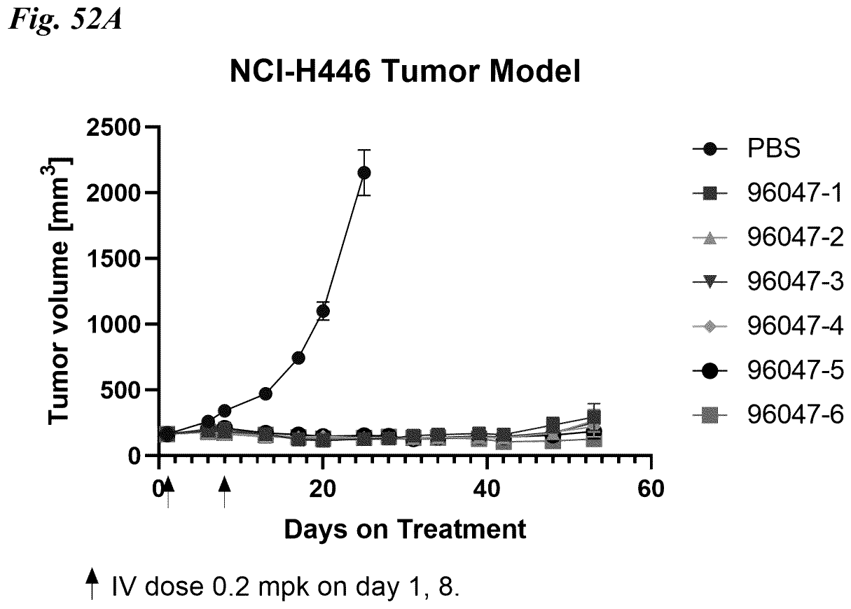

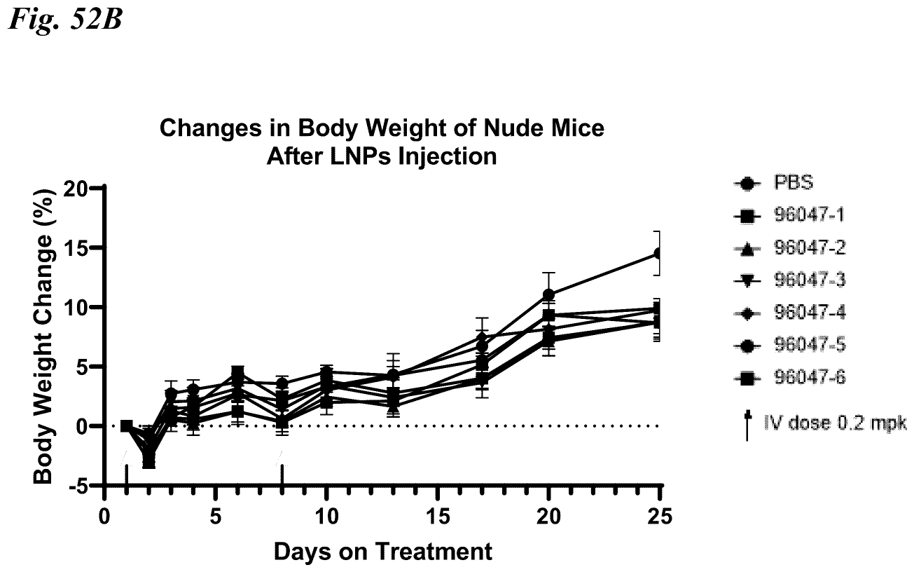

SVV/LNP compositions were used in a repeat dose IV mouse efficacy screen in an H446 tumor animal model. Tumor volume (FIG. 52A) and body weight (FIG. 52B) were measured at each time point. The results showed that all formulations demonstrated high anti-tumor efficacy and were well tolerated. LNPs comprising Brij molecules as PEG-lipid were similar in efficacy and tolerability as compared to LNPs comprising PEG2k-DPG as PEG-lipid.

### Example 18: LNPs Comprising Brij Displayed Altered Pharmacokinetic Characteristics In Vivo Upon Repeat Dosing

Lipid nanoparticles comprising SVV viral genome were prepared and characterized according to Table 13. The PEG-lipid in the LNP was PEG2k-DPG, PEG2k-DMG or BRIJ™ S100, respectively. Other lipid components include SS—OC, cholesterol, DSPC, and optionally b-sitosterol (960166). Additional characterization parameters are provided in Table 14.

TABLE 13
Formulation parameters for SVV-RNA LNPs

| | | SS-OC:Cholesterol:b- | Lipid | Input RNA | | |
|---|---|---|---|---|---|---|
| | PEG | sitosterol:DSPC:PEG- | conc. | conc. | | Flow |
| Lot# | lipid | lipid (mol %) | (mM) | (mg/mL) | N:P | ratio |
| 960161 | DPG-PEG2K | 49:28.5:0:22:0.5 | 40 | 0.298 | 14 | 3 |
| 960164 | DMG-PEG2K | 49:35.8:0:15:0.2 | 40 | 0.298 | 14 | 3 |
| 960166 | DMG-PEG2K | 49:29.5:10:11:0.5 | 40 | 0.298 | 14 | 3 |
| JD-200311-1 | BRIJ ™ S100 | 49:28.5:0:22:0.5 | 40 | 0.298 | 14 | 3 |

TABLE 14
SVV-RNA LNP characteristics

| Lot # | Size (nm) | PDI | [RNA] (μg/mL) | % EE | ZP (mv) |
|---|---|---|---|---|---|
| 960161 | 94 | 0.12 | 380 | 92 | −3.1 |
| 960164 | 114 | 0.11 | 382 | 92 | −2.3 |
| 960166 | 129 | 0.09 | 360 | 89 | −3.3 |
| JD-200311-1 | 132 | 0.11 | 235 | 88 | — |

PDI—polydispersity index;
% EE = Encapsulation Efficiency;
ZP = zeta potential

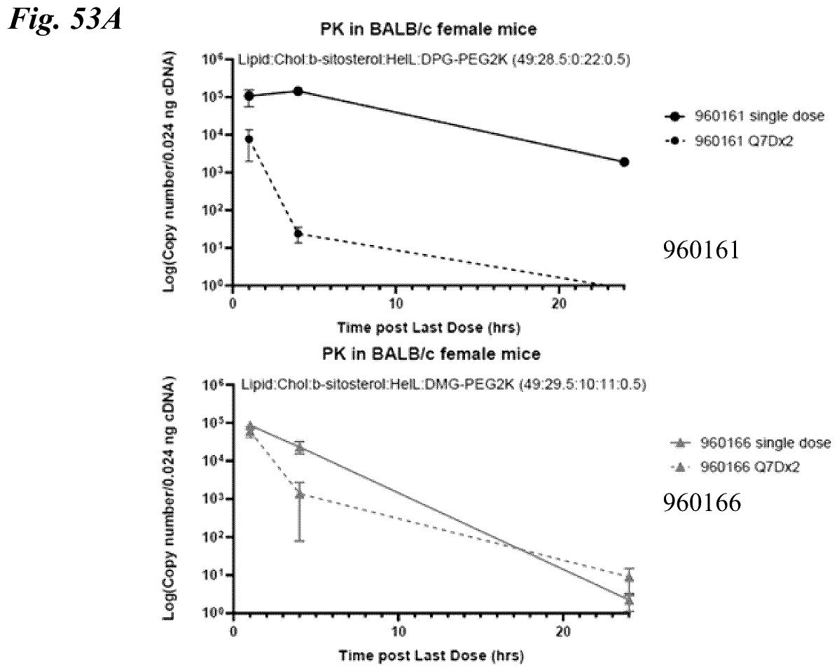

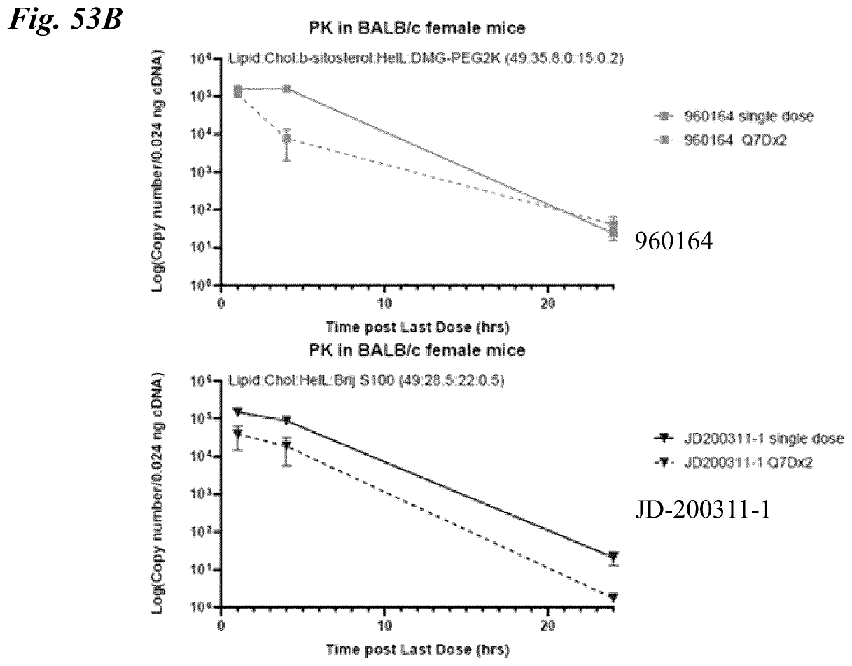

SVV/LNP compositions were used in a repeat dose (weekly dose schedule for 2 weeks, Q7×2) intravenous (IV) PK study in BALB/c female mice. For each formulation, the dose was 0.5 mg/kg and dose volume was 5 mL/kg. Copy number of RNA in serum post-dose was measured at multiple time points (1 hour, 4 hours, and 24 hours) post first dose (day 1) and post second dose (day 8). The results are shown in FIG. 53A and FIG. 53B. Solid lines show the pharmacokinetic profile of the first dose. Broken lines show the pharmacokinetic profile of the second dose.

Both formulation #960161 and #JD-200311-1 have lipid composition of SS—OC:Cholesterol:DSPC:PEG-lipid (49:28.5:22:0.5 mol %), and the PEG-lipid is DPG-PEG2k and BRIJ™ S100, respectively.

The results showed that LNP comprising PEG2k-DPG as PEG-lipid exhibited prolonged circulation post-first dose with rapid clearance within 4 hours upon the second dose. In contrast, LNP comprising BRIJ™ S100 as PEG-lipid exhibited an intermediate change in exposure post-first dose but maintained similar circulation characteristic and slopes of elimination upon the second dose.

### Example 19: Modification of RNA Acidifying Buffer Improves LNP Biophysical Properties

This example illustrates the encapsulation of non-replicating Seneca Valley virus (SVV) RNA (SVV-Neg) in LNP formulations with varying the RNA acidifying buffer to determine the effect changing the citrate concentration and pH would have on the LNP biophysical properties. LNPs in this example comprise a lipid composition of ionizable lipid (CAT):DSPC:cholesterol:PEG2k-DMG at 50:7:40:3 mol %. The lipid mixture in ethanol was mixed with SVV-Neg in RNA acidifying buffer (e.g., 50 mM citrate, pH 4) at a lipid-nitrogen-to-phosphate ratio (N:P) of 9 using a microfluidic device (Precision NanoSystems Inc.). Total lipid concentration was set to 20 mM.

LNPs were dialyzed against 50 mM phosphate, pH 6.0, for 12-16 h, and secondary dialysis was performed against 50 mM HEPES, 50 mM NaCl, 263 mM sucrose, pH 7.3, for 4-24 h at room temperature. Post-dialyzed LNPs were concentrated using 100 kDa AMICON@ ULTRA CENTRIFUGAL filters (MilliporeSigma) and sterile filtered using 0.2 μm syringe filters. Samples were then characterized and diluted as needed. Upon dilution, a 5 w/v % glycerol spike was added if samples were then stored at −20° C.

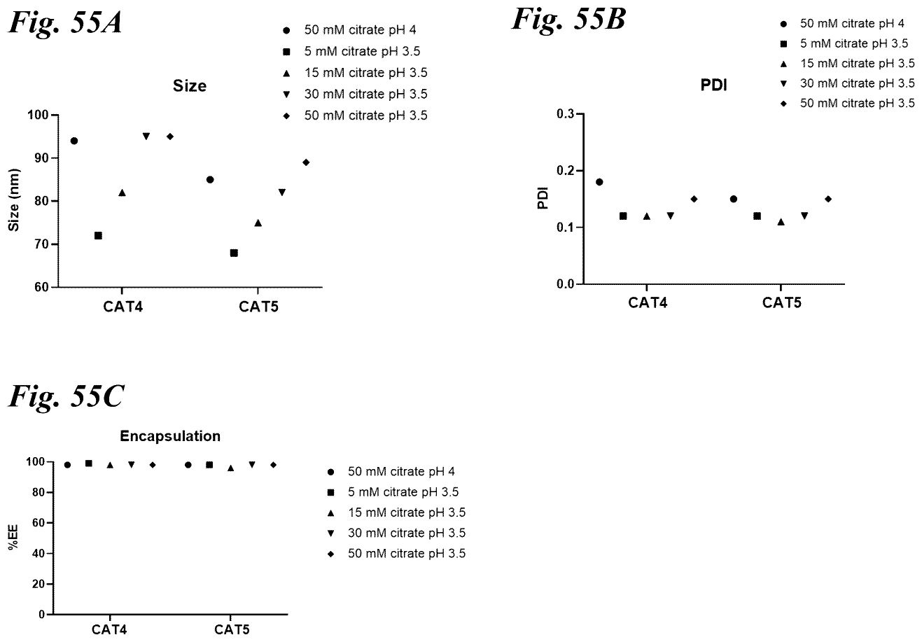

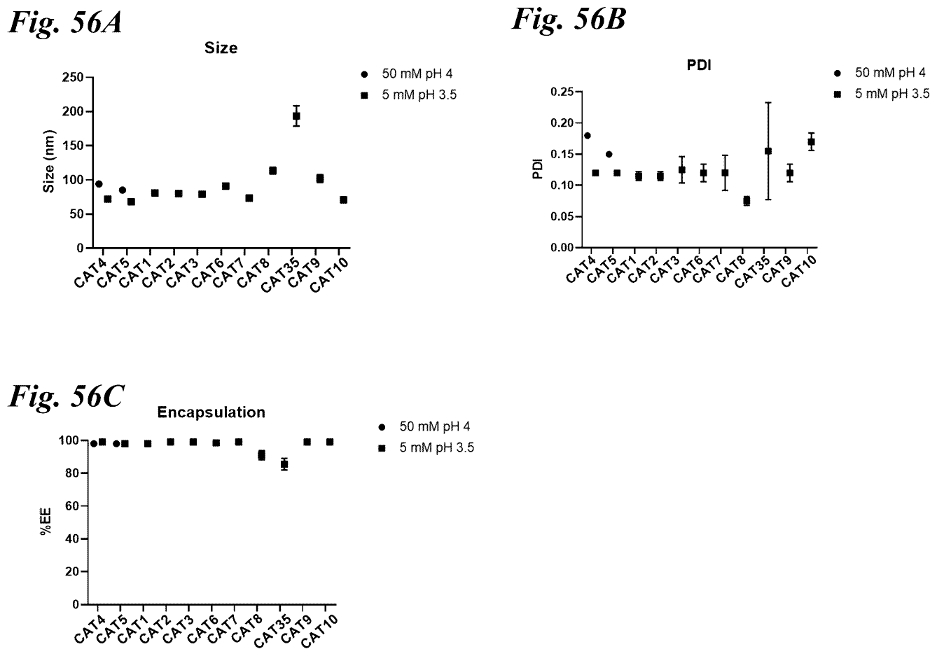

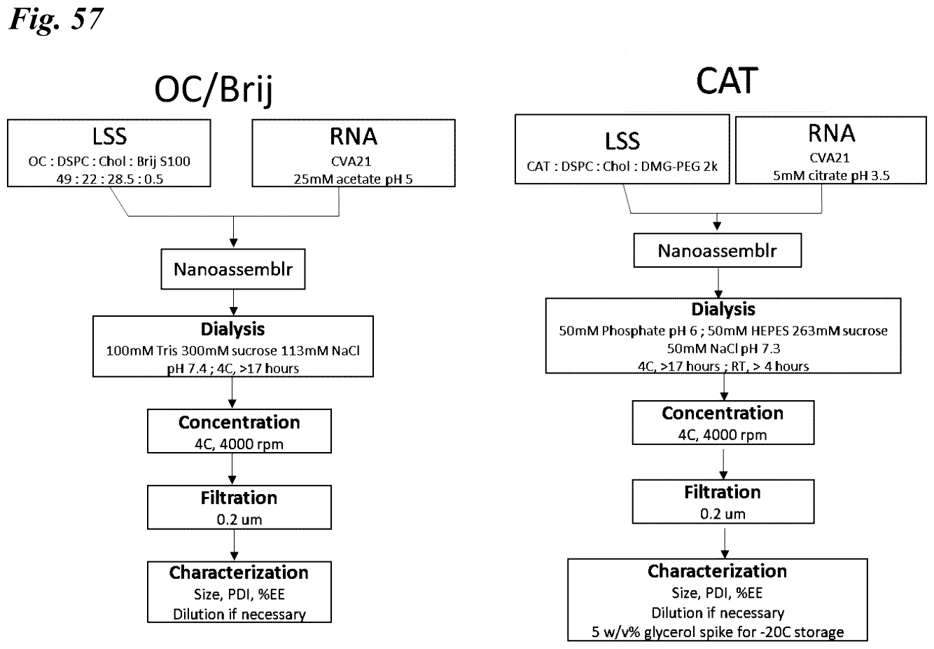

LNPs were characterized for particle size by dynamic light scattering (DLS and polydispersity index (PDI). CAT4 and CAT5 formulations were tested with RNA acidifying buffer: (1) 50 nM citrate pH4; (2) 5 mM citrate pH 3.5; (3) 15 mM citrate pH 3.5; (4) 30 mM citrate pH 3.5; and (5) 50 mM citrate pH 3.5. FIG. 55A, FIG. 55B, and FIG. 55C depict the particle size, PDI, and encapsulation efficiency of the LNPs. Further, CAT1 to CAT3, CAT6 to CAT10, and CAT35 LNP formulations were made with the 5 mM citrate pH 3.5 buffer (FIG. 56A, FIG. 56B, and FIG. 56C).

The results suggested changing the RNA acidifying buffer (e.g., lowering salt concentration) resulted in smaller particle size and PDI.

### Example 20: In Vivo Studies of LNPs Comprising Different Ionizable Lipids

The in vivo pharmacodynamics and anti-tumor efficacy of Seneca Valley virus (SVV)-RNA encapsulated in LNP was evaluated in a mouse model for small cell lung cancer (SCLC).

In this example, the RNA molecules encoding SVV viral genomes and a NanoLuc luciferase (NLuc) were encapsulated in LNPs prepared according to Table 22 below. NLuc is a luciferase enzyme that produces luminescent signal when provided with the substrate furimazine. The LNPs were dialyzed overnight in 100 mM tris 300 mM sucrose 113 mM NaCl pH 7.4 at 5° C. Alternatively, the LNPs were dialyzed against 50 mM phosphate, pH 6.0, for 12-16 h and secondary dialysis was performed against 50 mM HEPES, 50 mM NaCl, 263 mM sucrose, pH 7.3, for 4-24 h at room temperature. Post-dialyzed LNP formulations were concentrated, filtered, characterized, and optionally diluted.

TABLE 22
LNP formulations for in vivo studies

| | | | Ionizable | | | | |
|---|---|---|---|---|---|---|---|
| | Ionizable | | lipid:Cholesterol:DSPC:PEG- | | Size | | |
| Formulation | Lipid | PEG-Lipid | lipid (mol %) | Acidifying Buffer | (nm) | PDI | % EE |
| CAT1/DMG | CAT1 | PEG₂ₖ-DMG | 50:40:7:3 | 5 mm citrate, pH 3.5 | 75 | 0.13 | 97 |
| CAT2/DMG | CAT2 | PEG₂ₖ-DMG | 50:40:7:3 | 5 mm citrate, pH 3.5 | 73 | 0.18 | 98 |
| CAT3/DMG | CAT3 | PEG₂ₖ-DMG | 50:40:7:3 | 5 mm citrate, pH 3.5 | 74 | 0.17 | 98 |
| CAT4/DMG | CAT4 | PEG₂ₖ-DMG | 50:40:7:3 | 5 mm citrate, pH 3.5 | 80 | 0.16 | 97 |
| CAT5/DMG | CAT5 | PEG₂ₖ-DMG | 50:40:7:3 | 5 mm citrate, pH 3.5 | 73 | 0.18 | 96 |
| CAT6/DMG | CAT6 | PEG₂ₖ-DMG | 50:40:7:3 | 5 mm citrate, pH 3.5 | 86 | 0.2 | 98 |
| CAT7/DMG | CAT7 | PEG₂ₖ-DMG | 50:40:7:3 | 5 mm citrate, pH 3.5 | 72 | 0.14 | 97 |
| CAT8/DMG | CAT8 | PEG₂ₖ-DMG | 50:40:7:3 | 5 mm citrate, pH 3.5 | 120 | 0.07 | 90 |
| CAT9/DMG | CAT9 | PEG₂ₖ-DMG | 50:40:7:3 | 5 mm citrate, pH 3.5 | 87 | 0.19 | 98 |
| CAT10/DMG | CAT10 | PEG₂ₖ-DMG | 50:40:7:3 | 5 mm citrate, pH 3.5 | 71 | 0.21 | 97 |
| CAT11/DMG | CAT11 | PEG₂ₖ-DMG | 50:40:7:3 | 5 mm citrate, pH 3.5 | 81 | 0.17 | 96 |
| CAT12/DMG | CAT12 | PEG₂ₖ-DMG | 50:40:7:3 | 5 mm citrate, pH 3.5 | 86 | 0.33 | 89 |
| CAT13/DMG | CAT13 | PEG₂ₖ-DMG | 50:40:7:3 | 5 mm citrate, pH 3.5 | 79 | 0.12 | 97 |
| CAT14/DMG | CAT14 | PEG₂ₖ-DMG | 50:40:7:3 | 5 mm citrate, pH 3.5 | 81 | 0.32 | 91 |
| CAT15/DMG | CAT15 | PEG₂ₖ-DMG | 50:40:7:3 | 5 mm citrate, pH 3.5 | 110 | 0.24 | 98 |
| CAT16/DMG | CAT16 | PEG₂ₖ-DMG | 50:40:7:3 | 5 mm citrate, pH 3.5 | 75 | 0.2 | 95 |
| CAT17/DMG | CAT17 | PEG₂ₖ-DMG | 50:40:7:3 | 5 mm citrate, pH 3.5 | 73 | 0.52 | 97 |
| CAT19/DMG | CAT19 | PEG₂ₖ-DMG | 50:40:7:3 | 5 mm citrate, pH 3.5 | 67 | 0.21 | 98% |
| CAT20/DMG | CAT20 | PEG₂ₖ-DMG | 50:40:7:3 | 5 mm citrate, pH 3.5 | 66 | 0.21 | 98% |
| CAT24/DMG | CAT24 | PEG₂ₖ-DMG | 50:40:7:3 | 5 mm citrate, pH 3.5 | 64 | 0.37 | 97% |
| CAT31/DMG | CAT31 | PEG₂ₖ-DMG | 50:40:7:3 | 5 mm citrate, pH 3.5 | 67 | 0.17 | 98% |
| CAT7/Brij | CAT7 | Brij S100 | 54.5:25:20:0.5 | 5 mm citrate, pH 3.5 | 124 | 0.14 | 82% |
| CAT18/DMG | CAT18 | PEG₂ₖ-DMG | 50:40:7:3 | 5 mm citrate, pH 3.5 | 92 | 0.19 | 95 |
| CAT21/DMG | CAT21 | PEG₂ₖ-DMG | 50:40:7:3 | 5 mm citrate, pH 3.5 | 68 | 0.31 | 96 |
| CAT22/DMG | CAT22 | PEG₂ₖ-DMG | 50:40:7:3 | 5 mm citrate, pH 3.5 | 65 | 0.32 | 96 |
| CAT23/DMG | CAT23 | PEG₂ₖ-DMG | 50:40:7:3 | 5 mm citrate, pH 3.5 | 95 | 0.16 | 96 |
| CAT25/DMG | CAT25 | PEG₂ₖ-DMG | 50:40:7:3 | 5 mm citrate, pH 3.5 | 68 | 0.4 | 96 |
| CAT26/DMG | CAT26 | PEG₂ₖ-DMG | 50:40:7:3 | 5 mm citrate, pH 3.5 | 68 | 0.32 | 97 |
| CAT27/DMG | CAT27 | PEG₂ₖ-DMG | 50:40:7:3 | 5 mm citrate, pH 3.5 | 62 | 0.38 | 95 |
| CAT28/DMG | CAT28 | PEG₂ₖ-DMG | 50:40:7:3 | 5 mm citrate, pH 3.5 | 68 | 0.21 | 98 |
| CAT29/DMG | CAT29 | PEG₂ₖ-DMG | 50:40:7:3 | 5 mm citrate, pH 3.5 | 60 | 0.35 | 97 |
| CAT30/DMG | CAT30 | PEG₂ₖ-DMG | 50:40:7:3 | 5 mm citrate, pH 3.5 | 64 | 0.34 | 97 |
| CAT32/DMG | CAT32 | PEG₂ₖ-DMG | 50:40:7:3 | 5 mm citrate, pH 3.5 | 62 | 0.23 | 98 |
| CAT34/DMG | CAT34 | PEG₂ₖ-DMG | 50:40:7:3 | 5 mm citrate, pH 3.5 | 69 | 0.22 | 99 |
| CAT7/DMG | CAT7 | PEG₂ₖ-DMG | 50:40:7:3 | 5 mm citrate, pH 3.5 | 72 | 0.47 | 99 |

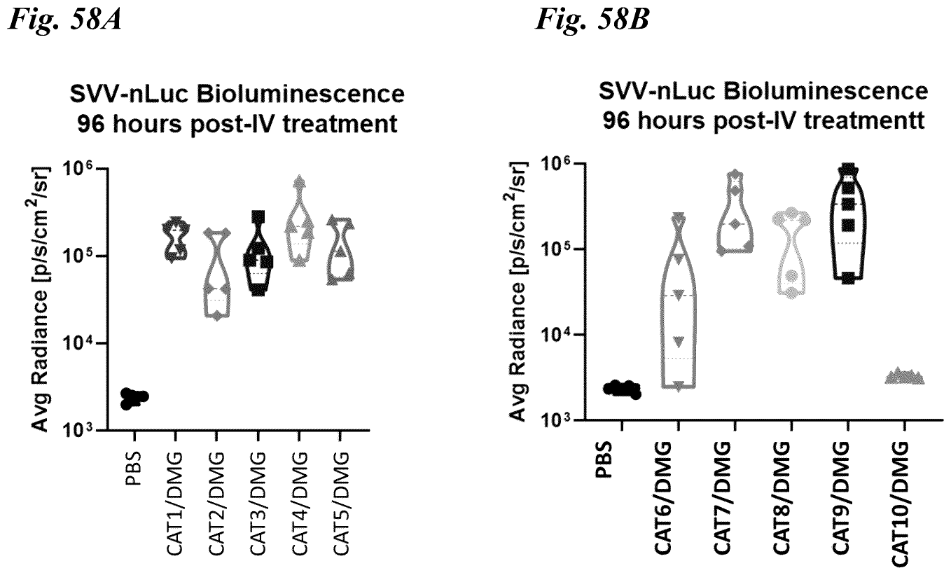

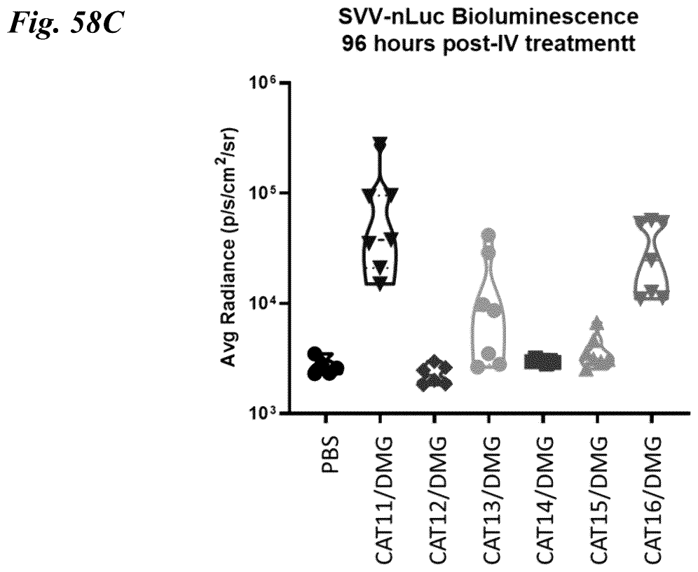

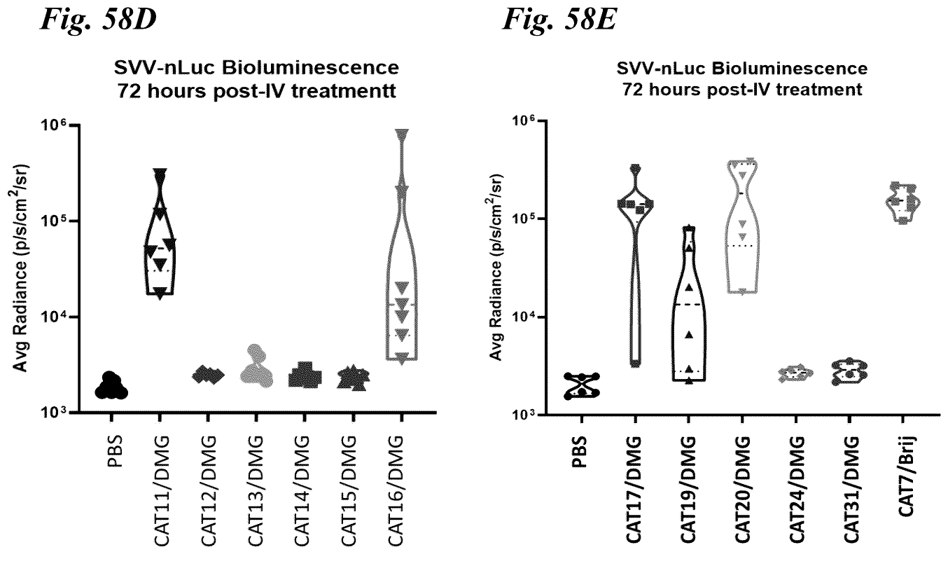

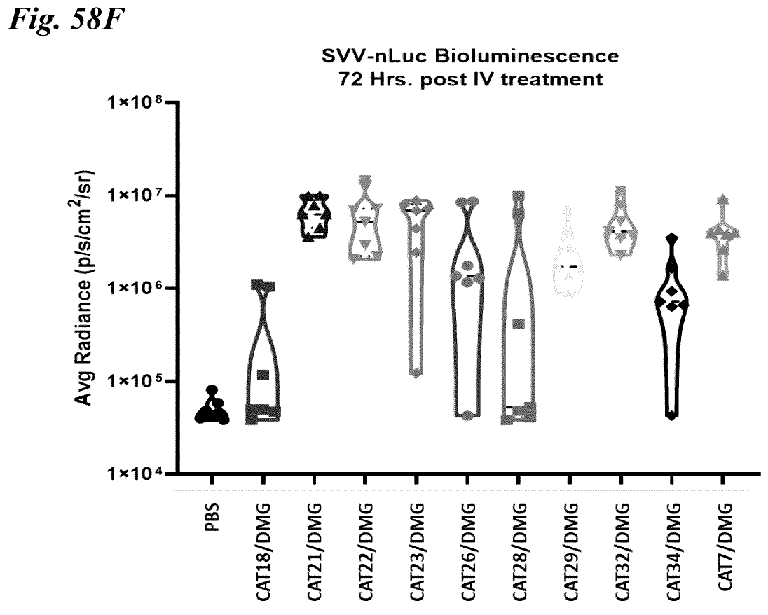

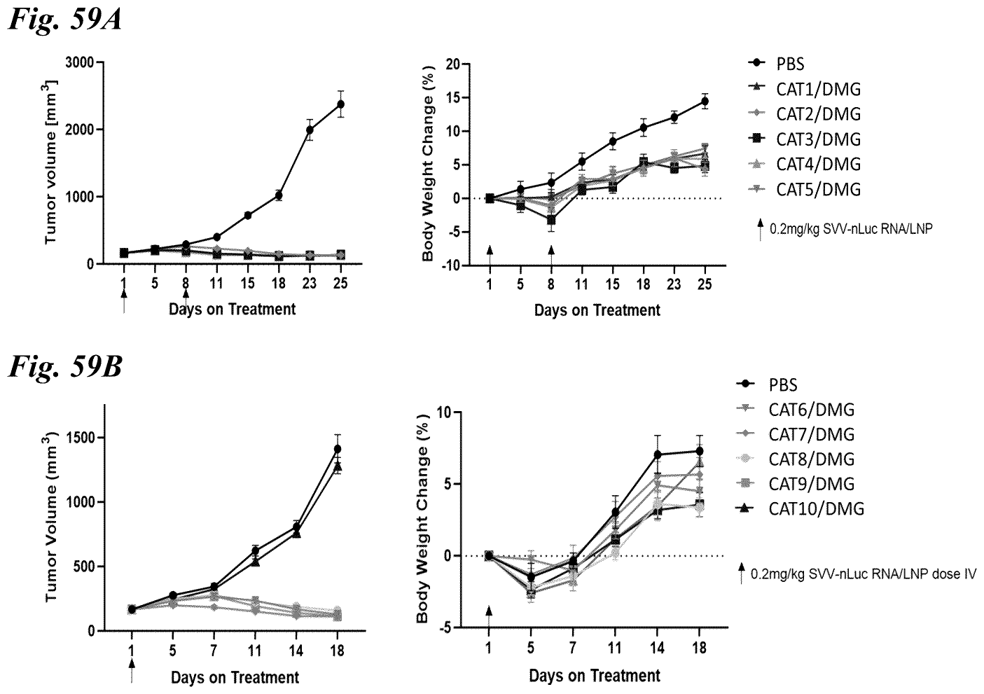

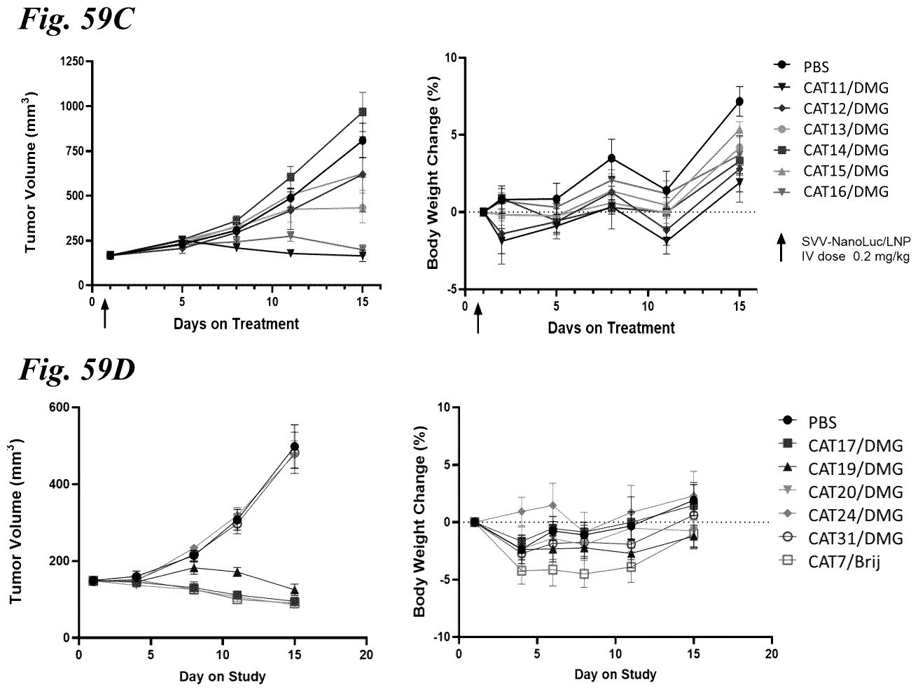

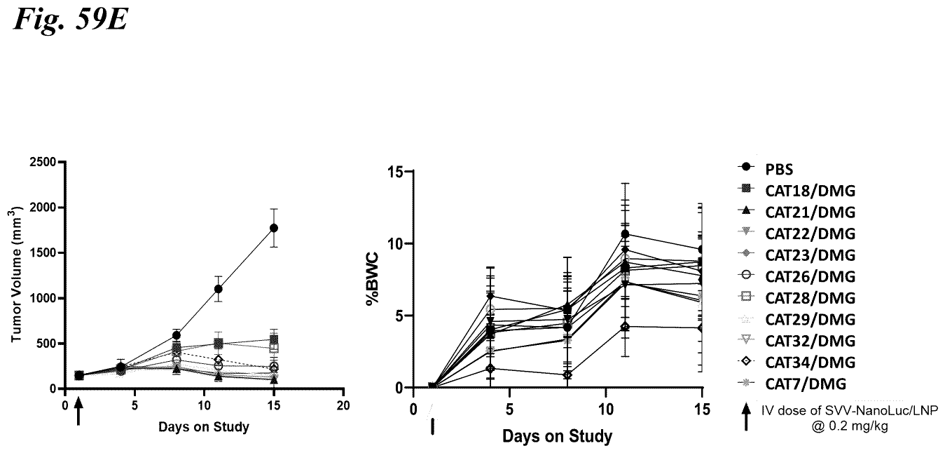

NCI-H446 human SCLC cells (5×10⁶ cells/0.1 mL in a 1:1 mixture of serum-free PBS and Matrigel®) were subcutaneously inoculated in the right flank of 8-week-old female athymic nude mice (Charles River Laboratories). When median tumor size reached approximately 150 mm³ (120-180 mm³ range), mice were intravenously administered 0.2 mg/kg of PBS or the LNPs comprising SVV-RNA on day 1 or on days 1 and 8. Bioluminescence (BLI) was assessed 96 h post-dose utilizing optical imagine IVIS Lumina (PerkinElmer), and the signal was quantified using Molecular Imaging software (FIGS. 58A-58F). Tumor volume and body weight were assessed 3 times per week (FIGS. 59A-59E).

Tumor regression after two 0.2 mg/kg doses was observed for the CAT1 to CAT5 formulations (FIG. 59A, left), and all formulations were well-tolerated (FIG. 59A, right). Tumor regression at a single 0.2 mg/kg dose was observed for the CAT6-CAT9, CAT11, CAT16-CAT17, CAT19-CAT24, CAT26, CAT29, CAT32, and CAT34 formulations (FIGS. 59B-59E, left), and all formulations were well-tolerated (FIGS. 59B-59E, right). Tumor growth inhibition was observed with CAT12-CAT13, CAT5, CAT18, and CAT28 formulations (FIGS. 59B-59E, left), and all formulations were well-tolerated (FIGS. 59B-59E, right).

### Example 21: In Vivo Studies of LNPs Comprising CAT7 and Different PEG-Lipids

Alternative PEG-lipids used in this and the following examples are listed in Table 28 below:

TABLE 28
PEG-Lipids

| | Chemical | |
|---|---|---|
| Name | Name | Formula |
| CHM- 001 | octadecyl 2-(poly (ethylene glycol) 2000)- acetate | 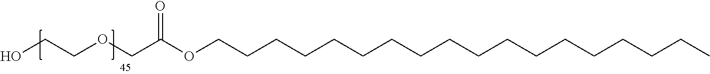 |
| CHM- 004 | octadecyl 2-(methyl- poly (ethylene glycol) | 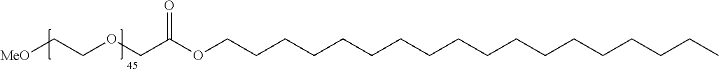 |
| | 2000)- | |
| | acetate | |
| CHM- 005 | Hexadecyl 2-(poly (ethylene glycol) 2000)- acetate | 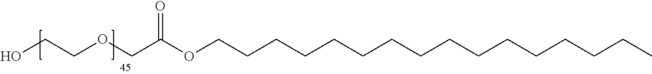 |
| CHM- 006 | tetradecyl 2-(poly (ethylene glycol) 2000)- acetate | 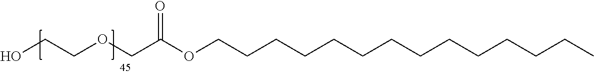 |
| CHM- 012 | Poly (ethylene glycol)2000 N-octadecyl carbamate | 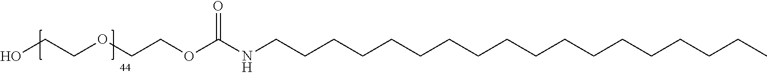 |

The in vivo pharmacodynamics and anti-tumor efficacy of SVV-RNA encapsulated in LNP with varying lipid compositions was evaluated in a mouse model for small cell lung cancer (SCLC). The RNA molecule encoding SVV viral genomes and NLuc were encapsulated in LNPs prepared according to Table 23 below, following a similar procedure described in Example 19. Total lipid concentration was set to 20 mM, and the lipid-nitrogen-to-phosphate ratio (N:P) was 9.

TABLE 23
LNP formulations for in vivo studies

| | | | Ionizable | | | | |
|---|---|---|---|---|---|---|---|
| | Ionizable | | lipid:Cholesterol:DSPC:PEG- | | Size | | |
| Formulation | Lipid | PEG-Lipid | lipid (mol %) | Acidifying Buffer | (nm) | PDI | % EE |
| CAT7/DMG_1 | CAT7 | PEG₂ₖ-DMG | 50:40:7:3 | 5 mm citrate, pH 3.5 | 60.4 | 0.34 | 98 |
| CAT7/CHM1_1 | CAT7 | CHM-001 | 54.6:25.1:20.1:0.25 | 5 mm citrate, pH 3.5 | 123.3 | 0.09 | 97 |
| CAT7/DMG_2 | CAT7 | PEG₂ₖ-DMG | 44.5:50:5:0.5 | 5 mm citrate, pH 3.5 | 116.5 | 0.12 | 97 |
| CAT7/DMG_3 | CAT7 | PEG₂ₖ-DMG | 40:50:8.75:1.25 | 5 mm citrate, pH 3.5 | 66.3 | 0.14 | 98 |
| CAT7/DMG_4 | CAT7 | PEG₂ₖ-DMG | 60:25:14.5:0.5 | 5 mm citrate, pH 3.5 | 93.5 | 0.12 | 97 |
| CAT7/DMG_5 | CAT7 | PEG₂ₖ-DMG | 60:34.3:5:0.7 | 5 mm citrate, pH 3.5 | 117.9 | 0.16 | 97 |
| CAT7/CHM6_1 | CAT7 | CHM-006 | 50:42.5:7:0.5 | 5 mm citrate, pH 3.5 | 102.3 | 0.13 | 98 |
| CAT7/CHM6_2 | CAT7 | CHM-006 | 58:33.5:7:1.5 | 5 mm citrate, pH 3.5 | 103.3 | 0.2 | 96 |
| CAT7/CHM6_3 | CAT7 | CHM-006 | 58:34.5:7:0.5 | 5 mm citrate, pH 3.5 | 117.6 | 0.17 | 98 |

The pharmacodynamics (assessed via a bioluminescence assay) and tumor growth inhibition ability of the SVV-NanoLuc-encapsulated LNPs was evaluated as described in Example 20.

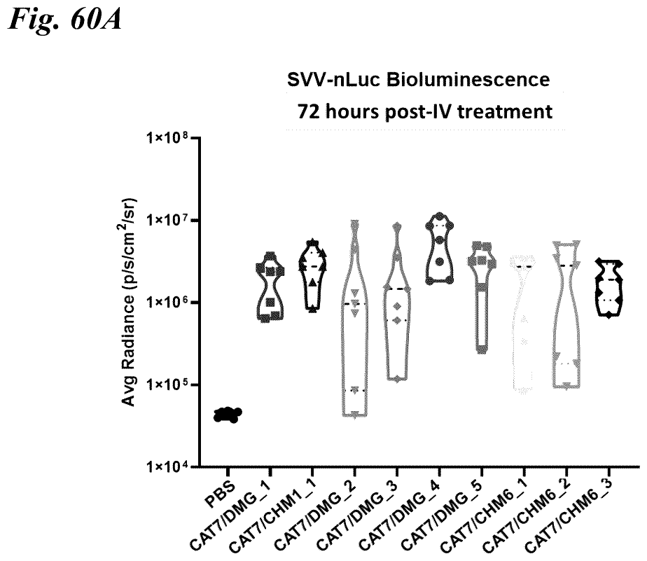

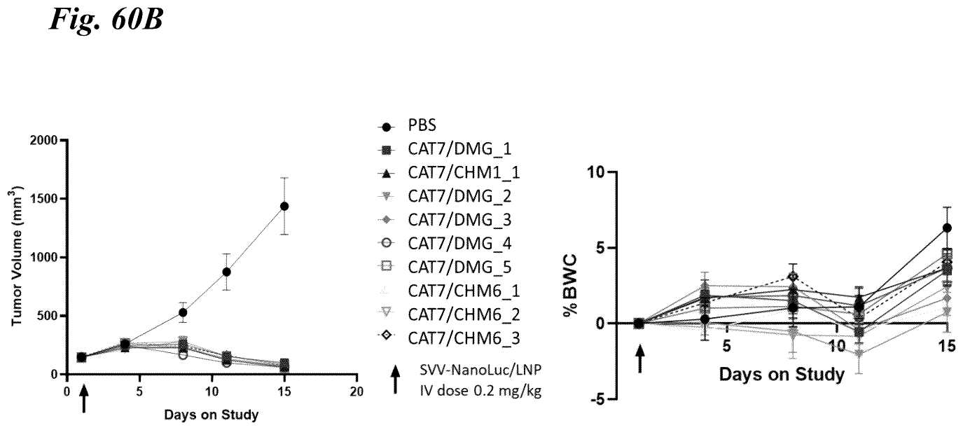

Nanoluciferase is detectable at 72 hours post-injection, indicative of continuous SVV (FIG. 60A). Complete tumor regression at a single 0.2 mg/kg dose was observed for all tested formulations, and all formulations were well-tolerated (FIG. 60B).

### Example 22: Pharmacokinetics Evaluation of LNP Formulations

The pharmacokinetics (PK) of Coxsackievirus A21 (CVA21)-RNA-encapsulating LNP formulations were evaluated in rats.

In this example, the RNA molecules encoding CVA-21 viral genomes were encapsulated in LNPs prepared according to Table 24 below, following the similar procedure as described in Example 19.

TABLE 24
LNP formulations for pharmacokinetics studies

| | | | | | | | | | Dosing |
|---|---|---|---|---|---|---|---|---|---|
| | | | Ionizable | | | | | | Schedule |
| | Ionizable | | lipid:DSPC:Cholesterol:PEG- | Acidifying | Size | | | | (base on |
| Formulation | Lipid | PEG-Lipid | lipid (mol %) | Buffer | (nm) | PDI | % EE | Payload | RNA conc.) |
| OC/CHM1 | Coatsome | CHM-001 | 49:22:28.5:0.5 | 25 mM | 83 | 0.25 | 97 | SVV-NEG-RNA | 1 mg/kg, |
| | SS-OC | | | acetate, pH 5 | | | | | Q2W2 |
| OC/CHM4 | Coatsome | CHM-004 | 49:22:28.5:0.5 | 25 mM | 77 | 0.18 | 98 | SVV-NEG-RNA | 1 mg/kg, |
| | SS-OC | | | acetate, pH 5 | | | | | Q2W2 |
| OC/CHM5 | Coatsome | CHM-005 | 49:22:28.5:0.5 | 25 mM | 74 | 0.2 | 99 | SVV-NEG-RNA | 1 mg/kg, |
| | SS-OC | | | acetate, pH 5 | | | | | Q2W2 |
| OC/CHM6 | Coatsome | CHM-006 | 49:22:28.5:0.5 | 25 mM | 84 | 0.2 | 98 | SVV-NEG-RNA | 1 mg/kg, |
| | SS-OC | | | acetate, pH 5 | | | | | Q2W2 |
| OC/CHM12 | Coatsome | CHM-012 | 49:22:28.5:0.5 | 25 mM | 73 | 0.15 | 99 | SVV-NEG-RNA | 1 mg/kg, |
| | SS-OC | | | acetate, pH 5 | | | | | Q2W2 |
| OC/Brij | Coatsome | BRIJ ™ S100 | 49:22:28.5:0.5 | 25 mM | 69 | 0.18 | 98 | SVV-NEG-RNA | 1 mg/kg, |
| | SS-OC | | | acetate, pH 5 | | | | | Q1W2; |
| | | | | | | | | | 1 mg/kg, |
| | | | | | | | | | Q2W2 |
| OC/DMG | Coatsome | PEG₂ₖ-DMG | 49:22:28.5:0.5 | 25 mM | 70 | 0.24 | 98 | SVV-NEG-RNA | 1 mg/kg, |
| | SS-OC | | | acetate, pH 5 | | | | | Q2W2 |
| OC/ DPG | Coatsome | PEG₂ₖ-DPG | 49:22:28.5:0.5 | 25 mM | 69 | 0.21 | 98 | SVV-NEG-RNA | 1 mg/kg, |
| | SS-OC | | | acetate, pH 5 | | | | | Q2W2 |
| OC/Brij | Coatsome | BRIJ ™ S100 | 49:22:28.5:0.5 | 25 mM | 142 | 0.25 | 94 | CVA21-RNA | 0.3 mg/kg, |
| | SS-OC | | | acetate, pH 5 | | | | | Q2W2 |
| CAT7/DMG_6 | CAT7 | PEG₂ₖ-DMG | 40:20:39.5:0.5 | 5 mM citrate, | 90.6 | 0.10 | 98 | CVA21-RNA | 0.3 mg/kg, |
| | | | | pH 3.5 | | | | | Q2W2 |
| CAT7/DMG_3 | CAT7 | PEG₂ₖ-DMG | 40:8.75:50:1.25 | 5 mM citrate, | 82.0 | 0.14 | 98 | CVA21-RNA | 0.3 mg/kg, |
| | | | | pH 3.5 | | | | | Q2W2 |
| CAT7/DMG_5 | CAT7 | PEG₂ₖ-DMG | 60:5:34.3:0.7 | 5 mM citrate. | 156.7 | 0.12 | 97 | CVA21-RNA | 0.3 mg/kg, |
| | | | | pH 3.5 | | | | | Q2W2 |
| CAT7/CHM1_1 | CAT7 | CHM-001 | 54.6:20.1:25.1:0.25 | 5 mM citrate, | 106.9 | 0.11 | 97 | CVA21-RNA | 0.3 mg/kg, |
| | | | | pH 3.5 | | | | | Q2W2 |
| CAT7/CHM6_4 | CAT7 | CHM-006 | 50.1:7:42.6:0.25 | 5 mM citrate | 131.1 | 0.10 | 98 | CVA21-RNA | 0.3 mg/kg, |
| | | | | pH 3.5 | | | | | Q2W2 |
| CAT7/Brij | CAT7 | BRIJ ™ S100 | 54.5:20:25:0.5 | 5 mM citrate, | 116 | 0.2 | 88 | CVA21-RNA | 0.3 mg/kg, |
| | | | | pH 3.5 | | | | | Q2W2 |
| OC/DMG | Coatsome | PEG₂ₖ-DMG | 49:22:28.5:0.5 | 25 mM | 110 | 0.28 | 94 | CVA21-RNA | 0.3 mg/kg, |
| | SS-OC | | | acetate, pH 5 | | | | | Q2W2 |
| CAT7/CHM6_4 | CAT7 | CHM-006 | 50:7:40:3 | 5 mM citrate, | 102.6 | 0.37 | 98 | CVA21-RNA | 0.3 mg/kg, |
| | | | | pH 3.5 | | | | | Q2W2 |
| CAT11/DMG | CAT11 | PEG₂ₖ-DMG | 50:7:40:3 | 5 mM citrate | 79.8 | 0.31 | 98 | CVA21-RNA | 0.3 mg/kg, |
| | | | | pH 3.5 | | | | | Q2W2 |
| CAT11/Brij | CAT11 | BRIJ ™ S100 | 54.5:20:25:0.5 | 5 mM citrate, | 122.9 | 0.18 | 85 | CVA21-RNA | 0.3 mg/kg, |
| | | | | pH 3.5 | | | | | Q2W2 |

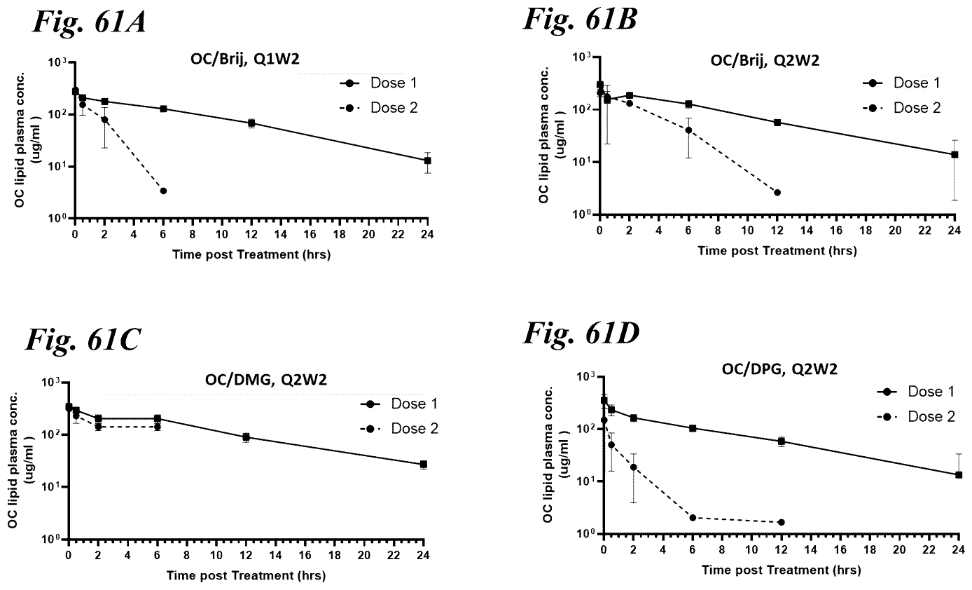

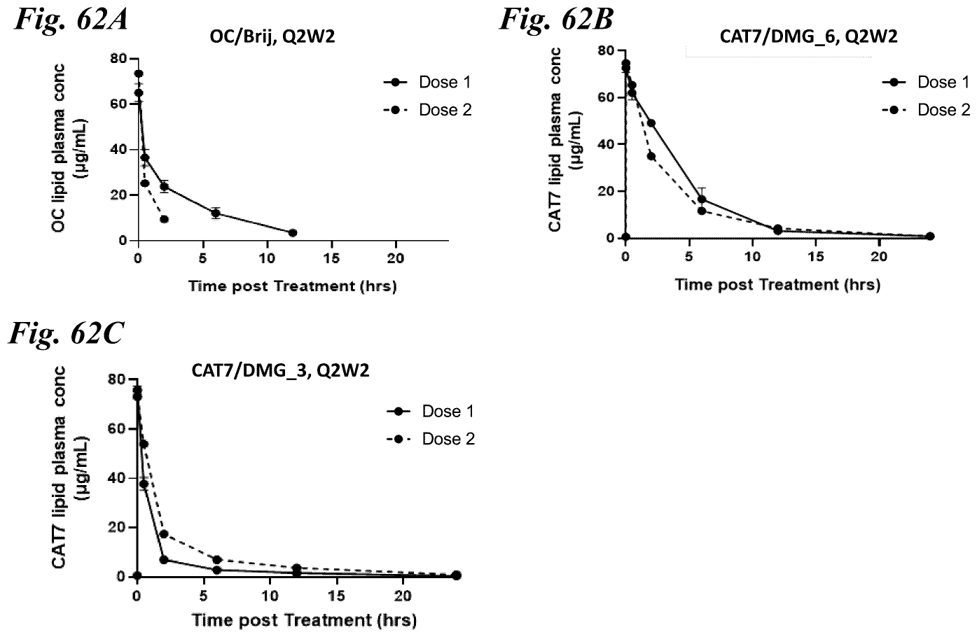

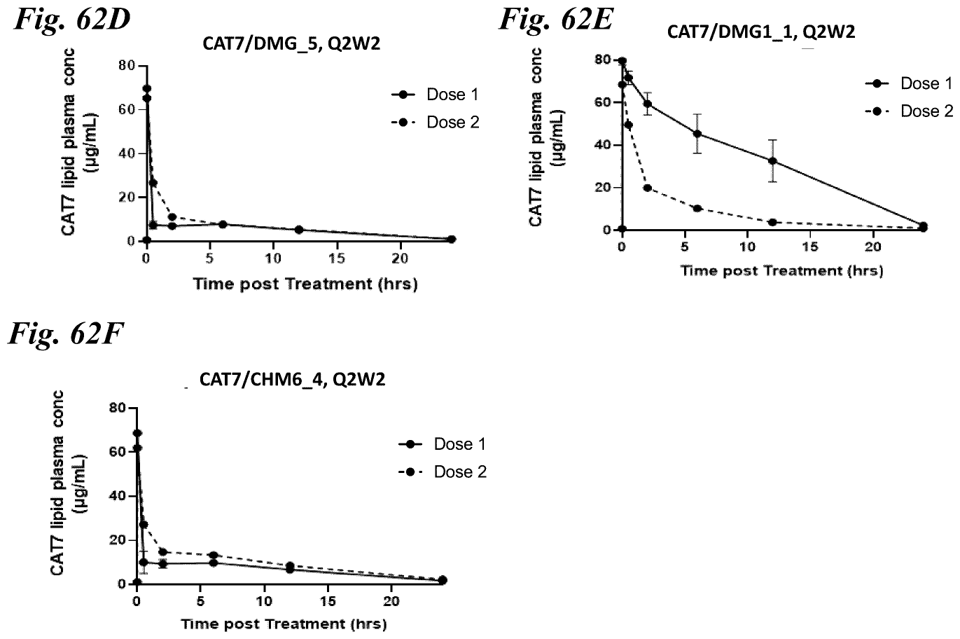

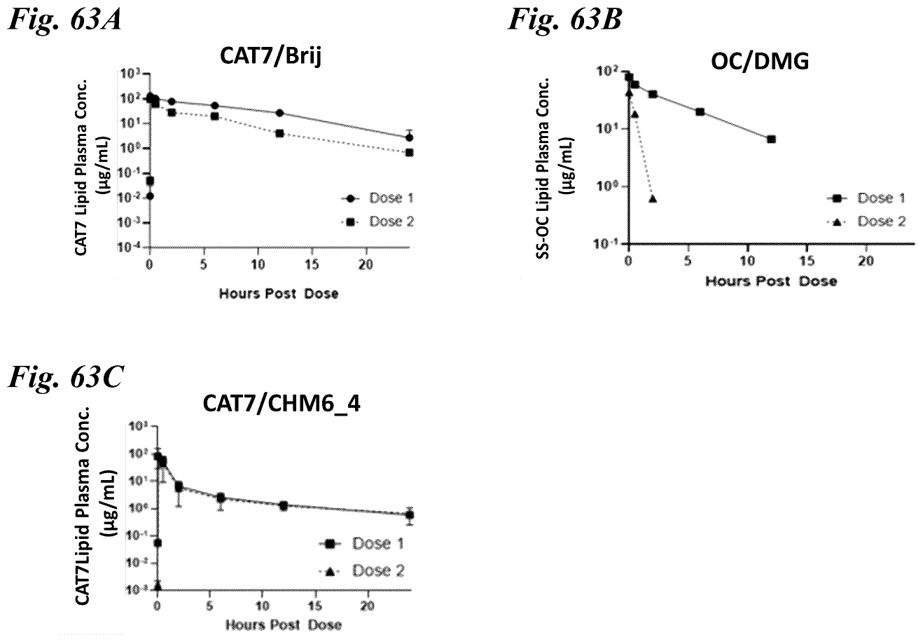

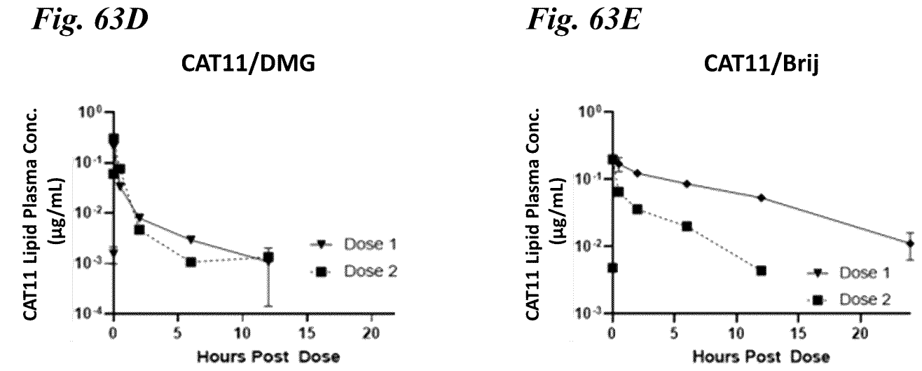

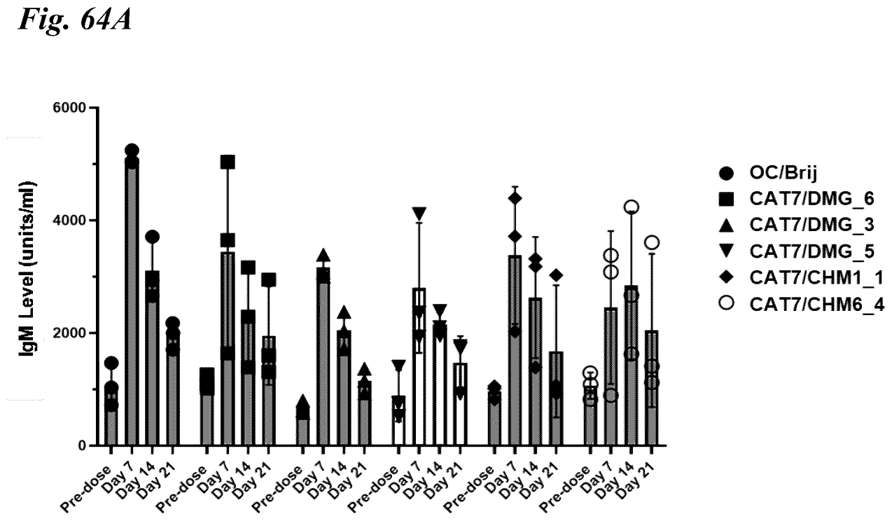

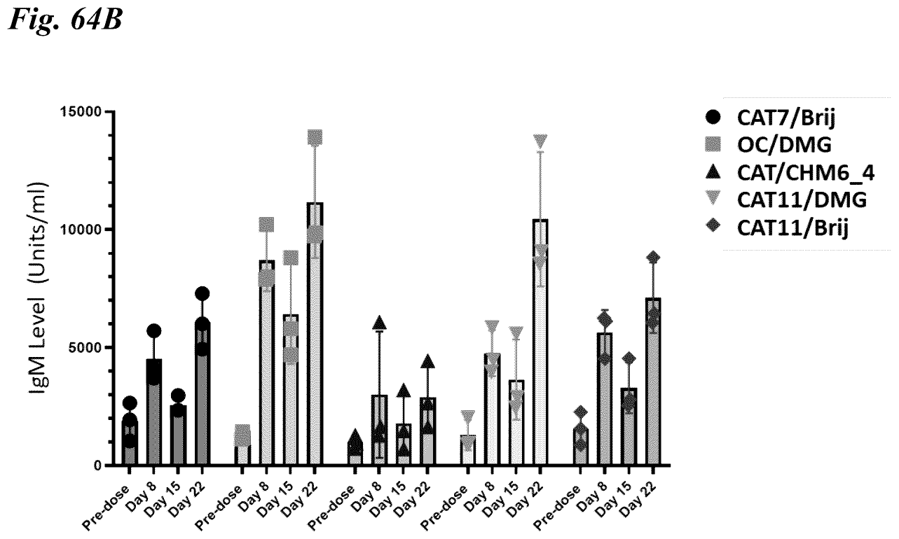

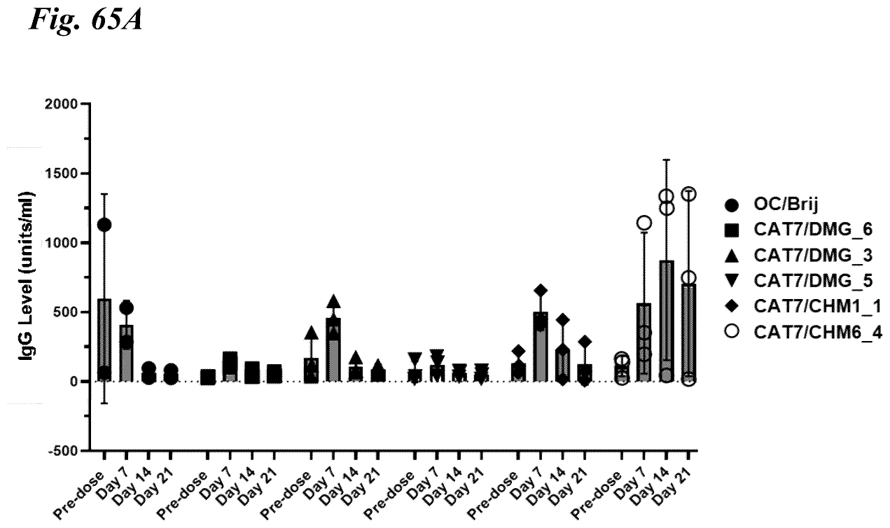

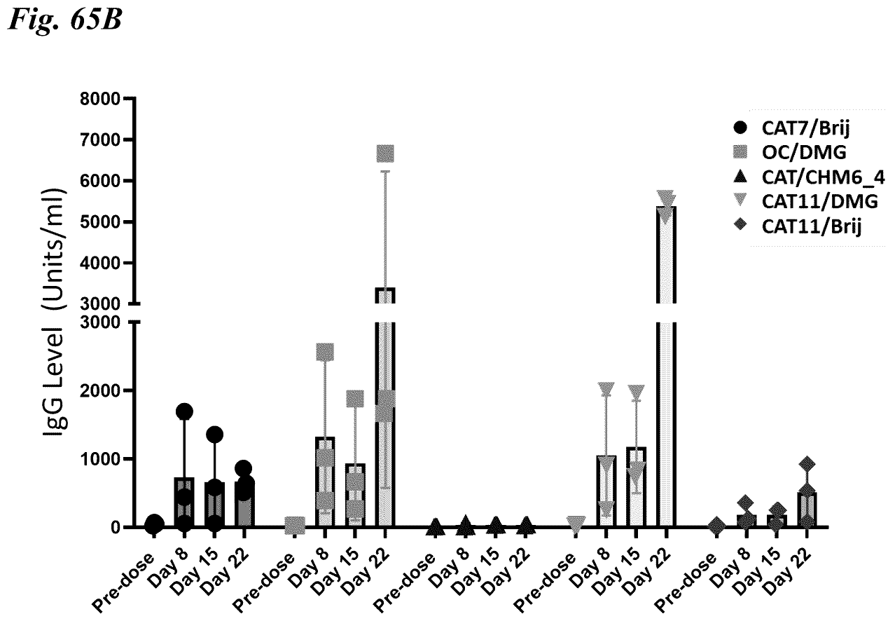

Naïve female Sprague Dawley, JVC rats (age: 12 weeks) were intravenously administered 1 or 0.3 mg/kg of viral genomes comprised in the LNPs on days 1 and 15 (Q2W2) or on day 1 and day 8 (Q1W2). Plasma samples were collected at the predetermined times. The concentration of the ionizable lipid comprised in the LNPs (SS—OC, CAT7, or CAT11) in plasma were measured by LC-MS (FIGS. 61A-61D, 62A-62F, and 63A-63E) and the pharmacokinetics parameters were calculated and summarized in Table 25-1 and Table 25-2. IgM and IgG levels were analyzed by enzyme-linked immunoassay (ELISA) (FIGS. 64A-64B and FIGS. 65A-65B).

TABLE 25-1
Pharmacokinetics parameters

| | | | T_{1/2} | Tₘₐₓ | AUC_{INF} | AUC_{LAST} | C₀ | CL | Cₘₐₓ | Vₛₛ |
|---|---|---|---|---|---|---|---|---|---|---|
| Formulation | Dose # | Dose # | (h) | (h) | (h*μg/mL) | | (μg/mL) | (mL/h/kg) | (μg/mL) | (mL/kg) |
| OC/CHM1 | 1 | Day 1 | 4.92 | 0.02 | 1980.57 | 1915.72 | 368.81 | 6.06 | 364.94 | 43.17 |
| | 2 | Day 15 | 1.95 | 0.02 | 296.06 | 294.78 | 182.03 | 40.53 | 173.21 | 160.23 |
| OC/CHM4 | 1 | Day 1 | 3.51 | 0.5 | 2313.35 | 2284.79 | 344.1 | 5.19 | 401.04 | 27.54 |
| | 2 | Day 15 | 1.87 | 0.02 | 456.71 | 454.71 | 206.18 | 26.27 | 200.34 | 106.16 |
| OC/CHM5 | 1 | Day 1 | 4.54 | 0.02 | 2565.94 | 2499.28 | 404.68 | 4.68 | 403.35 | 30.67 |
| | 2 | Day 15 | 4.25 | 0.02 | 830.22 | 705.19 | 212.04 | 14.45 | 208.59 | 94.25 |
| OC/CHM6 | 1 | Day 1 | 5.04 | 0.02 | 3252.74 | 3130.05 | 477.19 | 3.69 | 472.62 | 26.03 |
| | 2 | Day 15 | 2.74 | 0.02 | 1240.69 | 1239.1 | 220.02 | 9.67 | 216.1 | 51.98 |
| OC/CHM12 | 1 | Day 1 | 4.3 | 0.02 | 2753.28 | 2692.79 | 431.4 | 4.36 | 431.02 | 26.56 |
| | 2 | Day 15 | 5.92 | 0.02 | 1459.81 | 1122.65 | 282.05 | 8.22 | 274.99 | 43.17 |
| OC/Brij | 1 | Day 1 | 6.25 | 0.02 | 2016.16 | 1890.9 | 306.42 | 5.95 | 299.62 | 51.87 |
| | 2 | Day 8 | 1.92 | 0.02 | 725.02 | 717.72 | 210.78 | 16.55 | 209.51 | 49.68 |
| OC/Brij | 1 | Day 1 | 5.89 | 0.02 | 2109.85 | 1998.58 | 278.63 | 5.69 | 276.04 | 48.38 |
| | 2 | Day 15 | 0.95 | 0.02 | 383.47 | 378.75 | 305.55 | 31.29 | 298.73 | 45.63 |
| OC/DMG | 1 | Day 1 | 7.1 | 0.02 | 2918.23 | 2641.81 | 346.95 | 4.11 | 345.08 | 42.14 |
| | 2 | Day 15 | 2.83 | 0.02 | 1020.25 | 783.99 | 321.7 | 11.76 | 318.13 | 47.55 |
| OC/DPG | 1 | Day 1 | 5.91 | 0.02 | 1929.25 | 1815.2 | 358.99 | 6.22 | 353.9 | 52.8 |
| | 2 | Day 15 | 1.96 | 0.02 | 140.25 | 135.55 | 155.02 | 85.56 | 149.29 | 202.16 |

TABLE 25-2
Pharmacokinetics parameters

| | Ionizable | | | | | | | |
|---|---|---|---|---|---|---|---|---|
| | Lipid Dose | | T_{1/2} | AUC₀₋₂ | AUC_{INF} | CL | Vₛₛ | Cₘₐₓ |
| Formulation | (mg/kg) | Dose # | (h) | h*μg/mL | | (mL/h/kg) | (mL/kg) | μg/mL |
| OC/Brij | 3.6 | Dose 1 | 2.8 | 69.01 | 183.79 | 19.59 | 81.85 | 65.06 |
| | 3.6 | Dose 2 | 0.6 | 42.84 | 48.22 | 74.65 | 65.31 | 73.5 |
| CAT7/DMG_6 | 6.5 | Dose 1 | 2.6 | 116.08 | 308.26 | 20.96 | 93.47 | 72.58 |
| | 6.5 | Dose 2 | 2.2 | 107.39 | 263.81 | 24.5 | 117.35 | 74.66 |
| CAT7/DMG_3 | 6.5 | Dose 1 | 0.6 | 54.21 | 95.67 | 67.55 | 275.46 | 75.79 |
| | 6.5 | Dose 2 | 1.8 | 79.42 | 181.21 | 35.66 | 185.34 | 73.04 |
| CAT7/DMG_5 | 6.5 | Dose 1 | 0.8 | 24.98 | 127.23 | 50.79 | 416.32 | 69.77 |
| | 6.5 | Dose 2 | 4.2 | 48.38 | 166.82 | 38.74 | 304.37 | 65.38 |
| CAT7/CHM1_1 | 6.5 | Dose 1 | 5 | 135.22 | 730.39 | 8.85 | 68.69 | 79.67 |
| | 6.5 | Dose 2 | 2.3 | 77.67 | 204.63 | 31.58 | 176.45 | 68.46 |
| CAT7/CHM6_4 | 6.5 | Dose 1 | 0.9 | 29.79 | 162.35 | 39.8 | 338.16 | 68.68 |
| | 6.5 | Dose 2 | 5.5 | 51.38 | 246.32 | 26.23 | 249.88 | 61.97 |
| CAT7/Brij | 6.5 | Dose 1 | 4.55 | 192.83 | 834.37 | 7.74 | 53.92 | 128.22 |
| | 6.5 | Dose 2 | 2.9 | 103.72 | 284.41 | 22.72 | 111.78 | 98.63 |
| OC/DMG | 3.6 | Dose 1 | 3.52 | 107.34 | 329.48 | 10.93 | 57.26 | 79.49 |
| | 3.6 | Dose 2 | 0.32 | 22.39 | 22.68 | 158.73 | 76.28 | 43.77 |
| CAT7/CHM6_4 | 6.5 | Dose 1 | 3.91 | 61.15 | 101.08 | 63.93 | 306.33 | 93.43 |
| | 6.5 | Dose 2 | 2.17 | 67.3 | 109.03 | 59.27 | 242.92 | 81.12 |
| CAT11/DMG | 6.6 | Dose 1 | 1.92 | 0.07 | 0.11 | 60435.32 | 138804.45 | 0.21 |
| | 6.6 | Dose 2 | 1.47 | 0.12 | 0.13 | 50064.5 | 47336.6 | 0.3 |
| CAT11/Brij | 6.6 | Dose 1 | 6.76 | 0.31 | 1.57 | 4203.47 | 38959.22 | 0.19 |
| | 6.6 | Dose 2 | 2.58 | 0.13 | 0.32 | 20537.25 | 82150.98 | 0.2 |

LNP formulations with different ratios and/or types of PEG-lipids displaying varying T_{1/2}, exposure, and clearance after multiple doses. These data indicate that the LNP compositions can be adapted to meet the need of various therapeutic payloads for long to short exposure.

Anti-PEG IgM level after dosing the LNP formulations was low and decreased from day 7 to 21 (FIG. 64A and FIG. 64B). Anti-PEG IgG was also low and did not significantly increase with multiple dose, indicating a low potential for immunogenicity (FIG. 65A and FIG. 65B). Among the tested formulations, LNPs comprising CAT7 as the ionizable lipid and CHM-006 as the PEG-lipid were observed with the lowest IgM and IgG levels.

### Example 23: Formulation of LNPs Encapsulating mRNA

SS—OC:Cholesterol:DSPC:PEG-lipid LNPs encapsulating mRNA at a N:P ratio of about 8:1 to 20:1 are prepared. The PEG-lipid is PEG2k-DPG, PEG2k-DMG or BRIJ™ S100. Total lipid concentration is about 10 to about 60 mM. Formulations are mixed and dialyzed, and concentrated. Size is measured by dynamic light scattering and encapsulation efficiency is measured by RiboGreen. The results show that BRIJ™ S100 could be used in replacement of PEG2k-DPG or PEG2k-DMG for mRNA LNP formulation.

mRNA LNP formulations in this Example are tested for pharmacokinetic characteristics upon repeat dosing via intravenous administration in mice. Copy number of RNA in serum post-dose is measured at predetermined time point. The results show that LNPs formulated using BRIJ™ S100 exhibits a reduced clearance rate upon the second dose compared to LNPs formulated using PEG-2k DPG or PEG2k-DMG.

### Example 24: Formulated of LNPs Encapsulating mRNAs

This example illustrates the encapsulation of mRNAs in lipid nanoparticle (LNP) formulations. LNPs in this example comprise a lipid composition of CAT7:DSPC:cholesterol:CHM-006 at 54.5:20:25:0.5 mol %. The lipid mixture in ethanol was mixed with human erythropoietin (hEPO) mRNAs or bi-specific T cell engager (BiTE)-encoding mRNAs in RNA acidifying buffer (5 mM citrate, pH 3.5). Total lipid concentration was set to 20 mM, and the lipid-nitrogen-to-phosphate ratio (N:P) was 9.

LNPs were dialyzed against 50 mM phosphate, pH 6.0, for 12-16 h and secondary dialysis was performed against 50 mM HEPES, 50 mM NaCl, 263 mM sucrose, pH 7.3, for 4-24 h at room temperature. Post-dialyzed LNPs were concentrated using 100 kDa AMICON@ ULTRA CENTRIFUGAL filters (MilliporeSigma) and then sterile concentrated using 0.2 μm syringe filters. Samples were then characterized and diluted as needed. Upon dilution, a 5 w/v % glycerol spike was added if samples were stored at −20° C.

LNP sizes were measured by DLS, and the encapsulation efficacy was measured using a fluorescence-based RiboGreen assay (Table 26).

TABLE 26
LNP-formulated mRNAs

| | Ionizable | | | | | |
|---|---|---|---|---|---|---|
| | Lipid | | CAT:DSPC:Cholesterol:PEG₂ₖ- | Size | | |
| mRNA | (CAT) | PEG-Lipid | DMG (mol %) | (nm) | PDI | % EE |
| hEPO | CAT7 | CHM-006 | 54.5:20:25:0.5 | 85 | 0.14 | 97 |
| BiTE | CAT7 | CHM-006 | 54.5:20:25:0.5 | 86 | 0.13 | 97 |
| hEPO | CAT7 | CHM-006 | 54.5:20:25:0.5 | 87 | 0.16 | 97 |
| BiTE | CAT7 | CHM-006 | 54.5:20:25:0.5 | 88.5 | 0.13 | 98 |

### Example 25: Pharmacokinetics of LNP-Formulated mRNA

The PK of mRNA-encapsulating LNP formulations (Table 26) were evaluated in mice.

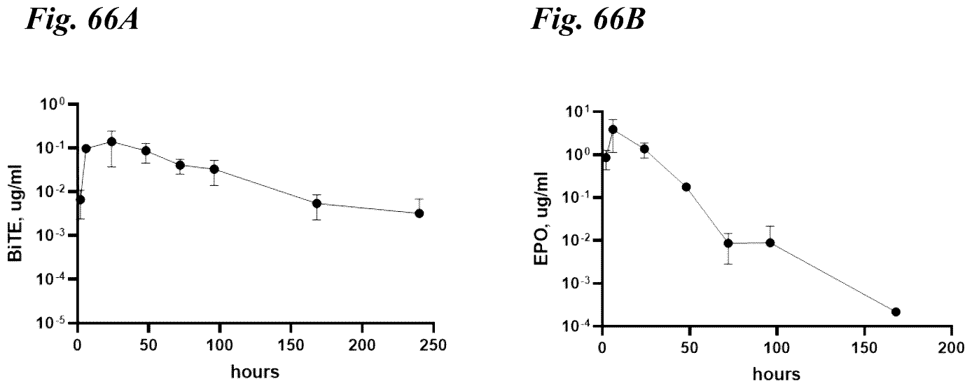

Naïve female Balb/c mice were dosed with 1 mg/kg of the LNPs. 3 mice were bled at each predetermined timepoints and plasma was frozen at −80° C. for later analysis. Plasma levels of hEPO and BiTE were measured by Meso Scale Discovery (MSA) electrochemiluminescence (ECL) assays (FIG. 66A and FIG. 66B). High levels of protein expression and prolonged exposure were observed.

### Example 26: LNP-Formulated RNAs with Varying Lengths

LNP formulations encapsulating RNA with various lengths were prepared according to Table 27 below, following a similar procedure as described in Example 24.

TABLE 27
LNP formulations

| RNA | | | | Ionizable | | | | |
|---|---|---|---|---|---|---|---|---|
| length | | Ionizable | | lipid:DSPC:Cholesterol:PEG- | Acidifying | Size | | |
| (kb) | Formulation | Lipid | PEG-Lipid | lipid (mol %) | Buffer | (nm) | PDI | % EE |
| 5.9 | OC/Brij | Coatsome | BRIJ ™ S100 | 49:22:28.5:0.5 | 25 mM acetate, pH 5 | 77.9 | 0.26 | 98% |
| | | SS-OC | | | | | | |
| 14.2 | OC/Brij | Coatsome | BRIJ ™ S100 | 49:22:28.5:0.5 | 25 mM acetate, pH 5 | 93.5 | 0.23 | 95% |
| | | SS-OC | | | | | | |
| 12.6 | OC/Brij | Coatsome | BRIJ ™ S100 | 49:22:28.5:0.5 | 25 mM acetate, pH 5 | 131.9 | 0.32 | 92% |
| | | SS-OC | | | | | | |
| 5.9 | CAT7/DMG | CAT7 | PEG₂ₖ-DMG | 54.5:20:25:0.5 | 5 mM citrate, pH 3.5 | 80.4 | 0.05 | 99% |
| 14.2 | CAT7/DMG | CAT7 | PEG₂ₖ-DMG | 54.5:20:25:0.5 | 5 mM citrate, pH 3.5 | 99.8 | 0.22 | 93% |
| 12.6 | CAT7/DMG | CAT7 | PEG₂ₖ-DMG | 54.5:20:25:0.5 | 5 mM citrate, pH 3.5 | 99.3 | 0.21 | 93% |

The data show that LNPs maintained good biophysical properties (e.g., small size and PDI, high % EE) despite the variable length of the encapsulated RNA.

### Example 27: Formulation Studies and Modeling of LNPs Comprising CAT7

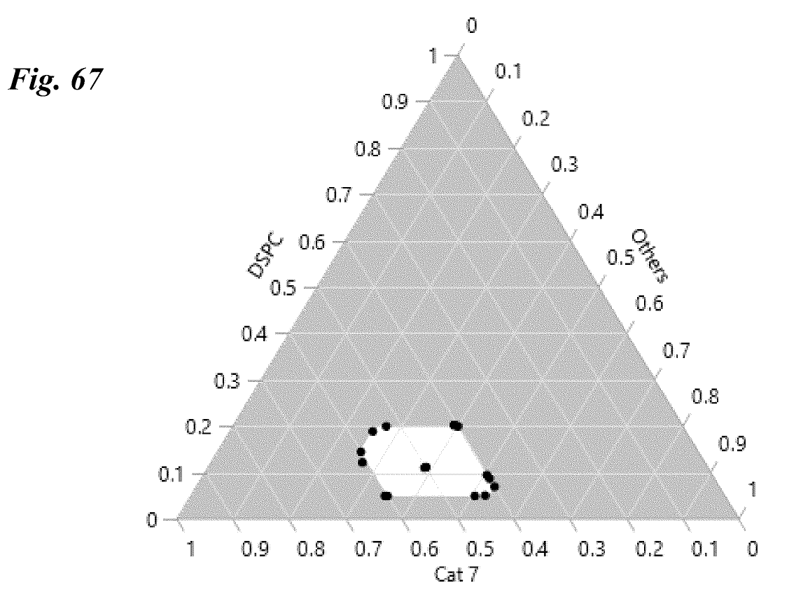

A-optimal criterion (Jones et al. 2021) was used to design formulation studies of LNPs comprising CAT7 (FIG. 67) and yielded 20 design of experiment (DOE) runs (Table 29). The total lipid concentration was set to 20 mM and the N:P ratio to 9. The design space tested LNPs comprising 40-60 mol % ionizable lipid of CAT7, 5-20 mol % helper lipid of DSPC, 25-50 mol % structural lipid of cholesterol, and 0.25-3% PEG-lipid of DMG-PEG2000 or CHM-001.

TABLE 29
Design of Experiment for CAT7 LNPs

| DOE | | |
|---|---|---|
| Run | Composition | Mol % |
| 1 | CAT7:DSPC:Cholesterol:PEG₂ₖ-DMG | 50:11.25:36.75:2 |
| 2 | CAT7:DSPC:Cholesterol:PEG₂ₖ-DMG | 40:20:39.5:0.5 |
| 3 | CAT7:DSPC:Cholesterol:PEG₂ₖ-DMG | 60:5:32:3 |
| 4 | CAT7:DSPC:Cholesterol:CHM-001 | 60.9:12.2:25.4:1.5 |
| 5 | CAT7:DSPC:Cholesterol:CHM-001 | 40.1:9.5:50.1:0.25 |
| 6 | CAT7:DSPC:Cholesterol:PEG₂ₖ-DMG | 44.5:5:50:0.5 |
| 7 | CAT7:DSPC:Cholesterol:PEG₂ₖ-DMG | 40:8.75:50:1.25 |
| 8 | CAT7:DSPC:Cholesterol:CHM-001 | 60.5:5.0:33.5:0.88 |
| 9 | CAT7:DSPC:Cholesterol:PEG₂ₖ-DMG | 60:14.5:25:0.5 |
| 10 | CAT7:DSPC:Cholesterol:CHM-001 | 40.4:20.2:38.6:0.83 |
| 11 | CAT7:DSPC:Cholesterol:PEG₂ₖ-DMG | 52.75:20:25:2.25 |
| 12 | CAT7:DSPC:Cholesterol:CHM-001 | 42.6:5.1:50.8:1.52 |
| 13 | CAT7:DSPC:Cholesterol:CHM-001 | 55.7:18.9:25.1:0.25 |
| 14 | CAT7:DSPC:Cholesterol:CHM-001 | 50.4:11.1:37.2:1.27 |
| 15 | CAT7:DSPC:Cholesterol:PEG₂ₖ-DMG | 40:7:50:3 |
| 16 | CAT7:DSPC:Cholesterol:CHM-001 | 60.2:5:34.6:0.25 |
| 17 | CAT7:DSPC:Cholesterol:PEG₂ₖ-DMG | 40:20:37:3 |
| 18 | CAT7:DSPC:Cholesterol:CHM-001 | 40.1:9.5:50.1:0.25 |
| 19 | CAT7:DSPC:Cholesterol:PEG₂ₖ-DMG | 60:5:34.3:0.7 |
| 20 | CAT7:DSPC:Cholesterol:PEG₂ₖ-DMG | 40.6:20.3:37.6:1.52 |

Within the parameters of the reliable design space, the DOE optimal composition was determined to be CAT7:DSPC:Cholesterol:PEG-lipid with the mol % ratio of 54.5:2025:0.5.

A Self-Validated Ensemble Modeling (SVEM) method (Lemkus et al. 2021) was used to formulate a model for predicting biophysical characteristics of LNPs with varying compositions and identifying and fine-tuning LNP systems for different desired outcomes. In developing the model, the aim was to minimize PDI (weighted as 1) and size (weighted as 0.1).

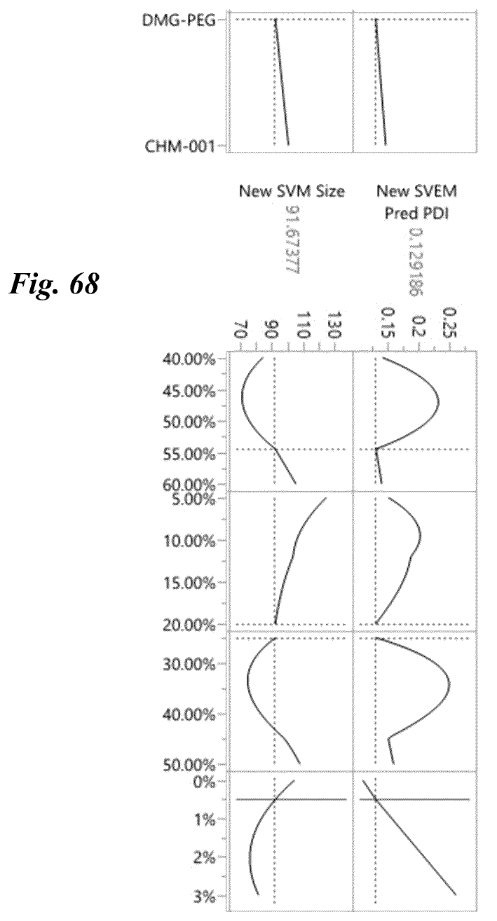

The resulting prediction profilers are shown in FIG. 68. Quadratic (curvature or non-linear) relationships are seen for CAT7, DSPC, and Cholesterol. CAT7 composition seems to significantly impact the PDI, with an increasing trend initially starting from 40 mol %, followed by a downward trend which stabilized at ˜55 mol %. Higher DSPC seems to favor a drop in both PDI and the size. Cholesterol follows a pattern very similar to CAT7 for both PDI and the size, but the model picks a lower molar composition. Increasing PEG-lipid composition is associated with a steep increase in observed PDI.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

While preferred embodiments of the present disclosure have been shown and described herein; it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A lipid nanoparticle (LNP) comprising a synthetic RNA viral genome encoding an oncolytic Coxsackievirus virus, wherein the Coxsackievirus is a CVA21 strain selected from the Kuykendall strain, the EF strain and the KY strain.

2. The LNP of claim 1, wherein the Coxsackievirus is the CVA21-KY strain, and wherein the CVA21-KY strain comprises a polynucleotide sequence having at least 80%, at least 85%, at least 90%, at least 95%, or 100% sequence identity to SEQ ID NO: 5.

3. The LNP of claim 1, wherein the Coxsackievirus is the CVA21-EF strain, and wherein the CVA21-EF strain comprises a polynucleotide sequence having at least 80%, at least 85%, at least 90%, at least 95%, or 100% sequence identity to SEQ ID NO: 9.

4. The LNP of claim 1, wherein the Coxsackievirus comprises a 5′ UTR (IRES) sequence having at least 80%, at least 85%, at least 90%, at least 95%, or 100% sequence identity to SEQ ID NO: 6 or 10.

5. The LNP of claim 1, wherein the Coxsackievirus comprises a P1 sequence having at least 80%, at least 85%, at least 90%, at least 95%, or 100% sequence identity to SEQ ID NO: 7 or 11.

6. The LNP of claim 1, wherein the Coxsackievirus comprises a 3D sequence having at least 80%, at least 85%, at least 90%, at least 95%, or 100% sequence identity to SEQ ID NO: 8 or 12.

7. The LNP of any one of claims 1-6, wherein the synthetic RNA viral genome does not comprise a polynucleotide sequence having more than 95%, more than 90%, more than 85%, or more than 80% sequence identity to SEQ ID NO: 1.

8. A lipid nanoparticle (LNP) comprising a synthetic RNA viral genome encoding an oncolytic Seneca Valley Virus (SVV), wherein the synthetic RNA viral genome comprises a polynucleotide sequence having at least 80%, at least 85%, at least 90%, at least 95%, or 100% sequence identity to SEQ ID NO: 68.

9. The LNP of claim 8, wherein the synthetic RNA viral genome comprises a 5′ UTR (IRES) sequence having at least 80%, at least 85%, at least 90%, at least 95%, or 100% sequence identity to nucleic acids 1-670 of SEQ ID NO: 68.

10. The LNP of claim 8 or 9, wherein the synthetic RNA viral genome encodes a SVV VP2 protein comprising a S177A mutation.

11. The LNP of any one of claims 1-10, wherein delivery of the LNP to a cell results in production of viral particles by the cell, and wherein the viral particles are infectious and lytic.

12. The LNP of any one of claims 1-11, wherein the synthetic RNA viral genome further comprises a heterologous polynucleotide encoding an exogenous payload protein.

13. The LNP of any one of claims 1-11, further comprising a second recombinant RNA molecule encoding an exogenous payload protein.

14. The LNP of claim 12 or 13, wherein the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2).

15. The LNP of claim 14, wherein the nitroreductase is NfsB or NfsA.

16. The LNP of claim 14, wherein the reovirus FAST protein is ARV p14, BRV p15, or a p14-p15 hybrid.

17. The LNP of claim 12 or 13, wherein the exogenous payload protein is a fluorescent protein, an enzymatic protein, a cytokine, a chemokine, an antigen-binding molecule capable of binding to a cell surface receptor, or a ligand for a cell-surface receptor.

18. The LNP of claim 17, wherein:
a) the cytokine is selected from GM-CSF, IFNγ, IL-2, IL-7, IL-12, IL-18, IL-21, and IL-36γ;
b) the ligand for a cell-surface receptor is Flt3 ligand or TNFSF14; or
c) the chemokine is selected from CXCL10, CCL4, CCL21, and CCL5.

19. The LNP of claim 17, wherein the antigen-binding molecule is capable of binding to and inhibiting an immune checkpoint receptor.

20. The LNP of claim 19, wherein the immune checkpoint receptor is PD-1.

21. The LNP of claim 17, wherein the antigen-binding molecule is capable of binding to a tumor antigen.

22. The LNP of claim 21, wherein the antigen binding molecule is a bispecific T cell engager molecule (BiTE) or a bispecific light T cell engager molecule (LiTE).

23. The LNP of claim 21 or 22, wherein the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1.

24. The LNP of claim 21 or 22, wherein the tumor antigen is DLL3 or EpCAM.

25. The LNP of any one of claims 1-24, wherein the synthetic RNA viral genome and/or the recombinant RNA molecule comprises a microRNA (miRNA) target sequence (miR-TS) cassette, wherein the miR-TS cassette comprises one or more miRNA target sequences.

26. The LNP of claim 25, wherein the one or more miRNAs are selected from miR-124, miR-1, miR-143, miR-128, miR-219, miR-219a, miR-122, miR-204, miR-217, miR-137, miR-142, and miR-126.

27. The LNP of claim 26, wherein the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

28. The LNP of any one of claims 1-27, wherein the LNP comprises a cationic lipid, a helper lipid, a structural lipid, and a PEG-lipid.

29. The LNP of claim 28, wherein the cationic lipid is a compound of Formula (I): 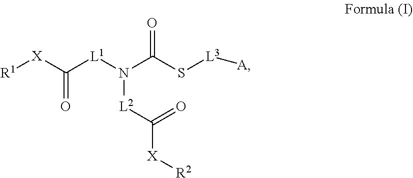
or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

30. The LNP of claim 29, wherein the number of carbon atoms between the S of the thiolate and the closest N comprised in A is 2-4.

31. The LNP of claim 29 or 30, wherein the cationic lipid is a compound of Formula (I-a): 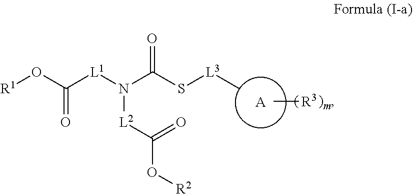
or a pharmaceutically acceptable salt or solvate thereof, wherein:
m is 0, 1, 2, 3, 4, 5, or 6.

32. The LNP of any one of claims 29-31, wherein A is an optionally substituted 5-6-membered heterocyclyl ring.

33. The LNP of claim 29, wherein the cationic lipid is 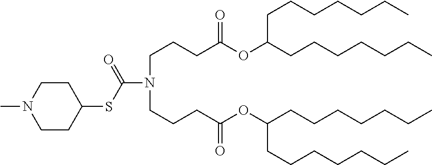 [SMILES: CCCCCCCC(CCCCCCC)OC(=O)CCCN(CCCC(=O)OC(CCCCCCC)CCCCCCC)C(=O)SC1CCN(C)CC1]
or a pharmaceutically acceptable salt or solvate thereof.

34. The LNP of claim 28, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME@SS—OP, Di((Z)-non-2-en-1-yl)9-((4-dimethylamino)butanoyl)oxy)heptadecanedioate (L-319), or N-(2,3-dioleoyloxy)propyl)-N,N,N-trimethylammonium chloride (DOTAP).

35. The LNP of any one of claims 28-34, wherein the helper lipid is selected from 1,2-distearoyl-sn-glycero-3-phosphocholine (DSPC); 1,2-dilauroyl-sn-glycero-3-phosphoethanolamine (DLPE); 1,2-dioleoyl-sn-glycero-3-phosphocholine (DOPC); and 1,2-dioleoyl-sn-glycero-3-phosphoethanolamine (DOPE).

36. The LNP of claim 28, wherein the cationic lipid is 1,2-dioleoyl-3-trimethylammonium-propane (DOTAP), and wherein the helper lipid is 1,2-Dilauroyl-sn-glycero-3-phosphoethanolamine (DLPE) or 1,2-Dioleoyl-sn-glycero-3-phosphoethanolamine (DOPE).

37. The LNP of any one of claims 28-36, wherein the structural lipid is cholesterol.

38. The LNP of any one of claims 28-37, wherein the PEG-lipid is a compound of Formula (A″): 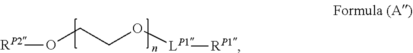
or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃,
and wherein the LNP has a molar ratio of about 0.001% to about 5% PEG-lipid.

39. The LNP of claim 38, wherein L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

40. The LNP of claim 38, wherein L^{P1}″ is a bond.

41. The LNP of any one of claims 38-40, wherein R^{P2}″ is hydrogen.

42. The LNP of any one of claims 28-37, wherein the PEG-lipid is a compound of Formula (B): 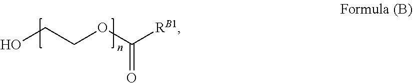
or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

43. The LNP of any one of claims 28-37, wherein the PEG-lipid is selected from 1,2-distearoyl-sn-glycero-3-phosphoethanolamine-N-[amino(polyethyleneglycol)] (DSPE-PEG); 1,2-dipalmitoyl-rac-glycerol methoxypolyethylene glycol (DPG-PEG); 1,2-distearoyl-rac-glycero-3-methylpolyoxyethylene (DSG-PEG); 1,2-distearoyl-rac-glycero-3-methylpolyoxyethylene (DSG-PEG); 1,2-dimyristoyl-rac-glycero-3-methylpolyoxyethylene (DMG-PEG); and 1,2-dimyristoyl-rac-glycero-3-methylpolyoxyethylene (DMG-PEG), or 1,2-distearoyl-sn-glycero-3-phosphoethanolamine-N-[amino(polyethylene glycol)] (DSPE-PEG-amine).

44. The LNP of any one of claims 28-37, wherein the PEG-lipid is selected from 1,2-distearoyl-sn-glycero-3-phosphoethanolamine-N-[amino(polyethyleneglycol)-5000] (DSPE-PEG5K); 1,2-dipalmitoyl-rac-glycerol methoxypolyethylene glycol-2000 (DPG-PEG2K); 1,2-distearoyl-rac-glycero-3-methylpolyoxyethylene-5000 (DSG-PEG5K); 1,2-distearoyl-rac-glycero-3-methylpolyoxyethylene-2000 (DSG-PEG2K); 1,2-dimyristoyl-rac-glycero-3-methylpolyoxyethylene-5000 (DMG-PEG5K); and 1,2-dimyristoyl-rac-glycero-3-methylpolyoxyethylene-2000 (DMG-PEG2K).

45. The LNP of claim 28, wherein the cationic lipid comprises COATSOME® SS—OC, wherein the helper lipid comprises DSPC, the structural lipid comprises cholesterol (Chol) and wherein the PEG-lipid comprises DPG-PEG2000.

46. The LNP of claim 28, wherein the cationic lipid comprises COATSOME® SS—OC, wherein the helper lipid comprises DSPC, the structural lipid comprises cholesterol (Chol) and wherein the PEG-lipid is a compound of Formula (A″): 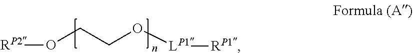
or a pharmaceutically acceptable salt thereof, wherein:
n is an integer between 10 to 200, inclusive of all endpoints;
L^{P1}″ is a bond;
R^{P1}″ is C₅-C₂₅ alkyl or C₅-C₂₅ alkenyl; and
R²″ is hydrogen, and
wherein the LNP has a molar ratio of about 0.001% to about 5% PEG-lipid.

47. The LNP of any one of claims 28-37 and 46, wherein the PEG-lipid is selected from the group consisting of BRIJ™ S100, BRIJ™ S20, BRIJ™ 020 and BRIJ™ C₂₀.

48. The LNP of any one of claims 28-37 and 46, wherein the PEG-lipid is BRIJ™ S100.

49. The LNP of any one of claims 45-48, wherein the ratio of SS—OC:DSPC:Chol:PEG-lipid (as a percentage of total lipid content) is A:B:C:D, wherein A+B+C+D=100%, and wherein
a. A=40%-60%, B=10%-25%, C=20%-30%, and D=0.01%-3%;
b. A=45%-50%, B=20%-25%, C=25%-30%, and D=0.01%-1%; or
c. A=about 49%, B=about 22%, C=about 28%, and D=about 0.5%

50. The LNP of any one of claims 45-48, wherein the ratio of SS—OC:DSPC:Chol:PEG-lipid (as a percentage of total lipid content) is A:B:C:D, wherein A+B+C+D=100%, and wherein
a. A=40%-60%, B=10%-30%, C=20%-45%, and D=0%-3%;
b. A=40%-60%, B=10%-30%, C=25%-45%, and D=0.01%-3%;
c. A=45%-55%, B=10%-20%, C=30%-40%, and D=1%-2%;
d. A=45%-50%, B=10%-15%, C=35%-40%, and D=1%-2%; or
e. A=about 49%, B=about 11%, C=about 38%, and D=about 1.5%.

51. The LNP of any one of claims 45-48, wherein the ratio of SS—OC:DSPC:Chol:PEG-lipid (as a percentage of total lipid content) is about A:B:C:D, wherein A+B+C+D=100%, and wherein
a. A=45%-65%, B=5%-20%, C=20%-45%, and D=0%-3%;
b. A=50%-60%, B=5%-15%, C=30%-45%, and D=0.01%-3%;
c. A=55%-60%, B=5%-15%, C=30%-40%, and D=1%-2%;
d. A=55%-60%, B=5%-10%, C=30%-35%, and D=1%-2%; or
e. A=about 58%, B=about 7%, C=about 33%, and D=about 1.5%.

52. A lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (A″): 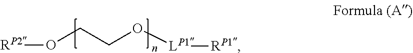
or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

53. A lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (A″): 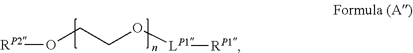
or a pharmaceutically acceptable salt thereof, wherein:
n is an integer between 10 to 200, inclusive of all endpoints;
L^{P1}″ is a bond, —[(CH₂)₀₋₃—C(O)O]₁₋₃—, —(CH₂)₀₋₃—C(O)O—(CH₂)₁₋₃—OC(O)—, or —C(O)N(H)—;
R^{P1}″ is C₅-C₂₅ alkyl or C₅-C₂₅ alkenyl; and
R^{P2}″ is hydrogen or —CH₃, and
wherein the LNP has a molar ratio of about 0.001% to about 5% PEG-lipid.

54. The LNP of claim 52 or 53, wherein R¹ is C₁₆-C₁₈ alkyl or C₁₆-C₁₈ alkenyl.

55. The LNP of any one of claims 52-54, wherein L^{P1}″ is a bond, —CH₂C(O)O—, —CH₂CH₂C(O)O—, —CH₂C(O)OCH₂C(O)O—, —CH₂C(O)OCH₂CH₂OC(O)—, or —C(O)N(H)—.

56. The LNP of any one of claims 52-54, wherein L^{P1}″ is a bond.

57. The LNP of any one of claims 52-56, wherein R^{P2}″ is hydrogen.

58. The LNP of claim 52 or 53, wherein the PEG-lipid is a compound of Formula (A″-f1), Formula (A″-f2), or Formula (A″-f3): 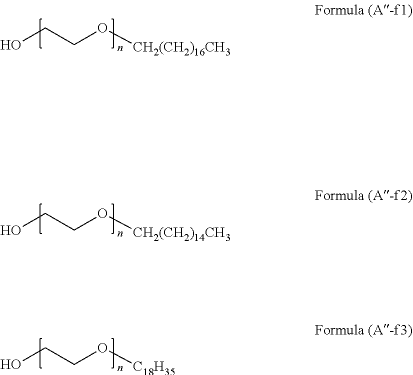
or a pharmaceutically acceptable salt thereof.

59. A lipid nanoparticle (LNP), comprising:
a. a synthetic RNA viral genome encoding a Seneca Valley virus (SVV); and
b. a cationic lipid, a helper lipid, a structural lipid, and a PEG-lipid, wherein the PEG-lipid is a compound of Formula (B): 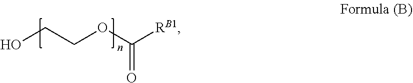
or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

60. A lipid nanoparticle (LNP), comprising:
a. a synthetic RNA viral genome encoding a Coxsackievirus; and
b. a cationic lipid, a helper lipid, a structural lipid, and a PEG-lipid, wherein the PEG-lipid is a compound of Formula (B): 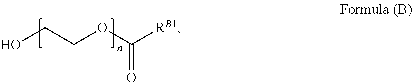
or a pharmaceutically acceptable salt thereof, wherein:
n is an integer between 10 to 200, inclusive of all endpoints; and
R^{B1} is C₅-C₂₅ alkyl or C₅-C₂₅ alkenyl, and
wherein the LNP has a molar ratio of about 0.001% to about 5% PEG-lipid.

61. The LNP of claim 59 or 60, wherein R¹ is C₁₅-C₁₇ alkyl or C₁₅-C₁₇ alkenyl.

62. The LNP of claim 59 or 60, wherein the PEG-lipid is a compound of Formula (B-a) or Formula (B-b): 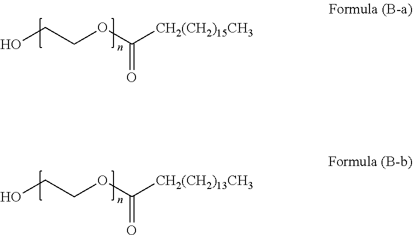
or a pharmaceutically acceptable salt thereof.

63. The LNP of any one of claims 52-62, wherein n is on average about 20, about 40, about 50, or about 100.

64. The LNP of any one of claims 52-62, wherein n is on average about 100.

65. The LNP of any one of claims 52-64, wherein the PEG-lipid comprise a PEG moiety having an average molecular weight of about 200 daltons to about 10,000 daltons, about 500 daltons to about 7,000 daltons, or about 800 daltons to about 6,000 daltons.

66. The LNP of any one of claims 52-65, wherein the PEG-lipid is selected from the group consisting of HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃.

67. The LNP of any one of claims 52-66, wherein the LNP induces a reduced immune response in vivo as compared to a control LNP lacking the PEG-lipid of Formula (A″) and/or a ionizable lipid of Formula (I), optionally wherein a PEG-lipid in the control LNP is PEG2K-DPG or PEG2K-DMG.

68. The LNP of claim 67, wherein the immune response is accelerated blood clearance (ABC) of the LNP and/or an anti-PEG IgM response.

69. The LNP of any one of claims 52-68, wherein the cationic lipid is a compound of Formula (I): 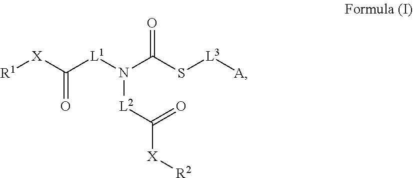
or a pharmaceutically acceptable salt or solvate thereof, wherein:
A is —N(CH₂R^{N1})(CH₂R^{N2}) or a 4-7-membered heterocyclyl ring containing at least one N, wherein the 4-7-membered heterocyclyl ring is optionally substituted with 0-6 R³;
each X is independently —O—, —N(R¹)—, or —N(R²)—;
R¹ is selected from the group consisting of optionally substituted C₁-C₃₁ aliphatic and steroidyl;
R² is selected from the group consisting of optionally substituted C₁-C₃₁ aliphatic and steroidyl;
R³ is optionally substituted C₁-C₆ aliphatic;
R^{N1} and R^{N2} are each independently hydrogen, hydroxy-C₁-C₆ alkyl, C₂-C₆ alkenyl, or a C₃-C₇ cycloalkyl;
L¹ is selected from the group consisting of an optionally substituted C₁-C₂₀ alkylene chain and a bivalent optionally substituted C₂-C₂₀ alkenylene chain;
L² is selected from the group consisting of an optionally substituted C₁-C₂₀ alkylene chain and a bivalent optionally substituted C₂-C₂₀ alkenylene chain; and
L³ is a bond, an optionally substituted C₁-C₆ alkylene chain, or a bivalent optionally substituted C₃-C₇ cycloalkylene; and
with the proviso that when A is —N(CH₃)(CH₃) and X is O, L³ is not an C₁-C₆ alkylene chain.

70. The LNP of claim 69, wherein the number of carbon atoms between the S of the thiolate and the closest N comprised in A is 2-4.

71. The LNP of claim 69 or 70, wherein the cationic lipid is a compound of Formula (I-a): 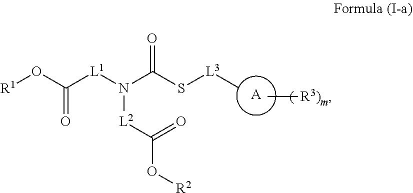
or a pharmaceutically acceptable salt or solvate thereof, wherein:
m is 0, 1, 2, 3, 4, 5, or 6.

72. The LNP of any one of claims 69-71, wherein A is an optionally substituted 5-6-membered heterocyclyl ring.

73. The LNP of claim 69, wherein the cationic lipid is 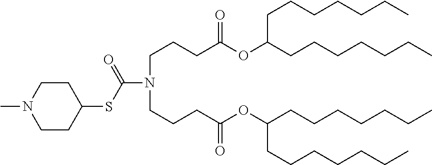 [SMILES: CCCCCCCC(CCCCCCC)OC(=O)CCCN(CCCC(=O)OC(CCCCCCC)CCCCCCC)C(=O)SC1CCN(C)CC1]
or a pharmaceutically acceptable salt or solvate thereof.

74. The LNP of any one of claims 52-68, wherein the cationic lipid is selected from DLinDMA, DLin-KC2-DMA, DLin-MC3-DMA (MC3), COATSOME® SS-LC (former name: SS-18/4PE-13), COATSOME® SS-EC (former name: SS-33/4PE-15), COATSOME® SS—OC, COATSOME® SS—OP, Di((Z)-non-2-en-1-yl)9-((4-dimethylamino)butanoyl)oxy)heptadecanedioate (L-319), N-(2,3-dioleoyloxy)propyl)-N,N,N-trimethylammonium chloride (DOTAP), or a mixture thereof.

75. The LNP of any one of claims 52-68, wherein the cationic lipid is a compound of Formula (II-1a): 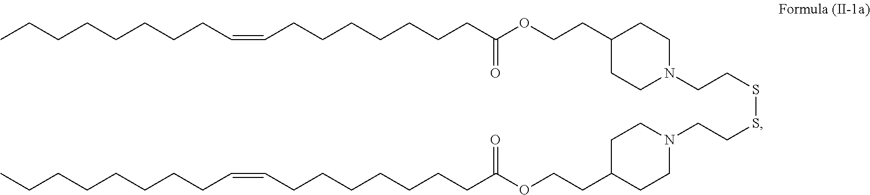 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]
a compound of Formula (II-2a):

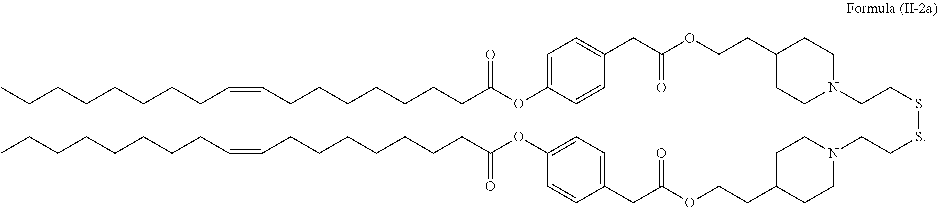 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

76. The LNP of any one of claims 52-75, wherein the cationic lipid is a compound of Formula (II-1a), the structural lipid is cholesterol, the helper lipid is DSPC, and the PEG-lipid is BRIJ™ S100.

77. The LNP of any one of claims 52-75, wherein the cationic lipid is a compound of Formula (II-1a), the structural lipid is cholesterol, the helper lipid is DSPC, and the PEG-lipid is MYRJ™ S100, MYRJ™ S50, or MYRJ™ S40.

78. The LNP of any one of claims 52-77, wherein the LNP comprises a molar ratio of about 0.1% to about 2% PEG-lipid, such as about 0.2% to about 0.8 mol %, about 0.4% to about 0.6 mol %, about 0.7% to about 1.3%, or about 1.2% to about 1.8% PEG-lipid.

79. The LNP of any one of claims 52-78, wherein the LNP comprises a molar ratio of about 0.2% to about 0.8%, or about 0.5% PEG-lipid.

80. The LNP of any one of claims 52-78, wherein the LNP comprises a molar ratio of about 1.2% to about 1.8%, or about 1.5% PEG-lipid.

81. The LNP of any one of claims 52-80, wherein the LNP has a molar ratio of about 44% to about 54% cationic lipid, about 19% to about 25% helper lipid, about 24% to about 33% structural lipid, and about 0.2% to about 0.8% PEG-lipid.

82. The LNP of any one of claims 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol: DSPC PEG-lipid is 49:28.5:22:0.5.

83. The LNP of any one of claims 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol: DSPC PEG-lipid is 49:27.5:22:1.5.

84. The LNP of any one of claims 52-81, wherein the LNP comprises a compound of Formula (II-1a), cholesterol, DSPC, and a PEG-lipid selected from HO-PEG100-CH₂(CH₂)₁₆CH₃, HO-PEG20-CH₂(CH₂)₁₆CH₃, HO-PEG20-CH₂(CH₂)₁₄CH₃, HO-PEG20-C₁₈H₃₅, HO-PEG100-C(O)—CH₂(CH₂)₁₃CH₃, HO-PEG50-C(O)—CH₂(CH₂)₁₃CH₃, HO-PEG40-C(O)—CH₂(CH₂)₁₃CH₃, HO-PEG100-C(O)—CH₂(CH₂)₁₅CH₃, HO-PEG40-C(O)—CH₂(CH₂)₁₅CH₃, and HO-PEG50-C(O)—CH₂(CH₂)₁₅CH₃, wherein the molar ratio of compound of Formula (II-1a): cholesterol: DSPC PEG-lipid is 49:38.5:11:1.5.

85. The LNP of any one of claims 52-84, wherein the LNP has a lipid-nitrogen-to-phosphate (N:P) ratio of about 1 to about 25.

86. The LNP of any one of claims 52-85, wherein the LNP has a N:P ratio of about 14.

87. The LNP of any one of claims 1-86, wherein hyaluronan is conjugated to the surface of the LNP.

88. A pharmaceutical composition comprising a plurality of lipid nanoparticles according to any one of claims 1-87.

89. The pharmaceutical composition of claim 88, wherein the plurality of LNPs have an average diameter of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

90. The pharmaceutical composition of claim 88, wherein the plurality of LNPs have an average diameter of about 50 nm to about 120 nm.

91. The pharmaceutical composition of claim 88, wherein the plurality of LNPs have an average diameter of about 50 nm, 60 nm, 70 nm, 80 nm, 90 nm, 100 nm, 110 nm, or about 120 nm.

92. The pharmaceutical composition of claim 88, wherein the plurality of LNPs have an average diameter of about 100 nm.

93. The pharmaceutical composition of any one of claims 88-92, wherein the plurality of LNPs have an average zeta-potential of between about 40 mV to about −40 mV, about 20 mV to about −20 mV, about 10 mV to about −10 mV, about 5 mV to about −5 mV, or about 20 mV to about −40 mV.

94. The pharmaceutical composition of any one of claims 88-92, wherein the plurality of LNPs have an average zeta-potential of less than about 5 mV, less than about 0 mV, less than about −5 mV, less than about −10 mV, less than about −20 mV, less than about −30 mV, less than about −35 mV, or less than about −40 mV.

95. The pharmaceutical composition of any one of claims 88-92, wherein the plurality of LNPs have an average zeta-potential of between about −50 mV to about −20 mV, about −40 mV to about −20 mV, about −30 mV to about −10 mV, about −20 mV to about 0 mV, about −15 mV to about 5 mV, or about −10 mV to about 10 mV.

96. The pharmaceutical composition of claim 94 or 95, wherein the plurality of LNPs have an average zeta-potential of about −30 mV, about −31 mV, about −32 mV, about −33 mV, about −34 mV, about −35 mV, about −36 mV, about −37 mV, about −38 mV, about −39 mV, or about −40 mV.

97. The pharmaceutical composition of any one of claims 88-96, wherein administering the pharmaceutical composition to a subject delivers the recombinant RNA polynucleotide to a target cell of the subject, and wherein the recombinant RNA polynucleotide produces an infectious oncolytic virus capable of lysing the target cell of the subject.

98. The pharmaceutical composition of claim 97, wherein the target cell is a cancerous cell.

99. The pharmaceutical composition of any one of claims 88-98, wherein the composition is formulated for intravenous and/or intratumoral delivery.

100. The pharmaceutical composition of any one of claims 88-99, wherein the composition has a duration of therapeutic effect in vivo greater than that of a composition lacking the PEG-lipid of Formula (A″) and/or a ionizable lipid of Formula (I).

101. The pharmaceutical composition of claim 99 or 100, wherein the composition has a duration of therapeutic effect in vivo of about 1 hour or longer, about 2 hours or longer, about 3 hours or longer, about 4 hours or longer, about 5 hours or longer, about 6 hours or longer, about 7 hours or longer, about 8 hours or longer, about 9 hours or longer, about 10 hours or longer, about 12 hours or longer, about 14 hours or longer, about 16 hours or longer, about 18 hours or longer, about 20 hours or longer, about 25 hours or longer, about 30 hours or longer, about 35 hours or longer, about 40 hours or longer, about 45 hours or longer, or about 50 hours or longer.

102. The pharmaceutical composition of claim 99 or 100, wherein the composition has a half-life and/or an AUC in vivo greater than or equal to that of a pre-determined threshold value.

103. The pharmaceutical composition of any one of claims 88-102, wherein the encapsulation efficiency of the synthetic RNA viral genome by the LNP is at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

104. The pharmaceutical composition of any one of claims 88 to 103, wherein the composition has a total lipid concentration of about 10 mM, about 20 mM, about 30 mM, about 40 mM, or about 50 mM.

105. The pharmaceutical composition of any one of claims 88-104, wherein the composition is formulated at a pH of about 2.5, about 3, about 3.5, about 4, about 4.5, about 5, about 5.5, or about 6.

106. The pharmaceutical composition of any one of claims 88 to 105, wherein the composition is formulated for multiple administrations.

107. The pharmaceutical composition of claim 106, wherein a subsequent administration is administered at least 3 days, at least 5 days, at least 7 days, at least 9 days, at least 11 days, at least 14 days, or at least 21 days after a first administration.

108. The pharmaceutical composition of any one of claims 88 to 107, further comprising a pharmaceutically acceptable carrier.

109. A recombinant RNA molecule comprising a synthetic RNA viral genome encoding an oncolytic Coxsackievirus virus, wherein the Coxsackievirus is a CVA21 strain selected from the Kuykendall strain, the EF strain and the KY strain.

110. The recombinant RNA molecule of claim 109, wherein the Coxsackievirus is the CVA21-KY strain, and wherein the CVA21-KY strain comprises a polynucleotide sequence having at least 80%, at least 85%, at least 90%, at least 95%, or 100% sequence identity according to SEQ ID NO: 5.

111. The recombinant RNA molecule of claim 109, wherein the Coxsackievirus is the CVA21-EF strain, and wherein the CVA21-EF strain comprises a polynucleotide sequence having at least 80%, at least 85%, at least 90%, at least 95%, or 100% sequence identity according to SEQ ID NO: 9.

112. The recombinant RNA molecule of claim 109, wherein the Coxsackievirus comprises a 5′ UTR (IRES) sequence having at least 80%, at least 85%, at least 90%, at least 95%, or 100% sequence identity according to SEQ ID NO: 6 or 10.

113. The recombinant RNA molecule of claim 109, wherein the Coxsackievirus comprises a P1 sequence having at least 80%, at least 85%, at least 90%, at least 95%, or 100% sequence identity according to SEQ ID NO: 7 or 11.

114. The recombinant RNA molecule of claim 109, wherein the Coxsackievirus comprises a 3D sequence having at least 80%, at least 85%, at least 90%, at least 95%, or 100% sequence identity according to SEQ ID NO: 8 or 12.

115. The recombinant RNA molecule of any one of claims 109-114, wherein the synthetic RNA viral genome does not comprise a polynucleotide sequence having more than 95%, more than 90%, more than 85%, or more than 80% sequence identity according to SEQ ID NO: 1.

116. The recombinant RNA molecule of any one of claims 109-115, wherein the recombinant RNA molecule does not comprise an RNA viral genome having 100% sequence identity to that of a wildtype Coxsackievirus virus.

117. A recombinant RNA molecule comprising a synthetic RNA viral genome encoding a Seneca Valley virus (SVV), wherein the SVV comprises is a chimeric SVV, and wherein the synthetic RNA viral genome comprises a polynucleotide sequence having at least 80%, at least 85%, at least 90%, at least 95%, or 100% sequence identity to SEQ ID NO: 68.

118. The recombinant RNA molecule of any one of claims 109-117, further comprising a microRNA (miRNA) target sequence (miR-TS) cassette inserted into the polynucleotide sequence encoding the oncolytic virus, wherein the miR-TS cassette comprises one or more miRNA target sequences, and wherein expression of one or more of the corresponding miRNAs in a cell inhibits replication of the encoded virus in the cell.

119. The recombinant RNA molecule of claim 118, wherein the one or more miRNAs are selected from miR-124, miR-1, miR-143, miR-128, miR-219, miR-219a, miR-122, miR-204, miR-217, miR-137, miR-142, and miR-126.

120. The recombinant RNA molecule of claim 119, wherein the miR-TS cassette comprises:
a. one or more copies of a miR-124 target sequence, one or more copies of a miR-1 target sequence, and one or more copies of a miR-143 target sequence;
b. one or more copies of a miR-128 target sequence, one or more copies of a miR-219a target sequence, and one or more copies of a miR-122 target sequence;
c. one or more copies of a miR-128 target sequence, one or more copies of a miR-204 target sequence, and one or more copies of a miR-219 target sequence; or
d. one or more copies of a miR-217 target sequence, one or more copies of a miR-137 target sequence, and one or more copies of a miR-126 target sequence.

121. The recombinant RNA molecule of any one of claims 109-120, wherein the recombinant RNA molecule is capable of producing a replication-competent oncolytic virus when introduced into a cell by a non-viral delivery vehicle.

122. The recombinant RNA molecule of claim 121, wherein the cell is a mammalian cell.

123. The recombinant RNA molecule of claim 122, wherein the cell is a mammalian cell present in a mammalian subject.

124. The recombinant RNA molecule of any one of claims 118-123, wherein the one or more miR-TS cassettes is incorporated into the 5′ untranslated region (UTR) or 3′ UTR of one or more viral genes.

125. The recombinant RNA molecule of any one of claims 118-123, wherein the one or more miR-TS cassettes is incorporated into the open reading frame (ORF), the 5′ untranslated region (UTR), or the 3′ UTR of one or more viral genes.

126. The recombinant RNA molecule of any of claims 109-125, wherein the recombinant RNA molecule is inserted into a nucleic acid vector.

127. The recombinant RNA molecule of claim 126, wherein the nucleic acid vector is a replicon.

128. The recombinant RNA molecule of claims 109-127, wherein the synthetic RNA viral genome further comprises a heterologous polynucleotide encoding an exogenous payload protein.

129. The recombinant RNA molecule of claim 128, wherein the exogenous payload protein comprises or consists of a MLKL 4HB domain, a Gasdermin D N-terminal fragment, a Gasdermin E N-terminal fragment, a HMGB1 Box B domain, a SMAC/Diablo, a Melittin, a L-amino-acid oxidase (LAAO), a disintegrin, a TRAIL (TNFSF10), a nitroreductase, a reovirus FAST protein, a leptin/FOSL2, an α-1,3-galactosyltransferase, or an adenosine deaminase 2 (ADA2).

130. The LNP of claim 129, wherein the nitroreductase is NfsB or NfsA.

131. The LNP of claim 129, wherein the reovirus FAST protein is ARV p14, BRV p15, or a p14-p15 hybrid.

132. The recombinant RNA molecule of claim 128, wherein the exogenous payload protein is a fluorescent protein, an enzymatic protein, a cytokine, a chemokine, an antigen-binding molecule capable of binding to a cell surface receptor, or a ligand capable of binding to a cell surface receptor.

133. The recombinant RNA molecule of claim 132, wherein
a) the cytokine is selected from GM-CSF, IFNγ, IL-2, IL-7, IL-12, IL-18, IL-21, and IL-36γ;
b) the ligand for a cell-surface receptor is Flt3 ligand or TNFSF14;
c) the chemokine is selected from CXCL10, CCL4, CCL21, and CCL5.

134. The recombinant RNA molecule of claim 132, wherein the antigen-binding molecule is capable of binding to and inhibiting an immune checkpoint receptor.

135. The recombinant RNA molecule of claim 134, wherein the immune checkpoint receptor is PD-1.

136. The recombinant RNA molecule of claim 132, wherein the antigen-binding molecule is capable of binding to a tumor antigen.

137. The recombinant RNA molecule of claim 136, wherein the antigen binding molecule is a bispecific T cell engager molecule (BiTE) or a bispecific light T cell engager molecule (LiTE).

138. The recombinant RNA molecule of claim 136 or 137, wherein the tumor antigen is a viral antigen selected from HBV-core (Hepatitis B core antigen), HBV-pol, HbS-Ag, HPV E6, HPV E7, Merkel cell polyoma large T antigen, and Epstein Barr virus antigen EBNA2 or BZLF1.

139. The recombinant RNA molecule of claim 136 or 137, wherein the tumor antigen is DLL3 or EpCAM.

140. A recombinant DNA template comprising from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence.

141. A recombinant DNA molecule comprising from 5′ to 3′, a promoter sequence, a 5′ junctional cleavage sequence, a polynucleotide sequence encoding an RNA molecule comprising a synthetic RNA viral genome, a poly-A tail, and a 3′ junctional cleavage sequence, wherein the RNA molecule is selected from any one of claims 109-139.

142. The recombinant DNA molecule of claim 140 or 141, comprising a leader sequence between the promoter sequence and the 5′ junctional cleavage sequence.

143. A recombinant DNA molecule comprising from 5′ to 3′, a promoter sequence, a leader sequence, a 5′ junctional cleavage sequence, a polynucleotide sequence encoding a recombinant RNA molecule comprising a synthetic RNA viral genome, a poly-A tail, and a 3′ junctional cleavage sequence.

144. The recombinant DNA molecule of claim 142 or 143, wherein the leader sequence is less than 100 bp in length.

145. The recombinant DNA molecule of any one of claims 140-144, wherein the promoter sequence is a T7 promoter sequence.

146. The recombinant DNA molecule of any one of claims 140-145, wherein the poly-A tail is about 50-90 bp in length or about 65-75 bp in length.

147. The recombinant DNA molecule of claim 145, wherein the poly-A tail is about 70 bp in length.

148. The recombinant DNA molecule of any one of claims 140-145, wherein the poly-A tail is about 10-50 bp, or 25-35 bp in length.

149. The recombinant DNA molecule of any one of claims 140-148, wherein the 5′ junctional cleavage sequence comprises or consists of a ribozyme sequence and the 3′ junctional cleavage sequence comprises or consists of a ribozyme sequence.

150. The recombinant DNA molecule of claim 149, wherein the 5′ ribozyme sequence is a hammerhead ribozyme sequence and wherein the 3′ ribozyme sequence is a hepatitis delta virus ribozyme sequence.

151. The recombinant DNA molecule of any one of claims 140-148, wherein the 5′ junctional cleavage sequence comprises or consists of an RNAseH primer binding sequence and the 3′ junctional cleavage sequence comprises or consists of a restriction enzyme recognition sequence.

152. The recombinant DNA molecule of any one of claims 140-148, wherein the 5′ junctional cleavage sequence comprises or consists of a ribozyme sequence and the 3′ junctional cleavage sequence comprises or consists of a restriction enzyme recognition sequence.

153. The recombinant DNA molecule of claim 152, wherein the 5′ ribozyme sequence comprises or consists of a hammerhead ribozyme sequence, a Pistol ribozyme sequence, or a modified Pistol ribozyme sequence.

154. The recombinant DNA molecule of any one of claims 140-153, wherein the 3′ junctional cleavage sequence comprises or consists of a Type IIS restriction enzyme recognition sequence.

155. The recombinant DNA molecule of any one of claims 140-154, wherein the RNA molecule encodes the RNA viral genome of a Coxsackievirus (CVA).

156. The recombinant DNA molecule of claim 155, wherein the Coxsackievirus is a CVA21 strain.

157. The recombinant DNA molecule of any one of claims 155-156, wherein the leader sequence comprises or consists of a polynucleotide sequence having at least 70%, at least 80%, at least 90%, at least 95%, or 100% sequence identity according to SEQ ID NO: 14 or 15.

158. The recombinant DNA molecule of any one of claims 155-157, wherein the 5′ junctional cleavage sequence comprises or consists of a Pistol ribozyme sequence having at least 80%, at least 90%, or 100% sequence identity to SEQ ID NO: 18, and wherein the P2 motif of the 5′ ribozyme sequence has the polynucleotide sequence of “TTTT”.

159. The recombinant DNA molecule of any one of claims 155-157, wherein the 5′ junctional cleavage sequence comprises or consists of a Pistol ribozyme sequence having at least 80%, at least 90%, or 100% sequence identity to SEQ ID NO: 17, and wherein the P2 motif of the 5′ ribozyme sequence has the polynucleotide sequence of “TTTA”.

160. The recombinant DNA molecule of any one of claims 155-159, wherein the 3′ junctional cleavage sequence comprises or consists of a BsmBI recognition sequence.

161. The recombinant DNA molecule of any one of claims 155-159, wherein the 3′ junctional cleavage sequence comprises or consists of a BsaI recognition sequence.

162. The recombinant DNA molecule of claim 156, wherein the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsmBI recognition sequence.

163. The recombinant DNA molecule of claim 156, wherein the promoter sequence is a T7 promoter sequence, wherein the leader sequence consists of a polynucleotide sequence according to SEQ ID NO: 15, wherein the 5′ junctional cleavage sequence comprises or consists of a Pistol ribozyme sequence according to SEQ ID NO: 18, wherein the poly-A tail is about 70 bp in length, and wherein the 3′ junctional cleavage sequence comprises or consists of a BsaI recognition sequence.

164. The recombinant DNA molecule of any one of claims 140-154, wherein the RNA molecule encodes the RNA viral genome of a Seneca Valley virus (SVV).

165. The recombinant DNA molecule of claim 164, wherein the leader sequence comprises or consists of a polynucleotide sequence having at least 70%, at least 80%, at least 90%, at least 95%, or 100% sequence identity according to any one of SEQ ID NO: 53-63.

166. The recombinant DNA molecule of claim 164, wherein the leader sequence comprises or consists of a polynucleotide sequence having at least 70%, at least 80%, at least 90%, at least 95%, or 100% sequence identity according to SEQ ID NO: 58.

167. The recombinant DNA molecule of any one of claims 164 to 166, wherein the 5′ ribozyme sequence is a Pistol ribozyme sequence having at least 80%, at least 85%, at least 90%, at least 95%, or 100% sequence identity to SEQ ID NO: 64 or 65, and wherein the P2 motif of the 5′ ribozyme sequence has the polynucleotide sequence of “TCAA” or “TTAA”.

168. The recombinant DNA molecule of any one of claims 164 to 167, wherein the RNA viral genome comprises a 5′ UTR (IRES) sequence having at least 80%, at least 85%, at least 90%, at least 95%, or 100% sequence identity to nucleic acids 1-670 of SEQ ID NO: 68.

169. The recombinant DNA molecule of any one of claims 164 to 168, wherein the 3′ junctional cleavage sequence comprises or consists of a SapI recognition sequence.

170. The recombinant DNA molecule of claim 164, wherein the promoter sequence is a T7 promoter sequence, wherein the leader sequence consists of a polynucleotide sequence according to SEQ ID NO: 53, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence.

171. The recombinant DNA molecule of claim 164, wherein the promoter sequence is a T7 promoter sequence, wherein the leader sequence consists of a polynucleotide sequence according to SEQ ID NO: 58, wherein the 5′ junctional cleavage sequence comprises or consists of a Pistol ribozyme sequence according to SEQ ID NO: 64, wherein the poly-A tail is about 70 bp in length, and wherein the 3′ junctional cleavage sequence comprises or consists of a SapI recognition sequence.

172. The recombinant DNA molecule of any one of claims 140-171, wherein the recombinant DNA molecule does not comprise additional nucleic acid within the region spanning the promoter sequence and the 3′ junctional cleavage sequence.

173. A method of producing a recombinant RNA molecule, comprising in vitro transcription of the DNA molecule of any one of claims 140-172 and purification of the resulting recombinant RNA molecule.

174. The method of claim 173, wherein the recombinant RNA molecule comprises 5′ and 3′ ends that are native to the oncolytic virus encoded by the synthetic RNA viral genome.

175. A composition comprising an effective amount of the recombinant RNA molecule of any one of claims 109-139, and a carrier suitable for administration to a mammalian subject.

176. A particle comprising the recombinant RNA molecule of any one of claims 109-139.

177. The particle of claim 176, wherein the particle is biodegradable.

178. The particle of claim 177, wherein the particle is selected from the group consisting of a nanoparticle, an exosome, a liposome, and a lipoplex.

179. The particle of claim 178, wherein the exosome is a modified exosome derived from an intact exosome or an empty exosome.

180. A pharmaceutical composition comprising a plurality of particles according to any one of claims 176-179.

181. The pharmaceutical composition of claim 180, wherein the plurality of particles have an average size of about 50 nm to about 500 nm, about 150 nm to about 500 nm, about 200 nm to about 500 nm, about 300 nm to about 500 nm, about 350 nm to about 500 nm, about 400 nm to about 500 nm, about 425 nm to about 500 nm, about 450 nm to about 500 nm, or about 475 nm to about 500 nm.

182. The pharmaceutical composition of claim 180 wherein the plurality of particles have an average size of about 50 nm to about 120 nm.

183. The pharmaceutical composition of claim 180 wherein the plurality of particles have an average size of about 50 nm, 60 nm, 70 nm, 80 nm, 90 nm, 100 nm, 110 nm, or about 120 nm.

184. The pharmaceutical composition of claim 180 wherein the plurality of particles have an average size of about 100 nm.

185. The pharmaceutical composition of any one of claims 180-184, wherein the plurality of particles have an average zeta-potential of between about 40 mV to about −40 mV, about 20 mV to about −20 mV, about 10 mV to about −10 mV, about 5 mV to about −5 mV, or about 20 mV to about −40 mV.

186. The pharmaceutical composition of any one of claims 180-184, wherein the plurality of particles have an average zeta-potential of less than about 5 mV, less than about 0 mV, less than about −5 mV, less than about −10 mV, less than about −20 mV, less than about −30 mV, less than about −35 mV, or less than about −40 mV.

187. The pharmaceutical composition of any one of claims 180-186, wherein the plurality of particles have an average zeta-potential of between about −50 mV to about −20 mV, about −40 mV to about −20 mV, about −30 mV to about −10 mV, about −20 mV to about 0 mV, about −15 mV to about 5 mV, or about −10 mV to about 10 mV.

188. The pharmaceutical composition of any one of claims 180-186, wherein the plurality of particles have an average zeta-potential of about −30 mV, about −31 mV, about −32 mV, about −33 mV, about −34 mV, about −35 mV, about −36 mV, about −37 mV, about −38 mV, about −39 mV, or about −40 mV.

189. The pharmaceutical composition of any one of claims 180-188, wherein delivery of the composition to a subject delivers the encapsulated recombinant RNA molecule to a target cell, and wherein the encapsulated recombinant RNA molecule produces an infectious virus capable of lysing the target cell.

190. An inorganic particle comprising the recombinant RNA molecule of any one of claims 109-139.

191. The inorganic particle of claim 190, wherein the inorganic particle is selected from the group consisting of a gold nanoparticle (GNP), gold nanorod (GNR), magnetic nanoparticle (MNP), magnetic nanotube (MNT), carbon nanohorn (CNH), carbon fullerene, carbon nanotube (CNT), calcium phosphate nanoparticle (CPNP), mesoporous silica nanoparticle (MSN), silica nanotube (SNT), or a starlike hollow silica nanoparticle (SHNP).

192. A composition comprising the inorganic particle of any one of claims 190-191, wherein the average diameter of the particles is less than about 500 nm, is between about 50 nm and 500 nm, is between about 250 nm and about 500 nm, or is about 350 nm.

193. The LNP of any one of claims 1-87, the particle of any one of claims 176-179, or the inorganic particle of any one of claims 190-191, further comprising a second recombinant RNA molecule encoding a payload molecule.

194. The LNP, particle, or inorganic particle of claim 193, wherein the second recombinant RNA molecule is a replicon.

195. A pharmaceutical composition comprising the LNP of any one of claims 1-87, the particle of any one of claims 176-179, or the inorganic particle of any one of claims 190-191, wherein the composition is formulated for intravenous and/or intratumoral delivery.

196. The pharmaceutical composition of claim 195, wherein the target cell of the LNP, the particle, or the inorganic particle is a cancerous cell.

197. A method of killing a cancerous cell comprising exposing the cancerous cell to the particle of any one of claims 1-87, 176-179, or 190-191, the recombinant RNA molecule of any one of claims 109-139, or compositions thereof, under conditions sufficient for the intracellular delivery of the particle to said cancerous cell, wherein the replication-competent virus produced by the encapsulated polynucleotide results in killing of the cancerous cell.

198. The method of claim 197, wherein the replication-competent virus is not produced in non-cancerous cells.

199. The method of claim 197 or 198, wherein the method is performed in vivo, in vitro, or ex vivo.

200. A method of treating a cancer in a subject comprising administering to a subject suffering from the cancer an effective amount of the particle of any one of claims 1-87, 176-179, or 190-191, the recombinant RNA molecule of any one of claims 109-139, or compositions thereof.

201. The method of claim 200, wherein the particle or composition thereof is administered intravenously, intranasally, intratumorally, intraperitoneally, or as an inhalant.

202. The method of claim 200, wherein the particle or composition thereof is administered intratumorally and/or intravenously.

203. The method of any one of claims 200-202, wherein the particle or composition thereof is administered to the subject repeatedly.

204. The method of any one of claims 200-203, wherein the subject is a mouse, a rat, a rabbit, a cat, a dog, a horse, a non-human primate, or a human.

205. The method of any of claims 200-204, wherein the cancer is lung cancer, breast cancer, colon cancer, or pancreatic cancer, and wherein the synthetic RNA viral genome comprises a polynucleotide sequence derived from the KY strain.

206. The method of any of claims 200-204, wherein the cancer is bladder cancer, renal cell carcinoma, ovarian cancer, gastric cancer or liver cancer, and wherein the synthetic RNA viral genome comprises a polynucleotide sequence derived from the EF strain.

207. The method of any one of claims 197-204, wherein the cancer is selected from lung cancer, breast cancer, ovarian cancer, cervical cancer, prostate cancer, testicular cancer, colorectal cancer, colon cancer, pancreatic cancer, liver cancer, renal cell carcinoma, gastric cancer, head and neck cancer, thyroid cancer, malignant glioma, glioblastoma, melanoma, B-cell chronic lymphocytic leukemia, multiple myeloma, monoclonal gammopathy of undetermined significance (MGUS), Merkel cell carcinoma, diffuse large B-cell lymphoma (DLBCL), sarcoma, a neuroblastoma, a neuroendocrine cancer, a rhabdomyosarcoma, a medulloblastoma, a bladder cancer, and marginal zone lymphoma (MZL).

208. The method of any of claims 197-204, wherein the cancer is selected from the groups consisting of lung cancer, breast cancer, colon cancer, pancreatic cancer, bladder cancer, renal cell carcinoma, ovarian cancer, gastric cancer and liver cancer.

209. The method of any of claims 197-204, wherein the cancer is renal cell carcinoma, lung cancer, or liver cancer.

210. The method of claim 205, 207, or 208, wherein the lung cancer is small cell lung cancer or non-small cell lung cancer (e.g., squamous cell lung cancer or lung adenocarcinoma).

211. The method of any of claims 206, 207, and 208, wherein the liver cancer is hepatocellular carcinoma (HCC) (e.g., Hepatitis B virus associated HCC).

212. The method of claim 207, wherein the prostate cancer is treatment-emergent neuroendocrine prostate cancer.

213. The method of any one of claims 197-204, wherein the cancer is lung cancer, liver cancer, prostate cancer (e.g., CRPC-NE), bladder cancer, pancreatic cancer, colon cancer, gastric cancer, breast cancer, neuroblastoma, renal cell carcinoma, ovarian cancer, rhabdomyosarcoma, medulloblastoma, neuroendocrine cancer, Merkel cell carcinoma, or melanoma.

214. The method of any one of claims 197-204, wherein the cancer is small cell lung cancer (SCLC) or neuroblastoma.

215. A method of treating a cancer in a subject in need thereof comprising administering an effective amount of a CVA21-EF virus to the subject.

216. A method of treating a cancer in a subject in need thereof comprising administering an effective amount of a CVA21-KY virus to the subject

217. A method of treating a cancer in a subject in need thereof comprising administering an effective amount of a CVA21-Kuykendall virus to the subject.

218. The method of any one of claims 215-217, wherein the virus is administered intratumorally and/or intravenously.

219. The method of any one of claims 197-218, further comprising administering an immune checkpoint inhibitor to the subject.

220. The method of claim 219, wherein the immune checkpoint inhibitor is an inhibitor of PD-1.

221. The method of any one of claims 197-218, further comprising administering an engineered immune cell comprising an engineered antigen receptor.

222. A method of treating a cancer in a subject in need thereof, comprising administering a therapeutically effective amount of an oncolytic Coxsackievirus, wherein the Coxsackievirus is a CVA21 strain, or a polynucleotide encoding the CVA21 to the subject, wherein the cancer is classified as sensitive to CVA21 infection based on the expression of ICAM-1 and/or the percentage of ICAM-1 positive cancer cells.

223. A method of treating a cancer in a subject in need thereof, comprising:
(a) determining the expression level of ICAM1 and/or the percentage of ICAM-1 positive cancer cells in the cancer;
(b) classifying the cancer as sensitive to Coxsackievirus 21 (CVA21) infection based on the expression of ICAM-1 and/or the percentage of ICAM-1 positive cancer cells determined in (a); and
(c) administering a therapeutically effective amount of CVA21 or a polynucleotide encoding the CVA21 to the subject if the cancer is classified as sensitive to CVA21 infection in step (b).

224. A method of selecting a subject suffering from a cancer for treatment with a Coxsackievirus 21 (CVA21) or a polynucleotide encoding the CVA21, comprising:
(a) determining the expression level of ICAM-1 and/or the percentage of ICAM-1 positive cancer cells in the cancer;
(b) classifying the cancer as sensitive to CVA21 infection based on the expression level of ICAM-1 and/or the percentage of ICAM1 positive cancer cells as determined in (a);
(c) selecting the subject for treatment with the CVA21 or the polynucleotide encoding the CVA21 if the cancer is classified as sensitive to CVA21 infection in (b); and
(d) administering the CVA21 or the polynucleotide encoding the CVA21 to the selected subjects

225. The method of any one of claims 222-224, wherein the CVA21 strain is CVA21-KY.
